SEQUENCE LISTING (16 sequences)

SEQ ID NO: 1 - protein, 1164 aa, Homo sapiens
MPTMRRTVSEIRSRAEGYEKTDDVSEKTSLADQEEVRTIFINQPQLTKFCNNHVSTAKYNIITFLPRFLYSQFRRAANSFFLFIALLQQIPDVSPTGRYTTLVPLLFILAVAAIKEIIEDIKRHKADNAVNKKQTQVLRNGAWEIVHWEKVAVGEIVKVTNGEHLPADLISLSSSEPQAMCYIETSNLDGETNLKIRQGLPATSDIKDVDSLMRISGRIECESPNRHLYDFVGNIRLDGHGTVPLGADQILLRGAQLRNTQWVHGIVVYTGHDTKLMQNSTSPPLKLSNVERITNVQILILFCILIAMSLVCSVGSAIWNRRHSGKDWYLNLNYGGASNFGLNFLTFIILFNNLIPISLLVTLEVVKFTQAYFINWDLDMHYEPTDTAAMARTSNLNEELGQVKYIFSDKTGTLTCNVMQFKKCTIAGVAYGHVPEPEDYGCSPDEWQNSQFGDEKTFSDSSLLENLQNNHPTAPIICEFLTMMAVCHTAVPEREGDKIIYQAASPDEGALVRAAKQLNFVFTGRTPDSVIIDSLGQEERYELLNVLEFTSARKRMSVIVRTPSGKLRLYCKGADTVIYDRLAETSKYKEITLKHLEQFATEGLRTLCFAVAEISESDFQEWRAVYQRASTSVQNRLLKLEESYELIEKNLQLLGATAIEDKLQDQVPETIETLMKADIKIWILTGDKQETAINIGHSCKLLKKNMGMIVINEGSLDGTRETLSRHCTTLGDALRKENDFALIIDGKTLKYALTFGVRQYFLDLALSCKAVICCRVSPLQKSEVVEMVKKQVKVVTLAIGDGANDVSMIQTAHVGVGISGNEGLQAANSSDYSIAQFKYLKNLLMIHGAWNYNRVSKCILYCFYKNIVLYIIEIWFAFVNGFSGQILFERWCIGLYNVMFTAMPPLTLGIFERSCRKENMLKYPELYKTSQNALDFNTKVFWVHCLNGLFHSVILFWFPLKALQYGTAFGNGKTSDYLLLGNFVYTFVVITVCLKAGLETSYWTWFSHIAIWGSIALWVVFFGIYSSLWPAIPMAPDMSGEAAMLFSSGVFWMGLLFIPVASLLLDVVYKVIKRTAFKTLVDEVQELEAKSQDPGAVVLGKSLTERAQLLKNVFKKNHVNLYRSESLQQNLLHGYAFSQDENGIVSQSEVIRAYDTTKQRPDEW

SEQ ID NO: 2 - protein, 1251 aa, Homo sapiens
MSTERDSETTFDEDSQPNDEVVPYSDDETEDELDDQGSAVEPEQNRVNREAEENREPFRKECTWQVKANDRKYHEQPHFMNTKFLCIKESKYANNAIKTYKYNAFTFIPMNLFEQFKRAANLYFLALLILQAVPQISTLAWYTTLVPLLVVLGVTAIKDLVDDVARHKMDKEINNRTCEVIKDGRFKVAKWKEIQVGDVIRLKKNDFVPADILLLSSSEPNSLCYVETAELDGETNLKFKMSLEITDQYLQREDTLATFDGFIECEEPNNRLDKFTGTLFWRNTSFPLDADKILLRGCVIRNTDFCHGLVIFAGADTKIMKNSGKTRFKRTKIDYLMNYMVYTIFVVLILLSAGLAIGHAYWEAQVGNSSWYLYDGEDDTPSYRGFLIFWGYIIVLNTMVPISLYVSVEVIRLGQSHFINWDLQMYYAEKDTPAKARTTTLNEQLGQIHYIFSDKTGTLTQNIMTFKKCCINGQIYGDHRDASQHNHNKIEQVDFSWNTYADGKLAFYDHYLIEQIQSGKEPEVRQFFFLLAVCHTVMVDRTDGQLNYQAASPDEGALVNAARNFGFAFLARTQNTITISELGTERTYNVLAILDFNSDRKRMSIIVRTPEGNIKLYCKGADTVIYERLHRMNPTKQETQDALDIFANETLRTLCLCYKEIEEKEFTEWNKKFMAASVASTNRDEALDKVYEEIEKDLILLGATAIEDKLQDGVPETISKLAKADIKIWVLTGDKKETAENIGFACELLTEDTTICYGEDINSLLHARMENQRNRGGVYAKFAPPVQESFFPPGGNRALIITGSWLNEILLEKKTKRNKILKLKFPRTEEERRMRTQSKRRLEAKKEQRQKNFVDLACECSAVICCRVTPKQKAMVVDLVKRYKKAITLAIGDGANDVNMIKTAHIGVGISGQEGMQAVMSSDYSFAQFRYLQRLLLVHGRWSYIRMCKFLRYFFYKNFAFTLVHFWYSFFNGYSAQTAYEDWFITLYNVLYTSLPVLLMGLLDQDVSDKLSLRFPGLYIVGQRDLLFNYKRFFVSLLHGVLTSMILFFIPLGAYLQTVGQDGEAPSDYQSFAVTIASALVITVNFQIGLDTSYWTFVNAFSIFGSIALYFGIMFDFHSAGIHVLFPSAFQFTGTASNALRQPYIWLTIILTVAVCLLPVVAIRFLSMTIWPSESDKIQKHRKRLKAEEQWQRRQQVFRRGVSTRRSAYAFSHQRGYADLISSGRSIRKKRSPLDAIVADGTAEYRRTGDS

SEQ ID NO: 3 - DNA, 2736 bp, Homo sapiens
aaaaactccattcgaacccatggagcagaaaaccaccgacatctactctatgagtgctgggcctcctggggcgtgtggtataaataccaacctttggatcttgtaaggcggtactttggagagaagattgggttatattttgcctggttgggctggtacaccggcatgctcttcccagctgccttcattggattgtttgtctttttgtatggcgtcaccactctggatcacagccaagtcagtaaagaagtctgccaagctacagatatcatcatgtgtcctgtgtgtgataaatactgtccattcatgaggctgtcagacagctgtgtatatgccaaggtaacccacctttttgacaatggagccactgtcttctttgctgttttcatggcagtctgggcaacagttttcctggagttttggaaaagacggcgagcagtaattgcttatgactgggatttgatagactgggaagaagaggaggaagaaatacgaccccagtttgaagccaagtattccaagaaagagcggatgaatccaatttctggaaagccagaaccttatcaagcatttacagataaatgcagcagacttatcgtttctgcatctggaatattttttatgatctgcgtggtgattgctgccgtgttcgggatcgtcatttaccgggtggtgactgtcagcactttcgctgcctttaagtgggcgttaatcaggaataactctcaggttgcaaccacagggactgctgtgtgcatcaacttctgtatcattatgttgctgaatgtgctctatgaaaaagttgccctgcttctgacgaatttagaacagcctcgcacagagtctgagtgggagaacagcttcaccctgaaaatgtttctttttcagtttgtcaatctgaacagctccacattttacatcgcattcttcctcggaagatttacaggacacccaggtgcctacttgaggctgataaacaggtggagactagaagagtgccaccctagtggatgccttattgatctgtgtatgcaaatgggtattataatggtgctaaagcagacctggaataatttcatggaacttggctacccgttaattcagaattggtggactagaagaaaagtacgacaagaacatggacctgaaaggaaaataagtttcccacaatgggaaaaggactataaccttcagccgatgaatgcctatggactcttcgatgaatacttagaaatgattcttcagtttggattcacaactatctttgtggcagcttttcccctagcaccacttctggccttactgaataacataattgaaattcgacttgatgcttacaaatttgtcacacagtggaggagacctttagcttcaagggccaaagacataggaatttggtatggaattcttgaaggcattggaattctctctgttatcacaaatgcatttgtcatagcgataacatctgactttatccctcgcttggtgtatgcttataagtatggaccttgtgcaggccaaggagaagctgggcaaaagtgcatggttggctatgtgaatgccagcttgtctgtatttcgaatttctgactttgagaaccgatctgagcctgaatctgatggcagtgagttctcggggactcctcttaagtactgcagataccgggactaccgtgacccgcctcattcactggtgccctatggctacacactgcagttttggcatgtcctagctgctcgattagcttttatcattgtctttgagcacctcgtgttttgtataaagcacctcatttcgtatctgatcccagacctcccaaaagacctaagggatcgaatgagaagagagaagtacttgattcaggagatgatgtatgaagcagaactggaacgtctccagaaggaacgaaaggagaggaagaagaatggaaaagcacaccacaacgagtggccgtgaccataaaatagtccctttccaggccaaggacctgaattctgtttacttcttctggctgtgcaaaagcacactcaagtgaatgactaaaaatgcaaccacagtgcatgttgcagataccggcggccgcaggaggggcagcatccagtagaggactggcgttggagtcacactgctgtgaaatcacgttgcagtccagcgcacaattgctatctatccatagaccattcttgaccaagcaagcatgcacattatgggcagttacattctcaagtttttaaaatcaaggggaacttgtatactgggcctgtttttcagcctgtttgctaccttttttgcattctatcccatgtgaattttacagacactgggctaaaaagggtattcagacacatggacacacattcctagaatgtcatcatatggtcctaattccatgtcaccaagaacacagacaagaccctgtttacaactttttctttccttttttttaattttagacctttctgagaagattattatatatgacatatctatagctatgtgtatggccatagatgtatttctgtgtgtacatatgtatagtcatgtattcctgcatatgtacatacaaatacagagatatataaagtacatagaaattccttacttgtaaatagccaaaaagtactgacatgagtgaattttcacatttaaatagtcatcaatatgaagccatgattaatgcttgtataatgtgatgcaataaaatttaaaataaatttctgcacatggaatattttc

SEQ ID NO: 4 - protein, 2261 aa, Homo sapiens
  misc_feature (780)..(780): Xaa can be any naturally occurring amino acid
  misc_feature (1555)..(1555): Xaa can be any naturally occurring amino acid
  misc_feature (1648)..(1648): Xaa can be any naturally occurring amino acid
  misc_feature (1974)..(1974): Xaa can be any naturally occurring amino acid
  misc_feature (2168)..(2168): Xaa can be any naturally occurring amino acid
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFAXLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAXKQMKKHLKLAKDSSADRFLNSLGRFMTGLDTRNNVKVWFNNKGWHAISSFLNVINNAILRANLQKGENPSHYGITAFNHPLNLTKQQLSEVAXMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNXNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVXKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 5 - DNA, 1660 bp, Homo sapiens
tctatgaaaccaacatacatggcgtttgcatcacagttggagtcagatgtgagcccggagggcaggtgtctggcttgtccacccggaagccctgagggcagctgttcccactggctctgctgaccttgtgccttggacggctgtcctcagcgaggggccgtgcacccgctcctgagcagcgccatgggcctgctggccttcctgaagacccagttcgtgctgcacctgctggtcggctttgtcttcgtggtgagtggtctggtcatcaacttcgtccagctgtgcacgctggcgctctggccggtcagcaagcagctctaccgccgcctcaactgccgcctcgcatactcactctggagccaactggtcatgctgctggagtggtggtcctgcacggagtgtacactgttcacggaccaggccacggtagagcgctttgggaaggagcacgcagtcatcatcctcaaccacaacttcgagatcgacttcctctgtgggtggaccatgtgtgagcgcttcggagtgctggggagctccaaggtcctcgctaagaaggagctgctctacgtgcccctcatcggctggacgtggtactttctggagattgtgttctgcaagcggaagtgggaggaggaccgggacaccgtggtcgaagggctgaggcgcctgtcggactaccccgagtacatgtggtttctcctgtactgcgaggggacgcgcttcacggagaccaagcaccgcgttagcatggaggtggcggctgctaaggggcttcctgtcctcaagtaccacctgctgccgcggaccaagggcttcaccaccgcagtcaagtgcctccgggggacagtcgcagctgtctatgatgtaaccctgaacttcagaggaaacaagaacccgtccctgctggggatcctctacgggaagaagtacgaggcggacatgtgcgtgaggagatttcctctggaagacatcccgctggatgaaaaggaagcagctcagtggcttcataaactgtaccaggagaaggacgcgctccaggagatatataatcagaagggcatgtttccaggggagcagtttaagcctgcccggaggccgtggaccctcctgaacttcctgtcctgggccaccattctcctgtctcccctcttcagttttgtcttgggcgtctttgccagcggatcacctctcctgatcctgactttcttggggtttgtgggagcagcttcctttggagttcgcagactgataggagtaactgagatagaaaaaggctccagctacggaaaccaagagtttaagaaaaaggaataattaatggctgtgactgaacacacgcggccctgacggtggtatccagttaactcaaaaccaacacacagagtgcaggaaaagacaattagaaactatttttcttattaactggtgactaatattaacaaaacttgagccaagagtaaagaattcagaaggcctgtcaggtgaagtcttcagcctcccacagcgcagggtcccagcatctccacgcgcgcccgtgggaggtgggtccggccggagaggcctcccgcggacgccgtctctccagaactccgcttccaagagggacctttggctgctttctctccttaaacttagatcaaattttaaaaaaaaaaaaaaa

SEQ ID NO: 6 - DNA, 1774 bp, Homo sapiens
tgaacccagccggctccatctcagcttctggtttctaagtccatgtgccaaaggctgccaggaaggagacgccttcctgagtcctggatctttcttccttctggaaatctttgactgtgggtagttatttatttctgaataagagcgtccacgcatcatggacctcgcgggactgctgaagtctcagttcctgtgccacctggtcttctgctacgtctttattgcctcagggctaatcatcaacaccattcagctcttcactctcctcctctggcccattaacaagcagctcttccggaagatcaactgcagactgtcctattgcatctcaagccagctggtgatgctgctggagtggtggtcgggcacggaatgcaccatcttcacggacccgcgcgcctacctcaagtatgggaaggaaaatgccatcgtggttctcaaccacaagtttgaaattgactttctgtgtggctggagcctgtccgaacgctttgggctgttagggggctccaaggtcctggccaagaaagagctggcctatgtcccaattatcggctggatgtggtacttcaccgagatggtcttctgttcgcgcaagtgggagcaggatcgcaagacggttgccaccagtttgcagcacctccgggactaccccgagaagtattttttcctgattcactgtgagggcacacggttcacggagaagaagcatgagatcagcatgcaggtggcccgggccaaggggctgcctcgcctcaagcatcacctgttgccacgaaccaagggcttcgccatcaccgtgaggagcttgagaaatgtagtttcagctgtatatgactgtacactcaatttcagaaataatgaaaatccaacactgctgggagtcctaaacggaaagaaataccatgcagatttgtatgttaggaggatcccactggaagacatccctgaagacgatgacgagtgctcggcctggctgcacaagctctaccaggagaaggatgcctttcaggaggagtactacaggacgggcaccttcccagagacgcccatggtgcccccccggcggccctggaccctcgtgaactggctgttttgggcctcgctggtgctctaccctttcttccagttcctggtcagcatgatcaggagcgggtcttccctgacgctggccagcttcatcctcgtcttctttgtggcctccgtgggagttcgatggatgattggtgtgacggaaattgacaagggctctgcctacggcaactctgacagcaagcagaaactgaatgactgactcagggaggtgtcaccatccgaagggaaccttggggaactggtggcctctgcatatcctccttagtgggacacggtgacaaaggctgggtgagcccctgctgggcacggcggaagtcacgacctctccagccagggagtctggtctcaaggccggatggggaggaagatgttttgtaatctttttttccccatgtgctttagtgggctttggttttctttttgtgcgagtgtgtgtgagaatggctgtgtggtgagtgtgaactttgttctgtgatcatagaaagggtattttaggctgcaggggagggcagggctggggaccgaaggggacaagttcccctttcatcctttggtgctgagttttctgtaacccttggttgccagagataaagtgaaaagtgctttaggtgagatgactaaattatgcctccaagaaaaaaaaattaaagtgcttttctgggtcaaaaaaaaaaaaaaa

SEQ ID NO: 7 - DNA, 3000 bp, Homo sapiens
  misc_feature (173)..(173): n is a, c, g, or t
gggcggacctaaaggggctcgggccgctcgggccgggaatggcggcggcggccgagcccggggcccgcgcctggctgggcggcggctccccgcgccccggcagcccggcctgcagccccgtgctgggctcaggaggccgcgcgcgcccggggccggggccggggccgggacgngaccgagcgggcggcgtcagagcccgggcccgtgccgcgccgggacacagcttccggaaggtgacgctcaccaagcccaccttctgccacctctgctccgacttcatctgggggctggccggcttcctgtgcgacgtctgcaatttcatgtctcatgagaagtgcctgaagcacgtgaggatcccgtgcacgagtgtggcacccagcctggtccgggttcctgtagcccactgcttcggcccccgggggctccacaagcgcaagttctgtgctgtctgccgcaaggtcctggaggcaccggcgctccactgcgaagtgtgtgagctgcacctccacccagactgtgtgcccttcgcctgcagtgactgccgccagtgccaccaggatgggcaccaggatcacgacacccatcaccaccactggcgggaggggaacctgccctcgggagcgcgctgcgaggtctgcaggaagacgtgcggctcctctgacgtgctggccggcgtgcgctgcgagtggtgcggggtccaggcgcactccctctgctccgcggcactggctcccgagtgtggcttcgggcgtctgcgctccctggtcctgcctcccgcgtgcgtgcgccttctgcccggcggcttcagcaagacgcagagcttccgcatcgtggaggccgcggagccgggcgaggggggcgacggcgccgacgggagcgctgccgtgggtccaggcagagagacacaggcaactccggagtccgggaagcaaacgctgaagatctttgatggcgacgacgcggtgagaagaagccagttccgcctcgtcacggtgtcccgcctggccggtgccgaggaggtgctggaggccgcactgcgggcccaccacatccccgaggaccctggccacctggagctgtgccggctgcccccttcctctcaggcctgtgacgcctgggctgggggcaaggctgggagtgctgtgatctcggaggagggcagaagccccgggtccggcgaggccacgccagaggcctgggtcatccgggctctgccgcgggcccaggaggtcctgaagatctaccctggctggctcaaggtgggcgtggcctacgtgtccgtgcgagtgacccctaagagcacggctcgctctgtggtgctggaggtcctgccgctgctcggccgccaggccgagagtcccgagagcttccagctggtggaggtggcgatgggctgcaggcacgtccagcggacgatgctgatggacgaacagcccctgctggaccggctacaggacatccggcagatgtctgtgcggcaggtgagccagacgcggttctacgtggcagagagcagggatgtagccccgcacgtctccctgtttgttggcggcctgcctcccggcctgtctcccgaggagtacagcagcctgctgcatgaggccggggctaccaaagccaccgtggtgtccgtgagtcacatctactcctcccaaggcgcggtagtgttggacgttgcctgctttgcggaggccgagcggctgtacatgctgctgaaggacatggctgtgcggggccggctgctcactgccctggtgctccccgacctgctgcacgcgaagctgcccccagacagctgtcccctccttgtgttcgtgaaccccaagagtggaggcctcaagggccgagacctgctctgcagcttccggaagctactgaaccctcatcaggtcttcgacctgaccaacggaggtcctcttcccgggctccacctgttctcccaggtgccctgcttccgggtgctggtgtgtggtggcgatggcactgtgggctgggtgcttggcgccctggaggagacacggtaccgactggcctgcccggagccttctgtggccatcctgcccctgggcacagggaatgaccttggtcgagtcctccgctggggggcgggctacagcggcgaggacccgttctccgtactgctgtctgtggacgaggccgacgccgtgctcatggaccgctggaccatcctgctggatgcccacgaagctggcagtgcagagaacgacacggcagacgcagagccccccaagatcgtgcagatgagtaactactgtggcattggcatcgacgcggagctgagcctggacttccaccaggcacgggaagaggagcctggcaagttcacaagcaggctgcacaacaagggtgtgtacgtgcgggtggggctgcagaagatcagtcactctcggagcctgcacaagcagatccggctgcaggtggagcggcaggaggtggagctgcccagtattgaaggcctcatcttcatcaacatccccagctggggctcgggggccgacctgtggggctccgacagcgacaccaggtttgagaagccacgcatggacgacgggctgctggaggttgtgggcgtgacgggcgtcgtgcacatgggccaggtccagggtgggctgcgctccggaatccggattgcccagggttcctacttccgagtcacgctcctcaaggccaccccggtgcaggtggacggggagccctgggtccaggccccggggcacatgatcatctcagctgctggccctaaggtgcacatgctgaggaaggccaagcagaagccgaggagggccgggaccaccagggatgcccgggcggatcgtgcgcctgcccctgagagcgatcctaggtaggggtggctggggcagcccaagggctcgagccatctctgctcccgccagccttgttttcaggtggtctggaggcagctccacgtcacacagtggctgtcatatattgaagttaccttcccactggaaaaaaaat

SEQ ID NO: 8 - DNA, 2215 bp, Homo sapiens
gtggctcggaccgccgcctgaatgtacctcgctcccgggagccggacggcccagtagggcgcactggaggacgctccgctgcgggagcctggacagtttttgacggtgcagtcttgctatatggtgtgagaaatggctgtaggaaacaacactcaacgaagttattccatcatcccgtgttttatatttgttgagcttgtcatcatggctgggacagtgctgcttgcctactacttcgaatgcactgacacttttcaggtgcatatccaaggattcttctgtcaggacggagacttaatgaagccttacccagggacagaggaagaaagcttcatcacccctctggtgctctattgtgtgctggctgccaccccaactgctattatttttattggtgagatatccatgtatttcataaaatcaacaagagaatccctgattgctcaggagaaaacaattctgaccggagaatgctgttacctgaaccccttacttcgaaggatcataagattcacaggggtgtttgcatttggactttttgctactgacatttttgtaaacgccggacaagtggtcactgggcacttaacgccatacttcctgactgtgtgcaagccaaactacaccagtgcagactgccaagcgcaccaccagtttataaacaatgggaacatttgtactggggacctggaagtgatagaaaaggctcggagatcctttccctccaaacacgctgctctgagcatttactccgccttatatgccacgatgtatattacaagcacaatcaagacgaagagcagtcgactggccaagccggtgctgtgcctcggaactctctgcacagccttcctgacaggcctcaaccgggtctctgagtatcggaaccactgctcggacgtgattgctggtttcatcctgggcactgcagtggccctgtttctgggaatgtgtgtggttcataactttaaaggaacgcaaggatctccttccaaacccaagcctgaggatccccgtggagtacccctaatggctttcccaaggatagaaagccctctggaaaccttaagtgcacagaatcactctgcgtccatgaccgaagttacctgagacgactgatgtgtcacaagctgttttttaaaatcatcttccaattctatacttcaaaacacacagttgctcaatgtcaaactgtgatgacaaatattacgtttatctagttagaagctaatgttttgtacattttttgtatgaggaagtgatgtagcttgccctgatttttttttttttttttggtcagctttaatatatttatgccagaattttaaaaccaacaaaattttcttgttcaagcgtgcattgaagaaccacatttattcaatggttgacgttgttttgtgatatttgtacacaaattttcttttctcagttttataaacacagaagtaaatataacaattcactttaaacttttattaccacagttgctgcctcctccagaatttttgaattttaataaaaggcaaacttttgagctgcaggaaggacaatgttggttaataataaatctcaaagtcaattgtagaaaaaaaattgtcttcaaaaagaatgttgcactctgatctcttaacaaattgttacgttcaaagtttaaagtgatatattaacaaagtcacctagttatacaaacaattgtcagagaattctggatttggagggtattggggttatatgattctttcttagataatggcctctactaaataactcaagatctttctggaatgtcttctggcaggcaggtgccactgtcagcttttctccaaaaagcagccaacatcagcctcccctgtcaactcaacagttttgtatctcatattatatggactttatatgaaaatgaatattttacagtttgcacagtattattttacagaaaaggaatcagagaatctacaacatagggccccagaacaacagtttcactttgtggcttttaattattctagaattttaactgcatctcatttttctagcatggtgagaactaatatgtaactcctttgattgaaggagctcttttgtccgtacctatcagaatgttttcttgacacttccatgttggctcttctcagctttttttgtacatattttttttttctaaagagaagaaaaagttatcacaaaatgtaaaaaaagaaaaaaaaaaaaaaaa

SEQ ID NO: 9 - DNA, 20 bp, Artificial Sequence
  synthetic construct; primer
cgtttccgggaagtgtccta

SEQ ID NO: 10 - DNA, 24 bp, Artificial Sequence
  synthetic construct; primer
gctagagatgacaaggaggatgga

SEQ ID NO: 11 - DNA, 20 bp, Artificial Sequence
  synthetic construct; primer
agtgctcagccttcgtacag

SEQ ID NO: 12 - DNA, 20 bp, Artificial Sequence
  synthetic construct; primer
aggcgcgtacaggattttgg

SEQ ID NO: 13 - DNA, 22 bp, Artificial Sequence
  synthetic construct; primer
tttgagggatttgggtctgaac

SEQ ID NO: 14 - DNA, 23 bp, Artificial Sequence
  synthetic construct; primer
cccctttaatcgttttgtctgct

SEQ ID NO: 15 - DNA, 20 bp, Artificial Sequence
  synthetic construct; primer
gggatgcccgtgcctgcaat

SEQ ID NO: 16 - DNA, 20 bp, Artificial Sequence
  synthetic construct; primer
ctggcagctacatggccccg

(19) United States
(12) Patent Application Publication
(10) Pub. No.: US 2020/0268788 A1
(43) Pub. Date: Aug. 27, 2020

(54) USE OF CYCLODEXTRINS IN DISEASES AND DISORDERS INVOLVING PHOSPHOLIPID DYSREGULATION

(71) Applicant: Asdera LLC

(72) Inventor: Knut M. Wittkowski, New York, NY (US)

(21) Appl. No.: 16/643,494

(22) PCT Filed: Aug. 28, 2018
(86) PCT No.: PCT/US2018/048414

§ 371 Date: Feb. 28, 2020

Related U.S. Application Data
(60) Provisional application No. 62/551,193, filed on Aug. 28, 2017.
(60) Provisional application No. 62/565,053, filed on Sep. 28, 2017.
(60) Provisional application No. 62/573,658, filed on Oct. 17, 2017.
(60) Provisional application No. 62/586,826, filed on Nov. 15, 2017.
(60) Provisional application No. 62/643,694, filed on Mar. 15, 2018.
(60) Provisional application No. 62/679,912, filed on Jun. 3, 2018.

(51) Int. Cl. A61K 31/724; A61K 47/12; A61K 9/00; A61P 35/00

(52) U.S. Cl. CPC A61K 31/724; A61P 35/00; A61K 9/0053; A61K 47/12

(57) ABSTRACT

The present disclosure provides certain compositions and methods that may be useful in the treatment and/or prevention of a malignant, neurodegenerative, cardiovascular, metabolic, inflammatory, autoimmune, or viral disease or disorder, such as carcinomas, Alzheimer's and Parkinson's disease, multiple sclerosis, Paget's disease, or other aspects of aging, such as atherosclerosis or type-2 diabetes. In some such embodiments, compositions are provided that contain at least one cyclodextrin active agent, such as alpha-cyclodextrin, or an analogue or derivative thereof. In some embodiment the composition is a clathrate of HP-aCD and sodium caprate or caprylate.

### RELATED APPLICATIONS

This application claims the benefit of priority to U.S. Provisional Application No. 62/679,912, filed Jun. 3, 2018, U.S. Provisional Application No. 62/643,694, filed Mar. 15, 2018, U.S. Provisional Application No. 62/586,826, filed Nov. 15, 2017, U.S. Provisional Application No. 62/573,658, filed Oct. 17, 2017, and U.S. Provisional Application No. 62/565,053, filed Sep. 28, 2017, and U.S. Provisional Application No. 62/551,193, filed Aug. 28, 2017. The entire contents of the aforementioned applications are incorporated by reference in their entireties.

### BACKGROUND

As the human population is aging, the prevalence of age-related conditions, Error! Reference source not found. cancers and neurodegenerative diseases increases, yet interventions to prevent or treat these conditions are lacking or have undesirable side effects. Currently, about 5 million US individuals have Alzheimer's disease and about 1 million have Parkinson's disease. As life expectancy increases, the prevalence of neurodegenerative diseases or disorders also increases. Ten percent of people age 65 and older and 15% of people age 75 or older have Alzheimer's disease. Alzheimer's disease also disproportionately affects women, who comprise two thirds of Americans with Alzheimer's disease. Alzheimer's disease is also more likely to affects members of African American and Hispanic communities than Caucasian communities. Patients with Alzheimer's disease have few treatment options. Three recent phase 3 studies of BACE1 inhibitors have failed. Similarly, patients with Parkinson's disease initially benefit from treatment of motor symptoms (levodopa), but become non-responsive over time.

Treatments of malignant diseases or disorders are also limited and suffer from numerous drawbacks. Treatments generally include local therapy (for instance: surgery with or without radiation in breast cancer, surgery or radiation in prostate cancer) and adjuvant systemic therapy (hormonal therapy, chemotherapy, and biologic agents) for cancer cells that may have spread. Radiation and chemotherapy often cause substantial side-effects including, but not limited to nausea and hair loss. Hormone therapy for prostate cancer includes anti-androgens. For some hormone-receptor-positive forms of breast cancer, selective estrogen receptor modulators (SERM), such as tamoxifen and raloxifene, and aromatase inhibitors, such as exemestane and anastrozole, can interfere with disease progression. Monoclonal antibodies, such as trastuzumab and pertuzumab, are approved for the treatment of HER2 positive cancer. For patients with triple-negative (absence of estrogen, progesterone, and the Her2/neu receptor), treatment options are limited.

A common thread between neurodegenerative disorders and malignancies are lysosomal dysfunction. Lysosomal dysfunction is implicated in a diverse range of disorders and diseases, including genetic diseases. In some cases, high levels of endocytosis have been suggested as a pleiotropic factor in the etiology underlying malignant and neurodegenerative processes, including, but not limited to a role of endocytosis in cancer, and “deranged” endocytosis in Alzheimer's disease, where endocytic pathway abnormalities precede Aβ deposition and inhibition of endocytosis reduces amyloid precursor protein (“APP”) internalization and immediately lowers Aβ levels in vivo.

While beneficial in early life, high levels of endocytosis may become detrimental with age. Enlarged macrophages have been identified in atherosclerosis, stearohepatitis, and cyctic fibrosis, and impaired phospholipid efflux increases accumulation of lipids in macrophages. Activation of the early endocytic pathway has been observed in autoimmune-diseases, such as systemic lupus erythematosus (SLE), inflammatory bowel disease, and arthritis. Attenuation of phosphoinisitides restrains autoimmune disease. Inhibition of PI3K signaling has been shown to benefit inflammatory/autoimmune diseases and hematological malignancies. Overall, dysregulation of the lipid raft have been linked to poor quality of life with aging.

Phosphoinisitides (PIPs), which regulate endocytosis, have been mentioned in genetic diseases, In particular, PI3K inhibitors have been shown to be effective in a variety of inflammatory and autoimmune diseases. Several attempts to inhibit or activate individual phosphatases or kinases, however, have failed to result in successful therapies. Hence, in spite of this strong evidence for involvement of endo-/pinocytosis in all these diseases and for involvement of PIPs, effective modulators of endocytosis are lacking.

Because “derailed endocytosis” has been linked to cancers, neurodegenerative diseases, and other “pathological conditions”, the same treatment that prevents metastases in cancer is expected to also prevent accumulation of undegraded macromolecules in neurodegenerative diseases and other many other age-related conditions, including formation of foam cells in atherosclerosis via macropinocytosis. Yet, effective treatments to regulate endocytosis are lacking. Therefore, there is a need for effective treatments for the symptoms of disorders and diseases that involve dysfunction in lysosomal pathways.

Broadly acting [cyclin-dependent kinase (CDK)] inhibitors yielded largely disappointing results, and only a small number of patients benefit from phosphoinositide 3-kinase (PI3K) inhibitors. Recently, HP-β-CD has been proposed for the treatment of cancers, but extracting cholesterols not only from cancer, but also from outer hair cell, may cause permanent hearing loss.

2-Hydroxypropyl-β-cyclodextrin (HP-β-CD) is “generally recognized as safe” (GRAS) as food additives and frequently used as an excipient with parenteral use to form water soluble compounds with lipophilic drugs. Parenteral and intrathecal HP-β-CD has been used for the treatment of Niemann-Pick disease type C (NPC), where its function is to extract excess cholesterol, a lipid molecule, from lysosomes. As cholesterol is implicated in many other processes, including cell growth, HP-β-CD has also been suggested as a treatment of various cancers. In most cases cholesterol depletion by [HP-β-]CD had been suggested, although the precise mechanism of action remains unverified.

It has not yet been appreciated that α-cyclodextrin and its derivatives, such as hydroxypropyl-α-cyclodextrin (HP-α-CD), may effectively treat malignancies and neurodegenerative disorders without the undesirable side effects, such as hair and hearing loss caused by beta-cyclodextrin.

### SUMMARY

Through the application of computational biostatistics and decision strategies to data from genome-wide association studies (GWAS), we have identified functionally related collections of genes and determined that endocytosis, which is controlled by the phosphoinisitides (PIP) comprising the generated by the PI cycle, is involved in the metastases process of certain types of cancers such as breast or prostate cancer. In this regard, we established that breast cancer risk is conferred not only by (rare) variations in DNA damage repair genes, such as BRCA1/2, but more frequently by a global dysregulation of PI cycling and that endocytosis, which is known to be controlled by the PI cycle, is a critical component of local spread, migration, and invasion of cancer cells. See e.g., Example 1. Thus, in one aspect, provided herein are methods of targeting the PI cycle as a means for treating diseases or conditions causing a dysfunctional lysosomal pathway and/or elevated endocytosis, phagocytosis, or pinocytosis in a subjet, in which the diseases or condition may be caused by e.g., aberrant expression of a gene or combination of genes as identified e.g., by the novel GWAS approach (see e.g., Example 1) described herein. Such diseases or conditions include e.g., breast cancer (BC), Alzheimer's disease (AD), Parkinson's disease (PD), multiple sclerosis (MS), coronary artery disease (CAD), and Niemann-Pick type C disease (NPC). See e.g., Examples 2, 3, and 6-8.

Therapeutic agents known to have effects against animal models of carcinomas, neurodegenerative and several other age-related diseases are beta-cyclodextrins. These include e.g., methyl-β-cyclodextrin (MβCD) and 2-hydroxypropyl-β-cyclodextrin (HP-β-CD). See e.g., J Membr Biol. 2011 May, 241(1): 1-10; The Journal of Experimental Medicine, 209 (13), 2501-13, and the discussion set forth in Example 4. Traditionally, beta-cyclodextrins were believed to remove and to extract cholesterol (Chol) from cell membranes and/or organelles. For example, the scavenging of cholesterol and/or binding directly to Aβ or α-synuclein was believed to be the mode of action for beta-cyclodextrins in AD and PD. See The Journal of Experimental Medicine, 209 (13), 2501-13. Although beta-cyclodextrins have therapeutic potential, they are ototoxic, i.e., cause damage to the ear which can in permanent hearing loss. See J Assoc Res Otolaryngol, 16 (5), 599-611.

Here, having for the first time identifed the implication of the PI cycle and the genetic results for lysosomal function diseases (See e.g., Example 1), the scaveging of cholesterol can no longer be viewed as the primary mechansim upon which cyclodextrins such as beta-cyclodextrins function. See e.g., Example 1. Rather, our data shows that cyclodextrins act by scavenging phospholipids and, thereby, regulate endocytosis. For example, we have found that alpha-cyclodextrin (α-CD) restores derailed endocytosis in breast cancer and deranged endocytosis in PD and AD. See e.g., Example 9. We have also found that α-CD is more efficient than β-CD in solubilizing phospholipids (see Examples 10 and 11) and is less toxic and more effective in preventing the migration of human tumor cells (see Example 12). Therefore, in one aspect, provided herein are methods of using alpha-cyclodextrins (e.g., methyl-α-cyclodextrin (MαCD) or 2-hydroxypropyl-α-cyclodextrin (HP-α-CD)) for treating a subject having a disease, disorder, or condition that involves impaired lysosomal function. Such diseases and disorders are described herein and include e.g., breast cancer (BC), Alzheimer's disease (AD), Parkinson's disease (PD), multiple sclerosis (MS), coronary artery disease (CAD), and Niemann-Pick type C disease (NPC).

In addition to the above findings, we have further realized that the absorption of alpha-cyclodextrins (e.g., HP-α-CD) from the intestine can be improved by the complexation with fatty acids. For example, we have demonstrated that a clathrate of HP-α-CD and medium chain fatty acids substantially improved absorption. See Example 14. Accordingly, in one aspect, provided herein are pharmaceutical compositions comprising an alpha-cyclodextrin (e.g., HP-α-CD) and a medium chain fatty acid (e.g., capric acid), as well as and their use for treating a disease or disorder that involves impaired lysosomal function. Such diseases and disorders are described herein and include e.g., breast cancer (BC), Alzheimer's disease (AD), Parkinson's disease (PD), multiple sclerosis (MS), coronary artery disease (CAD), and Niemann-Pick type C disease (NPC).

In yet another aspect, we further identified that pharmaceutical compositions comprising an alpha-cyclodextrin (e.g., HP-α-CD) and a medium chain fatty acid (e.g., capric acid) can be used to treat anti-inflammatory diseases. See e.g., Example 15. Therefore, in one aspect, provided herein are pharmaceutical compositions comprising an alpha-cyclodextrin (e.g., HP-α-CD) and a medium chain fatty acid (e.g., capric acid) for use in treating inflammatory diseases.

Additional aspects are further described below and in the Detailed Description and Examples sections of the application. The description in each of the sections of this patent application is intended to be read in conjunction with the other sections. Furthermore, the various embodiments described in each of the sections of this patent application can combined in various different ways, and all such combinations are intended to fall within the scope of the present disclosure.

In one aspect, the present disclosure provides a method of treating a malignant, neurodegenerative, cardiovascular, metabolic, inflammatory, autoimmune, or viral disease or disorder in a subject, the method comprising administering to the subject an effective amount of a cyclodextrin (e.g., α-cyclodextrin (α-CD)), or an analogue or derivative thereof (e.g., 2-hydroxypropyl-alfa-cyclodextrin (HP-α-CD)), either alone or in combination with one or more additional active agents. In another aspect, the present disclosure provides a composition comprising α-CD, or an analogue or derivative thereof (e.g., 2-hydroxypropyl-alfa-cyclodextrin (HP-α-CD)), for use in the treatment of an epithelial cancer (carcinoma) or the treatment of Parkinson's, Alzheimer's, or Huntington's disease. In one aspect, the composition comprising HP-α-CD further comprises a medium-length chain fatty acid (MCFA), e.g., fatty acids having an aliphatic tail of 6-12 carbon atoms. In one aspect, the HP-α-CD and medium-length chain fatty acid (MCFA) in the composition form a clathrate. In such clathrates, the MCFAs are guests of HP-α-CD. In one the MCFA in the composition is caprate or salt thereof such as sodium caprate.

In one aspect, the present disclosure provides a method of improving one or more indicators or symptoms of a malignant, neurodegenerative, cardiovascular, metabolic, inflammatory, autoimmune, or viral disease or disorder in a subject, the method comprising administering to a subject exhibiting one or more indicators or symptoms of a malignant, neurodegenerative, cardiovascular, metabolic, inflammatory, autoimmune, or viral disease or disorder, an effective amount of α-CD, or an analogue or derivative thereof (e.g., 2-hydroxypropyl-alfa-cyclodextrin (HP-α-CD)), either alone or in combination with one or more additional active agents. Suitable indicators include, but are not limited to, results of a blood test (including, but not limited to circulating tumor DNA and/or prostate-specific antigen), an x-ray evaluation, the result of a physical examination (including, but not limited to a palpable tumor), a psychiatric evaluation, or a tissue biopsy for histological evaluation and/or determination of hormone receptor status. In one aspect, a symptom or indicator is selected from the group consisting of survival, disease-free survival, distant metastasis-free survival, results of a blood test (including, but not limited to circulating tumor DNA and prostate-specific antigen), an x-ray evaluation, the result of a physical examination (including, but not limited to a palpable tumor), or a tissue biopsy for histological evaluation. In one embodiment, the “improving” comprises an increase of at least 1% in a measurement of the one or more indicators or symptoms. n another aspect, the present disclosure provides a composition comprising α-CD Error! Reference source not found., or an analogue or derivative thereof (e.g., 2-hydroxypropyl-alfa-cyclodextrin (HP-α-CD)), for use in the treatment of an epithelial cancer (carcinoma) or the treatment of Parkinson's, Alzheimer's, or Huntington's disease. In one aspect, the composition comprising HP-α-CD further comprises a medium-length chain fatty acid (MCFA), e.g., fatty acids having an aliphatic tail of 6-12 carbon atoms. In one aspect, the HP-α-CD and medium-length chain fatty acid (MCFA) in the composition form a clathrate. In such clathrates, the MCFAs are guests of HP-α-CD. In one the MCFA in the composition is caprate or salt thereof such as sodium caprate.

In some embodiments, the disclosure relates to a method of restoring the synthesis of sphingomyelin in a subject in need thereof, the method comprising administering a cyclodextrin (e.g., α-cyclodextrin (α-CD)), or an analogue or derivative thereof (e.g., 2-hydroxypropyl-alpha-cyclodextrin (HP-α-CD)), either alone or in combination with one or more additional active agents. In one aspect, the present disclosure provides a method of improving one or more indicators or symptoms of a malignant, neurodegenerative, cardiovascular, metabolic, inflammatory, autoimmune, or viral disease or disorder in a subject, the method comprising administering to a subject exhibiting one or more indicators or symptoms of a malignant, neurodegenerative, cardiovascular, metabolic, inflammatory, autoimmune, or viral disease or disorder, an effective amount of α-CDError! Reference source not found., or an analogue or derivative thereof (e.g., 2-hydroxypropyl-alpha-cyclodextrin (HP-α-CD)), either alone or in combination with one or more additional active agents. Suitable indicators include, but are not limited to, results of a blood test (including, but not limited to circulating tumor DNA and/or prostate-specific antigen), an x-ray evaluation, the result of a physical examination (including, but not limited to a palpable tumor), a psychiatric evaluation, or a tissue biopsy for histological evaluation and/or determination of hormone receptor status. In one aspect, a symptom or indicator is selected from the group consisting of survival, disease-free survival, distant metastasis-free survival, results of a blood test (including, but not limited to circulating tumor DNA and prostate-specific antigen), an x-ray evaluation, the result of a physical examination (including, but not limited to a palpable tumor), or a tissue biopsy for histological evaluation. In one embodiment, the “improving” comprises an increase of at least 1% in a measurement of the one or more indicators or symptoms.

In one aspect, the present disclosure provides a method of treating and/or preventing focal segmental glomerulosclerosis (FSGS) and nephrotic swelling in a subject in need thereof the method comprising administering to the subject an effective amount of α-cyclodextrin (α-CD), or an analogue or derivative thereof (e.g., 2-hydroxypropyl-alfa-cyclodextrin (HP-α-CD)), either alone or in combination with one or more additional active agents. In some embodiments, the disclosure relates to a method of treating and/or preventing kidney damage caused by excess or dysfunctional sphingolipid catabolism in a subject in need thereof, the method comprising administering to the subject an effective amount of α-cyclodextrin (α-CD), or an analogue or derivative thereof (e.g., 2-hydroxypropyl-alfa-cyclodextrin (HP-α-CD)), either alone or in combination with one or more additional active agents. In some embodiments, the disclosure relates to a method of restoring the synthesis of sphingomyelin in a subject in need thereof, the method comprising administering α-cyclodextrin (α-CD), or an analogue or derivative thereof (e.g., 2-hydroxypropyl-alfa-cyclodextrin (HP-α-CD)), either alone or in combination with one or more additional active agents.

In other embodiments the compositions described herein comprise resorption enhancers known in the art including, but not limited to bile salts (sodium glycocholate, sodium deoxycholate, sodium taurocholate, sodium fusidate, sodium glycodeoxycholate, sodium taurodihydrofusidate), surfactants (sodium lauryl sulfate, lysophosphatidylcholine, dioctyl sodium sulfosuccinate, laurenth-9, polysorbate, polyethyleneglycol-8-laurate, glyceryl monolaurate, glyceryl monocaprylate/caprate, glyceryl dicaprylate/caprate, saponin), fatty acids and derivatives (sorbitan laurate, sodium caprate, sucrose palmitate, lauroyl choline, sodium myristate, palmitoyl carnitine), glycerides (phospholipids, monohexanoin, medium chain glycerides), chelators (ethylene diamine tetraacetate (EDTA), disodium EDTA), salicylates (salicylic acid, sodium methoxysalicylate, acetylsalicylic acid), polymers or polysacharides (chitosan and chitosan derivatives, dextran, polyvinyl pyrrolidone (PVP), polycarbophil, sodium carboxymethylcellulose and their derivatives, inulin, pectin, chondroitin sulfate), others (azone, benzalkonium chloride, phenothiazines, nitric acid donors, menthol), newer (zonula occluden toxin, poly-1-arginines, soybean derivative glucosides, citicholine, amino acid peptides). See Biochimica et Biophysica Acta (BBA)—Biomembranes, 1788 (4), 892-910; International journal of pharmaceutics, 447 (0), 75-93; J Appl Pharmacol Sci, 2 (7), 34-39.

In some embodiments, the cyclodextrin is administered to the subject at a dose of about 2500 mg/kg α-CD bi-weekly (≈700 mg/kg/d), the same dose used for HP-β-cyclodextrin in two children with NPC for “over a year, with no discernable side effects” for a “targeted concentration of 0.1-1.0 mM.” (INDs 104,114 and 104,116, approval date: Apr. 13, 2009) In other embodiments, the dose will be adjusted over time to the highest dose not causing renal or hemolysis in the patient. In some embodiments, the cyclodextrin, such as α-CD, is administered to the subject at a dose of at least about 100 mg, at least about 200 mg, at least about 500 mg, at least about 1000 mg, at least about 2000 mg, at least about 5000 mg, or at least about 10,000 mg. In some embodiments, the cyclodextrin, such as α-CD, is administered to the subject at a dose in the range of from about 1 to about 10,000 mg, from about 1 to about 7,500 mg, from about 1 to about 5,000 mg, from about 1 to about 2,500 mg, from about 1 to about 1,000 mg, from about 1 to about 500 mg, from about 1 to about 200 mg, from about 200 to about 10,000 mg, from about 200 to about 4,000 mg, from about 200 mg about 2,000 mg, about 200 to about 1,000 mg, or about 200 to about 500 mg per day. In some such embodiments, each of the dosages described above is mg/kg/day. Additional dosages that may be used are provided in the Detailed Description section of this patent application.

In some embodiments, the intervention is to prevent a malignant, neurodegenerative, cardiovascular, metabolic, inflammatory, autoimmune, or viral disease or disorder in a subject not exhibiting disease-specific indicators or symptoms, except indicators of the subject to belong to an at-risk subpopulation. In some embodiments, the indicator will be age. In some embodiments, the indicator will be more than 30, 40, or 50 years of age.

In some embodiments the present disclosure provides a method of treating or preventing a malignant, neurodegenerative, cardiovascular, metabolic, inflammatory, autoimmune, or viral disease or disorder in a subject, the method comprising administering to the subject an effective amount of a drug reducing extracellular phospholipid. In some embodiment the malignant, neurodegenerative, cardiovascular, metabolic, inflammatory, autoimmune, or viral disease or disorder will be an epithelial cancer (carcinoma). In some embodiments the malignant, neurodegenerative, cardiovascular, metabolic, inflammatory, autoimmune, or viral disease or disorder will be breast cancer. In another embodiment the malignant, neurodegenerative, cardiovascular, metabolic, inflammatory, autoimmune, or viral disease or disorder will be Alzheimer's disease. In some embodiments the malignant, neurodegenerative, cardiovascular, metabolic, inflammatory, autoimmune, or viral disease or disorder malignant, neurodegenerative, cardiovascular, metabolic, inflammatory, autoimmune, or virus viral disease or disorder will be Parkinson's disease. In some embodiments, the malignant, neurodegenerative, cardiovascular, metabolic, inflammatory, autoimmune, or viral disease or disorder will be Huntington's disease. In some embodiments, the malignant, neurodegenerative, cardiovascular, metabolic, inflammatory, autoimmune, or viral disease or disorder is non-alcoholic steatohepatitis (NASH).

In some embodiments, the present disclosure provides various combinations of treatments, including pharmaceutical compositions. In some embodiments, cyclodextrins are used in combination with established pharmaceutical, radiological, or surgical interventions comprising cytotoxic interventions, receptor antagonists, monoclonal antibodies, radiation therapy, removal of tumor tissue, and the like.

In one embodiment, the subject is a human. In another embodiment the subject is an adult human. In some embodiments, the subject is in need of the treatment and/or has been identified as having a risk of developing a disease or disorder resulting from one or a plurality of cellular defects caused by lysosomal dysfunction.

In some embodiments, the composition comprises a clathrate of HP-α-CD and salt, such as sodium caprate.

### BRIEF DESCRIPTION OF THE DRAWINGS

FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. 6: QR-Plot of ssGWAS results for CGEM. The “null” projection (horizontal) ends at the median among the endpoints of the convex projections for individual chromosomes. Genes to the right of the vertical line are above the cut-off for study-specific genome-wide significance. Regions below (to the left) of the cut-off are given for descriptive purposes only.

FIG. 7 QR-Plot of muGWAS results for CGEM. (see FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

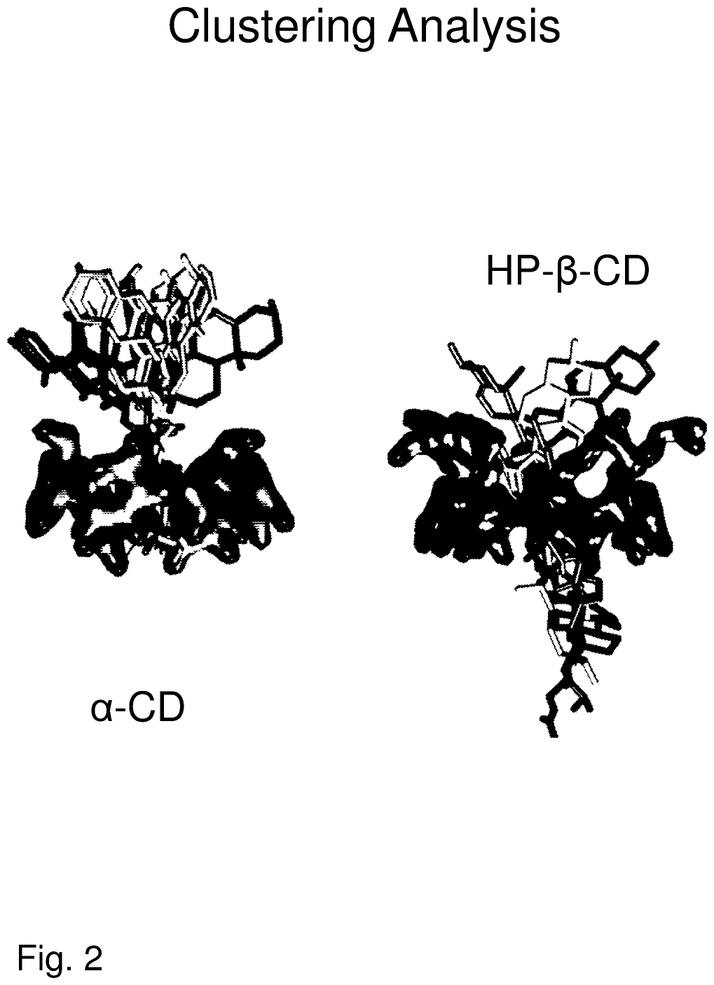

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

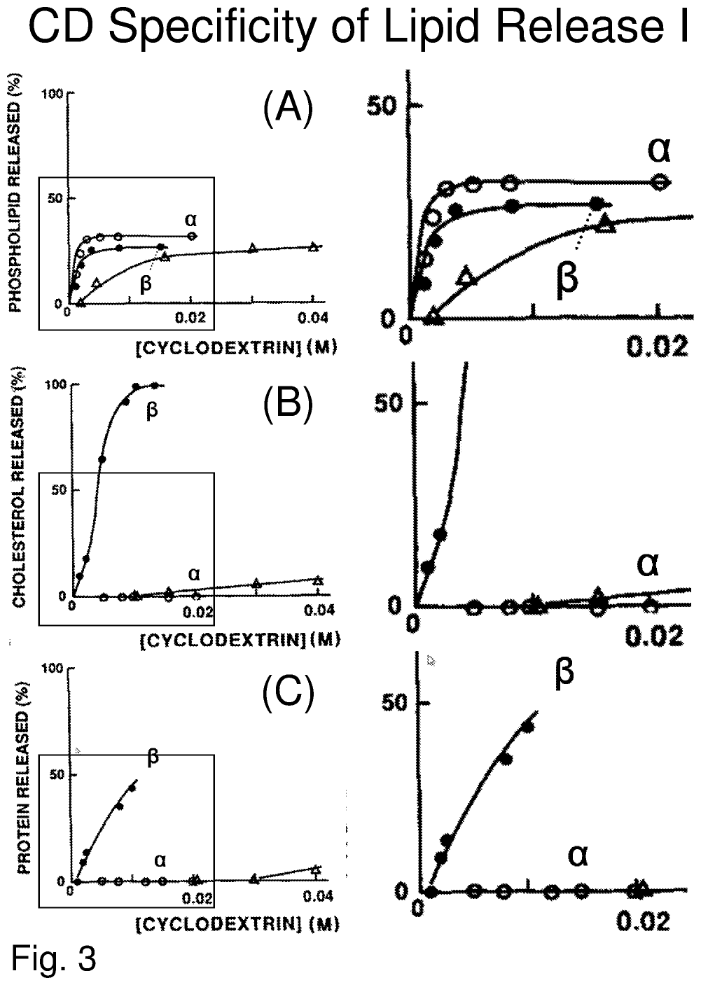

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

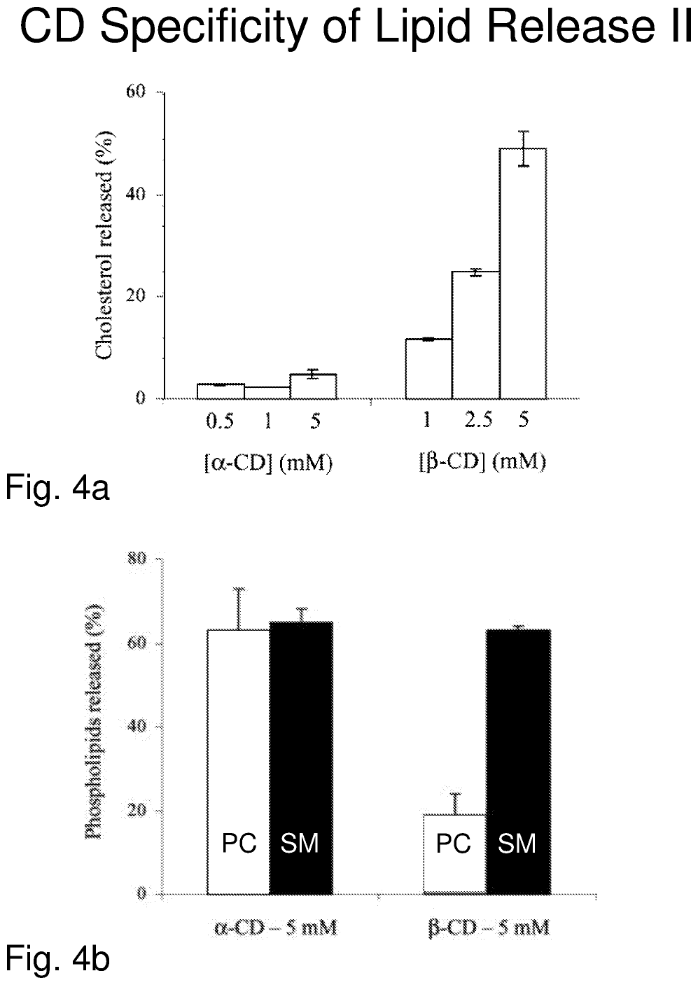

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

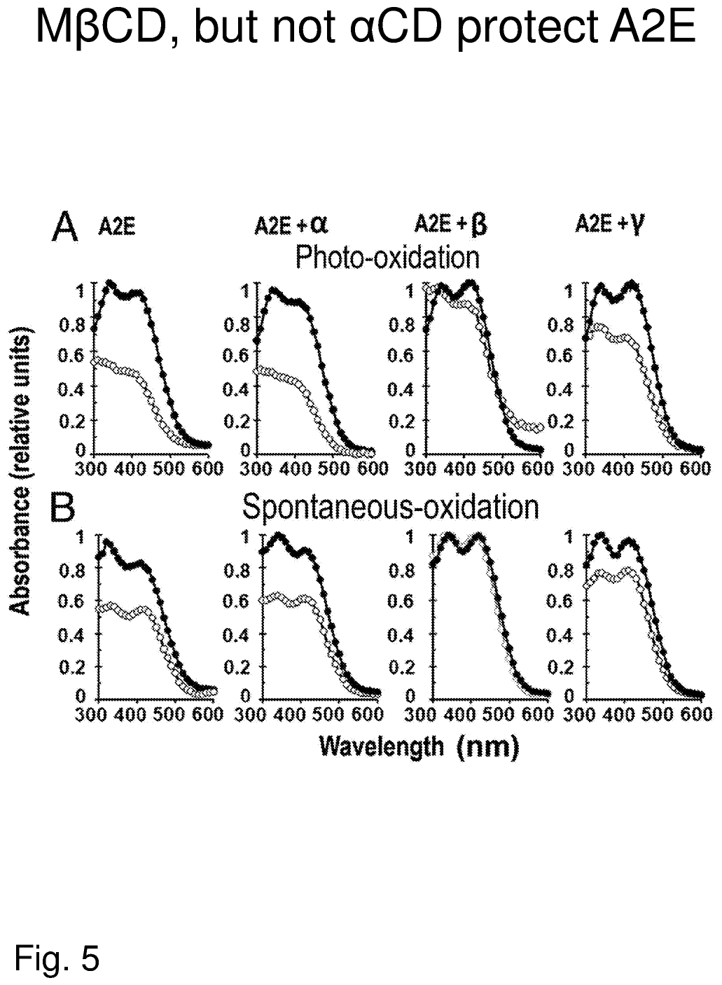

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. for legend)

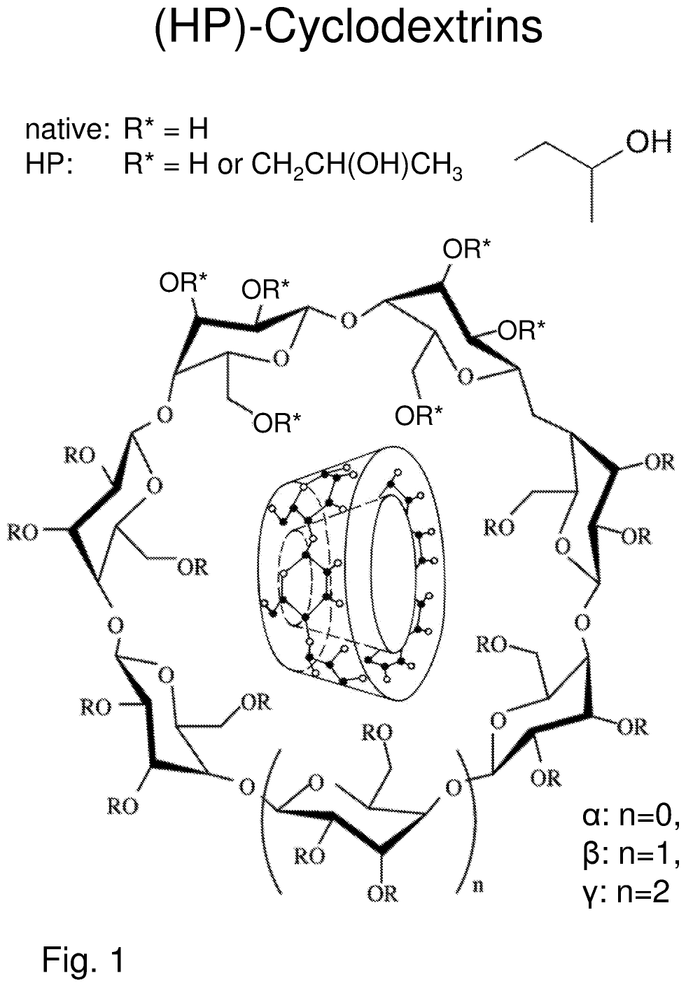

FIG. 8 QR-Plot of ssGWAS results for EPIC. (see FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. for legend)

FIG. 9 QR-Plot of muGWAS results for EPIC. (see FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. for legend)

FIG. 10 QR-Plot of ssGWAS results for PBCS. (see FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. for legend)

FIG. 11 QR-Plot of muGWAS results for PBCS. (see FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (◯) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. for legend)

FIG. 12: PI cycle. hosphatidyl-inositol (PI) cycle. Phosphatidyl-inositol (PI) is synthesized from myo-inositol (imported by HMIT) and phosphatidic acid (PA) (via CDP-DAG), which can be synthesized from lysophosphatic acid (LPA), PC, or (cytosolic) phosphatidyl serine (PS), or salvaged from IP₃ and DAG. It can also be synthesized from 1-acyl GPI. Arrows: PIPs are phosphorylated at a 3-, 4-, or 5- position by PI-kinases (left to right) and hydrolyzed by a plethora of phosphatases (right-to-left). Genes associated with BC in this GWAS are highlighted as inverted (bold: aGWS). Wide arrows in the center indicate the sequence of PIPs involved in EEC. Hexagons: PI/PIPs; PM: plasma membrane, CCV: clathrin-coated vesicle, UCV: uncoated vesicle, EE: early endosome, LE: late endosome LY: lysosome. Inverted gene names indicate genes associated with phosphatidylinositol signaling and/or endocytosis. Bold arrows indicate PIPs associated with endocytosis.

FIGS. 13A and 13B: Known relationship of genes implicated in muGWAS with stages in the process of endocytosis (FIG. 13A) and exocytosis/lysosomal function (FIG. 13B). Boxes: genes identified in the present disclosure by stage of endo-/exocytyosis: Formation of clathrin-coated vesicles (CCVs) and E3 ubiquitination, separation of inactive integrin (fast recycling) from active integrins (slow recycling), sorting between secretory, lysosomal, and (slow) recycling pathway, and lysosomal degradation. Underlined genes are known breast cancer promoters and suppressors, respectively. Clathrin-mediated endocytosis (CME) begins with co-assembly of the heterotetrameric clathrin adaptor complex AP-2 with clathrin at PI(4,5)P₂-rich plasma membrane (PM) sites. AP-2 in its open conformation recruits clathrin and additional endocytic proteins, many of which also bind to PI(4,5)P₂. Clathrin-coated pit (CCP) maturation may be accompanied by SHIP-2-mediated dephosphorylation of PI(4,5)P₂ to PI(4)P. Synthesis of PI(3,4)P₂ is required for assembly of the PX-BAR domain protein SNX9 at constricting CCPs and may occur in parallel with PI(4,5)P₂ hydrolysis to PI(4)P via synaptojanin, thereby facilitating auxilin-dependent vesicle uncoating by the clathrin-dependent recruitment and activation of PI3KC2α, a class II PI3-kinase. PI(3,4)P₂ may finally be converted to PI(3)P en route to endosomes by the 4-phosphatases INPP4A/B, effectors of the endosomal GTPase Rab5. Adapted from Posor, Y., Eichhorn-Grunig, M., and Haucke, V. (2015), ‘Phosphoinositides in endocytosis’, Biochim Biophys Acta, 1851 (6), 794-804. In the early endosome (EE), β1 integrins are sorted. Inactive integrins undergo fast “short loop” recycling; active integrins go to the late endosome (EE)/multivesicular body (MVB) for slow “long group” recycling (RAB11), lysosomal degeneration (unless rescued by RAB25/CLIC3), or secretion via the trans-Golgi-network (TGN) mediated by RAB9. Fast recycling of epidermal growth factor receptor drives proliferation, so one would expect gain-of-function mutations in FIG. 8a. See Tomas, Alejandra, Futter, Clare E., and Eden, Emily R. (2014), ‘EGF receptor trafficking: consequences for signaling and cancer’, Trends in Cell Biology, 24 (1), 26-34. Lysosomal and synaptic vesicle exocytosis share many similarities. Endolysosome-localized PIPs may regulate lysosomal trafficking (derived, in part from Kegg pathways hsa04144 and hsa04721). Adapted from Samie, M. A. and Xu, H. (2014), ‘Lysosomal exocytosis and lipid storage disorders’, J Lipid Res, 55 (6), 995-1009. See Bohdanowicz, M. and Grinstein, S. (2013), ‘Role of phospholipids in endocytosis, phagocytosis, and macropinocytosis’, Physiol Rev, 93 (1), 69-106; Hesketh, G. G., et al. (2014), ‘VARP is recruited on to endosomes by direct interaction with retromer, where together they function in export to the cell surface’, Dev Cell, 29 (5), 591-606; Mosesson, Yaron, Mills, Gordon B., and Yarden, Yosef (2008), ‘Derailed endocytosis: an emerging feature of cancer’, Nat Rev Cancer, 8 (11), 835-50; Schmid, Sandra L. and Mettlen, Marcel (2013), ‘Cell biology: Lipid switches and traffic control’, Nature, 499 (7457), 161-62.

FIG. 14: Endocytic mechanisms underlying tumor cell migration and invasion through tissue barriers. The diagram presents a motile cell, the advancing lamellipodium of which moves directionally (arrow). Focal adhesions (FAs) are schematically shown, and integrin heterodimers are present at these. Cell migration necessitates polarized endocytosis and trafficking of FA complexes. Integrin internalization is controlled by dynamin, which is activated by microtubules (not shown), and protein kinases, such as FAK and protein kinase Cα (PKCα). Both clathrin- and caveolin 1 (CAV1)-coated domains of the plasma membrane are involved in internalization of integrin. Once in early endosome (EE), integrins may be sorted for degradation in lysosomes, recycled to the plasma membrane through a RAB4-dependent route, or transported to the perinuclear recycling compartment (PNRC). Recycling from the PNRC requires Rab 11 family members, such as RAB25, and for some integrin heterodimers, also the protein kinase B (PKB)—GSK3β (glycogen synthase kinase-β) axis, ARF6 or certain isoforms of PKC. Human tumors often aberrantly express RAB25, display a specific repertoire of growth factor-induced integrin heterodimers or present abnormally stabilized microtubules, which promote trafficking of integrins. FAK, Integrin, RAB25, and PKB have functions associated with oncogenesis and/or display aberrant expression in human tumors. Modified from Mosesson, Yaron, Mills, Gordon B., and Yarden, Yosef (2008), ‘Derailed endocytosis: an emerging feature of cancer’, Nat Rev Cancer, 8 (11), 835-50.

FIG. 15: EEC in Alzheimer's disease (AD). APP is synthesized in the ER, transported to the TGN, and inserted into the plasma membrane via secretory vesicles. Cell-surface APP can be internalized to endosomes from which it can either be recycled back to the cell surface or delivered to lysosomes for degradation. Within the EE, the acidic environment favors production of Aβ, which can be degraded in lysosomes by cathepsins, accumulated in EEs, or released to extracellular spaces via exocytosis. Modified from Chen, X., et al. (2014), ‘Altered Cholesterol Intracellular Trafficking and the Development of Pathological Hallmarks of Sporadic AD’, J Parkinsons Dis Alzheimers Dis, 1 (1).

FIG. 16: EEC in Parkinson's disease (PD) Following endocytic entry, cargo is transported to early endosomes. From there, cargo can recycle back to the plasma membrane, either directly or via recycling endosomes. Alternatively, cargo can be retained in the EEs, which will form LEs/MVBs, and fuse with lysosomes for degradation. In parallel, cargo are transported between EEs and the trans-Golgi network (TGN). Alterations in these processes lead to dysfunctional lysosomes and accumulation of undegraded macromolecules, toxic to the cell. Adapted from Schreij, A. M., Fon, E. A., and McPherson, P. S. (2015), ‘Endocytic membrane trafficking and neurodegenerative disease’, Cell Mol Life Sci.

FIG. 17: Macropinocytosis in Atherosclerosis (CAD) Following endocytic entry, LDL is transported to the lysosome for degradation. Deficiencies in the lysosomal process lead to accumulation of LDL. Macrophages (MΦs) turn into foam cells, which accumulate to form atherosclerotic plaques.

FIG. 18A-C: Lysosomal Dysfunction in Cancer (A), AD/PD (B, and CAD (C). Published results show overlapping genetic risk factors for lysosomal dysfunction across diseases, leading to reduced lysosomal clearance ( [symbol] ) and, in cancer, increases in recovery of integrins from the lysosome.

FIG. 19: Viruses “Hijacking” Endocytosis. Viral particles depend on clathrin-mediated endocytosis for entry into cells. CCV: clathrin-coated vesicle. Modified from FIG. 2 in (Blaising J, Polyak S J, et al. (2014) Antiviral research 107:84-94).

FIG. 20: MβCD in AD Mouse Cells. MβCD (MbetaCD) caused an increase in the levels of α secretase cleavage products C83 (A) and a decrease in the levels of intracellular APPsβ (A and B). APPsα levels were increased in the medium from MβCD-treated cells, while APPsβ levels did not change (C and D). (adopted from (Cole S L, Grudzien A, et al. (2005) J Biol Chem 280:18755-70)).

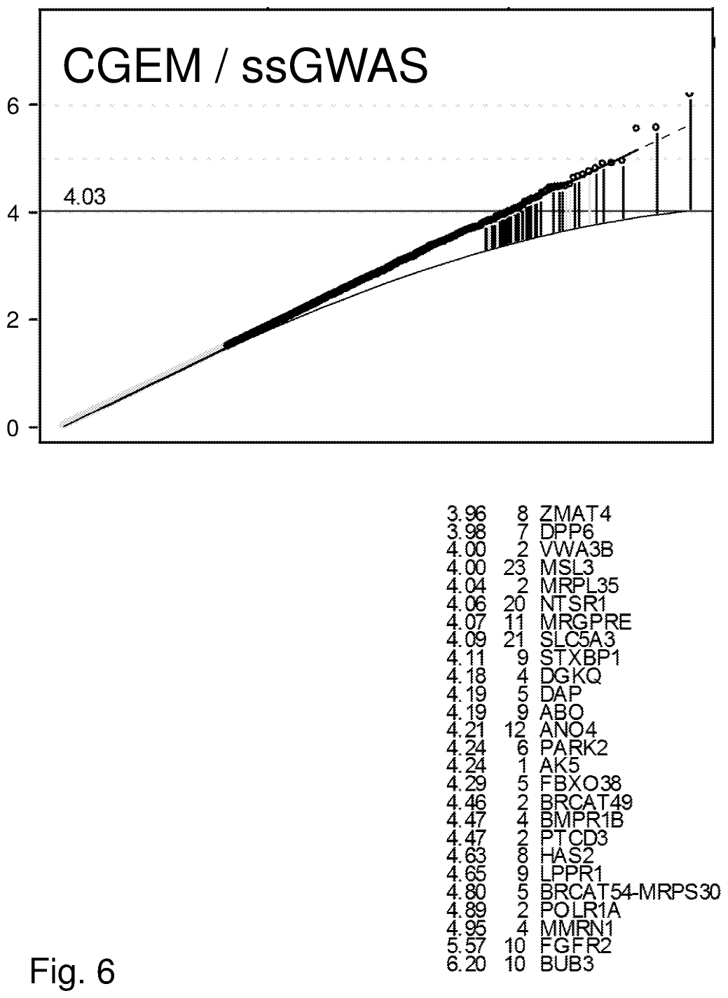

FIG. 21: Error! Reference source not found. Clearance of a-Syn. Error! Reference source not found.-mediated clearance of α-syn aggregates does not depend on the ability of Error! Reference source not found. to alter cholesterol levels. H4/α-syn-GFP cells untreated or treated with Error! Reference source not found. (1 mM) or Error! Reference source not found.-cholesterol complex (1 mM) for 24 h. Representative images are reported. FIG. 21A) Immunofluorescense microscopy analyses of TFEB subcellular localization using a FLAG-specific anti-body. Scale bar represents 10 μm. FIG. 21B) α-syn-GFP fluorescense microscopy analyses. Scale bar represents 20 μm. Modified from (Kilpatrick K, Zeng Y, et al. (2015) PLoS One 10:e0120819) FIG. 6.

FIG. 22: Cholesterol Accumulation in Mouse Brain. Unesterified cholesterol (UC) accumulation in the brain cells of 3-week-old mice treated with different CDs. Sample fluorescence photomicrographs of dorsal neocortex from untreated stained with filipin to detect UC. Cortical layers are marked by roman numerals. Virtually, all neurons in untreated Npc1^{-/-} mice show positive cytoplasmic staining of UC (white spots), where as those in Wt mice are negative. HPβCD shows highly effective reduction in UC storage. HPαCD UC storage, albeit clearly reduced, was reported by the authors as “grossly equivalent to untreated mice”. Modified from (Davidson C D, Fishman Y I, et al. (2016) Ann Clin Transl Neurol 3:366-80, FIG. 1).

FIG. 23: CDs in Lysosomal Storage Diseases. Effect of cyclodextrins using lysotracker red staining to indicate enlarged lysosomes in ML111 fibroblasts. Wolman (LAL), TSD, Fabry (GLA), Farber (AC), MPSIIIB: mucopolysaccharidose II type B (NAGLU), NPA: Nieman-Pick type A (ASM), HBCD: HPβCD (Kleptose) According to the inventors, “the other [cyclodextrins] did not have that profound an effect”,(McKew J, Zheng W E I, et al. (2014)) It was overlooked, however, that, the effect of 6 mM Error! Reference source not found. (1/5 of the MBCD dose) was more profound than the effect of 8 mM HPβCD. Modified from (McKew J, Zheng W E I, et al. (2014)).

FIG. 24: Pino-/Phago-/Endocytosis: Macropinocytosis, phagocytosis, and endocytosis are regulated in the same fashion by the PI-cycle and lysosomal dysfunction in all three processing pathways causes substrates with unwanted function to be excreted. See Sole-Domenech, S., et al. (2016), ‘The endocytic pathway in microglia during health, aging and Alzheimer's disease’, Ageing Res Rev, 32, 89-103, FIG. 1.

FIG. 25: HPβCD treatment activating autophagy. HPβCD activates autophagy. Administration of Error! Reference source not found. results in activation of transcription factor EB TFEB. Upon translocation from the cytoplasm to the nucleus, TFEB regulates the expression of genes involved in biogenesis and fusion of lyso- and autophago-somes. As a result, Error! Reference source not found. administration results in enhanced clearance of the autophagy substrate ceroid lipo-pigment. The mechanism by which Error! Reference source not found. improves TFEB activation, however, was not understood (adapted from (Song W, Wang F, et al. (2014) J Biol Chem 289:10211-22)).

FIG. 26: Activation of TFEB by HP-beta-Cycoldextrin.

FIG. 27: Phago-/Endocytosis in Multiple Sclerosis (MS). The vicious cycle in the etiology of MS starts with microglia phagocytosing probes of myelin from the healthy myelin sheath. If the lyosome (LY) is losing its ability to discard all of these probes, some of them are presented as antigens to T- and B-cells, which then cross the blood-brain barrier to activate peripheral MΦ. These MΦs then infiltrate the CNS and endocytose myelin for destruction. Modified from Luo, C., et al. (2017), ‘The role of microglia in multiple sclerosis’, Neuropsychiatr Dis Treat, 13, 1661-67; Salter, M. W. and Stevens, B. (2017), ‘Microglia emerge as central players in brain disease’, Nat Med, 23 (9), 1018-27. Equivalent mechanisms are involved in AD and Rett syndrome. See Prinz, M. and Priller, J. (2014), ‘Microglia and brain macrophages in the molecular age: from origin to neuropsychiatric disease’, Nat Rev Neurosci, 15 (5), 300-12.

FIG. 28: Methyl-Beta Cyclodextrin (MβCD) Restores Surface Tension. “Minimum surface tension during dynamic cycle 20 with BLES containing 27 mg/ml BLES in control CBS buffer (checkers bars), 50% w/w FFA (Oleic acid [, OA]) or (MβCD) in control CBS buffer (horizontal bars) or buffer containing 40 mg/ml (MβCD) (solid black bars). (**p≤0.01, ***p≤0.001). BLES with FFA or (MβCD) shows marked impairment, which is repaired to normal functionality in the presence of (MβCD). Cholesterol was present at very low concentrations (˜2.1%) in these experiments.” Modified from Supplementary Figure S6 in (Gunasekara L, Al-Saiedy M, et al. (2017) Journal of cystic fibrosis: official journal of the European Cystic Fibrosis Society)

FIG. 29: HaCaT Proliferation. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 30: HaCaT Caspase 3/7 Activity. Influence of cyclodextrin on Caspase 3/7 activity of spontaneously immortalized aneuploid human (HaCaT) keratinocytes. Mean values after incubation (24 h), normalized on the control and the protein content, of at least six independent measurements. W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 31: HaCaT Lactose Dehydrogenase. Influence of cyclodextrin on lactose-dehydrogenase (LDH) of spontaneously immortalized aneuploid human (HaCaT) keratinocytes. Mean values after incubation (48 h), normalized on the control and the protein content, of at least six independent measurements. W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 32: Solubility Enhancements by Hydroxypropyl-cyclodextrins (HP-CD). Enhancement of apparent solubilities of lipids by HP-CD. Bar denoted “0” represents apparent solubilities in isotonic phosphate-buffered saline; those denoted by α, β, and γ represent the solubilities in the same saline but supplemented with the respective HP-CD at 5% (w/w) concentration. Modified from: (Irie T, Fukunaga K, et al. (1992) J Pharm Sci 81:524-8), FIG. 1.

FIG. 33: Nephrotoxicity of Error! Reference source not found. and β-CD). The 2017 assessment by the EMA (and several other authors) of similar risks associated with parenteral administration of Error! Reference source not found. and β-CD) in rats is based on a single study (Frank D W, Gray J E, et al. (1976) Am J Pathol 83:367-82).

FIG. 34: Ototoxicity of CDs. Ototoxicity of different CDs as assessed by auditory brainstem response (ABR) recordings at 12 weeks of age. Plot of hearing thresholds for individual mice reveals minute variability across mice treated with a particular CD with the exception of HPγCD, in which hearing thresholds were more variable. Modified from (Davidson C D, Fishman Y I, et al. (2016) Ann Clin Transl Neurol 3:366-80, FIG. 5B).

FIG. 35: Wound healing by cyclodextrins in breast cancer cell lines. Dashed horizontal line indicates inhibition of wound healing in HPαCD at 1 and 4 mM respectively. ANOVA P values are shown for HPαCD vs HPβCD with MCF-7 and MDA-MB-231 as (fixed) blocks:

1 mM HPαCD vs 1 mM HPβCD, p=0.0001

1 mM HPαCD vs 2 mM HPβCD, p=0.0252

4 mM HPαCD vs 4 mM HPβCD, p=0.0442

(Modified from (Wittkowski K M, Dadurian C, et al. (2018) PLoS One 13:e0199012).

FIG. 36: Wound Healing Assay: Modified from Cell BioLabs Inc., Assay CBA-120.

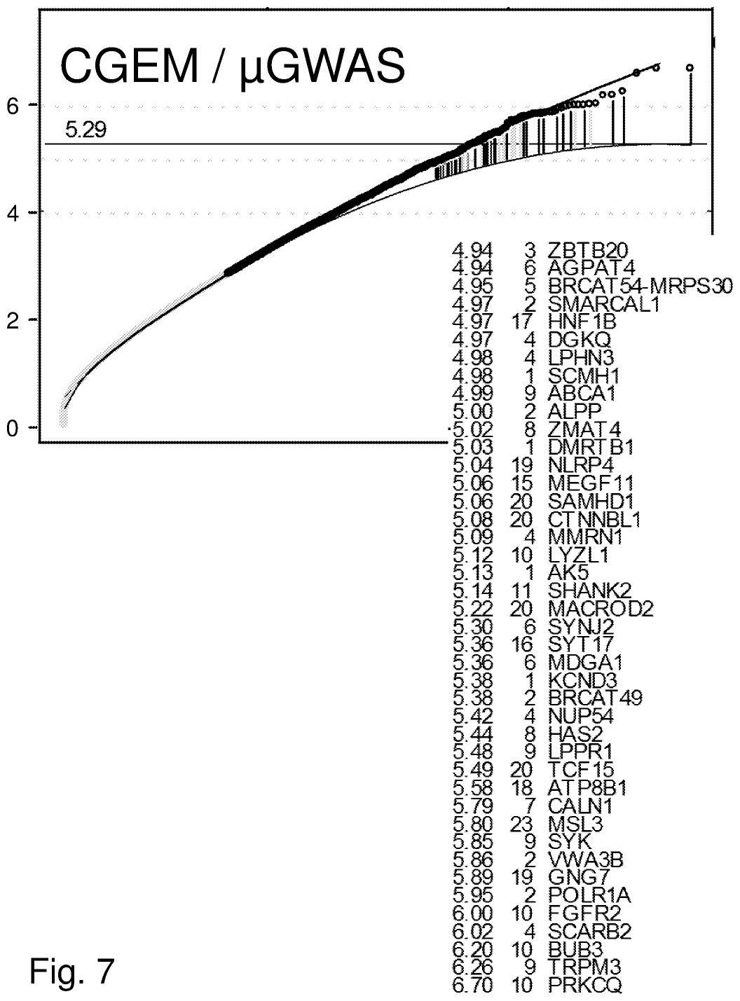

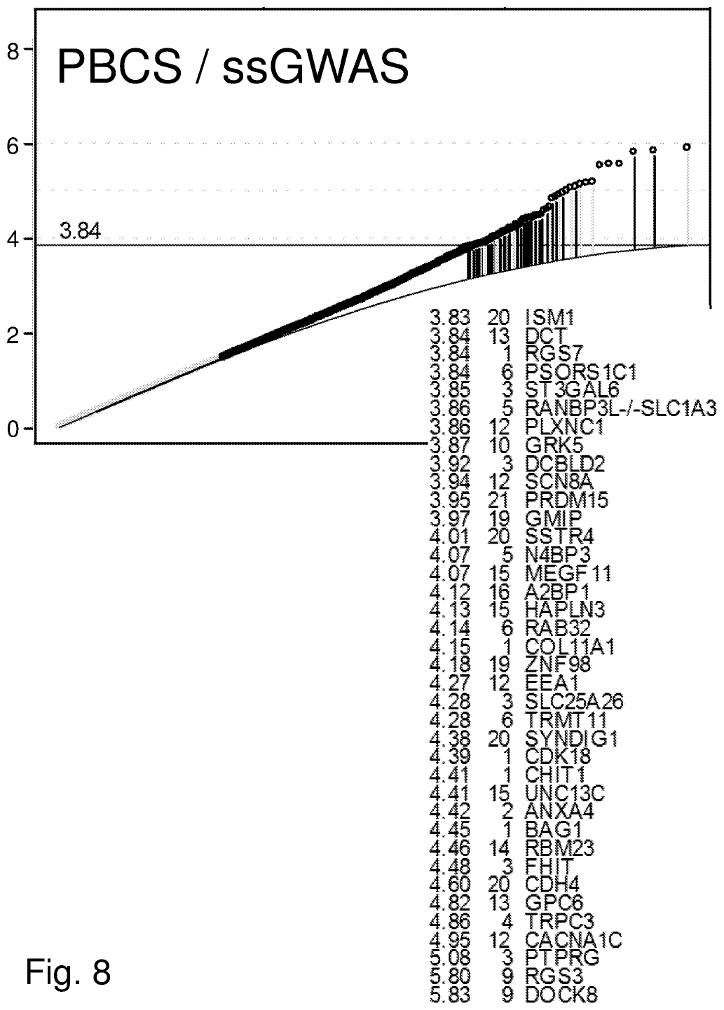

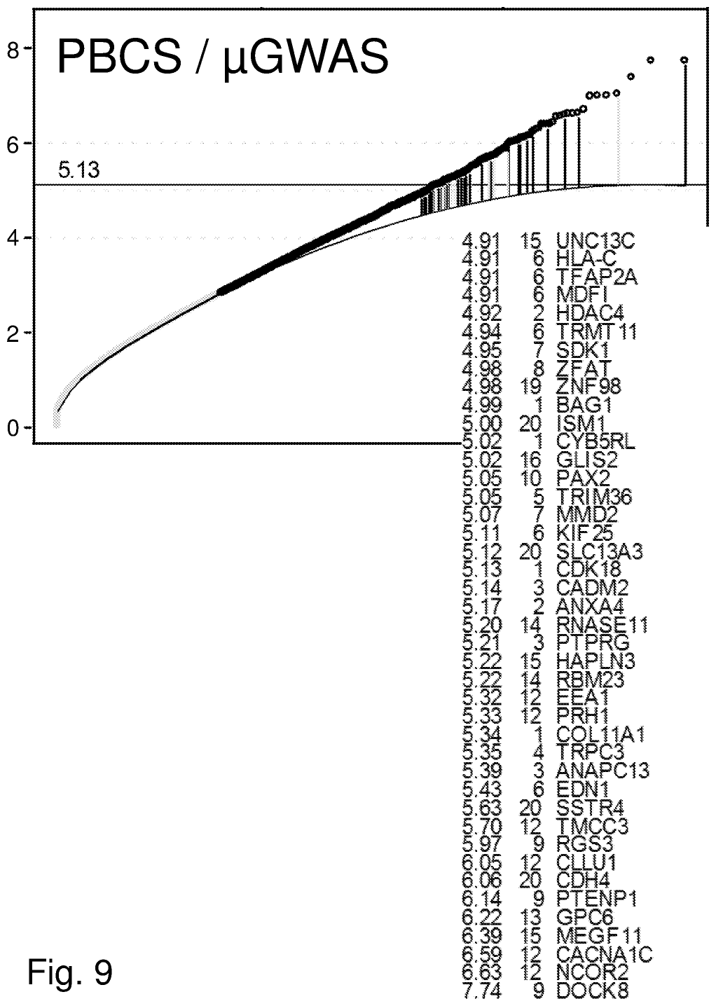

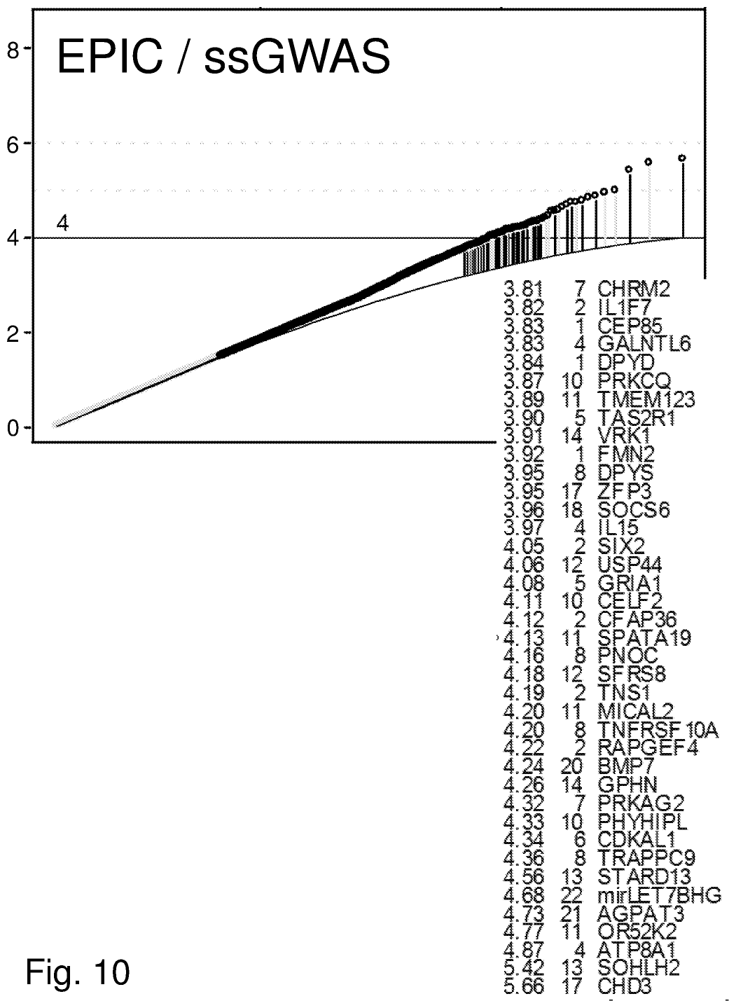

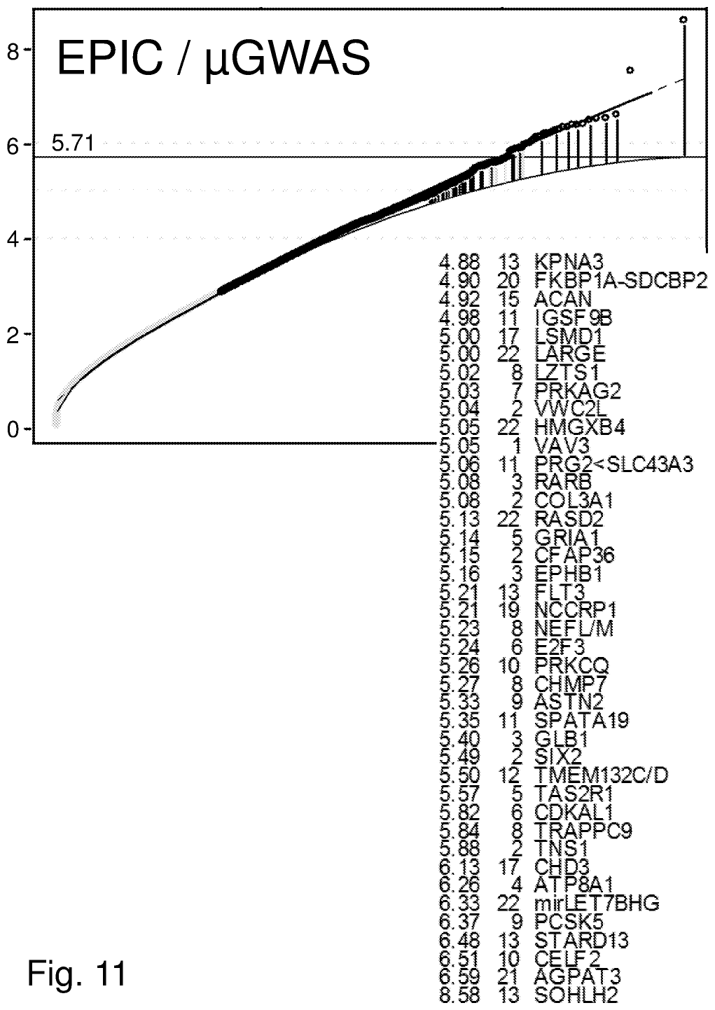

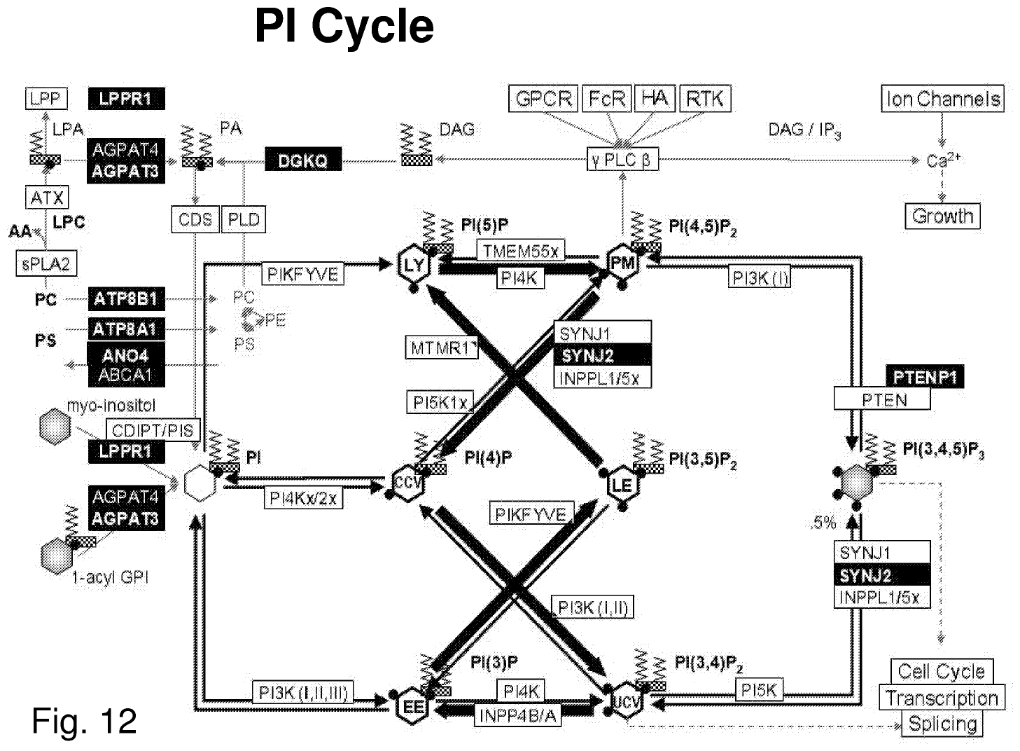

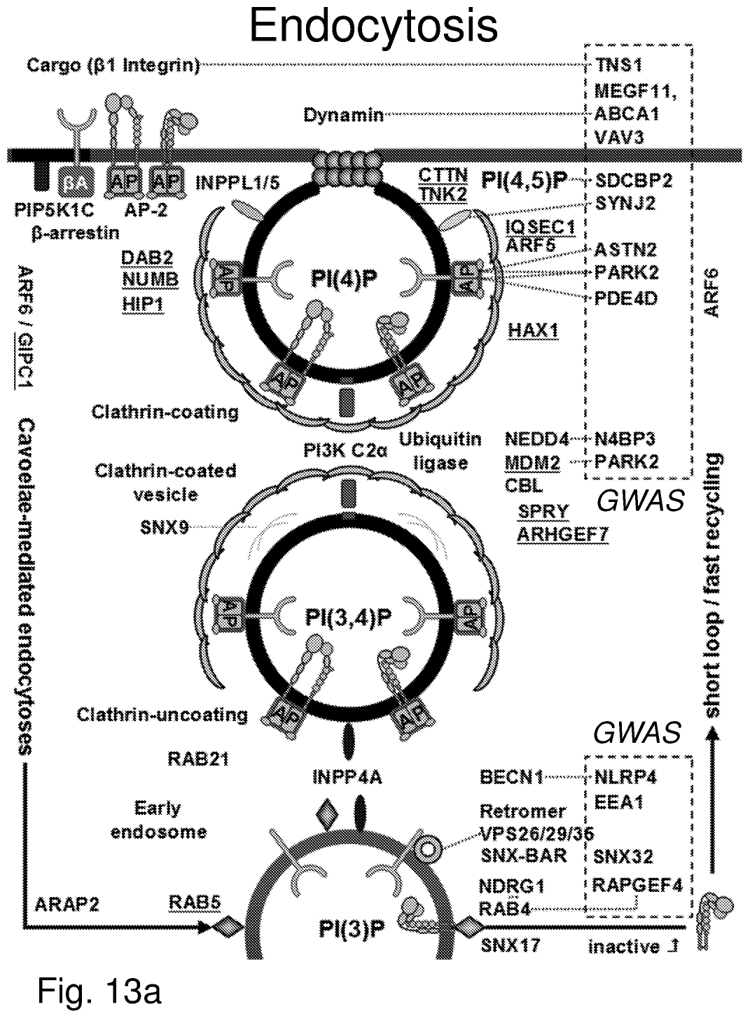

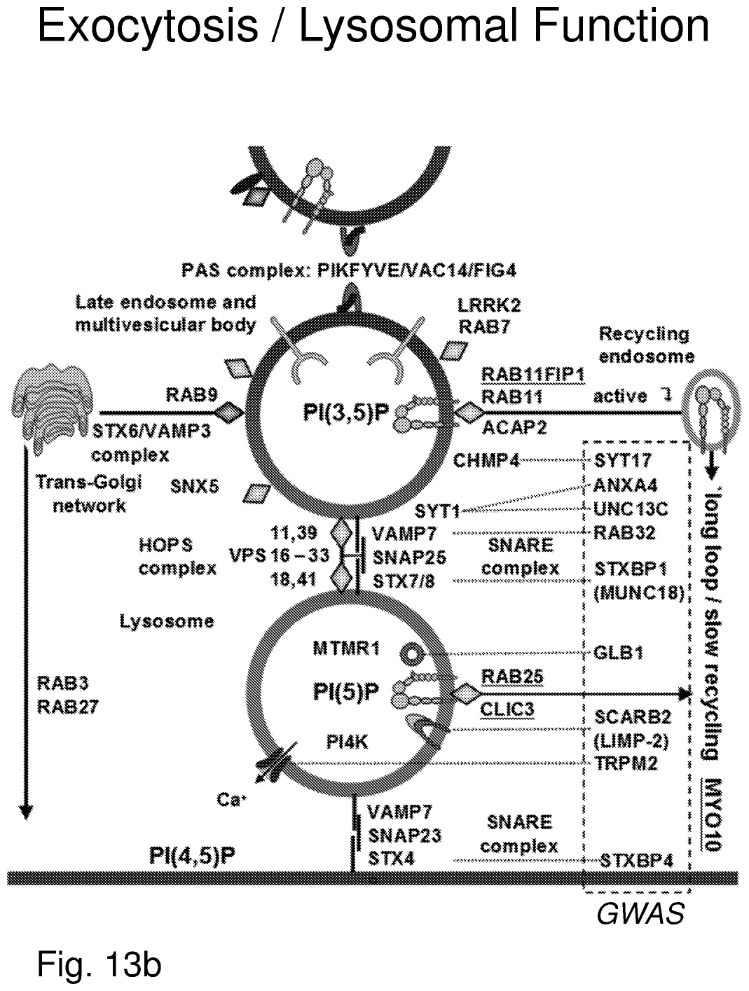

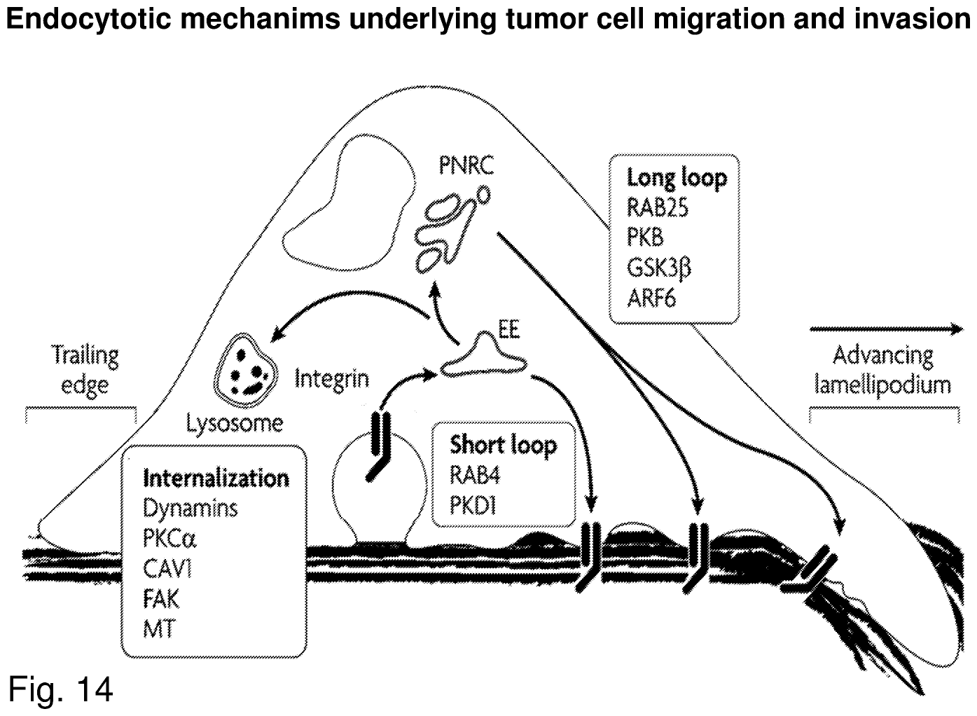

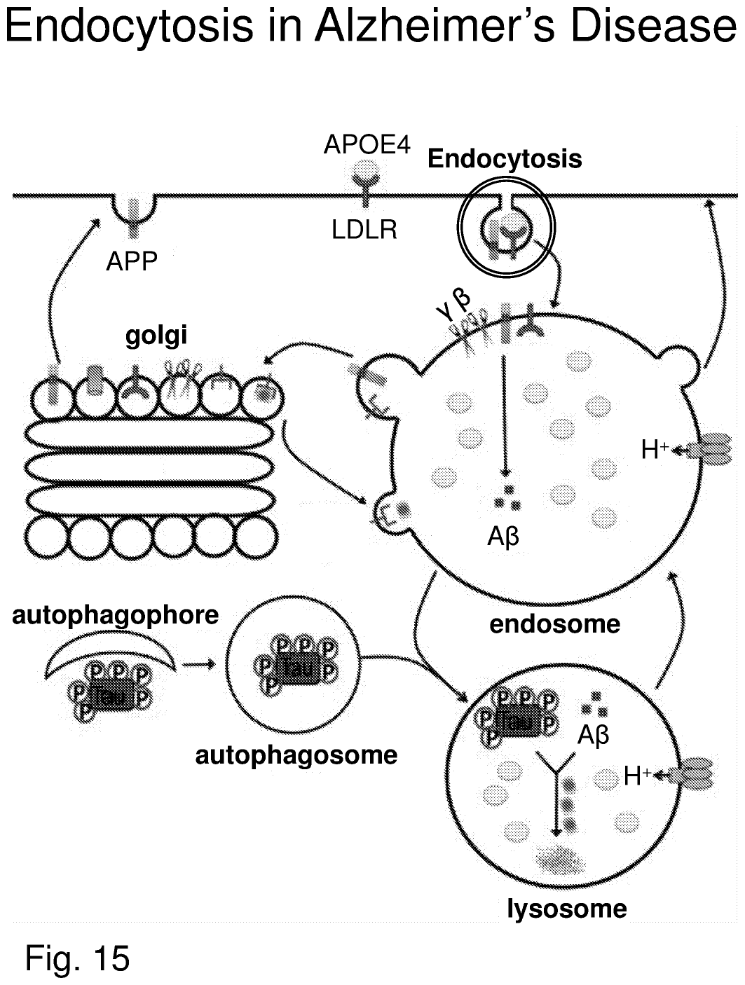

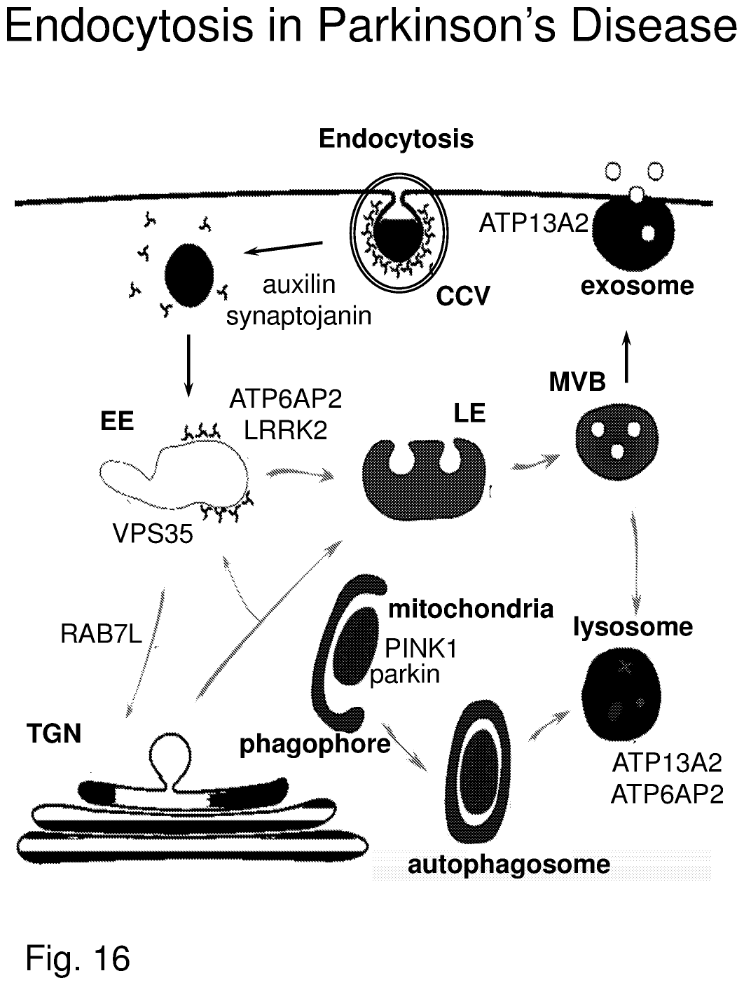

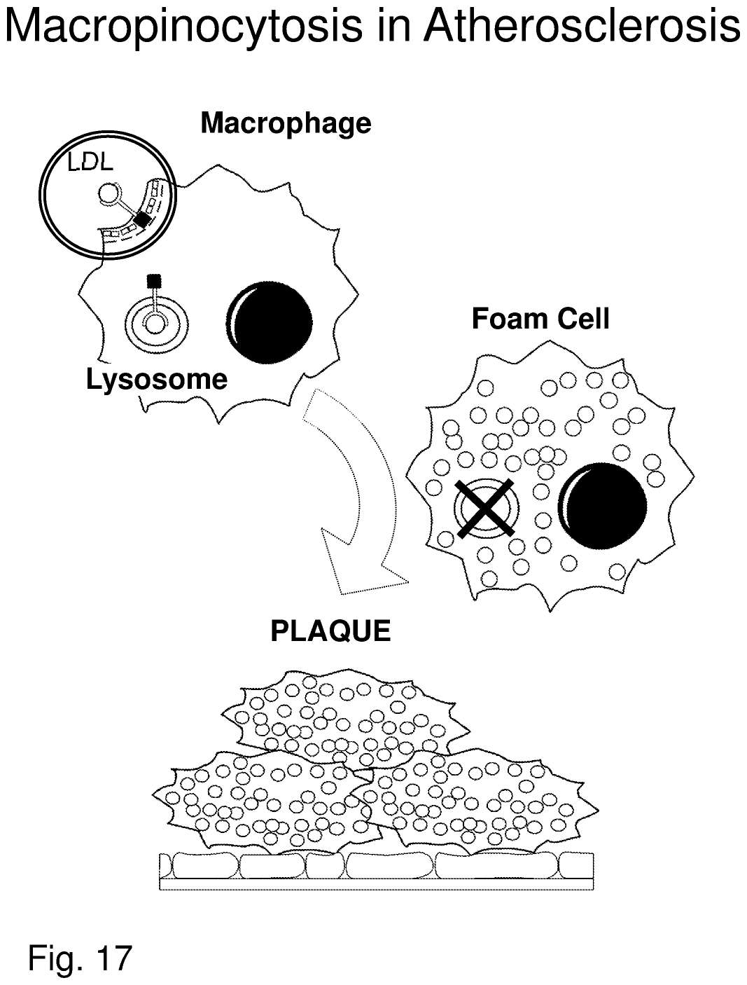

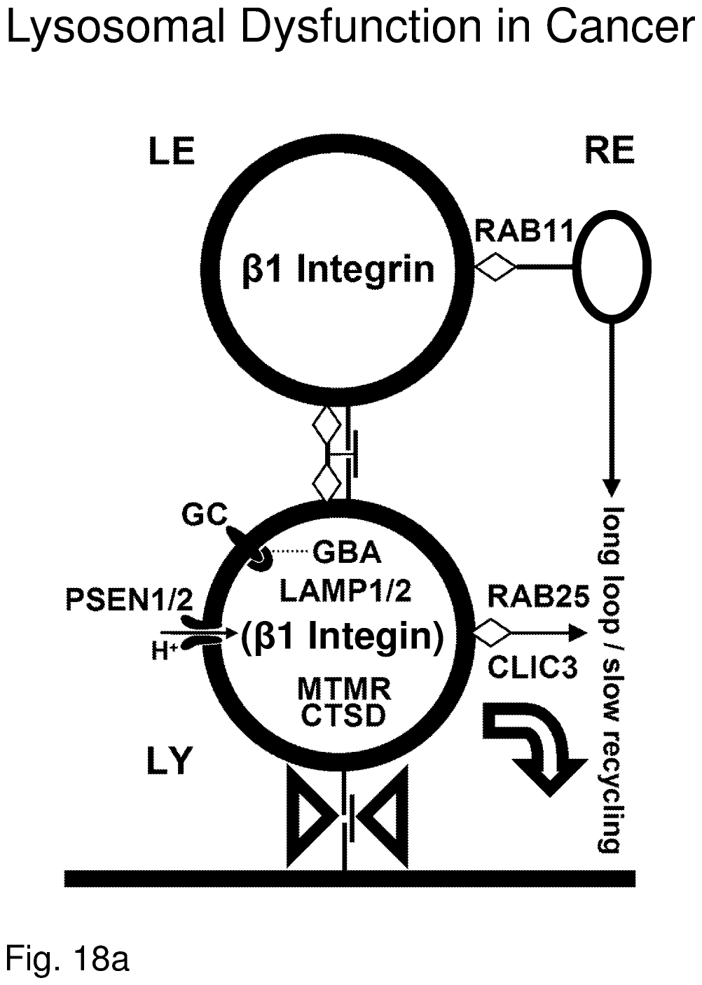

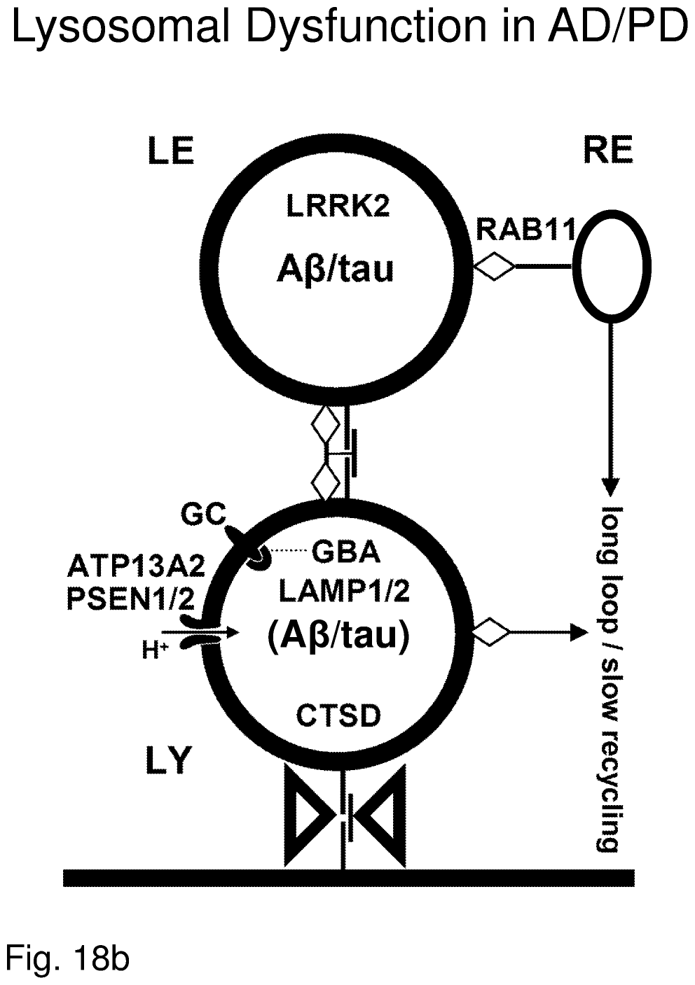

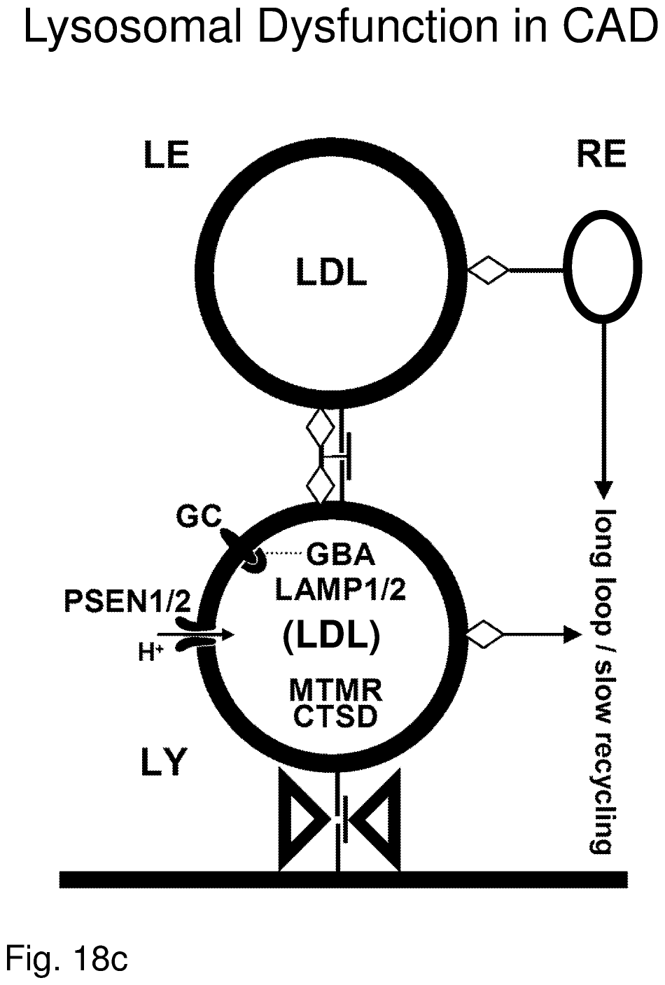

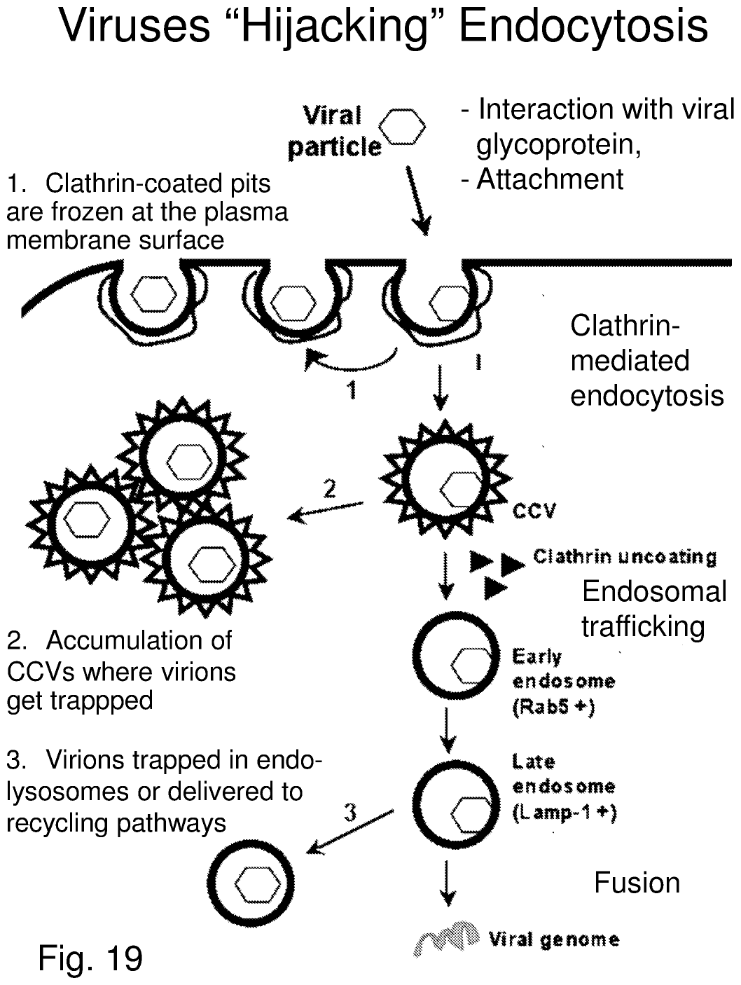

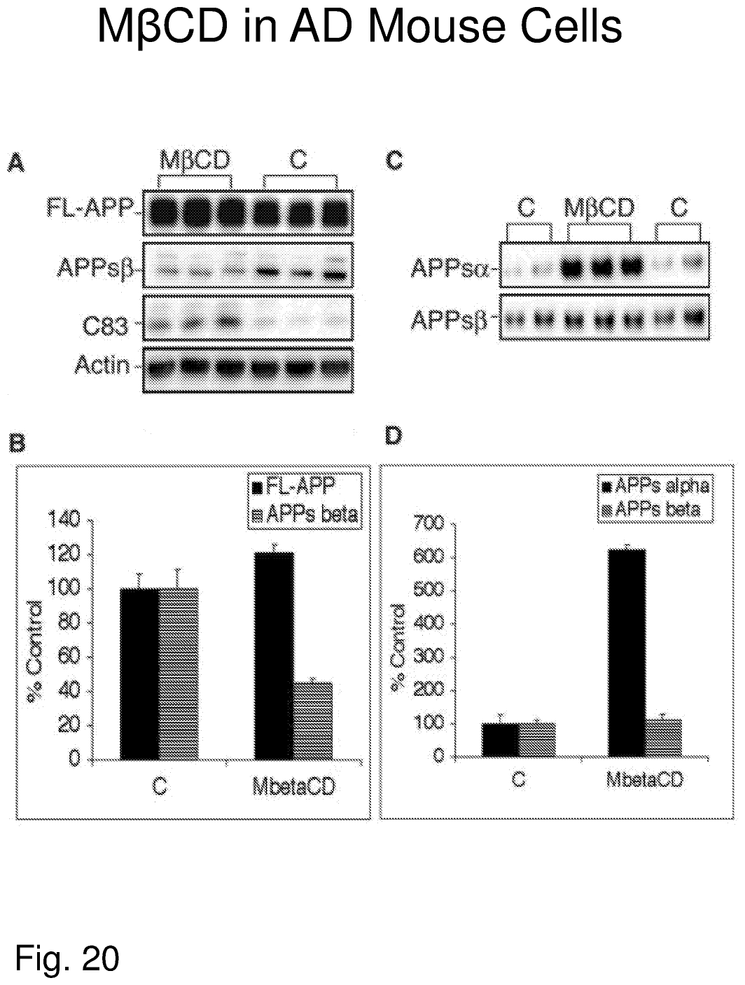

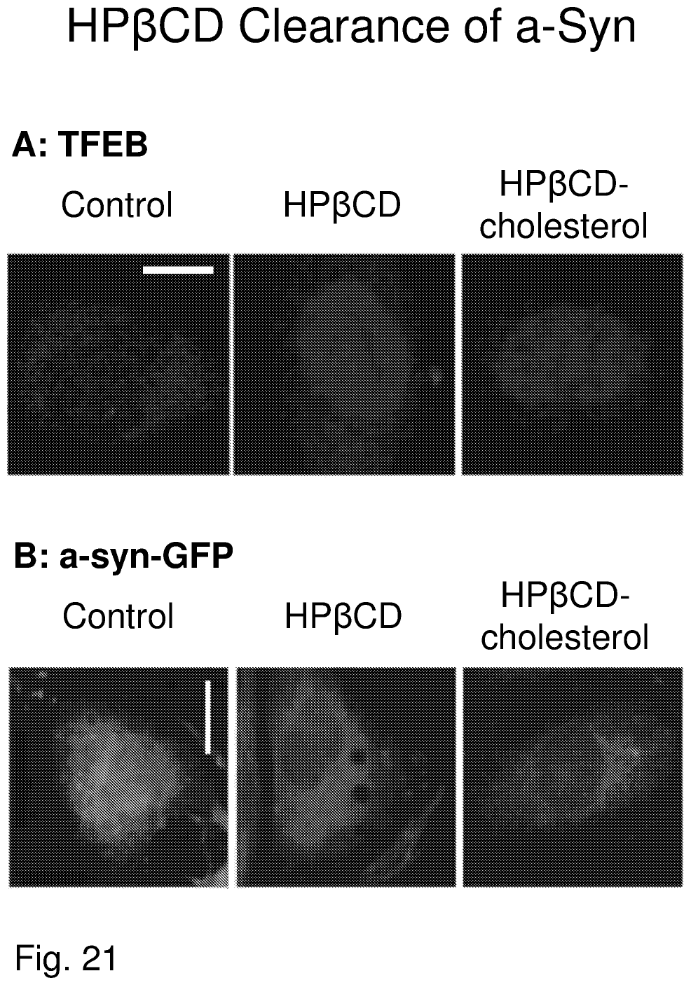

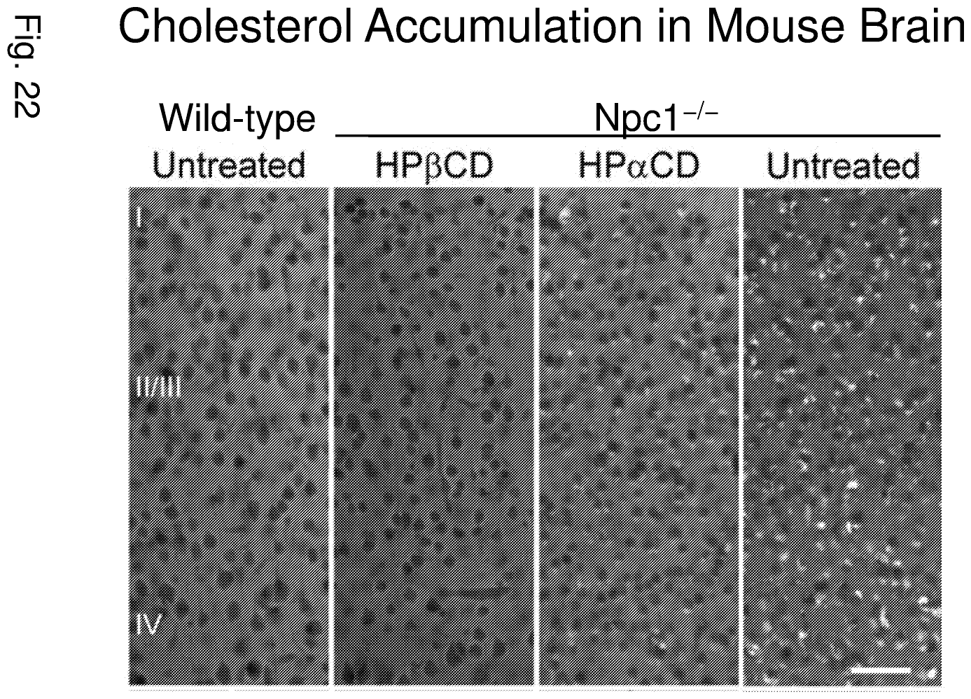

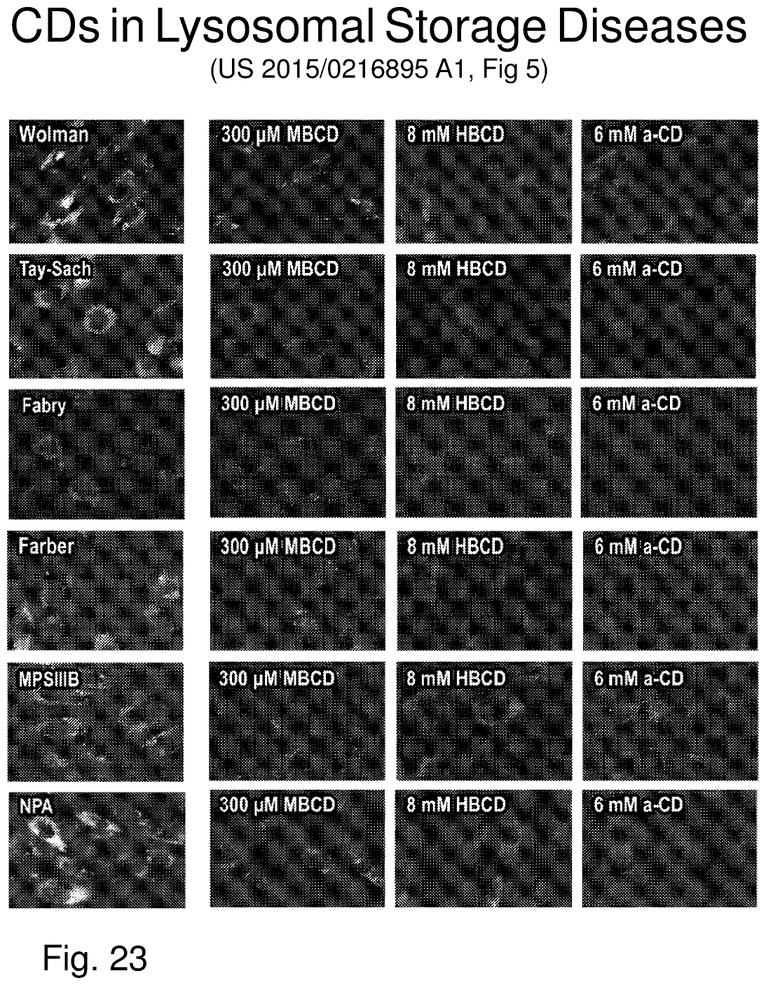

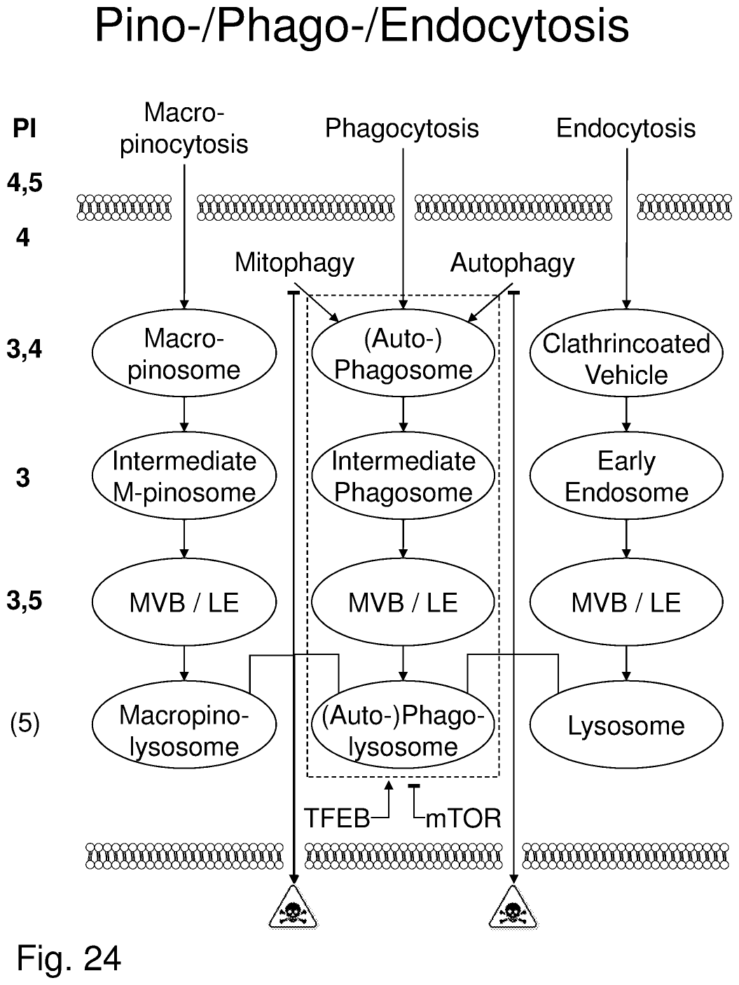

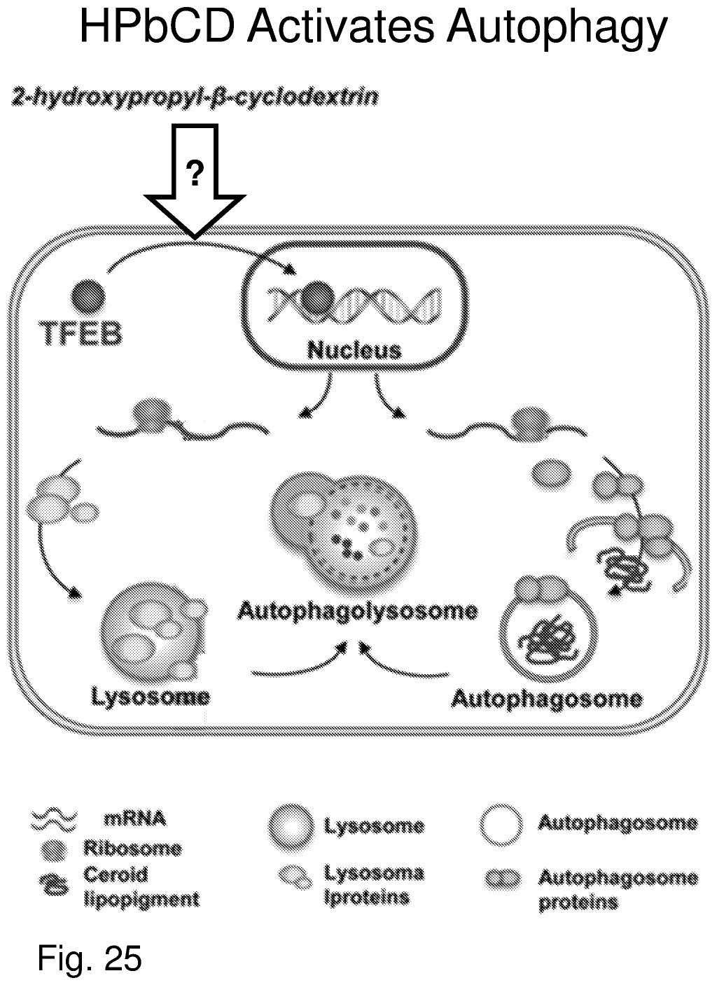

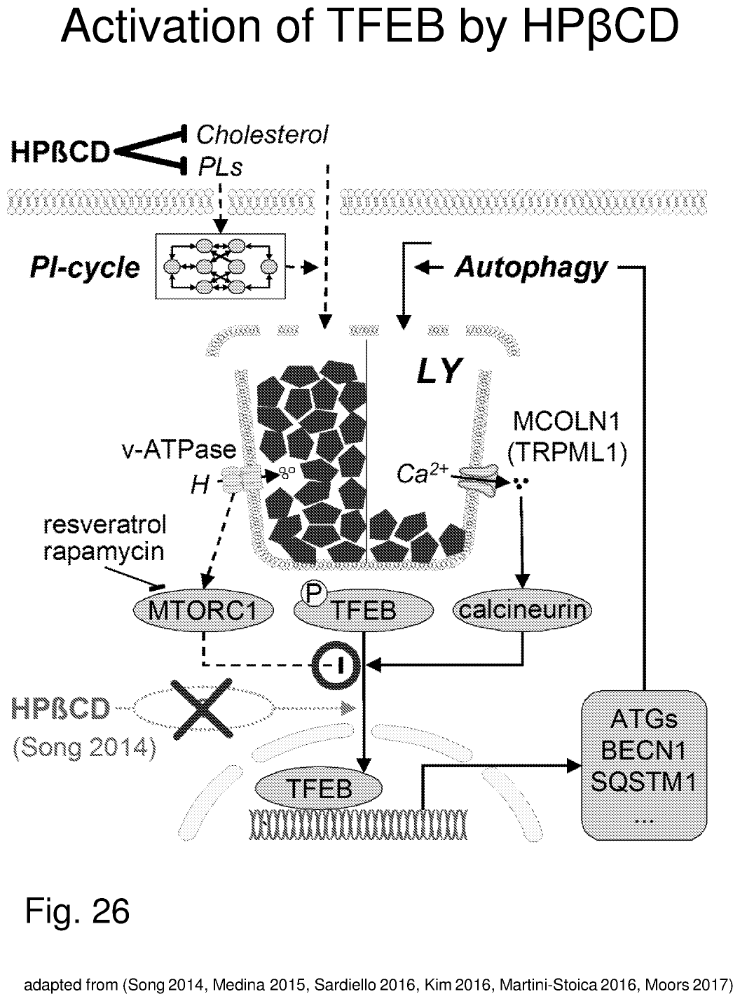

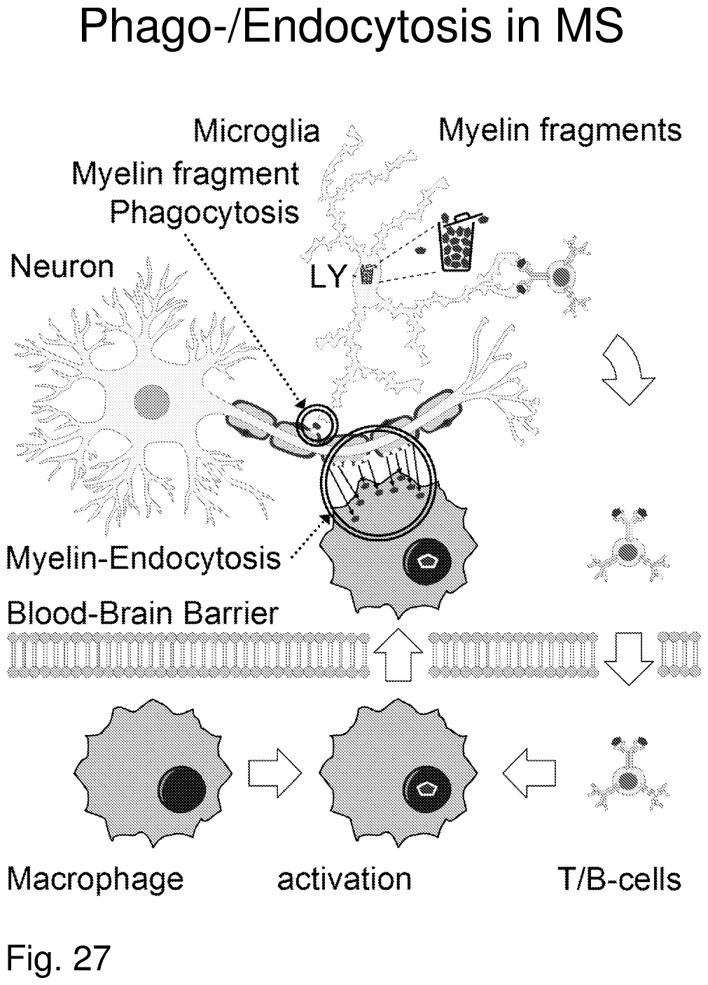

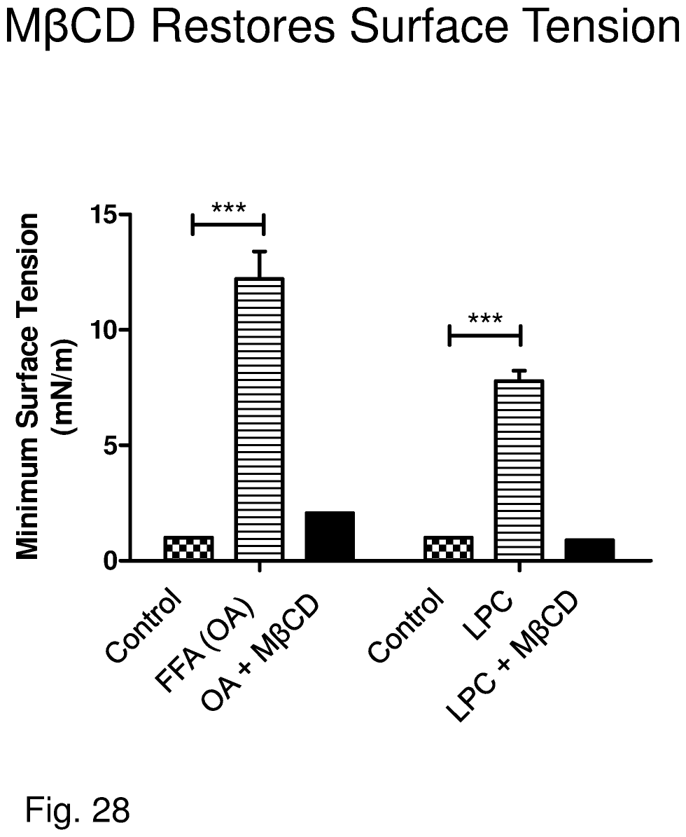

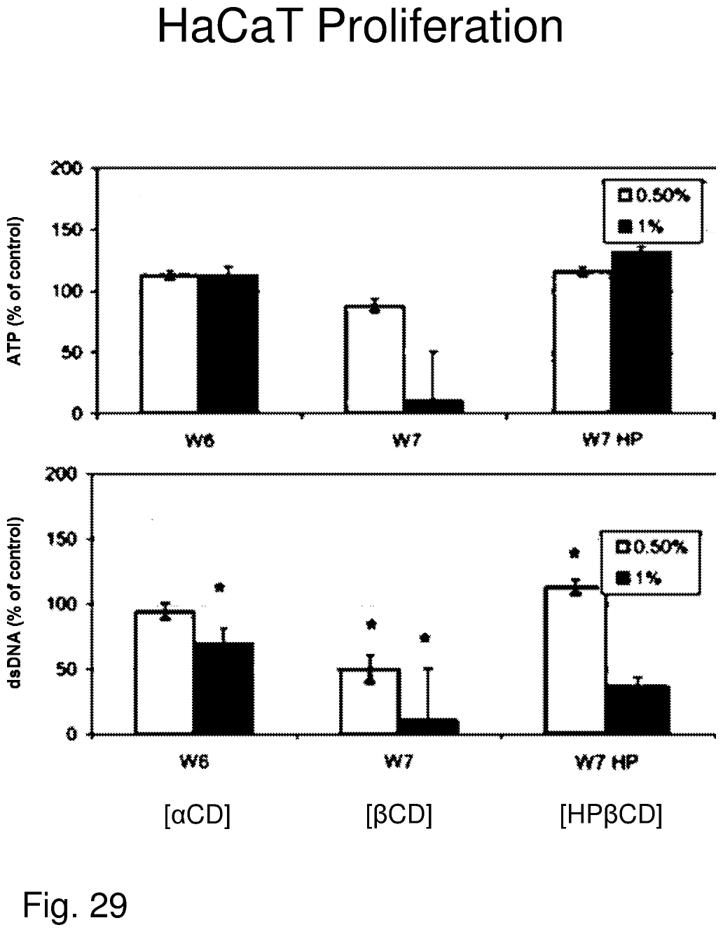

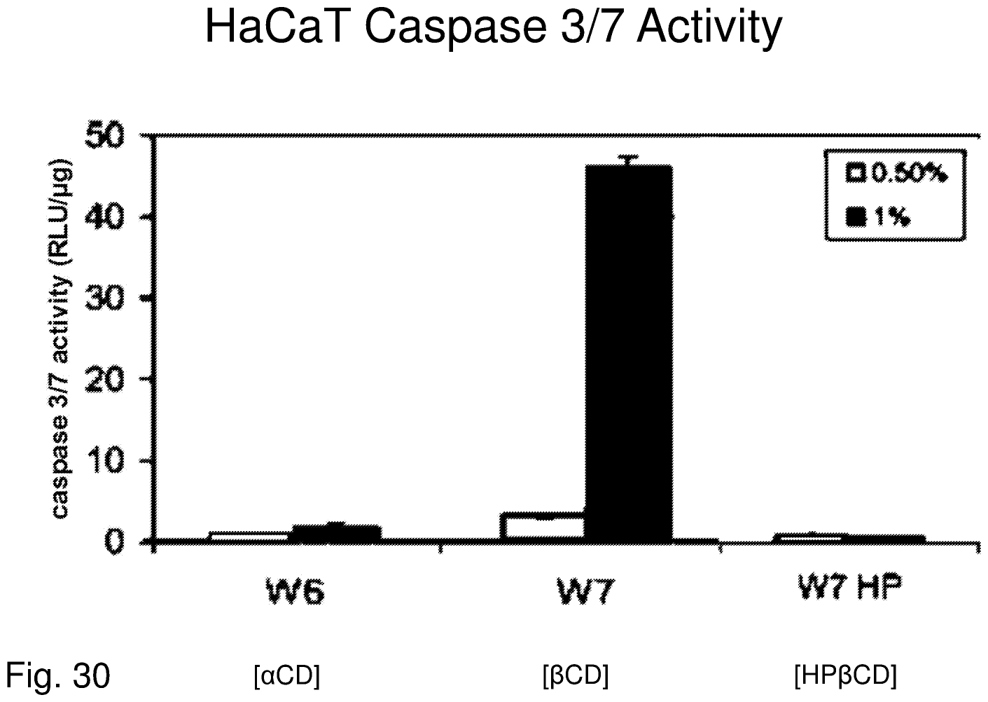

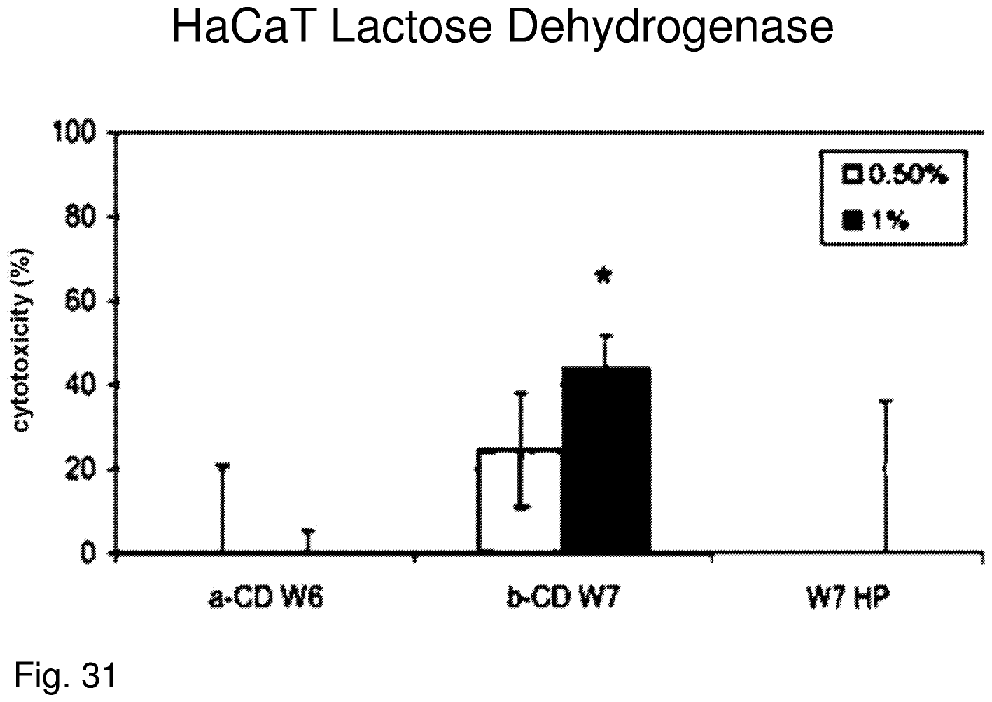

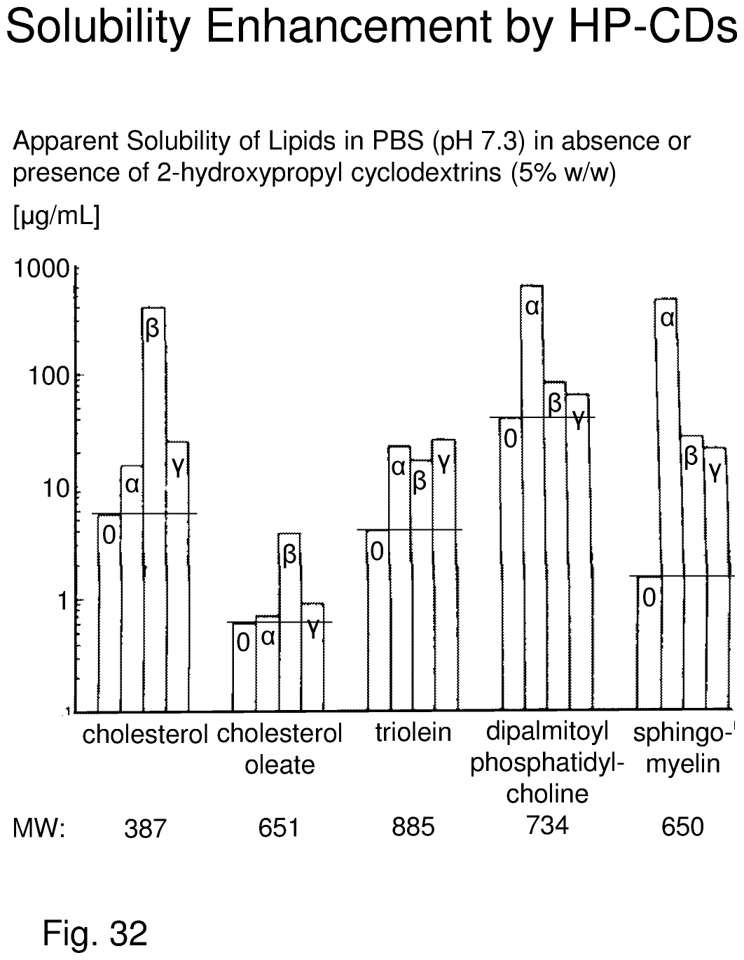

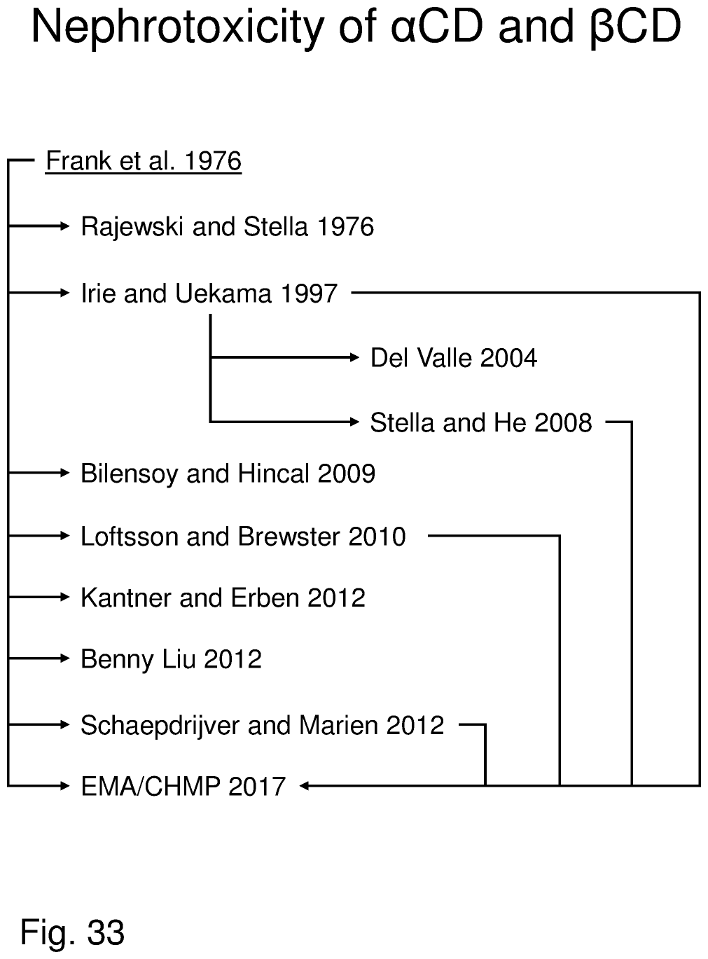

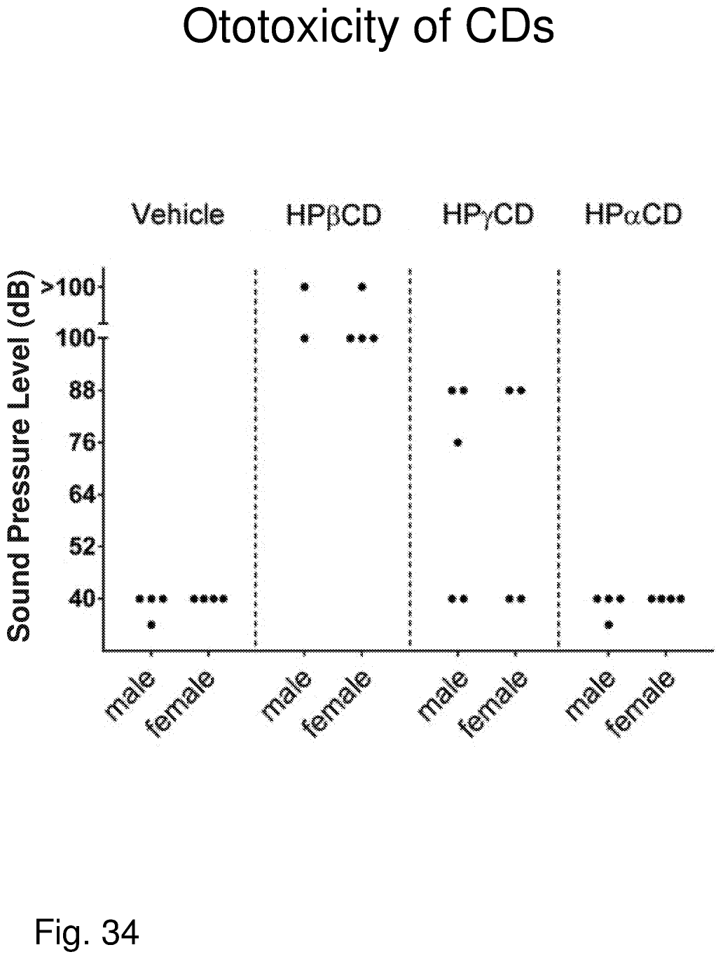

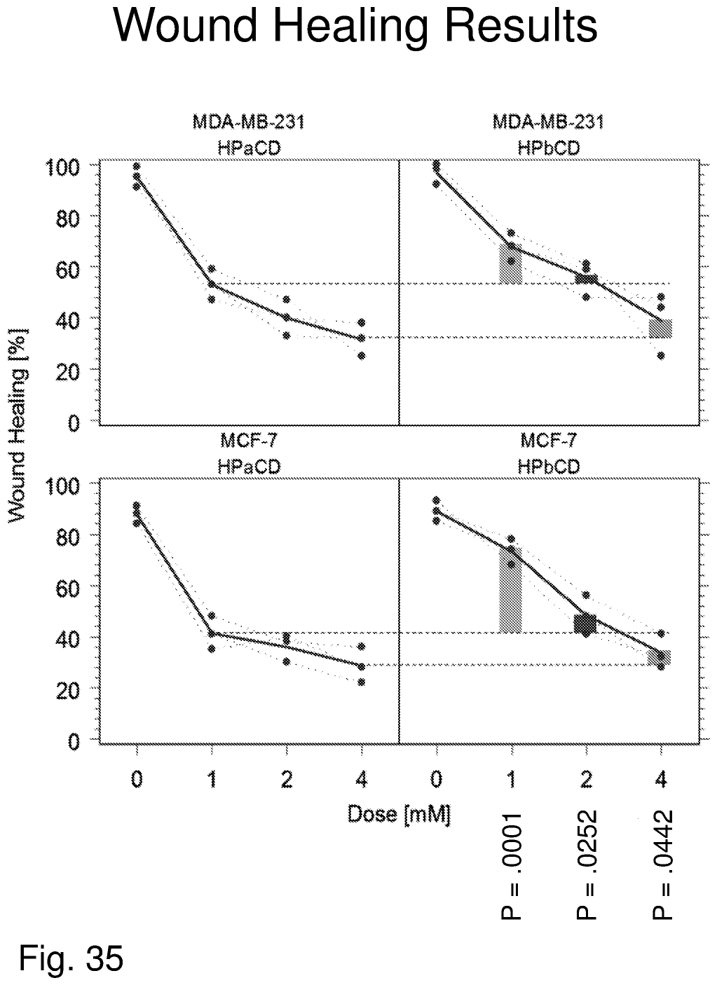

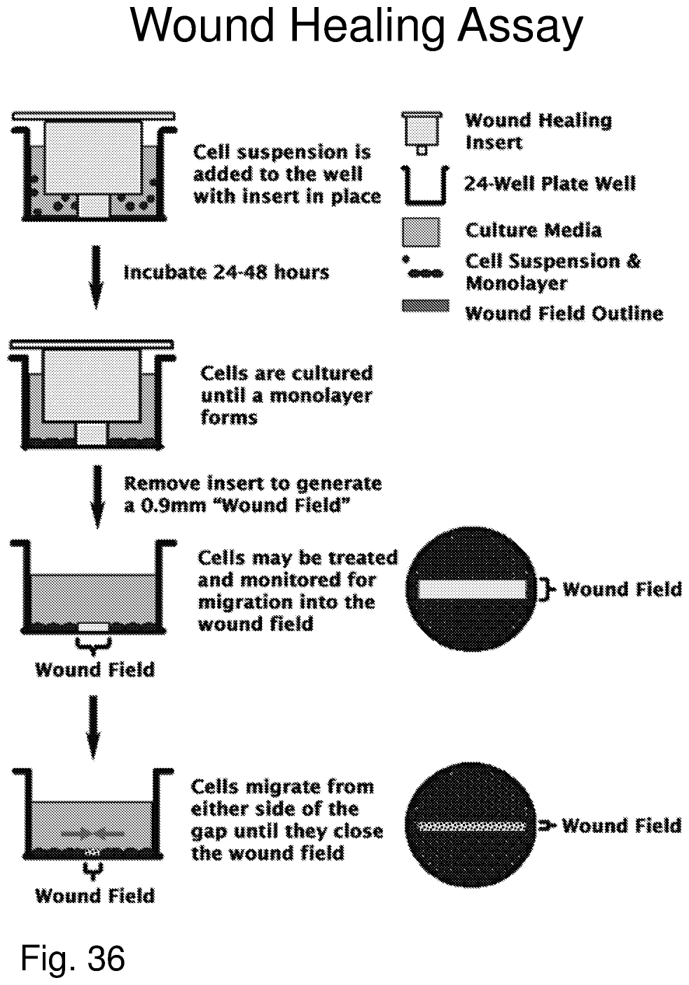

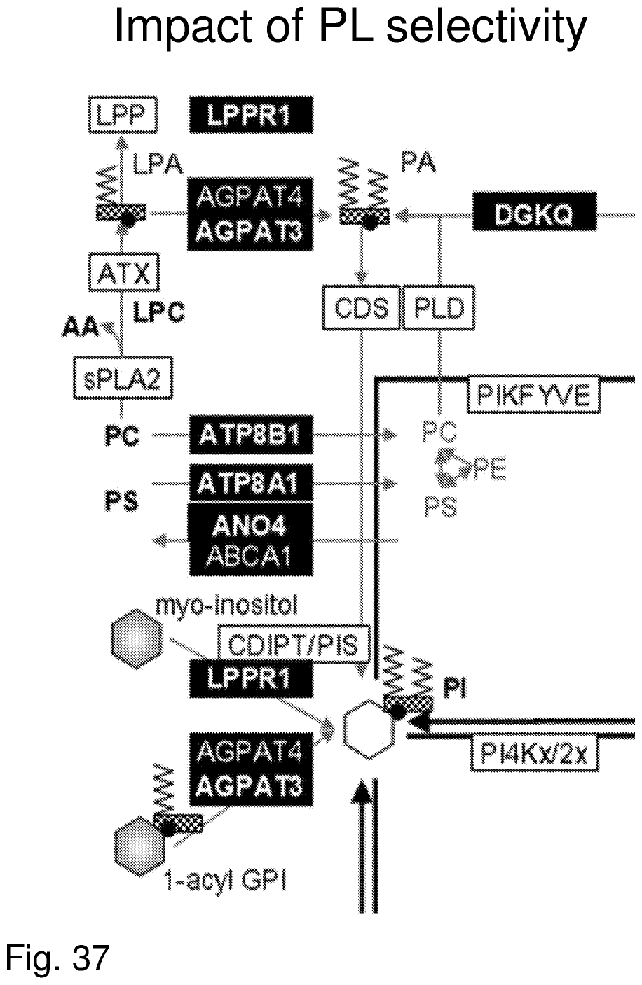

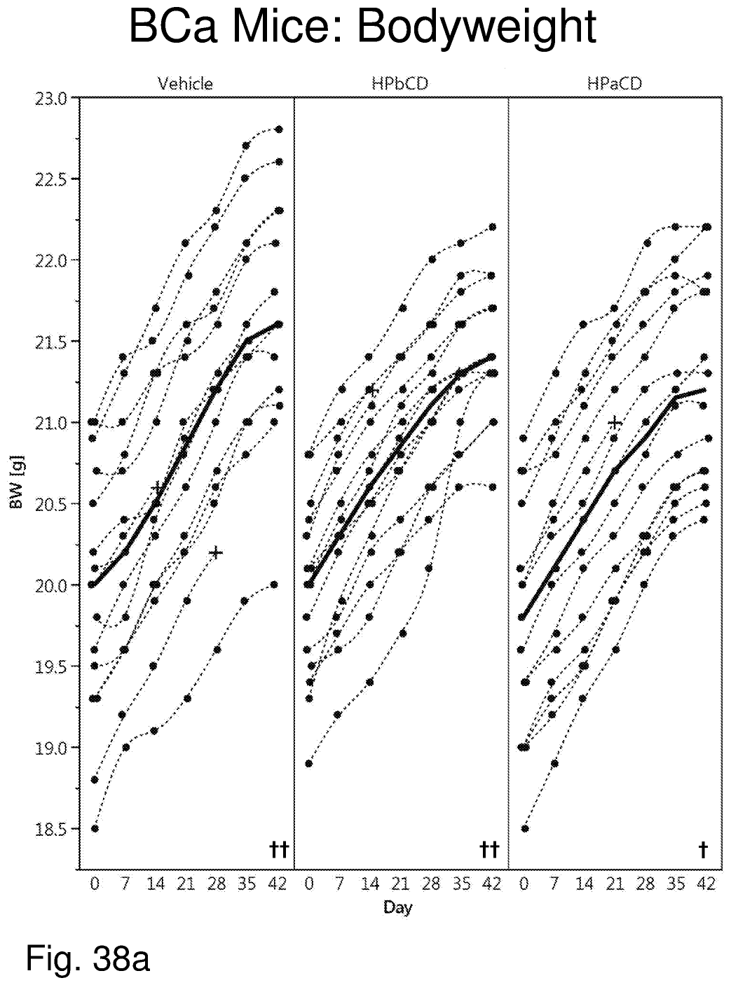

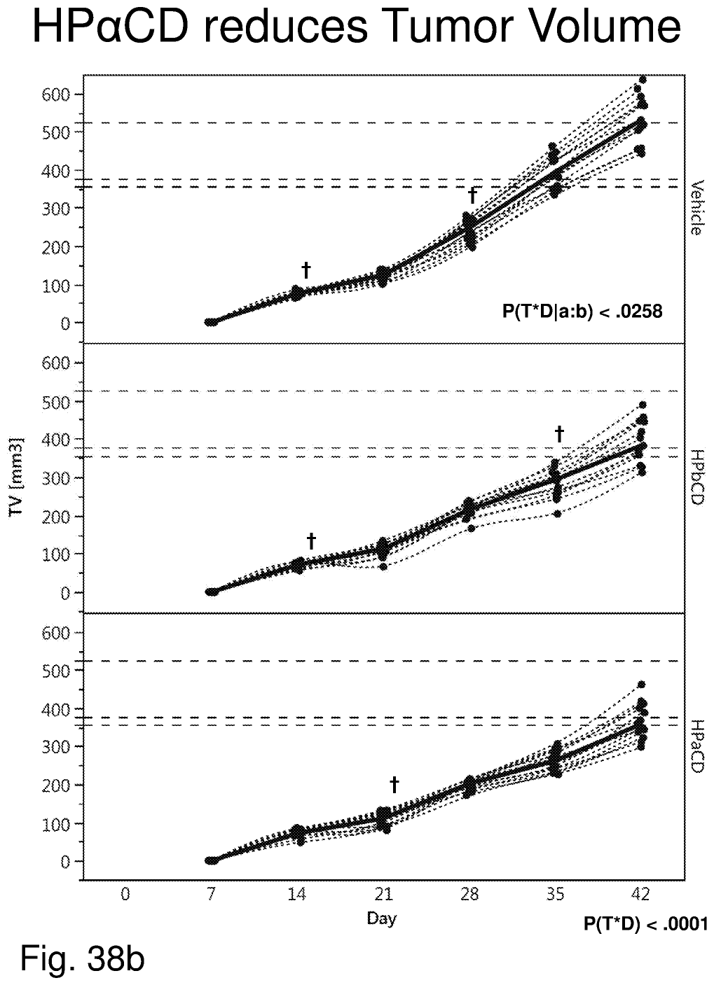

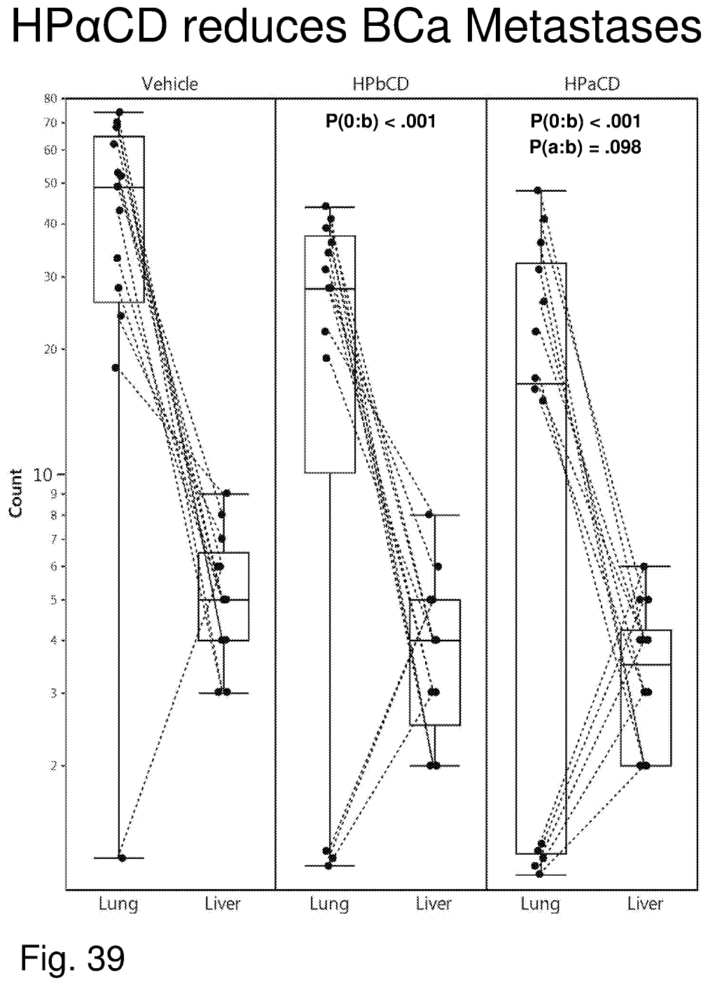

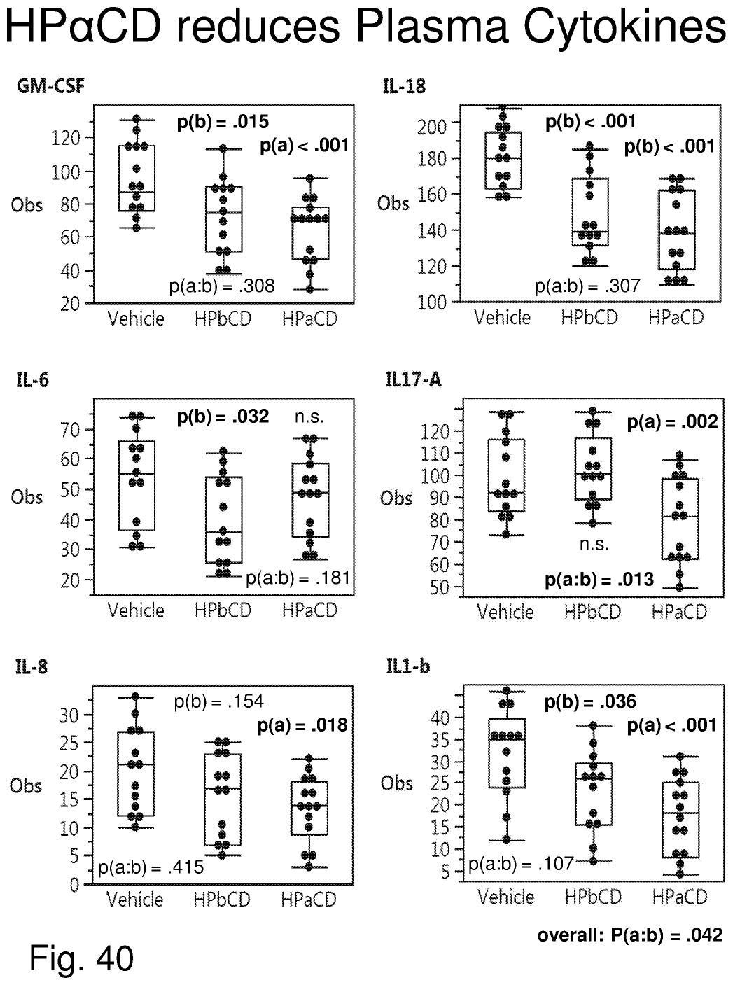

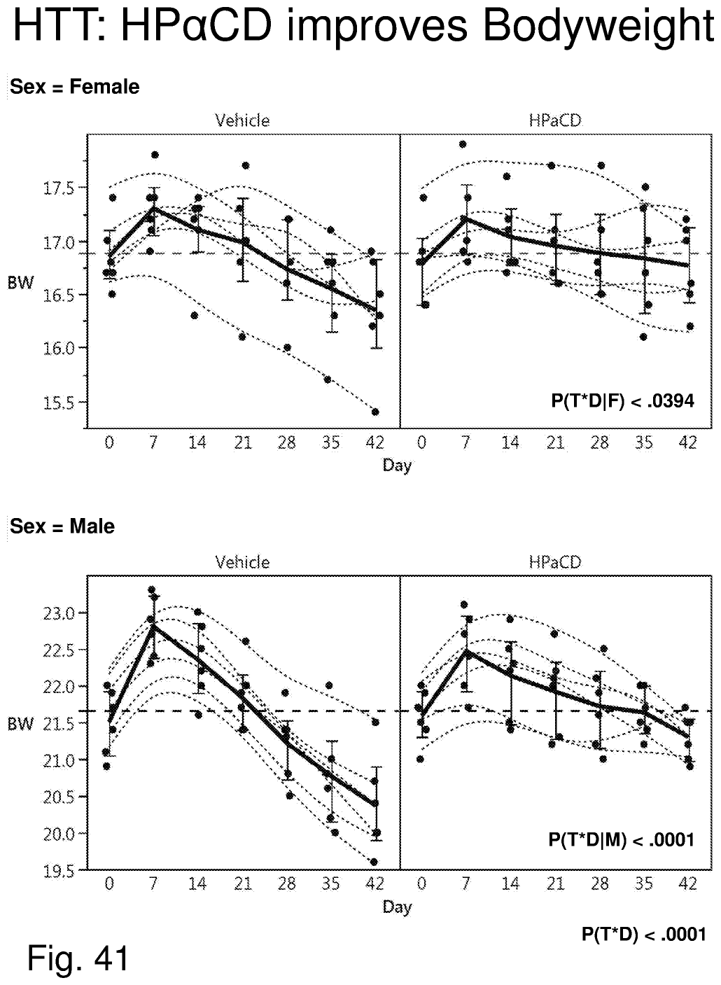

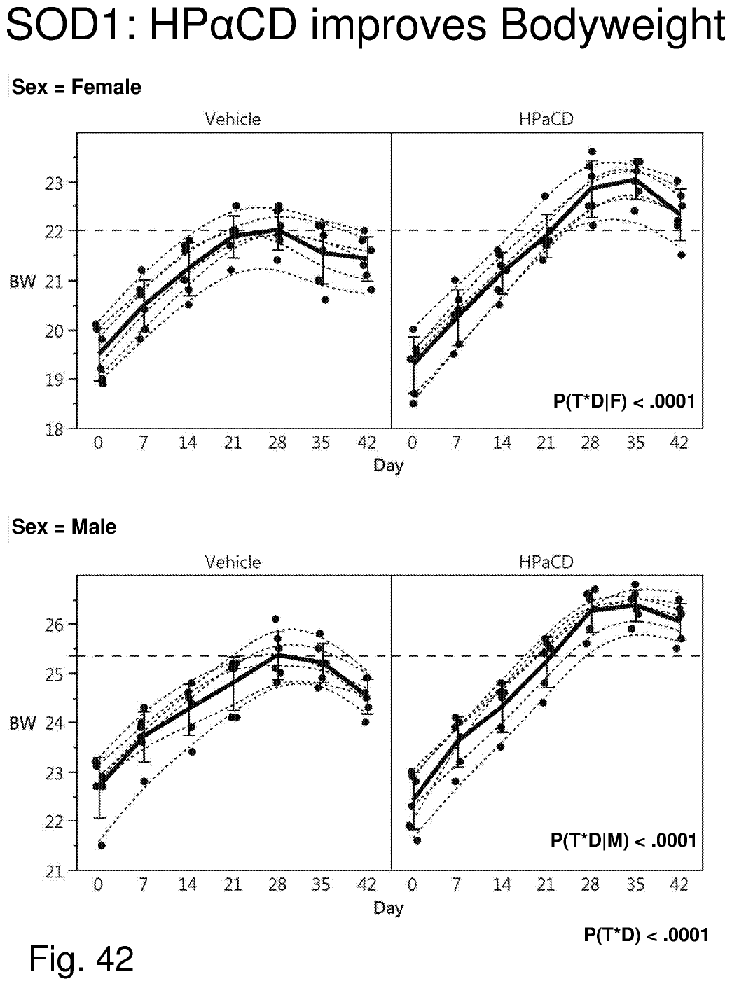

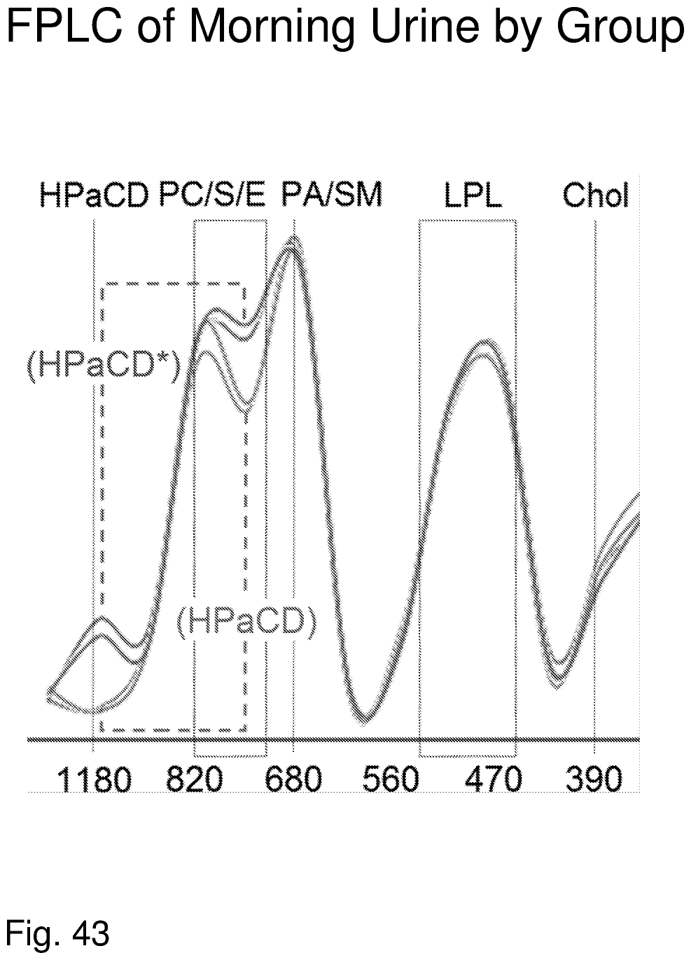

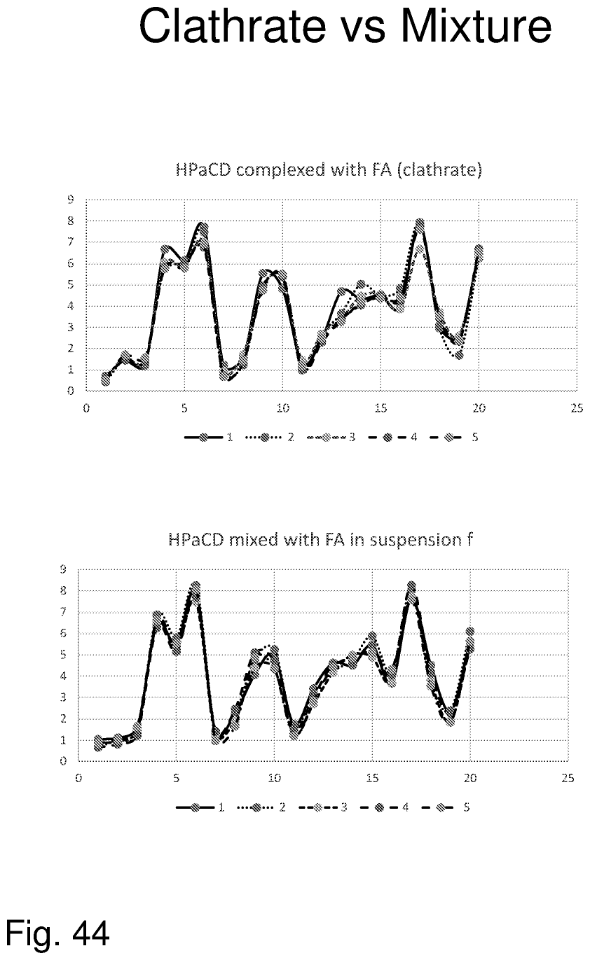

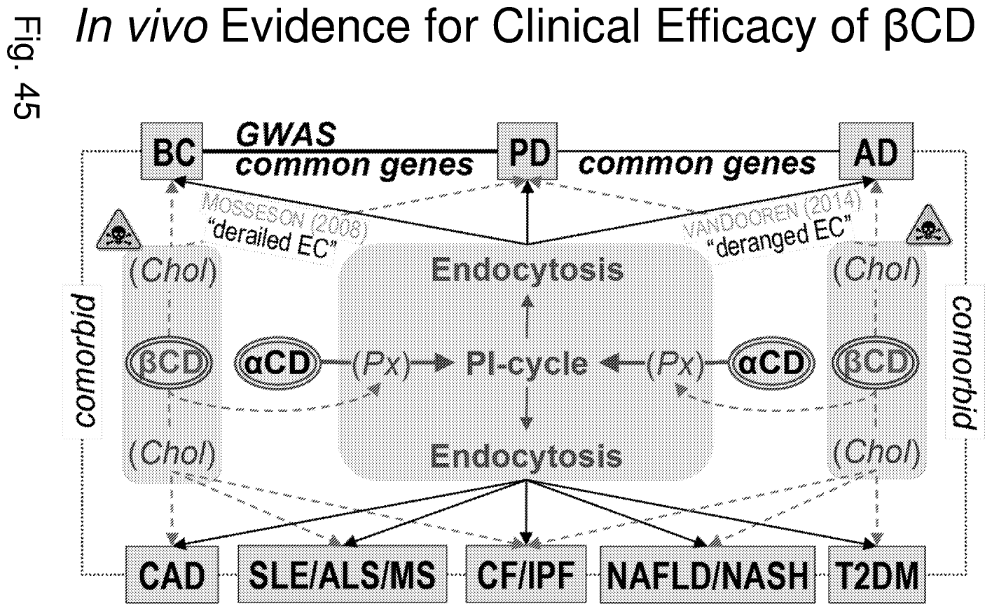

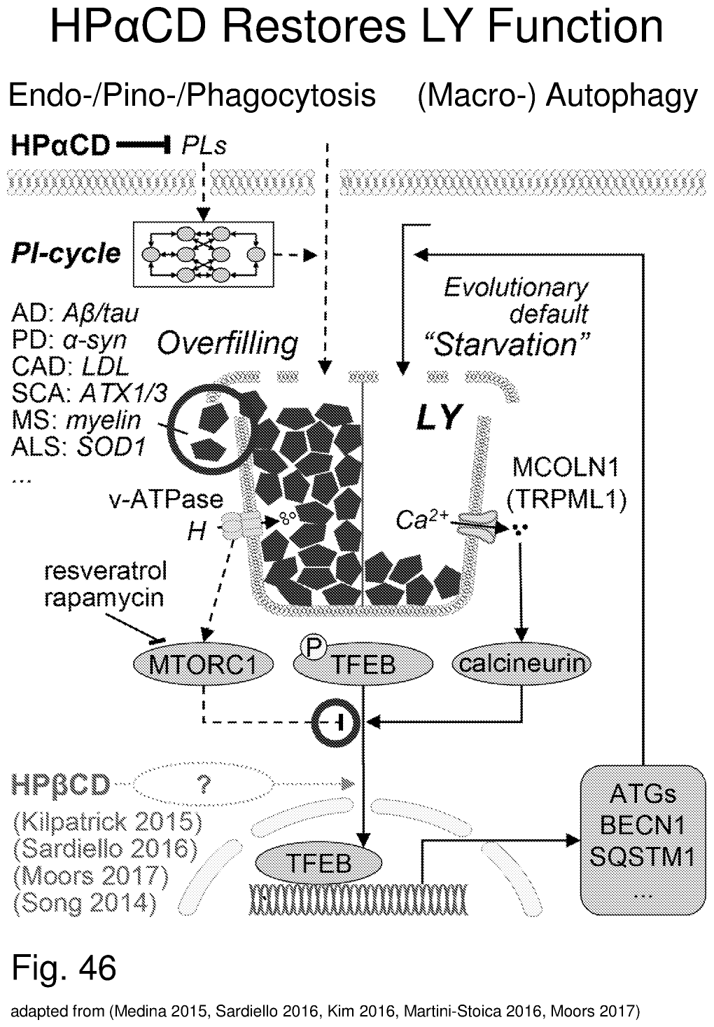

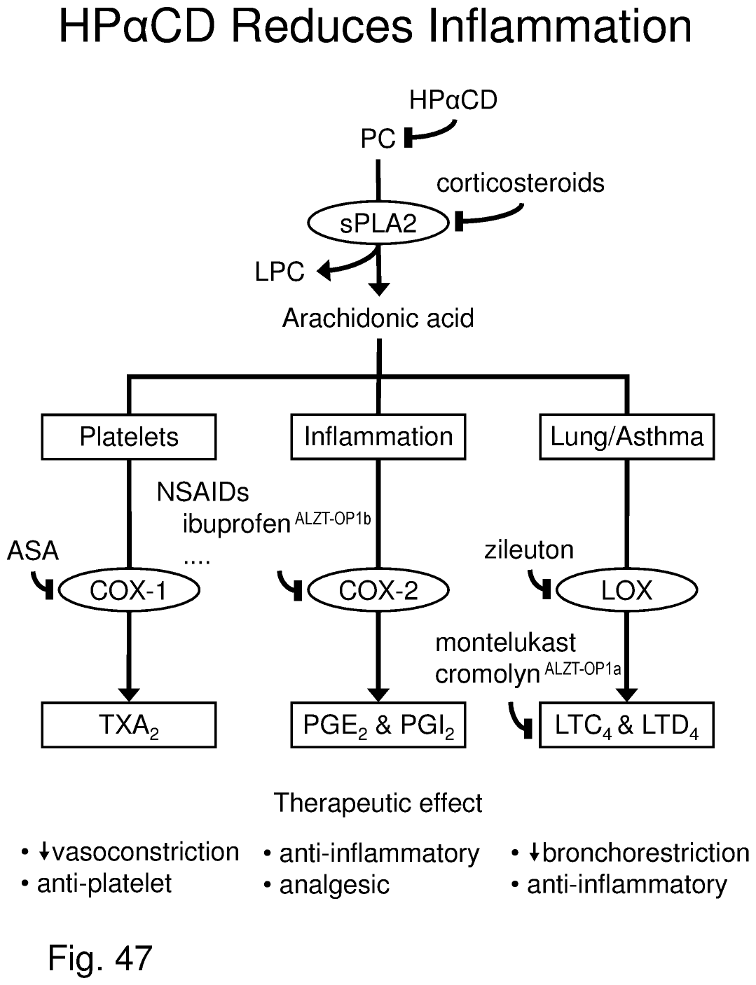

FIG. 37: Impact of PL selectivity: HP-α-CDs* only scavenged PC and PS (and, potentially PI), but neither PA, nor lysophospholipids including but not limited to LPC and LPA. (background: upper left corner of FIG. 7)

FIG. 38A and FIG. 38B: BCa Body Weight (A) and Tumor Volume (B) by Treatment. Individual curves and medians are shown. FIG. 38A: ‘†’ at the bottom and ‘+’ in the curves indicates death for unknown reason. FIG. 38B: Both HP-α-CD and Error! Reference source not found. are effective (overall treatment effect Day*Treatment (D*T)<0.001, ANOCA with Mice as a random factor). HP-α-CD is more effective than Error! Reference source not found. (pairwise treatment effect Day*Treatment (D*Tla:b)=0.0258, ANOCA with mice as a random factor).

FIG. 39: BCa Lung and Liver Metastases. P-values are from pairwise comparisons of bivariate (lung/liver) data via u-statistics for multivariate data (Wittkowski K M, Lee E, et al. (2004) Stat Med 23:1579-92).

FIG. 40: BCa Plasma Cytokines. Individual P-values are derived from two sample t-tests p(b): HPbCD v. Vehicle, p(a): HPαCD v. Vehicle, p(a:b): HPαCD v. HPbCD. Overall P-value for all six cytokines is calculated via u-statistics for multivariate data (Wittkowski K M, Lee E, et al. (2004) Stat Med 23:1579-92).

FIG. 41: HTT Mice: Bodyweight. Estimates are means±SD, P-values for Treatment*Day interaction (P(T*D)) overall and by sex (F: female, M: male) are derived from mixed model ANOVA with mice as random factor.

FIG. 42: SOD1 Mice: Bodyweight. Estimates are means±SD, P-values for Treatment*Day interaction (P(T*D)) overall and by sex (F: female, M: male) are derived from mixed effects ANOVA with mice as a random factor.

FIG. 43: FPLC of Morning Urine: HP-α-CD was recovered from morning urine after intake of the clathrate (full circles), but the unmodified HP-α-CD (open circles) was not excreted. More phospholipids (mostly PC, but potentially also PE) was excreted after taking the clathrate, but neither cholesterol (Chol), as expected), nor any lyso-PL (surprising).

FIG. 44: FPLC of Morning Urine II: A: This Figure like FIG. 43A shows that more of the clathrate is absorbed from the intestine (categolry 2 on the left side and more phosphatidylcholine is extracted into urine than with the suspension proposed as a penetration enhancer to gtet large molecules absorbed from the intestine (Tuvia, Shmuel, et al. (2014), ‘A Novel Suspension Formulation Enhances Intestinal Absorption of Macromolecules Via Transient and Reversible Transport Mechanisms’, Pharmaceutical Research, 31 (8), 2010-21.)

FIG. 45: In vivo Evidence for Clinical Efficacy of βCDs. Efficacy of βCD in animal models of BC, PD, AD, CAD, SLE/ALS/MS, CF/IPF, NAFLD/NASH, and T2DM was consistently attributed to the ability of βCD to scavenge cholesterol (Chol, dashed arrows), which carries the now well-known risk of cholesterol-mediated ototoxicity. The mechanism by which depletion of cholesterol should improve the various phenotypes, however, was rarely explained. Clinical results also showed phospholipid upregulation in several of these diseases. βCDs, however, also scavenge phospholipids (Px) and, thus, also downregulate the PI cycle (center), which direszctly benefits the various disease phenotypes (solid arrows).

FIG. 46: HPαCD Restores LY Function: HPβCD have been shown to activate transcription factor EB (TFEB), yet the mechanism of action is unknown (Song W, Wang F, et al. (2014) J Biol Chem 289:10211-22; Sardiello M (2016) Ann N Y Acad Sci 1371:3-14; Moors T E, Hoozemans J J, et al. (2017) Mol Neurodegener 12:11; Kilpatrick K, Zeng Y, et al. (2015) PLoS One 10:e0120819). The unexpected genetics results confirmed by the urinalysis results adapted from (Sardiello M (2016) Ann N Y Acad Sci 1371:3-14; Moors T E, Hoozemans J J, et al. (2017) Mol Neurodegener 12:11; Medina D L, Ballabio A (2015) Autophagy 11:970-1; Kim S, Choi K J, et al. (2016) Sci Rep 6:24933; Martini-Stoica H, Xu Y, et al. (2016) Trends Neurosci 39:221-34)

### DETAILED DESCRIPTION OF EMBODIMENTS

The present disclosure is based, in part, on the discovery of certain disease-relevant collections of genes based on a reanalysis of three independent sets of breast cancer genetic data, which are available for analysis from the National Institutes of Health's dbGaP collection. This reanalysis differed from the earlier analyses by using a novel computational biostatistics method. Transl Psychiatry, 4, e354. As described in detail in the Examples, this method (Wittkowski 2008) addresses the following four points, which prior analyses of the same data using conventional bioinformatics approaches failed to consider: (i) non-additive relationships between risk alleles and incidence, (ii) cis-epistatic interaction, (iii) correlation between significance and minor allele frequency or “MAF” and (iv) non-randomization bias. It also addresses multiplicity adjustment for diplotype length, a problem arising from the use of a wide-locus approach. By addressing these points, the same strategy previously identified two novel collections of autism-specific genes. Transl Psychiatry, 4, e354.

One collection of genes presented herein (FIG., upper left corner) comprises eight genes whose roles include providing the phosphatidyl inositol (PI) cycle with its substrate, PI. In AD, “PI is one of only 10 serum lipids that can accurately predict memory loss in as much as 90% of cases, 2 years before the onset of dementia symptoms.” Biochim Biophys Acta, 1851 (8), 1066-82. The second gene cluster comprises genes directly associated with endocytosis, a process controlled by PI-phosphatases (PIPs) (Table 5, column PI/EC). The stages include, but are not limited to invagination and forming of clathrin-coated vesicles and early endosomes (hosphatidyl-inositol (PI) cycle. Phosphatidyl-inositol (PI) is synthesized from myo-inositol (imported by HMIT) and phosphatidic acid (PA) (via CDP-DAG), which can be synthesized from lysophosphatic acid (LPA), PC, or (cytosolic) phosphatidyl serine (PS), or salvaged from IP3 and DAG. It can also be synthesized from 1-acyl GPI. Arrows: PIPs are phosphorylated at a 3-, 4-, or 5- position by PI-kinases (left to right) and hydrolyzed by a plethora of phosphatases (right-to-left). Genes associated with BC in this GWAS are highlighted as inverted (bold: aGWS). Wide arrows in the center indicate the sequence of PIPs involved in EEC. Hexagons: PI/PIPs; PM: plasma membrane, CCV: clathrin-coated vesicle, UCV: uncoated vesicle, EE: early endosome, LE: late endosome LY: lysosome. Inverted gene names indicate genes associated with phosphatidylinositol signaling and/or endocytosis. Bold arrows indicate PIPs associated with endocytosis.

Based on this discovery, the present disclosure provides, in part, a shift in the focus for cancer treatments from controlling cell growth, a process involved in many vital functions even in older subjects, to a more to controlling migration and invasion of cells, a process of relevance primarily during prenatal and early postnatal development. Based on these findings, as presented in detail herein, in some embodiments the present disclosure provides compositions and methods for treating a malignant, neurodegenerative, cardiovascular, metabolic, inflammatory, autoimmune, or viral disease or disorder a malignant, neurodegenerative, cardiovascular, metabolic, inflammatory, autoimmune, or viral disease or disorder by modulating the PI cycle and its activity. Furthermore, in some embodiments, and based in part of the involvement of PIs, the present disclosure provides compositions and methods that may be useful for the treatment of a malignant, neurodegenerative, cardiovascular, metabolic, inflammatory, autoimmune, or viral disease or disorder during periods, such as adulthood, where cell migration, including neuronal growth, has mostly ceased, while cell growth, such as growth of hair, skin, and the like, continues and where other cellular mechanism may decline in an age-dependent manner.

In another part, the present disclosure is based on the reevaluation of interpretations of published findings, which are commonly believed to assert that α-CD is as nephrotoxic as β-cyclodextrin, although nephrotoxicity was primarily demonstrated for β-cyclodextrin, which has substantially lower aqueous solubility than α-CD, and, thus, a higher risk of forming the long cytoplasmic crystals seen in the kidneys of rats. For instance, lower toxicity of αCD v. βCD—typically at the level of the safer HP βCD—was shown in HaCaT cells proliferation, caspase activity, and lactose dehydrogenase.

In summary, “the [Joint Expert] Committee [on Food Additives (JECFA)] was reassured by the relatively low toxicity of this compound in animals and the fact that it was less toxic than beta-cyclodextrin, for which studies of human tolerance were available.” WHO/JECFA Food Additive Series 48, 1030 (2001).

In another embodiment, nephrotoxicity is further reduced by reducing the rate of delivery, the method comprising repeated doses per day, Am J Pathol, 83 (2), 367-82, administering the drug over several hours via a peristaltic pump, Pitha, J and Szente, L (1982), ‘Cyclodextrins and Congeners in Parenteral Applications’, Proceedings of the First International Symposium on Cyclodextrins (Springer Netherlands), 457-66, or administering the drug continuously via an implanted drug delivery system.

Some of the main embodiments of the present disclosure are described in the above Summary section of this application, as well as in the Examples, Figures, and Claims. This Detailed Description section provides additional description relating to the compositions and methods of the present disclosure, and is intended to be read in conjunction with all other sections of the present patent application, including the Summary, Examples, Figures, and Claims sections of the present application.

### I. Abbreviations and Definitions

The abbreviations “aCD” and “αCD” refers to alpha-cyclodextrin.

The abbreviation “AD” refers to Alzheimer's disease.

The abbreviation “AKT” refers to protein kinase B.

The abbreviations “bCD”and “βCD” refers to beta-cyclodextrin.

The abbreviation “Ca” refers to calcium.

The abbreviation “CD” refers to cyclodextrin.

The abbreviation “CDK” refers to cyclin-dependent kinase.

The abbreviation “CGEM” refers to Cancer Genetic Markers of Susceptibility.

The abbreviation “Chr” refers to chromosome.

The abbreviation “dbGaP” refers to database of Genotypes and Phenotypes.

The abbreviation “DNA” refers to deoxyribonucleic acid

The abbreviation “EC” refers to endocytosis.

The abbreviation “EE” refers to early endosome.

The abbreviation “EPIC” refers to European Prospective Investigation into Cancer.

The abbreviation “ER” refers to endoplasmatic reticulum

The abbreviation “FAK” refers to focal adhesion kinase.

The abbreviation “Fc” refers to fragment, crystallizable

The abbreviations “gCD” and “γCD” refers to gamma-cyclodextrin.

The abbreviation “GPCR” refers to G-protein coupled receptor.

The abbreviation “GWAS” refers to genome-wide association study.

The abbreviation “GRAS” refers to generally recognized as safe.

The abbreviation “HA” refers to Hyaluronic acid

The abbreviation “HER2/neu” refers to receptor tyrosine-protein kinase erbB-2.

The abbreviation “HLA” refers to human leukocyte antigen.

The abbreviation “HP” refers to hydroxypropyl.

The abbreviations “HPaCD” and “HPαCD” refers to hydroxypropyl-alpha-cyclodextrin.

The abbreviations “HPbCD” and “HPβCD” refers to hydroxypropyl-beta-cyclodextrin.

The abbreviation “IND” refers to investigational new drug.

The abbreviation “IPV” refers to inverse p-value.

The abbreviation “i.v.” refers to intravenous.

The abbreviation “LD” refers to linkage disequilibrium.

The abbreviation “LD₅₀” refers to median lethal dose.

The abbreviation “LE” refers to late endosome.

The abbreviation “MAF” refers to minor allele frequency.

The abbreviation “MAP refers to mitogen-activated protein.

The abbreviation “mTOR” refers to mechanistic target of rapamycin.

The abbreviation “muGWAS” refers to multivariate u-statistics GWAS.

The abbreviation “NIH” refers to National Institutes of Health.

The abbreviation “NPC” refers to Niemann Pick disease type C.

The abbreviation “PBCS” refers to Polish Breast Cancer Case-Control Study.

The abbreviation “PD” refers to Parkinson's disease.

The abbreviation “PI” refers to phosphatidylinositol.

The abbreviation “PIP” refers to phosphatidylinositol phosphate

The abbreviation “PIP” refers to PI(4)P.

The abbreviation “PIP2” refers to PI(4,5)P₂.

The abbreviation “PIP3” refers to PI(3,4,5)P₃.

The abbreviation “PM” refers to plasma membrane”

The abbreviation “PKB” refers to protein kinase B.

The abbreviation “PKC” refers to protein kinase C.

The abbreviation “QQ” refers to “quantile-quantile”.

The abbreviation “QR” refers to “quantile-rank”.

The abbreviation “RTK” refers to receptor tyrosine kinase.

The abbreviation “s.c.” refers to subcutaneous.

The abbreviation “SNP” refers to single nucleotide polymorphism.

The abbreviation “ssGWAS” refers to single-SNP genome-wide association study.

The abbreviation “TSC” refers to tuberous sclerosis.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary, Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one 01” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of +/−20%, +/−10%, +/−5%, +/−1%, +/−0.9%, +/−0.8%, +/−0.7%, +/−0.6%, +/−0.5%, +/−0.4%, +/−0.3%, +/−0.2% or +/−0.1% from the specified value, as such variations are appropriate to perform the disclosed methods.

As used herein, the terms “treat,” “treating,” and “treatment” encompass a variety of activities aiming at desirable changes in clinical outcomes. For example, the term “treat”, as used herein, encompasses any detectable improvement in one or more clinical indicators or symptom of a malignant, neurodegenerative, cardiovascular, metabolic, inflammatory, autoimmune, or viral disease or disorder—such as carcinomas, including, but not limited to, or neurodegenerative diseases, including, but not limited to, Parkinson's and Alzheimer's Disease, or coronary artery disease, including, but not limited to atherosclerosis, or a digestive disorder, including, but not limited to type-2 diabetes. For example, such terms encompass alleviating, abating, ameliorating, relieving, reducing, inhibiting, preventing, or slowing at least one clinical indicator or symptom, preventing additional clinical indicators or symptoms, reducing or slowing the progression of one or more clinical indicators or symptoms, causing regression of one or more clinical indicators or symptoms, relieving a condition caused by the disease or disorder, and the like. As used herein the terms “treat,” “treating,” and “treatment” encompass both prophylactic treatments and therapeutic treatments. In one aspect, “treat,” “treating,” and “treatment” refere to therapeutic treatment. In the case of prophylactic treatments, the methods and compositions provided herein can be used preventatively in subjects that do not yet exhibit any clear or detectable clinical indicators or symptoms of the disease or disorder but that are believed to be at risk of developing the disease or disorder, such as a malignant, neurodegenerative, cardiovascular, metabolic, inflammatory, autoimmune, or viral disease or disorder. In the case of therapeutic treatments, the methods and compositions provided herein can be used in subjects that already exhibit one or more clinical indicators or symptoms of the disease or disorder, such as a malignant, neurodegenerative, cardiovascular, metabolic, inflammatory, autoimmune, or viral disease or disorder. In the case of malignant or neurodegenerative diseases or disorders, various clinical indicators and symptoms are known to medical practitioners and those of skill in the art.

The terms “prevent” or “preventing” as used herein encompasses stopping a disease, disorder, or symptom from starting, as well as reducing or slowing the progression or worsening of a disease or disorder. For example, “preventing” breast cancer or prostate cancer includes, but is not limited to, inhibiting the formation of cancerous cells or inhibiting the metastasis of malignant growths.

The term “a malignant, neurodegenerative, cardiovascular, metabolic, inflammatory, autoimmune, or viral disease or disorder” is used herein in accordance with it usual usage in the art and includes, but is not limited to malignant disorders, such as carcinomas, breast cancer, prostate cancer, malignancies of the breast, and malignancies of the prostate, as well as neurodegenerative diseases and disorders, such as Alzheimer's disease, Parkinson's disease, Huntington's disease, and “a range of disorders including brain overgrowth syndromes, [and] Charcot-Marie-Tooth neuropathies.” Biochim Biophys Acta, 1851 (8), 1066-82.

As used herein, the term “cancer” or “hyperproliferative disease” is meant to refer to those diseases and disorders characterized by hyperproliferation of cells. Examples of hyperproliferative disease includes all forms of cancer, psoriasis, neoplasia, and hyperplasia.

The terms “subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term subject. The term “subject” is also used throughout the specification in some embodiments to describe an animal from which a cell sample is taken or an animal to which a disclosed cell or nucleic acid sequences have been administered. In some embodiment, the animal is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” description above and also include treatment of any lysosomal storage disease caused by dysfunction of the lysosomes. An appropriate “effective” amount in any individual case may be determined using standard techniques known in the art, such as a dose escalation study. In some embodiments, as used herein, the term “therapeutically effective amount” is meant to refer, in respect to cancer, to an amount of an active agent or combination of agents effective to ameliorate or prevent the symptoms, shrink tumor size, prevent progression of cancer from non-metastatic to metastatic disease, or prolong the survival of the patient being treated for cancer or neurodegenerative disease. Determination of a therapeutically effective amount is well within the capabilities of those skilled in the art, especially in light of the detailed disclosure provided herein.

In some embodiments, cyclodextrin or derivatives thereof or pharmaceutically acceptable salts thereof can be co-administered with other therapeutics and/or part of a treatment regimen that includes radiation therapy.

Pharmaceutically acceptable” refers to a component that is, within the scope of sound medical judgment, suitable for use in contact with the tissues of the subject, such as humans and other mammals, without undue toxicity, irritation, allergic response and the like, and are commensurate with a reasonable benefit/risk ratio. These include, but are not limited to, components which are approved or subject to approvable by a regulatory agency of the Federal or a state government or the corresponding agency in countries other than the United States, or that is listed in the U.S. Pharmacopoeia or other generally recognized pharmacopoeia for use in animals, and in one aspect, in humans.

“Pharmaceutically acceptable salt” refers to a salt of a compound of the present disclosure that is pharmaceutically acceptable and that possesses the desired pharmacological activity of a compound of the present disclosure. In particular, such salts are non-toxic may be inorganic or organic acid addition salts and base addition salts. Specifically, pharmaceutically acceptable non-toxic acids include hydrochloric acid, fumaric acid, maleic acid, succinic acid, acetic acid, citric acid, tartaric acid, carbonic acid, phosphoric acid, oxalic acid, dichloroacetic acid and the like. Pharmaceutically-acceptable non-toxic bases include such as sodium hydroxide, potassium hydroxide, choline hydroxide, sodium carbonate and the like.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The co-administration of therapeutics can be sequential in either order or simultaneous. In some embodiments cyclodextrin or derivatives thereof or pharmaceutically acceptable salts thereof is co-administered with more than one additional therapeutic. Examples of chemotherapeutics include common cytotoxic or cytostatic drugs such as for example: methotrexate (amethopterin), doxorubicin (adrimycin), daunorubicin, cytosinarabinoside, etoposide, 5-4 fluorouracil, melphalan, chlorambucil, and other nitrogen mustards (e.g. cyclophosphamide), cis-platin, vindesine (and other vinca alkaloids), mitomycin and bleomycin. Other chemotherapeutics include: purothionin (barley flour oligopeptide), macromomycin. 1,4-benzoquinone derivatives and trenimon. Anti-cancer antibodies, such as herceptin, and toxins are also examples of other additional therapeutics.

The therapeutic regimens can include sequential administration of cyclodextrin or derivatives thereof or pharmaceutically acceptable salts thereof and initiation of radiation therapy in either order or simultaneously. Those skilled in the art can readily formulate an appropriate radiotherapeutic regimen. Carlos A Perez & Luther W Brady: Principles and Practice of Radiation Oncology, 2nd Ed. JB Lippincott Co, Phila, 1992, which is incorporated herein by reference in its entirety describes radiation therapy protocols and parameters which can be used in the present disclosure.

When used in as part of the combination therapy the therapeutically effective amount of the inhibitor may be adjusted such that the amount is less than the dosage required to be effective if used without other therapeutic procedures.

In some embodiments, treatment with pharmaceutical compositions described herein are preceded by surgical intervention.

The disclosure also relates to methods of reducing the number of exosomes in a cancer cell by contacting said cancer cell with a therapeutically effective amount of a cyclodextrin.

The term “pharmaceutical composition” as used herein refers to a composition comprising at least one active agent as described herein (such as, for example, an (α-CD), and one or more other components suitable for use in pharmaceutical delivery such as a pharmaceutically acceptable carrier, stabilizer, diluent, dispersing agent, suspending agent, thickening agent, excipient, and the like. In some embodiments, the disclosure relates to a pharmaceutical composition comprising α-CD or derivative thereof such as HPα-CD and one or a plaurality of fatty acids and a pharamceutically acceptable carrier. In some embodiments, the disclosure relates to a pharmaceutical composition comprising α-CD or derivative thereof such as HPα-CD and one or a plurality of fatty acids, such that the α-CD or derivative thereof such as HPα-CD and one or a plaurality of fatty acids form a clathrate. In some embodiments, the pharmaceutical composition comprises a weight percent of α-CD or derivative thereof of about 10% and a weight percent of fatty acids of about 90%. In some embodiments, the pharmaceutical composition comprises a weight percent of α-CD or derivative thereof of about 20% and a weight percent of fatty acids of about 80%. In some embodiments, the pharmaceutical composition comprises a weight percent of α-CD or derivative thereof of about 30% and a weight percent of fatty acids of about 70%. In some embodiments, the pharmaceutical composition comprises a weight percent of α-CD or derivative thereof of about 40% and a weight percent of fatty acids of about 60%. In some embodiments, the pharmaceutical composition comprises a weight percent of α-CD or derivative thereof of about 50% and a weight percent of fatty acids of about 50%. In some embodiments, the pharmaceutical composition comprises a weight percent of α-CD or derivative thereof of about 60% and a weight percent of fatty acids of about 40%. In some embodiments, the pharmaceutical composition comprises a weight percent of α-CD or derivative thereof of about 70% and a weight percent of fatty acids of about 30%. In some embodiments, the pharmaceutical composition comprises a weight percent of α-CD or derivative thereof of about 80% and a weight percent of fatty acids of about 20%. In some embodiments, the pharmaceutical composition comprises a weight percent of α-CD or derivative thereof of about 90% and a weight percent of fatty acids of about 10%. In some embodiments, the pharmaceutical composition comprises a weight percent of fatty acids of from about 5% to about 15%. In some embodiments, the pharmaceutical composition comprises a weight percent of α-CD or derivative thereof from about 85% to about 95%.

The term “active agent” as used herein refers to a molecule that is intended to be used in the compositions and methods described herein and that is intended to be biologically active, for example for the purpose of treating a malignant, neurodegenerative, cardiovascular, metabolic, inflammatory, autoimmune, or viral disease or disorder. The term “active agent” is intended to include molecules that either are, or can be converted to a form that is, biologically active. For example, the term “active agent” includes pro-drugs and/or molecules that are inactive or lack the intended biological activity but that can be converted to a form that is active or has the intended biological activity.

As used herein, the term “sample” refers generally to a limited quantity of something which is intended to be similar to and represent a larger amount of that thing. In the present disclosure, a sample is a collection, swab, brushing, scraping, biopsy, removed tissue, or surgical resection that is to be tested for clinical indicators of a disease or disorder, such as a malignant or neurodegenerative disease or disorder. In some embodiments, samples are taken from a patient or subject that is believed to have a malignant, neurodegenerative, cardiovascular, metabolic, inflammatory, autoimmune, or viral disease or disorder. In some embodiments, a sample believed to contain clinical indicators of a disease or disorder, such as a malignant or neurodegenerative disease or disorder, is compared to a control sample that is known not to contain one or a plurality of clinical indicators of a disease or disorder, such as a malignant or neurodegenerative disease or disorder. In some embodiments, a sample believed to contain a clinical indicator of a disease or disorder, such as a malignant, neurodegenerative, cardiovascular, metabolic, inflammatory, autoimmune, or viral disease or disorder, is compared to a control sample that is known to not contain a clinical indicator of a disease or disorder, such as a malignant, neurodegenerative, cardiovascular, metabolic, inflammatory, autoimmune, or viral disease or disorder. In some embodiments, a sample believed to contain a clinical indicator of a disease or disorder, such as a malignant, neurodegenerative, cardiovascular, metabolic, inflammatory, autoimmune, or viral disease or disorder, is compared to a control sample that contains the same clinical indicators of a disease or disorder, such as a malignant, neurodegenerative, cardiovascular, metabolic, inflammatory, autoimmune, or viral disease or disorder.

The term “scavenge” as used herein means uptake or chemically combine with and transport to Error! Reference source not found. the kidney for excretion.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts of the embodiments include those formed from hydrochloric, hydrobromic, sulphuric, phosphoric, citric, tartaric, lactic, pyruvic, acetic, succinic, fumaric, maleic, methanesulphonic and benzenesulphonic acids.

In some embodiments, salts of the compositions comprising one or may be formed by reacting the free base, or a salt, enantiomer or racemate thereof, with one or more equivalents of the appropriate acid. In some embodiments, pharmaceutical acceptable salts of the present disclosure refer to amino acids having at least one basic group or at least one basic radical. In some embodiments, pharmaceutical acceptable salts of the present disclosure comprise a free amino group, a free guanidino group, a pyrazinyl radical, or a pyridyl radical that forms acid addition salts. In some embodiments, the pharmaceutical acceptable salts of the present disclosure refer to amino acids that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromaticaliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present disclosure may be found in their anhydrous form or as in hydrated crystalline form (i.e., complexed or crystallized with one or more molecules of water).

The term “soluble” or “water soluble” refers to solubility that is higher than 1/100,000 (mg/ml). The solubility of a substance, or solute, is the maximum mass of that substance that can be dissolved completely in a specified mass of the solvent, such as water. “Practically insoluble” or “insoluble,” on the other hand, refers to an aqueous solubility that is 1/10,000 (mg/ml) or less. Water soluble or soluble substances include, for example, polyethylene glycol. In some embodiments, the polypeptide described herein be bound by polyethylene glycol to better solubilize the composition comprising the peptide.

As used herein, percent “homology” or “sequence identity” is determined by using the stand-alone executable BLAST engine program for blasting two sequences (blZseq), which can be retrieved from the National Center for Biotechnology Information (NCBI) ftp site, using the default parameters (Tatusova and Madden, FEMS Microbiol Lett, 1999, 174, 247-250; which is incorporated herein by reference in its entirety).

Additional definitions and abbreviations are provided elsewhere in this patent specification or are well known in the art.

### II. Additional Description

Malignant, neurodegenerative, cardiovascular, metabolic, inflammatory, autoimmune or viral diseases or disorders are complex diseases involving many genes along common pathways. For instance, cardiovascular disease and dementia are known comorbidities of type-2 diabetes. Based in part on the findings that the phosphoinositides PI(3,4,5)P₃ and PI(3,4)P₂ are upregulated in malignant and neurological diseases, see Biochim Biophys Acta, 1851 (8), 1066-82, and activate Akt signaling. See Cell Signal, 20 (4), 684-94; Journal of Lipid Research, 50 (Suppl), S249-S54; Cancer Cell, 28 (2), 155-69. PI signaling is widely believed to be involved in cancer growth and neurodegeneration. In addition, INPP4B is known as a suppressor of some cancers, but as a risk factor for others, including, but not limited to, breast and pancreatic cancer. See Oncotarget, 7 (1), 5-6; Trends Mol Med, 21 (9), 530-32. PI3K signaling has also be implied in aging, cognitive decline, and Alzheimer's. See Exp Gerontol, 48 (7), 647-53. Still, “clinical results with single-agent PI3K inhibitors have been modest to date,” Annu Rev Med, 67, 11-28, in part because traditional GWAS have largely failed to elucidate the precise mechanism by which PI signaling contributes to cancer and neurodegenerative diseases.

From the results presented herein, a malignant, neurodegenerative, cardiovascular, metabolic, inflammatory, autoimmune, or viral disease or disorder as well as Alzheimer's and Parkinson's disease, atherosclerosis, and type-2 diabetes are characterized by hyperactivity within the phosphatidylinositol (PI) cycle. In some aspects the present disclosure provides methods of treatment of a malignant, neurodegenerative, cardiovascular, metabolic, inflammatory, autoimmune, or viral disease or disorder, cardiovascular, metabolic, and Parkinson's or Alzheimer's diseases, that comprise administering to a subject one or more therapeutically effective amounts of active agents that target PIPs to elicit changes in endo-/exocytosis thereby causing a dampening or decrease of cellular migration and infiltration or processing of proteins, including, but not limited to, APP, tau, and alpha-synuclein. Such active agents may specifically target particular PIPs or may act non-specifically on several different classes or types of PIPs to elicit a broad reduction of activity. Furthermore, such active agents could be, for example, compounds or drugs that are already being used safely in humans for other indications and could be repurposed for use in the treatment of a malignant, neurodegenerative, cardiovascular, metabolic, inflammatory, autoimmune, or viral disease or disorder.

Other proposed uses of cyclodextrins are in the treatment of neurodegenerative diseases, including, but not limited to neural ceroid lipofuscinoses (J Biol Chem, 289 (14), 10211-22), Parkinson's disease (PD), Alzheimer's disease (AD), frontotemporal dementia (FTD), and Huntington's disease (HD), stroke (Neuroreport, 23 (3), 134-8; Arch Med Res, 45 (8), 711-29), atherosclerosis (J Pharm Sci, 81 (6), 524-8; Sci Transl Med, 8 (333), 333ra50), fatty liver disease (Int J Mol Sci, 16 (9), 21056-69), respiratory diseases, including, but not limited to cystic fibrosis (Gunasekara, L., et al. (2017), ‘Pulmonary surfactant dysfunction in pediatric cystic fibrosis: Mechanisms and reversal with a lipid-sequestering drug’, J Cyst Fibros), asthma and pulmonary disease (U.S. Pat. No. 9,034,846 B2 to Cataldo et al.; Biochim Biophys Acta, 1859 (10), 1930-40), diabetes type 2 and insulin resistance (Am J Physiol Endocrinol Metab, 308 (4), E294-305), and protection from virus or bacterial infections (Physiological Reviews, 93 (3), 1019-137; J Virol, 78 (1), 33-41).

Contemplated genetic diseases, include, but are not limited to muscular and corneal dystrophy or myotubular neuropathy, cancer, including, but not limited to leukemias (ALL, AML, CML) and lymphomas (NHL), epilepsy, and thrombosis. See Biochimie, 125, 250-58. Neurological diseases linked to PIP dysregulation include, but are not limited to Friedreich's ataxia, Charcot-Marie-Tooth degenerative neuropathia, renal tubolopathies (Oculocerebrorenal syndrome of Lowe, Dent's disease), Andersen-Tawil syndrome, mucolipidosis, multiple sclerosis, Yunis-Varon syndrome, amytrophic lateral sclerosis, and ciliopathies. See Biochim Biophys Acta, 1851 (8), 1066-82. Additional diseases linked to PIP dysregulation are osteoporosis, cancers (including, but not limited to cervical cancer, leiomyosarcoma, gastric cancer, adenocarcinoma, lung cancer), cardiac hypertrophy, and autoimmune diseases (including, but not limited to rheumatoid arthritis), bipolar disorder and schizophrenia. Falasca, Marco (ed.), (2012), Phosphoinositides and Disease, ed. Peter K. Vogt (Current topics in microbiology and Immunology, Dordrecht: Springer).

The inventor contemplates that the cyclodextrins disclosed herein may be useful in the prevention and treatment of metastases, which are the most lethal aspects of malignant disorders. Breast cancer, for instance, is the most common non-cutaneous malignancy in women. In 2015, 231.840 U.S. women were newly diagnosed and an estimated 3.1 million breast cancer survivors are alive in the U.S. J Clin Oncol, 34 (6), 611-35. After lung cancer, it was the second most deadly, causing 40,290 deaths, 17,010 before the age of 65. “[W]omen who have one first-degree relative (mother, sister, or daughter) with a history of breast cancer are about twice as likely to develop breast cancer as women who do not have this family history.” American Cancer Society (2015), Cancer Facts & Figures 2015 (Atlanta: American Cancer Society). These results imply that a substantial portion of cancer risk is inherited, yet the known complexes of DNA damage repair genes BRCA1/BRCA2/PALB2 and MRE11A/RAD50/NBN/RINT1 as well as PIK3CA/PTEN, which is currently believed to be mainly involved in growth, explain only 10% of the incidence. Prostate cancer, in turn, is the most common cancer in men. In 2015, 220,800 U.S. men were newly diagnosed and 27,540 died. Many epithelial cancers (derived from endodermal or ectodermal tissue, including, but not limited to breast, prostate, lung, pancreas, and colon carcinoma) are known to share risk factors. For instance, “[b]oth [breast and prostate] require gonadal steroids for their development, and tum[o]rs that arise from them are typically hormone-dependent and have remarkable underlying biological similarities.” Nat Rev Cancer, 10 (3), 205-12. Prostate cancer is known to be genetically linked with breast cancer. “Prostate cancer diagnosed among first-degree family members increases a woman's risk of developing breast cancer.” Cancer, 121 (8), 1265-72. AR and BRCA2 are among the many genes affiliated with both forms of cancer. See Drug discovery today. Disease mechanisms, 9 (1-2), e19-e27 and Asian J Androl, 14 (3), 409-14, respectively.

Many other diseases are known to involve loss-of-function mutations in the lysosome, where many of the endocytosed molecules need to be degraded. In all cases, the disease phenotype would be improved by reducing the amount of endocytosed material that needs to be degraded (MPS: mucopolysaccharidosis, NCL: neuronal ceroid lipofuscinosis, MLD: metachromatic leukodystrophy):

ABCA2: acoustic neuroma; ABCB9: acute myeloid leukemia (AML); ACP2: acid phosphatase deficiency, keratoconus, amyotrophic lateral sclerosis, atrichia, amebiasis; ACP2: lysosomal acid phosphatase deficiency, keratoconus, amotrophic lateral sclerosis 8, mebiasis; ACP5: spondyloenchondrodysplasia, bone giant cell tumor, hairy cell leucoma, tooth resorption; AGA: aspartylglucosaminuria, fucosidosis; AP3B1: hermansky-pudlak syndrome, oculocutaneous albinism, storage pool platelet disease; AP3B2: paraneoplastic neurologic disorders; ARSA: MLD, arylsulfatase a deficiency, multiple sulfatase deficiency, mucosulfatidosis; ARSB: MPS VI, arylsulfatase b deficiency, mucosulfatidosis; ASAH1: Farber lipogranulomatosis, (Kugelberg-Welander) spinal muscular atrophy, (Erdheim-Chester) lipogranulomatosis,; ATP13A2/PARK9: NCL 12, Kufor-Rakeb syndrome, spastic paraplegia 78, juvenile onset Parkinson disease; ATP6AP2 : mental retardation (Mol Cell Neurosci, 66 (Pt A), 21-28); Parkinsonism with spasticity, lymphoytic choriomeningitis; ATP6V0A1: inferior myocardial infarction; ATP6V0A2: wrinkly skin syndrome, cutis laxa, myelophthisic anemia; ATP6V0A4: renal tubular acidosis, medullary sponge kidney; CD164: autosomal dominant deafness, pollen allergy, prostate cancer; CLC7: osteopetrosis; CLN1/PPT1: NCL, Batten disease, Santavuori-Haltia; CLN11/GRN: NCL, frontotemporal dementia, primary progressive aphasia; CLN3: NCL, secondary corneal edema, Spielmeyer-Vogt-Sjogren-Batten disease; CLN4/DNAJC5: NCL, CLN4 disease; CLN5: NCL; CLN5: NCL, Finnish late infantile; CLN6: NCL, Kufs disease; CLN7: NCL, macular dystrophy, neurotic disorder, depressive neurosis; CLN8: Norther epilepsy, progressive with mental retardation/Turkish late infantile; CTNS: nephropathic cystinosis, Fanconi syndrome; CTSA: galactosialidosis, glycoproteinosis, gonococcal salpingitis, triosephosphate isomerase deficiency, aspartylglucosaminuria; CTSB: occlusion of gallbladder, ileum cancer, pancreatitis, keratolytic winter erythema, breast cancer, Alzheimer's disease; CTSC: Papillon-Lefevre syndrome, Haim-munk syndrome, periodontitis, palmoplantar keratosis, actinic keratosis; CTSD: NCL, bone chondrosarcoma, endometrial clear cell adenocarconoma, breast diseases, Parkinson's disease (Parnetti 2017); CTSE: gastric adenocarcinoma, Rosai-Dorfman disease, histiocytosis; CTSF: NCL, Kufs disease, akinetic mutism, coma vigilans, cionorchiasis, oriental liver fluke disease; CTSG: Papillon-Lefevre syndrom, cutaneous mastocytosis, suppurative periapical periodontitis, vasculitis, Wegener granulomatosis; CTSH: thyroid crisis, intermittent explosive disorder, narcolepsy, fibrous meningioma, amyotrophic lateral sclerosis; CTSK: pycodystostosis, hypersensitivity pneumonitis, osteosclerosis, osteomyelitis, bone giant cell tumor; CTSL: vulva basal cell carcinoma, eccrine acrospiroma, fasciolopsiasis, tracheal cancer, rectosigmoid junction neoplasm; CTSO: breast cancer; CTSS: mandibular cancer, non-suppurative otitis media, cercerial dermatitis, subepithelial mucinous corneal dystropy, jaw cancer; CTSW: autoimmune atrophic gastritis; CTSZ: dacryoadenitis; DNASE2: calcific tendinitis, rheumatoid arthritis; FUCA1: fucosidosis, angiokeratoma, mucolipidosis III, laryngotracheitis; GAA: glycogen storage disease II (Pompe disease), acid maltase deficiency, Danon disease, alpha-1,4-glucosidase deficiency; GALC: Krabbe disease, leukodystrophy; GALNS: MPS IV, chondroosteodystrophy, Scheie syndrome, Kniest dysplasis; GBA: Gaucher disease, pseudo Gaucher disease, GBA-associated Parkinson's disease; GLA: Fabry disease, cramp-fasciculation syndrome, agiokeratoma, MPS VII; GLB1: gm1-gangliosidosis, MPS IVb; GM2A: GM2-gangliosidosis, Tay-Sachs disease, Sandhoff-disease, mucolipidosis II alpha/beta, I-cell disease; GNPTAB: mucolipidosis 2, mucolipidosis 3, MPS 3a; GNPTG: mucolipidosis III, pseudo-Hurler polydystrophy, articulation disorder; GNS: MPS III, multiple sulfatase deficiency, mucosulfatidosis; GUSB: MPS VII, hydrops fetalis, necrotizing ulcerative gingivitis, choledocholithiasis; GUSB: Sly disease, MSP VII, hydrops fetalis, necrolizing ulcerative gingivitis, choledocholithiasis; HEXA: Tay-Sachs disease, gm2 gangliosidosis; HEXB: Sandhoff disease, mucolipidosis IV, sphingolipidosis; HGSNAT: MPS IIIc, MPS IIIb, retinitis pigmentosa, Kluver-Bucy sundrome; HYAL1: MPS IX, bladder carcinoma, prostate cancer; IDS: MPS II, Hunter syndrome; IDUA: MPS I, Scheie syndrome, Hurler-sundrome; IGF2R: hepatocellular carcinoma, mucolipidosis II, inclusiojn-cell disease, colorectal cancer; LAAT1/PQLC2: cystinosis; LAMP1: Salla disease, Hermansky-Pudlak syndrome, lysosomal acid phosphatase deficiency, haemophagocytic symdrome; LAMP2: danon disease, cardiomyopathy, atrial standstill, glycogen storage disease II, lysosomal acid phosphatase deficiency; LAMP3/CD63: Hermansky-Pudlak syndrome, melanoma, Quebec platelet disorder, mast cell disease, Schwarzman phenomenon; LAMP4/CD68/SCARD1: (breast) granular cell tumor, follicular dendritic cell sarcoma, bacterial esophagitis, axillary lipoma; LAPTM4A: pain disorder; LAPTM4B: hepatocellular carcinoma; LGMN: schistosomiasis; LIMP2/SCARB2: progressive myoclonic epilepsy, Unverricht-Lundborg disease, hand-tooth-and-mouth disease, myoclonus; LIPA: Wolman disease, cholesterol ester storage disease, splenic abscess, familial hypercholesterolemia; LITAF: Charcot-Marie-Tooth hereditary neuropathy; LMBRD1: methylmalonic aciduria and homocystinuria, cblf type, hepatitis, transcobalamin II deficiency; LYPLA3/PLA2G15:; LYST: Chedliak-Higashi syndrom, exfoliation syndrome, Hermansky-Pudlak syndrome, dichromatosis symmetrica; M6PR: MPS IIIa, mucolipidosis II, NCL, Niemann-Pick disease; MANB: mannosidosis (alpha/beta); MCOLN1: mucolipidosis IV, ataxic cerebral palsy, sphingolipidosis, strabismus, mucolipin-1 deficiency; NAGA: Kanzaki disease, Schindler disease, neuroaxonal dystrophy, agiokeratoma; NAGLU: MPS III, Charcot-Marie-Tooth disease, acute pyelonephritis; NAGPA: familial persistent stuttering, articulation disorder, MPS IIIb, speech disorder, diabetic nephropathy; NAPSA: ovarian clear cell adenofibroma, Krukenberg/lung adeno/pulmonary adeno (Bishop 2010)/ovarian clear cell/carcinoma, malignant fibrous mesothelioma; NEU1: sialidosis, hydrops fetalis, parainfluence virus type 3; NPC1/2: Niemann-Pick disease C1/2; PSAP: atypical Gaucher disease, combined saposin deficiency, MLD, Krabbe disease; PSEN1 (Lee 2015, Sato 2017): frontotemporal dementia, Alzheimer's disease 3, familial acne inversa, dilated cardiomyopathy; RAB27A: Griscelli syndrome, hemophagocytic lymphohistiocytosis, Chedliak-Higashi syndrome; RAB7: Charcot-Marie-Tooth disease 2b (Mol Cell Neurosci, 66 (Pt A), 21-28); choroideremia, tabes dorsalis, NCL; SGSH: MPS III, Klver-Bucy syndrome, Sanfillipo syndrome; SLC11A1: Buruli ulcer, tuberculosis, typhoid fever; SLC11A2: microcytic anemia, hemosiderosis, anemia; SLC17A5/SIALIN: salla disease, sialic acid storage disorder, sialuria, fascioliasis; SMPD1: Niemann-Pick disease, acid sphingomyelinase deficiency, narcissistic personality disorder; SORT1: inclusion-cell disease, I-cell disease, myocardial infarction; SUMF1: multiple sulfatase deficiency, MLD, spinocerebellar ataxia, MPS VI; TPP1: NCL, spinocerebellar ataxia, Bielschowsky-Jansky disease.

### A) Active Agents

As further described in the Examples and other sections of the present application, agents that can be used in the present methods may reduce the concentration of phospholipids available to cells, including, but not limited to, neurons and tumor cells. In one embodiment, the agent is a cyclodextrin. In another embodiment, the cyclodextrin is α-CD. In yet another embodiment, the agent is hydroxypropyl- (HP-α-CD). In some embodiments, the agent is a clathrate of a α-CD or HP-α-CD. In some embodiments the agent is a clathrate of α-CD or HP-α-CD with a fatty acid. A “fatty acid” as used herein means a carboxylic acid with a long aliphatic chain, which is either saturated or unsaturated. In some embodiments, the aliphatic chain is from about 3 to about 70 carbons long. In some embodiments, the aliphatic chain is from 10 to about 30 carbons long. In some embodiments the fatty acid has one or more aliphatic chains branched or unbranched with one or more substituents. In some embodiments, the fatty acid comprises from about 6 to about 22 carbon atoms in its aliphatic chain. In some embodiments, any one of the bonds between each carbon atom may be in the cis or trans configuration.

Cyclodextrins are natural compounds formed during bacterial digestion of cellulose. They cannot be hydrolyzed by common amylases, but can be fermented by the intestinal microflora. They are poorly resorbed and are considered generally recognized as safe (GRAS) for oral administration. On Dec. 22, 2004, Error! Reference source not found. α-CD was declared GRAS for “use in selected foods, except meat and poultry, for fiber supplementation, as a carrier or stabilizer for flavors (flavor adjuvant), as a carrier or stabilizer for colors, vitamins and fatty acids and to improve mouthfeel in beverages (GRN No. 155, updated November 2016 as GRN 678 to reflect an expected 90^{th} percentile for intake of 420 mg/kg, or ˜30 g/d at 70 kg). An α-CD monograph is included in the U.S. Pharmacopeia/National Formulary (USP/NF25), the European Pharmacopoeia (EP 6.0), and the Handbook of Pharmaceutical Excipients. Cyclodextrins are exempted from the requirement of a tolerance under 40 CFR 180.950 when used in or on various food commodities. (FR 70 128 28780 Jul. 6, 2005). On Oct. 9, 2017, cyclodextrins were included in the Annex to the European Commission guideline on ‘Expedients in the labelling and package leaflet of medical products for human use’.

Cyclodextrins are compounds made up of (D-glucose) six (α-CD), seven (β-cyclodextrin), or eight (γ-cyclodextrin) sugar molecules bound together in a toroid (truncated cone) with a lipophilic inner and a hydrophilic outer surface (FIG. 17). This combination of features makes them suitable as an expedient to solubilize lipophilic drugs. Substitution of any of the hydroxyl groups, even by hydrophobic moieties, will result in a dramatic increase in water solubility. The main reason for the solubility enhancement is that chemical manipulation frequently transforms the crystalline cyclodextrins into amorphous mixtures of isomeric derivatives. For example, the aqueous solubility of β-cyclodextrin increases with increasing degree of methylation. The highest solubility is obtained when about two-thirds of the hydroxyl groups (i.e., 14 of 21) are methylated. Error! Reference source not found. substantially improves water solubility over β-CD) (Table 1, see also Table 3).

TABLE 1
Physicochemical characteristics of selected cyclodextrins

| | Error! | | | Error! |
|---|---|---|---|---|
| | Reference | | | Reference |
| | source not | | | source not |
| | found. | HP-α-CD | β-CD) | found. |
| Number of glucose units | 6 | 6 | 7 | 7 |
| Internal diameter | 4.5-5.2 | 4.7-5.3 | 6.0-6.5 | 6.0-6.5 |
| Solubility in water [g/l] | 145 | >500 | 18.5 | >600 |

With parenteral delivery, “the steady-state volume of distribution for β-cyclodextrin . . . in rats, rabbits, dogs, and humans corresponds well with the extracellular fluid volume of each species, suggesting that no deep compartments or storage in pools are involved.” J Pharm Sci, 86 (2), 147-62. “α- and β-cyclodextrin are excreted almost completely in an intact form into the urine” J Pharm Sci, 86 (2), 147-62. Common cyclodextrins obtained by the substitution of the R groups on the edge (rim) of the toroid (FIG., (Brewster M E, Loftsson T (2007) Advanced Drug Delivery Reviews 59:645-66)) include, but are not limited to, methyl (including randomly methylated) CH₃, 2-hydroxypropyl (Error! Reference source not found.): CH₂CHOHCH₃, sulfobutylether (CH₂)₄SO₃Na⁺, acetyl, succinyl, glucosyl, maltoseyl, carboxymethyl ether, phosphate ester, simple polymers, or carboxymethyl. Since both the number of substitutes and their location will affect the physicochemical properties of the [cyclodextrin] molecules, such as their aqueous solubility and complexing abilities, each derivative listed should be regarded as a group of closely related [cyclodextrin] derivatives.

“Chemical modifications have been made to CDs to increase their hydrophilic activity with the hope that the improved solubility would eliminate the renal toxicity [in rats].” (Irie T, Uekama K (1997) J Pharm Sci 86:147-62) “Substitution of any of the hydrogen bond forming hydroxyl groups, even by hydrophobic moieties such as methoxy and ethoxy functions, will result in a dramatic increase in water solubility . . . . The main reason for the solubility enhancement in these derivatives is that chemical manipulation frequently transforms the crystalline cyclodextrins into amorphous mixtures of isomeric derivatives.”(Loftsson T, Brewster M E (1996) J Pharm Sci 85:1017-25) “For example, the aqueous solubility of β-cyclodextrin . . . increases with increasing degree of methylation. The highest solubility is obtained when two-thirds of the hydroxyl groups (i.e., 14 of 21) are methylated.”(Loftsson T, Brewster M E (1996) J Pharm Sci 85:1017-25). 2-hydroxylpropyl-b-cyclodextrin (HP-b-CD), a hydroxyalkyl derivative, of β-CD, substantially improves water solubility (Table 3) While 1 g/kg β-cyclodextrin caused severe nephrosis in rats (Table 2), the same dose of HP-β-cyclodextrin did not cause adverse clinical signs. (Gould S, Scott R C (2005) Food Chem Toxicol 43:1451-9)

Common cyclodextrins obtained by the substitution of the R groups on the edge of the Error! Reference source not found. ring (FIG. 17, (Brewster M E, Loftsson T (2007) Advanced Drug Delivery Reviews 59:645-66)) include, but are not limited to,
methyl (including randomly methylated): CH₃,
2-hydroxypropyl (HP): CH₂CHOHCH₃,
Sulfobutylether: (CH₂)₄SO₃Na⁺
acetyl,
succinyl,
glucosyl,
maltoseyl,
carboxymethyl ether,
phosphate ester,
simple polymers, or
carboxymethyl.
   “Since both the number of substitutes and their location will affect the physicochemical properties of the cyclodextrin molecules, such as their aqueous solubility and complexing abilities, each derivative listed should be regarded as a group of closely related cyclodextrin derivatives.”(Loftsson T, Brewster M E (1996) J Pharm Sci 85:1017-25)

Cyclolab and Wacker AG sells suitable cyclodextrins, which may also be employed in the methods described herein. Active agents can be coupled to cyclodextrins (either directly or via a homo- or heterobifunctional crosslinker) depending on signs, symptoms, and/or disease to be treated. For example, piroxicam derivatives of α, β, and/or γ cyclodextrins can be employed where delivery of an NSAID would treat pain. In another nonlimiting example, in the treatment of multiple sclerosis cyclodextrins and/or HP cyclodextrins can be coupled (directly or using a crosslinker) to injectable medications (e.g., Avonex (interferon beta-1a), Betaseron (interferon beta-1b), Copaxone (glatiramer acetate), Extavia (interferon beta-1b), Glatopa, Plegridy (peginterferon beta-1a), Rebif (interferon beta-1a), Zinbryta (daclizumab)), oral medications (e.g., Aubagio (teriflunomide), Gilenya (fingolimod), Tecfidera (dimethyl fumarate)), or Infused medications (e.g., Lemtrada (alemtuzumab), Novantrone (mitoxantrone), Ocrevus (ocrelizumab), Tysabri (natalizumab)).

In regard to the number of active agents coupled to each cyclodextrin, it should be appreciated that each cyclodextrin can be coupled to one or more active agents, for example one per α-D-glucopyranoside unit. Accordingly, an α-cyclodextrin can be coupled to one, two, three, four, five, or six active agents. A α-cyclodextrin can be coupled to one, two, three, four, five, six, or seven active agents. A α-cyclodextrin can be coupled to one, two, three, four, five, six, seven, or eight active agents. The length of the crosslinker should be selected to allow coupling of the desired number of active agents and the size of such active agents. Each active agent coupled to each cyclodextrin can be the same or different. In another embodiment of the inventive subject matter, one or more hydroxypropyl groups of a HP-cyclodextrin are substituted with an active agent.

In lieu of, or in addition to, active agents, cyclodextrins and/or HP-cyclodextrins can optionally be coupled to carriers. Carriers include nanoparticles (e.g., gold nanoparticles, silica nanoparticles, carbon nanoparticles, etc.), liposomes (or surfactants used to make liposomes), polymers (synthetic and natural (carbohydrate, peptide/protein, nucleic acid), and hybrids and/or combinations thereof), and other cyclodextrins.

Advantageously, coupling cyclodextrins to carriers can increase the load if cyclodextrins/HP-cyclodextrin delivered to each cell. Without wishing to be bound by a particular hypothesis coupling cyclodextrins and/or cyclodextrins derivatives to carriers may also change the mechanism of cellular uptake to the endosomal uptake. Thus, the cyclodextrins and any active agent will be delivered to the lysosomes where it is desirable to complex cyclodextrins according to the inventive subject matter with lipids.

Low solubility in water, as with β-cyclodextrin, in particular, often results in precipitation of solid cyclodextrin complexes. “In addition, β- and δ-cyclodextrin form intramolecular hydrogen bonds between secondary OH groups, which detracts from hydrogen bond formation with surrounding water molecules [resulting in] low aqueous solubilities.”(Loftsson T, Brewster M E (1996) J Pharm Sci 85:1017-25)

Only in part related to the different physicochemical characteristics Table 1, Error! Reference source not found.s, β-CD)s, and γ-CD cyclodextrins have dramatically different biochemical function and, thus, different uses.
Only γ-CDs can be hydrolyzed by pancreatic amylases and saliva and are rapidly metabolized and absorbed in the small intestine.
β-CD)s and γ-CDs can bind fats at most at a 1:1 molar ratio, but Error! Reference source not found. can bind fat 9-times its weight.
Size of the cavity v. the molecule is a major determinant for the use of cyclodextrins. β-CD)s (and γ-CDs, to a lesser extent), but not Error! Reference source not found.s can bind sterols (including cholesterol) and steroids), because these molecules are too large to fit into the smaller cavity of Error! Reference source not found.s.
Methylated β-CD)s have a high affinity to common constituents of cell membranes and, thus, an extreme hemolytic effect, while Error! Reference source not found.s have very low effect on human erythrocytes.
The effect of the neuromuscular blocking drug rocuronium is reversed by the specifically designed γ-CD derivative sugammadex (octakis-(6-deoxy-6-S-mercaptopropionyl-γ-cyclodextrin sodium salt) to prevent residual paralysis.

For cyclosporin A, a mixture of Error! Reference source not found. and γ-CD is used to enhance the drug solubility without causing ocular irritation.

Only Error! Reference source not found. can be used for gold extraction through environmentally friendly co-precipitation.(Liu Z C, Samanta A, et al. (2016) Journal of the American Chemical Society 138:11643-53)

This “in-complete” list of differences between cyclodextrin demonstrates that effects seen by one cyclodextrin cannot be extrapolated to other cyclodextrins. In fact, there are only few examples, where more than one of the cyclodextrins is used for the same application:

Error! Reference source not found. is used, for instance,
As the active ingredient of Febreze®; the smaller Error! Reference source not found.s would trap too few malodourous molecules.
To remove cholesterol from whole egg, milk, the smaller Error! Reference source not found.s do not fit cholesterol

Error! Reference source not found.s are used
In foods based on vegan coconut milk powder (UK) as a replacement for sodium caseinate
In powdered alcohol
In Coenzyme Q10/L-Carnitine supplements to form complexes with the essential alpha linolic acid

Therapeutic uses also are highly specific. In one age related application (Nociari M M, Lehmann G L, et al. (2014) Proceedings of the National Academy of Sciences 111:E1402-E8), demonstrated that Error! Reference source not found. could bind A2E, a lipofuscin component, while Error! Reference source not found. could not (Only in part related to the different physicochemical characteristics Table 1, Error! Reference source not found.s, β-CD)s, and γ-CD cyclodextrins have dramatically different biochemical function and, thus, different uses.
Only γ-CDs can be hydrolyzed by pancreatic amylases and saliva and are rapidly metabolized and absorbed in the small intestine.
β-CD)s and γ-CDs can bind fats at most at a 1:1 molar ratio, but Error! Reference source not found. can bind fat 9-times its weight.
Size of the cavity v. the molecule is a major determinant for the use of cyclodextrins. β-CD)s (and γ-CDs, to a lesser extent), but not Error! Reference source not found.s can bind sterols (including cholesterol) and steroids), because these molecules are too large to fit into the smaller cavity of Error! Reference source not found.s.
Methylated β-CD)s have a high affinity to common constituents of cell membranes and, thus, an extreme hemolytic effect, while Error! Reference source not found.s have very low effect on human erythrocytes.
The effect of the neuromuscular blocking drug rocuronium is reversed by the specifically designed γ-CD derivative sugammadex (octakis-(6-deoxy-6-S-mercaptopropionyl-γ-cyclodextrin sodium salt) to prevent residual paralysis.(Booij LH (2009) Anaesthesia 64 Suppl 1:31-7)
For cyclosporin A, a mixture of Error! Reference source not found. and γ-CD is used to enhance the drug solubility without causing ocular irritation.
For intranasal insulin, DMβCD and Error! Reference source not found. are potent enhancers, but γ-CD, β-CD), and HPβCD are not.(Merkus F W, Verhoef J C, et al. (1991) Pharm Res 8:588-92; Shao Z, Krishnamoorthy R, et al. (1992) Pharmaceutical Research 9:1157-63)
Error! Reference source not found. but not β-CD) supplementation decreased atherosclerotic lesions in aorta in apoE-KO mice, although β-CD) but not Error! Reference source not found. supplementation decreases intestinal lipid absorption.(Sakurai T, Sakurai A, et al. (2017) Mol Nutr Food Res 61)
   Only Error! Reference source not found. can be used for gold extraction through environmentally friendly co-precipitation.(Liu Z C, Samanta A, et al. (2016) Journal of the American Chemical Society 138:11643-53)

This non-limiting list of differences between cyclodextrin demonstrates that effects seen by one cyclodextrin cannot be extrapolated to other cyclodextrins. In fact, there are only few examples, where more than one of the cyclodextrins is used for the same application:

Error! Reference source not found. is used, for instance,
As the active ingredient of Febreze®; the smaller Error! Reference source not found.s would trap too few malodourous molecules.
To remove cholesterol from whole egg, milk, the smaller Error! Reference source not found.s do not fit cholesterol

Error! Reference source not found.s are used
In foods based on vegan coconut milk powder (UK) as a replacement for sodium caseinate
In powdered alcohol
In Coenzyme Q10/L-Carnitine supplements to form complexes with the essential alpha linolic acid

Therapeutic uses also are highly specific. In one age related application (Nociari M M, Lehmann G L, et al. (2014) Proceedings of the National Academy of Sciences 111:E1402-E8), demonstrated that Error! Reference source not found. could bind A2E, a lipofuscin component, while Error! Reference source not found. could not (FIG.)

Rarely, if ever are several cyclodextrins used with the same type of substrates, attesting to the fact that their function is highly specific despite them comprising rings of sugars. One of the most important differences is the number of these sugars: “Cyclodextrin-Lipid Complexes: Size Matters”.(Szente L, Fenyvesi E (2017) Structural Chemistry 28:479-92).

### Cytotoxicity/Hemolysis

With parenteral delivery, “the steady-state volume of distribution for β-cyclodextrin . . . in rats, rabbits, dogs, and humans corresponds well with the extracellular fluid volume of each species, suggesting that no deep compartments or storage in pools are involved.” (Irie T, Uekama K (1997) J Pharm Sci 86:147-62) “α- and β-cyclodextrin are excreted almost completely in an intact form into the urine” (Irie T, Uekama K (1997) J Pharm Sci 86:147-62)Still cyclodextrins may cause two types of adverse events with parenteral delivery. First, they can accumulate in kidney cells, causing nephrotoxicity. Second, after the lipophilic drug is delivered, they form “a new lipid-containing compartment (or pool) in the aqueous phase into which [cellular lipid] compounds [are] extracted,” J Pharm Sci, 86 (2), 147-62, which could cause hemolysis.

Nephrotoxicity of parenteral cyclodextrins was shown in 1976. In the rat, LD₅₀ for α- and β-CD was determined as 1008 and 788 mg/kg, respectively, the same minimal dose at which changes were observed by light microscopy from a single dose. “Repeated administration of nephrotoxic doses [of β-cyclodextrin] resulted in extensive nephrosis.” Am J Pathol, 83 (2), 367-82. Nephrosis was not apparent in rats given 1, 2, 4, or 7 daily injections of 0.1 g/kg α-cyclodextrin; light microscopic lesions were found in one rat given 0.225 g/kg β-cyclodextrin daily for 4 days. Daily injections of 0.45 g/kg β-cyclodextrin resulted in severe nephrosis and produced no deaths. All rats given 0.9 g/kg β-cyclodextrin died within 4 days and [only] 1 died after 2 days of treatment with 1.0 g/kg α-cyclodextrin.” Am J Pathol, 83 (2), 367-82. “(see Table 2 for details). In summary,
Only one rat died from daily doses of 1.000 α-CD, while all rats died from daily doses of 0.900 β-cyclodextrin.
Electron microscopy was only conducted for β-cyclodextrin. Hence, it is not clear whether α-CD also causes crystals and ultrastructure alterations or whether this outcome is a result of the lower solubility in water in β-cyclodextrin (18.5) v. α-CD (145.0).

TABLE 2
Toxicity of cyclodextrins in rats

| | α-CD | β-cyclodextrin |
|---|---|---|
| | [g/kg] | [g/kg] |
| single dose i.v. LD₅₀ | 0.788/1.008 * | 0.788/1.008 * |
| nephrosis, | 0.100 no changes | 0.225 no changes |
| single dose s.c. | — | 0.450 no changes |
| | — | 0.670 changes seen |
| | 1.000 changes seen | 1.000 changes seen |
| nephrosis, | 0.100 no nephrosis | 0.225 one nephrosis |
| | | after 4 days |
| 1, 2, 3, 4, or 7 daily | — | 0.450 severe nephrosis |
| doses s.c. | | |
| | 1.000 severe | 0.900 all died within |
| | nephrosis one died | 4 days |
| | after 2 days | |
| long cytoplasmic crystals | — | 0.980 |
| ultrastructure alterations | — | 0.450 |
| . . . 24 hours after injection | | increased number of |
| | | vacuoles, |
| | | needle-like |
| | | microcrystals in |
| | | lysosomes from |
| | | nondegradable |
| | | CD |
| . . . 2-3 days after injection | — | extensive structural |
| | | alterations |
| . . . 72 hours after injection | — | irreversible injury |

Am J Pathol, 83 (2), 367-82.
Legend:
“—” not done,
“*” the paper is ambiguous about which drug caused either LD₅₀, see also Ribeek, Prinsen as quoted in (http://www.inchem.org/documents/jecfa/jecmono/v48je10.htm)

Low solubility in water, as with β-cyclodextrin, in particular, often results in precipitation of solid cyclodextrin complexes. “In addition, β- and δ-cyclodextrin form intramolecular hydrogen bonds between secondary OH groups, which detracts from hydrogen bond formation with surrounding water molecules [resulting in] low aqueous solubilities.” J Pharm Sci, 85 (10), 1017-25.

In four studies of outbred rodents (Riebeck 1990a/b/c, Prinsen 1991a, as quoted in WHO http://www.inchem.org/documents/jecfa/jecmono/v48je10.htm) macroscopic examination of dead and surviving animals either “did not reveal treatment-related alterations” (Riebeck 1990a/b/c) or “revealed a pale renal cortex” (Prinsen 1991a).

Although electron microscopy was performed only on β-cyclodextrin, but not in α-cyclodextrin, the abstract does not distinguish between the two cyclodextrins and, thus, α-CD is now commonly believed to be at least as nephrotoxic as β-cyclodextrin:
“Early studies showing the nephrotoxicity of the parent CDs [Frank et al. 1976]. . . ” See J Pharm Sci, 86 (2), 147-62 at p. 147 (citing Am J Pathol, 83 (2), 367-82).
“The renal toxicity of α-CD and β-CD after parenteral administration [Frank et al. 1976] . . . have been well documented [Irie and Uekama, 1997; Thompson 1997 (review only); Gould and Scott 2005 (β-CD only)].” See Toxicol Pathol, 36 (1), 30-42 at p. 31 (citing J Pharm Sci, 86 (2), 147-62; Am J Pathol, 83 (2), 367-82; Food Chem Toxicol, 43 (10), 1451-9).
“Both α-CD and β-CD showed renal toxicity after parenteral administration.” See Committee for Human Medicinal Products (CHMP) (2014), ‘Background review for cyclodextrins used as excipients’, Eurpean Medicines Agency, EMA/CHMP/333892/2013 at page 9.
“β-CD family (native, HPβCD and RAMEβ) was found [ . . . ] less toxic than α-CD family (native, HPαCD and RAMEα)[Monnaert et al. 2004].” Molecules, 21 (12).
“α-CD is the most toxic among the three native CDs.” Journal of Pharmacology and Experimental Therapeutics, 310 (2), 745-51.

However, when the dose “‘spike’ was spread to 6-8 hours . . . [d]oses of 1 g/kg not only did not result in animal deaths, but the dose did not even influence the growth of the young animals compared to controls—a clear sign of lack of toxic effects.”(Szejtli et al. 1981) Consistent with the latter results, “so far, however, there are no cases of kidney injury caused by cyclodextrins in humans.” Committee for Human Medicinal Products (CHMP) (2014), ‘Background review for cyclodextrins used as excipients’, Eurpean Medicines Agency, EMA/CHMP/333892/2013; see also Table 4.

α-CD is also well tolerated in cyclosporine eye-drops. Transplant Proc, 21 (1 Pt 3), 3150-2.

“Chemical modifications have been made to CDs to increase their hydrophilic activity with the hope that the improved solubility would eliminate the renal toxicity [in rats].” J Pharm Sci, 86 (2), 147-62. “Substitution of any of the hydrogen bond forming hydroxyl groups, even by hydrophobic moieties such as methoxy and ethoxy functions, will result in a dramatic increase in water solubility . . . . The main reason for the solubility enhancement in these derivatives is that chemical manipulation frequently transforms the crystalline cyclodextrins into amorphous mixtures of isomeric derivatives.” J Pharm Sci, 85 (10), 1017-25. “For example, the aqueous solubility of β-cyclodextrin . . . increases with increasing degree of methylation. The highest solubility is obtained when two-thirds of the hydroxyl groups (i.e., 14 of 21) are methylated.” J Pharm Sci, 85 (10), 1017-25. 2-hydroxylpropyl-b-cyclodextrin (HP-b-CD), a hydroxyalkyl derivative, of β-CD, substantially improves water solubility (Table 3) While 1 g/kg β-cyclodextrin caused severe nephrosis in rats (Table 2), the same dose of HP-β-cyclodextrin did not cause adverse clinical signs. Food Chem Toxicol, 43 (10), 1451-9.

Common cyclodextrins obtained by the substitution of the R groups on the edge of the α-CD ring (FIG. 17, J Pharm Sci, 85 (10), 1017-25) include, but are not limited to,
methyl (including randomly methylated): CH₃,
2-hydroxypropyl (HP): CH₂CHOHCH₃,
Sulfobutylether: (CH₂)₄SO₃Na⁺
acetyl,
succinyl,
glucosyl,
maltoseyl,
carboxymethyl ether,
phosphate ester,
simple polymers, or
carboxymethyl.
   “Since both the number of substitutes and their location will affect the physicochemical properties of the cyclodextrin molecules, such as their aqueous solubility and complexing abilities, each derivative listed should be regarded as a group of closely related cyclodextrin derivatives.” J Pharm Sci, 85 (10), 1017-25. In some embodiments, the disclosure relates to pharmaceutical composition comprising an α-CD deriviative which is any one of the above-identified compounds. In some embodiments, the disclosure relates to pharmaceutical composition comprising an α-CD deriviative which has the formula of FIG. 1 wherein each R is indpedently selected from H, CH2CH3, and CH2CH(OH)CH3 and n=0. In some embodiments, the disclosure relates to pharmaceutical composition comprising an 2 α-CD or alpha-cyclodextrin deriviatives which have the formula of FIG. 1 wherein each R is indpedently selected from H, CH2CH3, and CH2CH(OH)CH3 and n=0 molecules for every 1 fatty acid molecule, wherein the fatty acids are any one or more of the fatty acids disclosed herein. In some embodiments, the disclosure relates to any method disclosed herein, wherein the pharmaceutical compositions administered to a subject in need thereof is free of beta or gamma-cyclodextrin and/or a derivative thereof.

TABLE 3
Characteristics of selected cyclodextrins

| | | | | HP-β- |
|---|---|---|---|---|
| | α-CD | HP-α-CD | β-cyclodextrin | cyclodextrin |
| Number of | 6 | 6 | 7 | 7 |
| glucose | | | | |
| units | | | | |
| Solubility | 130-145 | ~500^{a} | 18.5 | >600 |
| in water | | | | |
| [g/l] | | | | |
| Internal | 4.7-5.2 | 4.7-5.2 | 6.0-6.4 | 6.0-6.4 |
| diameter | | | | |
| oral | 2-3% | | 0.6-2% | ≤3% |
| absorption | | | | |
| in rats | | | | |
| i.v. toxicity, | 1 died | | all died | |
| 1 g daily | after 2 d | | within 4 d | |
| half-life | | | | 1.7-1.9 |
| (t_{1/2}) [h] | | | | |
| Vol. of | | | | 0.2 |
| distribution | | | | |
| (V_{D}) | | | | |
| [1/kg] | | | | |
| Vₘₐₓ | 5.8 | | 166 | |
| acute i.v. | 0.5-0.8 | | 1 | 10 |
| toxicity in | | | | |
| rats [g/kg] | | | | |
| BBB | 1 | 2.5 | 2.5 | 2.5 |
| breakdown | | | | |
| [mM] | | | | |
| Transport | 21.5 | 16.5 | 26.7 | 9.3 |
| across BBB | (0.5 mM) | (1 mM) | (1 mM) | (1 mM) |
| [%, 2 h] | | | | |
| max marketed | 1.3* | | not | 16,000 |
| dose i.v. | | | suitable^{$} | |
| [mg/d] | | | | |

From J Pharm Sci, 85 (10), 1017-25; (Loftsson and Brewster 2010) J Pharm Pharmacol, 68 (5), 544-55.
*Prostavasin 80 mg/d × 4 wk (NCT00596752) @ 649.3 μg CD per 20 μg alprostadil (caverjet label)
^{$}Nat Rev Drug Discov, 3 (12), 1023-35.
^{a}http://cyclolab.hu/index.php/standard-grade-cyclodextrins/HPaCD
While 1 g/kg B-cyclodextrin caused severe nephrosis in rats (Table 1), the same dose of HP-β-cyclodextrin did not cause adverse clinical signs Food Chem Toxicol, 43 (10), 1451-9.
“8 hours after oral administration of 313 mg/kg ¹⁴C-BCD 3 μg of BCD was detected in 1000 μg of blood.”

In addition to the cyclodextrin derivatives noted above, Pocono Enterprise LLC sells several cyclodextrin products under the CAVCON brand. In addition to hydroxypropyl HP-β-cyclodextrin, sulfobutyl ether-β-cyclodextrin, hydroxyprolyl-γ-cyclodextrin, and methyl-β-cyclodextrins, CAVCON also sells mono-(6-amino-6-deoxy)-β-cyclodextrin, heptakis(6-amino-6-deoxy)-β-cyclodextrin, mono-(6-mercapto-6-deoxy)-β-cyclodextrin, hexakis-(6-mercapto-6-deoxy)-α-cyclodextrin, heptakis-(6-mercapto-6-deoxy)-β-cyclodextrin, octakis-(6-mercapto-6-Deoxy)-γ-cyclodextrin, mono-(6-(diethylenetriamine)-6-deoxy)-β-cyclodextrin, mono-(6-((triethylenetetraamine)-6-deoxy)-β-cyclodextrin, mono-(6-(tetraethylenepentamine)-6-deoxy)-β-cyclodextrin, mono-(6-p-toluenesulfonyl)-β-cyclodextrin, mono-(6-ethanediamine-6-deoxy)-β-cyclodextrin, mono-(6-(1,6-hexamethylenediamine)-6-deoxy)-β-cyclodextrin, mono-6-azido-6-deoxy-β-cyclodextrin, carboxymethyl-β-cyclodextrin, hexakis-(6-iodo-6-deoxy)-α-cyclodextrin, heptakis(6-iodo-6-deoxy)-β-cyclodextrin, and octakis-(6-iodo-6-deoxy)-γ-cyclodextrin. Commercially available cyclodextrins linked to active agents and polymers include salicylic acid-hydroxypropyl-β-cyclodextrin complex, menthol-hydroxypropyl-β-cyclodextrin complex, vanillin-β-cyclodextrin complex, piroxicam-β-cyclodextrin, soluble β cyclodextrin polymer crosslinked, and β cyclodextrin epichlorohydrin copolymer. Cyclolab also sells sutiable cyclodextrins, which may also be employed in the methods described herein.

It should be appreciated that several of these exemplary cyclodextrins derivatives include functionalities, which can be used to link the cyclodextrins to other active agents. For example, mercapto-cyclodextrins can be reacted with maleimide functionalized active agents and/or crosslinkers (homo- or hererobifuntional crosslinkers, e.g., bismaleimidoethane, 1,4-bismaleimidobutane, bismaleimidohexane, tris(2-maleimidoethyl)amine, 1,8-bismaleimido-diethyleneglycol, 1,11-bismaleimido-triethyleneglycol, N-α-maleimidoacet-oxysuccinimide ester, SM(PEG)24, succinimidyl iodoacetate, available from Thermo Fisher Scientific). Advantageously, disulfide-containing linker, such as dithiobismaleimidoethane and succinimidyl 3-(2-pyridyldithio)propionate, can be cleaved by reducing agents.

Amine functionalized cyclodextrins (e.g., diethylenetriamine, triethylenetetraamine, tetraethylenepentamine, ethanediamine, hexamethylenediamine derivatives, etc.) can be reacted with NHS esters to form amide bonds. Cyclodextrins functionalized with p-toluenesulfonyl and iodio leaving groups can undergo nucleophilic substitution reactions. Azide functionalized cyclodextrins can undergo Huisgen cycloaddition reactions with alkynes, (either copper catalyzed or copper free, e.g., using cyclooctynes). Without limitation, carboxy methyl groups can be further reacted to form amide and ester linkages. In some embodiments, the disclosure relates to pharmaceutical composition comprising any of the derivatives mentioned above in an alpha-cyclodextrin structure.

The active agent coupled to cyclodextrins (either directly or via a homo- or heterobifunctional crosslinker) is selected based on the signs, symptoms, and/or disease to be treated. For example, piroxicam derivatives of α, β, and/or γ cyclodextrins can be employed where delivery of an NSAID would treat pain. In another nonlimiting example, in the treatment of multiple sclerosis cyclodextrins and/or HP cyclodextrins can be coupled (directly or using a crosslinker) to injectable medications (e.g., Avonex (interferon beta-1a), Betaseron (interferon beta-1b), Copaxone (glatiramer acetate), Extavia (interferon beta-1b), Glatopa, Plegridy (peginterferon beta-1a), Rebif (interferon beta-1a), Zinbryta (daclizumab)), oral medications (e.g., Aubagio (teriflunomide), Gilenya (fingolimod), Tecfidera (dimethyl fumarate)), or Infused medications (e.g., Lemtrada (alemtuzumab), Novantrone (mitoxantrone), Ocrevus (ocrelizumab), Tysabri (natalizumab)).

In another embodiment of the inventive subject matter, the active agent treats Paget's disease. For example, treatments for Paget's disease include: Alendronate (Fosamax), Ibandronate (Boniva), Pamidronate (Aredia), Risedronate (Actonel), and Zoledronic acid (Zometa, Reclast).

In regard to the number of active agents coupled to each cyclodextrin, it should be appreciated that each cyclodextrin can be coupled to one or more active agents, for example one per α-D-glucopyranoside unit. Accordingly, an α-cyclodextrin can be coupled to one, two, three, four, five, or six active agents. A α-cyclodextrin can be coupled to one, two, three, four, five, six, or seven active agents. A α-cyclodextrin can be coupled to one, two, three, four, five, six, seven, or eight active agents. The length of the crosslinker should be selected to allow coupling of the desired number of active agents and the size of such active agents. Each active agent coupled to each cyclodextrin can be the same or different. In another embodiment of the inventive subject matter, one or more hydroxypropyl groups of a HP-cyclodextrin are substituted with an active agent.

In lieu of, or in addition to, active agents, cyclodextrins and/or HP-cyclodextrins can optionally be coupled to carriers. Carriers include nanoparticles (e.g., gold nanoparticles, silica nanoparticles, carbon nanoparticles, etc.), liposomes (or surfactants used to make liposomes), polymers (synthetic and natural (carbohydrate, peptide/protein, nucleic acid), and hybrids and/or combinations thereof), and other cyclodextrins.

Advantageously, coupling cyclodextrins to carriers can increase the load if cyclodextrins/HP-cyclodextrin delivered to each cell. Without wishing to be bound by a particular hypothesis coupling cyclodextrins and/or cyclodextrins derivatives to carriers may also change the mechanism of cellular uptake to the endosomal uptake. Thus, the cyclodextrins and any active agent will be delivered to the lysosomes where it is desirable to complex cyclodextrins according to the inventive subject matter with lipids.

### Cytotoxicity/Hemolysis

Phospholipids and cholesterol, the major building blocks of cell membranes, are both lipids. Hence, when cyclodextrins are given intravenously without having their lipophilic cavity filled (or after the lipophilic drug has been delivered), cyclodextrins can potentially extract phospholipids and cholesterol from membranes. “Several CDs have been demonstrated to cause cell lysis in different types of cells, indicating that the effect is not cell-type specific [(Irie and Uekama 1997)].”Toxicol Pathol, 36 (1), 30-42 (citing J Pharm Sci, 86 (2), 147-62).

In particular, CDs are known to induce shape changes of membrane invagination on erythrocytes and, at higher concentrations, induce hemolysis of human erythrocytes in the order of β-CD>α-CD>γ-CD.(Irie et al. 1982), i.e., the hemolytic activity of α-CD is lower than that of both β-CD and HP-β-CD. See J Pharm Sci, 86 (2), 147-62, FIG. 6. In addition, β-CD induces caspase-dependent apoptotic cell death in human keratinocytes on depletion of membrane cholesterol, whereas α-CD and HP-β-CD are not apoptotic to this type of cell. See Toxicol Pathol, 36 (1), 30-42.

Similarly, HP-α-CD and maltosyl-α-CD were found to be less cytotoxic than α-CD on heterogeneous human epithelial colorectal adenocarcinoma (Caco-2) cells. See Biol Pharm Bull, 24 (4), 395-402; Molecules, 20 (11), 20269-85.

From these observations, HP-cyclodextrins not only have less nephrotoxicity, but also less cytotoxicity/hemolysis.

2-hydroxypropyl-beta-cyclodextrin (HP-β-CD) is used as an expedient/solvent for a many lipophilic drugs, including the neurosteroid allopregnanolol. The cyclodextrin α-CD has been approved and is used as an expedient of alprostadil (a prostaglandin) for intracavernosal injection in the treatment of erectile dysfunction.

Still, less cytotoxicity overall may not be enough. A 2010 study showed that HP-β-CD causes permanent hearing loss in cats at doses of 4-8 g/kg, with raised concerns for the use of a cyclodextrin as a drug, rather than an expedient (smaller dose). See Pediatr Res, 68 (1), 52-6.

In 2014, it was observed that the reported benefit of the neurosteroid allopregnanolol in Niemann-Pick C (NPC) disease was, in fact, due to the solvent, 2-hydroxypropyl-beta-cyclodextrin (HP-β-cyclodextrin). See Journal of Lipid Research, 55 (8), 1609-21. It was demonstrated the β-cyclodextrin at therapeutic doses extracts cholesterol from cellular components, rather than from cell membranes as previously believed. In September 2015, a phase 2b/3 study of 2-hydroxypropyl-beta-cyclodextrin started in patients with neurologic manifestations of Niemann-Pick Type C1 disease. ClinicalTrials.gov, (Mar. 6, 2016), NCT02534844. The question was whether one could retain the benefit for treating NPC while avoiding risk of ototoxicity (“dead or deaf?”).

There is a positive correlation between the hemolytic activity of several CDs and their capacity to solubilize cholesterol,” J Pharm Sci, 86 (2), 147-62, suggesting that ototoxicity might be directly related to the extraction of cholesterol from outer hair cells.

The results of the genetic study reported herein show that progression in breast cancer, including metastases are caused, in part, by excessive the conversion of glycerophospholipids (LPC, PC, PS, PA) into PI, the substrate of the PI cycle, which regulates endocytosis by making phosphoinositides available. (LPC is also involved in surfactant inhibition in cyctic fibrosis, where “LPC or FFA mediated surfactant inhibition was reversed by MβCD, even in the relative absence of cholesterol [likely reflecting] the capacity of MβCD to sequester non-steroidal lipids in addition to cholesterol.” Gunasekara, L., et al. (2017), ‘Pulmonary surfactant dysfunction in pediatric cystic fibrosis: Mechanisms and reversal with a lipid-sequestering drug’, J Cyst Fibros.

All cyclodextrins have the ability to scavenge phospholipids. β-cyclodextrins, however, have often been chosen over α-CDs because they can be used as expedients for more (larger) drugs. The smaller α-CD (only six starch molecules, FIG. 18) has higher specificity for the smaller phospholipids , because cholesterol does not fit the smaller cavity of α-CD (FIG. 1). See European Journal of Biochemistry, 186 (1-2), 17-22; Journal of Lipid Research, 55 (8), 1609-21. The specificity of extraction of phospholipids, cholesterol, and proteins are shown in Error! Reference source not found.23. See European Journal of Biochemistry, 186 (1-2), 17-22. Among the phospholipids, α-CD scavenges glycerolipids, such as the glycerolipids, more effectively than sphingolipids In particular, α-CD is known to form complexes both around both the inositol head or the sn-2 chain of PI. See J Pharm Sci, 86 (8), 935-43. Hence, α-CD specifically downregulates the substrate for regulation of endocytosis via the PI cycle, thereby downregulating endocytosis with higher specificity than β-cyclodextrin. HPαCD retains the preference of α-CD for phospholipids over cholesterol.

Ototoxicity of β-cyclodextrin is believed to be caused by β-cyclodextrin depriving prestin of the cholesterol it needs to function in outer hair cells. See Biophysical Journal, 103 (8), 1627-36; Sci Rep, 6, 21973. As the cavity of α-CD is too small for cholesterol, α-CD and it's derivatives, including, but not limited to, HP-α-CD, avoid ototoxicity.

Long-term parenteral administration of HP-β-CD (200 mg/kg) was reported to decrease bone mineral density (BMD), which was associated with increased bone resorption, while a CD-bisphosphonate conjugate, alendronate-β-CD (ALN-β-CD) was shown to be bone-anabolic. See Toxicol Pathol, 40 (5), 742-50; Biomaterials, 29 (11), 1686-92.

B) Treatment of Cancer Patients with α-CDor Derivatives or Salts Thereof,

Several embodiments of the disclosure include the use of alpha-cyclodextrin and/or derivatives or pharmaceutically acceptable salt thereof.

Embodiments of the present disclosure are particularly useful to treat individuals who have cancer identified as having one or a plurality of cells with an abnormally high rate of endocytosis or exocytosis, or, in some embodiments, a dysfunctional PI cycle. In some embodiments, methods for treating an subject who has cancer comprise the steps of first identifying cancer as having a high rate of endocytosis or exocytosis, or, in some embodiments, a dysfunctional PI cycle, and then administering to such a subject a therapeutically effective amount of a cyclodextrins. In some preferred embodiments, the identification of cancer as having a high rate of endocytosis or exocytosis, or, in some embodiments, a dysfunctional PI cycle, is done by PET imaging, preferably using a fluorescent tag, antibody, or other agent that identifies mutations in the amino acid sequence of Table 6b or an amino acid sequence at least 70%, 80%, 90%, 95% 96%, 97%, 98%, or 99% homology to the amino acid sequences of Table 6b, In some embodiments, the cyclodextrin is effective to scavenge phospholipid in greater than 50% of cells in an in vitro migration assay at a concentration of less than 4 mM, 3 mM, 2 mM, or 1 mM. In some embodiments, the cyclodextrin or derivative or salt thereof is effective to inhibit migration of cells in greater than 50% of cells in an in vitro cell migration assay at a concentration of about 1 mM. In some embodiments, the cyclodextrin or derivative or salt or clathrate thereof is effective to reduce cell migration in greater than 50% of cells in an in vitro cell migration assay at a concentration from about 0.5 to about 1.5 mM.

In some embodiments of the present disclosure, methods for treating an individual who has been identified as having cancer comprise administering to such an individual a therapeutically effective amount of the cyclodextrin or derivative or salt thereof which is known to be effective to inhibit cell migration in greater than 50% of cells in an in vitro migration assay at a concentration of less than 1 mM. In some preferred embodiments, the cyclodextrin or derivative or salt thereof is effective to inhibit cell migration in greater than 50% of cells in an in vitro cell migration assay at a concentration of less than about 1.5 mM. In some preferred embodiments, the cyclodextrin or derivative or salt thereof is effective to inhibit cell migration in greater than about 50%, 60%, 70%, 80%, or 90% of cells in an in vitro cell migration assay at a concentration of about 1 mM, In some preferred embodiments, prior to administration of the cyclodextrin or derivative or salt thereof, the cancer is confirmed as being a cancer comprising one or a plurality of cells characterized by an abnormally high rate of endocytosis or exocytosis, or, in some embodiments, a dysfunctional PI cycle. The preferred method of doing so is be PET imaging, polymerase chain reaction (PCR) of a sample such as a biopsy.

Methods are provided for inhibiting, even partially, metastasis of a cancer cell. The methods comprise delivering to the cancer cell an amount of cyclodextrin or derivative or salt thereof effective to inhibit cell migration of the cell. The cyclodextrin or derivative or salt thereof used is effective to slow migration of a cancer cell in greater than 50% of cells in an in vitro cell migration assay at a concentration of less than about 2 mM or about 1.5 mM. In some embodiments, the cyclodextrin or derivative or salt thereof is effective to inhibit cell migration in greater than 50% of cells in an in vitro cell migration assay at a concentration of less than 1.5 mM. In some preferred embodiments, the cyclodextrin or derivative or salt thereof is effective to inhibit cell migration in greater than 50% of cells in an in vitro cell migration assay at a concentration of at or about less than 1.0 mM. In some embodiments, the treatment simultaneously reduces the ototoxicity of the treatment.

Embodiments of the present disclosure are particularly useful to treat patients who have cancer with cancer cells that have abnormally high rate of endocytosis or exocytosis, or, in some embodiments, a dysfunctional PI cycle. Such cancers include most cancers and generally exclude those cancers arising from tissues associated with lipid production such as liver cancer, and cancer involving fat cells. Cancer cells that have abnormally high rate of endocytosis or exocytosis, or, in some embodiments, a dysfunctional PI cycle, are generally limited to epithelial cell derived cancers. In some embodiments, cancer is from epithelial cells of the breast, colon, lung, or prostate. Thus, some methods described herein relate to methods of treating a cancer patient who has cancer that have abnormally high rate of endocytosis or exocytosis, or, in some embodiments, a dysfunctional PI cycle, wherein such methods comprise the step of administering to such patient or subject a therapeutically effective amount of cyclodextrin. In preferred embodiments, the cyclodextrin or derivative or salt thereof is known to be effective to slow cell migration in greater than 50% of cells in an in vitro cell migration assay at a concentration of less than 2 mM. In some preferred embodiments, the cyclodextrin or derivative or salt thereof is effective to induce apoptosis in greater than about 50%, 60%, 70%, 80%, or 90% of cells in an in vitro cell migration at a concentration of less than 1.5 mM. Cancer cells that have abnormally high rate of endocytosis or exocytosis, or, in some embodiments, a dysfunctional PI cycle, generally have dysfunction of enzymes of Table 5b and/or metabolize and/or uptake high levels of phospholipids around their microenvironment. In some preferred embodiments, prior to administration of cyclodextrin or derivative or pharmaceutically acceptable salt thereof, the cancer is confirmed as being a cancer characterized by an abnormally high rate of endocytosis or exocytosis, or, in some embodiments, a dysfunctional PI cycle. The preferred method of doing so is be PET imaging, PCR or immunohistochemistry of a sample.

Methods are provided for preventing or inhibiting the rate of metastases of a cancer cell characterized by an abnormally high rate of endocytosis or exocytosis, or, in some embodiments, a dysfunctional PI cycle, The methods comprise delivering to the cancer cell an amount of a cyclodextrin or derivative or pharmaceutically acceptable salt thereof effective to reduce cell migration of the cell. The cyclodextrin or derivative or pharmaceutically acceptable salt thereof used is effective to inhibit or reduce the rate of cell migration in greater than about 50%, 60%, 70%, 80%, or 90% of cells in an in who cell migration assay at a concentration of less than 1.5 mM. In some preferred embodiments, the disclosed treatment herein is effective to inhibit or reduce the rate of cell migration in greater than about 50%, 60%, 70%, 80%, or 90% of cells in an in vitro cell migration assay at a concentration of less than 1.1 mM. In some preferred embodiments, the disclosed treatment herein is effective to inhibit or reduce the rate of cell migration in greater than about 50%, 60%, 70%, 80%, or 90% of cells in an in vitro cell migration assay at a concentration of less than about 1.0 mM.

In some embodiments, any of the methods disclosed herein are free of administration of a cyclodextrin that scavenges cholesterol upon administration to a subject.

The production of cyclodextrins is relatively simple and involves treatment of ordinary starch with a set of easily available enzymes. Commonly, cyclodextrin glycosyltransferase (CGTase) is employed along with α-amylase. First starch is liquefied either by heat treatment or using a-amylase, then CGTase is added for the enzymatic conversion. CGTases can synthesize all forms of cyclodextrins, thus the product of the conversion results in a mixture of the three main types of cyclic molecules, in ratios that are strictly dependent on the enzyme used: each CGTase has its own characteristic (113:7 synthesis ratio. Purification of the three types of cyclodextrins takes advantage of the different water solubility of the molecules: β-CD, which is very poorly water-soluble (18.5 g/l or 16.3mM) (at 25° C.) can be easily retrieved through crystallization while the more soluble α- and γ-CDs (145 and 232 g/l respectively) are usually purified by means of expensive and time consuming chromatography techniques. As an alternative a “complexing agent” can be added during the enzymatic conversion step: such agents (usually organic solvents like toluene, acetone or ethanol) form a complex with the desired cyclodextrin which subsequently precipitates. The complex formation drives the conversion of starch towards the synthesis of the precipitated cyclodextrin, thus enriching its content in the final mixture of products. Wacker Chemie AG uses dedicated enzymes, that can produce alpha-, beta- or gamma-cyclodextrin specifically. This is very valuable especially for the food industry, as only alpha- and gamma-cyclodextrin can be consumed without a daily intake limit.

### B) Pharmaceutical Compositions and Routes of Administration

In some embodiments, the present disclosure provides compositions comprising any one or more of the active agents described herein, either alone or in combination, for example for use in treating a malignant, neurodegenerative, cardiovascular, metabolic, inflammatory, autoimmune, or viral disease or disorder. For example, in some embodiments, the present disclosure provides compositions comprising a cyclodextrin, or an analogue or derivative thereof, for example for use in treating breast or prostate cancer whose cells exhibit a lysosmal storage dysfunction.

In some embodiments, the present disclosure provides compositions comprising any one or more of the active agents described herein, either alone or in combination, for example for use in treating a malignant or neurodegenerative disease or disorder. For example, in some embodiments, the present disclosure provides compositions comprising a cyclodextrin, or an analogue or derivative thereof, for example for use in treating breast or prostate cancer.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., PIP, PIP2, PIP3), and/or reducing, eliminating, or slowing the progression of disease symptoms (e.g. symptoms of malignant, cardiovascular, or metabolic disorders or a neurodegeneration such as symptoms of Alzheimer's or Parkinson's disease). Determination of a therapeutically effective amount of a compound of the disclosure is well within the capabilities of those skilled in the art, especially in light of the detailed disclosure herein.

The pharmaceutical composition may be formulated by one having ordinary skill in the art with compositions selected depending upon the chosen mode of administration. Suitable pharmaceutical carriers are described in the most recent edition of Remington's Pharmaceutical Sciences, A. 0501, a standard reference text in this field.

Administering the pharmaceutical composition can be effected or performed using any of the various methods known to those skilled in the art. Systemic formulations include those designed for administration by injection, e.g. subcutaneous, intravenous, intramuscular, intrathecal or intraperitoneal injection, as well as those designed for transdermal, transmucosal, oral or pulmonary administration. In some embodiments, administration of the effective amount of pharmaceutical composition disclosed herein is not limited to any particular delivery system and includes, without limitation, parenteral (including subcutaneous, intravenous, intramedullary, intraarticular, intramuscular, or intraperitoneal injection), rectal, topical, transdermal, muscoal or oral (for example, in capsules, suspensions, or tablets) administration. In some embodiments, administration to a subject in need thereof occurs in a single dose or in repeat administrations, and in any of a variety of physiologically acceptable salt forms, or with an acceptable pharmaceutical carrier or additive as part of a pharmaceutical composition. In some embodiments, any suitable and physiological acceptable salt forms or standard pharmaceutical formulation techniques, dosages, and excipients are utilized. In some embodiments, the step of administering comprises administering the composition or pharmaceutical composition intravenously, intramuscularly, topically, intradermally, intramucosally, subcutaneously, sublingually, orally, intravaginally, intracavernously, intraocularly, intranasally, intrarectally, gastrointestinally, intraductally, intrathecally, subdurally, extradurally, intraventricular, intrapulmonary, into an abscess, intra articularly, into a bursa, subpericardially, into an axilla, intrauterine, into the pleural space, intraperitoneally, transmucosally, or transdermally. Pharmaceutical compositions described herein may be administered by a variety of routes including oral, buccal, sublingual, rectal, transdermal, subcutaneous, intravenous, intramuscular, intrathecal, intraperitoneal and intranasal. (Gaurav Tiwari, Ruchi Tiwari & Awani K. Rai, Cyclodextrins in delivery systems: Applications, J Pharm Bioallied Sci. 2010 April-June; 2(2): 72-79.) Depending on whether intended route of delivery is oral or parenteral, the active agents can be formulated as compositions that are, for example, either injectable, topical or oral compositions. Liquid forms of compositions may include a suitable aqueous or nonaqueous vehicle with buffers, suspending and dispensing agents, colorants, flavors and other suitable ingredients known in the art. Solid forms of compositions may include, for example, binders, excipients, lubricants, coloring agents, flavoring agents and other suitable ingredients known in the art. The active agents and pharmaceutical compositions described herein may also be administered in sustained release forms or from sustained release drug delivery systems known in the art.

The pharmaceutical composition may depend on the disease or condition and on whether the administration is to prevent or to treat the disease or condition. For instance, administration for prevention of several diseases or conditions, including, but not limited to cancer, may be orally, without penetration enhancers, and at a lower dose. Administration for a subject showing symptoms of cancer, such as triple-negative node-positive breast cancer, the administration may be parenteral, regioselective, and at higher dose. Methods for targeting may include, but are not limited to combination with folate or antifolates (methotexate, pemetrexed). Combination includes, but is not limited to esterification with or without linkers. Administration to a subject showing symptoms of a neurodegenerative disease may be intrathecally without penetration enhancers and modifications for targeting.

In some embodiments the compositions of the present disclosure are pharmaceutical compositions comprising one or more active agents, as described herein, together with one or more conventionally employed components suitable for use in pharmaceutical delivery such as carriers, stabilizers, diluents, dispersing agents, suspending agents, thickening agents, excipients, and the like, may be placed into the form of pharmaceutical formulations. Nonlimiting examples of such formulations include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration. The pharmaceutical compositions and formulations described herein may, where appropriate, be conveniently presented in discrete unit dosage forms and may be prepared by any of the methods well known in the art of pharmacy. Such methods include the step of bringing into association an active agent with liquid carriers, solid matrices, semi-solid carriers, finely divided solid carriers or combinations thereof, and then, if necessary, shaping the product into the desired delivery system. Unit dosage forms of a pharmaceutical composition or formulation preferably contain a predetermined quantity of active agent and other ingredients calculated to produce a desired therapeutic effect, such as an effective amount of a therapeutically effective amount. Typical unit dosage forms include, for example, prefilled, premeasured ampules or syringes of liquid compositions, or pills, tablets, capsules or the like for solid compositions.

For parenteral administration, the cyclodextrin or derivative thereof can be, for example, formulated as a solution, suspension, emulsion or lyophilized powder in association with a pharmaceutically acceptable parenteral vehicle or pharmaceutically acceptable carrier. Examples of such vehicles or carriers are water, saline, Ringer's solution, dextrose solution, 5% human serum albumin, Ringers dextrose, dextrose and sodium chloride, lactated Ringers and fixed oils, polyethylene glycol, polyvinyl pyrrolidone, lecithin (glycerophospholipids), arachis oil or sesame oil. Liposomes and nonaqueous vehicles such as fixed oils may also be used. The vehicle or lyophilized powder may contain additives that maintain isotonicity (e. g, sodium chloride, mannitol) and chemical stability (eg, buffers and preservatives). The formulation is sterilized by commonly used techniques. Parenteral dosage forms may be prepared using water or another sterile carrier. For example, a parenteral composition suitable for administration by injection is prepared by dissolving 1.5% by weight of active ingredient in 0.9% sodium chloride solution. Alternatively, the solution can be lyophilised and then reconstituted with a suitable solvent just prior to administration.

Pharmaceutically acceptable carriers are well known to those skilled in the art and include, but are not limited to, from about 0.01 to about 0.1 M or about 0.05 M phosphate buffer or about 0.8% saline. Intravenous carriers include fluid and nutrient replenishers, electrolyte replenishers such as those based on Ringer's dextrose, and the like. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media.

The pharmaceutical compositions can be prepared using conventional pharmaceutical excipients and compounding techniques. Oral dosage forms may be elixers, syrups, tablets, pills, dragees, capsules, liquids, gels, syrups, slurries, suspensions and the like, for oral ingestion by a patient to be treated. The typical solid carrier may be an inert substance such as lactose, starch, glucose, cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; binding agents, magnesium sterate, dicalcium phosphate, mannitol and the like. A composition in the form of a capsule can be prepared using routine encapsulation procedures For example, pellets containing the active ingredient can be prepared using standard carrier and then filled into a hard gelatin capsule; alternatively, a dispersion or suspension can be prepared using any suitable pharmaceutical carrier(s), for example, aqueous gums, celluloses, silicates or oils and the dispersion or suspension then filled into a soft gelatin capsule. Typical liquid oral excipients include ethanol, glycerol, glycerin, non-aqueous solvent, for example, polyethylene glycol, oils, or water with a suspending agent, preservative, flavoring or coloring agent and the like. All excipients may be mixed as needed with disintegrants, diluents, lubricants, and the like using conventional techniques known to those skilled in the art of preparing dosage forms. If desired, disintegrating agents may be added, such as the crosslinked polyvinylpyrrolidone, agar, or alginic acid or a salt thereof such as sodium alginate. If desired, solid dosage forms may be sugarcoated or coated using standard techniques, including, but not limited to the use of chitosan, to target specific regions of the gastrointestine tract. For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compositions of the disclosure may take the form of tablets, lozenges, and the like formulated in conventional manner. The compounds may also be formulated in rectal or vaginal compositions such as suppositories or enemas. A typical suppository formulation comprises a binding and/or lubricating agent such as polymeric glycols, glycerides, gelatins or cocoa butter or other low melting vegetable or synthetic waxes or fats. For administration by inhalation, the compounds for use according to the present disclosure are conveniently delivered in the form of an aerosol spray from pressurized packs or a nebulizer, with the use of a suitable propellant, e. g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Even the larger βCD was seen to be suitable for inhalation. See Arch Med Res, 45 (8), 711-29.

The formulations may also be a depot preparation which can be administered by implantation (for example subcutaneously or intramuscularly) or by intramuscular injection. In such embodiments, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used. Certain organic solvents such as dimethylsulfoxide also may be employed, although usually at the cost of greater toxicity. Additionally, the compounds may be delivered using a sustainedrelease system, such as semipermeable matrices of solid polymers containing the therapeutic agent. Various of sustained-release materials have been established and are well known by those skilled in the art. Sustained release capsules may, depending on their chemical nature, release the compounds for a few weeks up to over 100 days. Depending on the chemical nature and the biological stability of the therapeutic reagent, additional strategies for protein stabilization may be employed.

The compounds described herein may also be formulated for parenteral administration by bolus injection or continuous infusion and may be presented in unit dose form, for instance as ampoules, vials, small volume infusions or prefilled syringes, or in multi-dose containers with an added preservative.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

### C) Dosages

The dose of an active agent described herein may be calculated based on studies in humans or other mammals carried out to determine efficacy and/or effective amounts of the active agent (see section E, Clinical Outcomes, below). The dose amount and frequency or timing of administration may be determined by methods known in the art and may depend on factors such as pharmaceutical form of the active agent and route of administration, and patient characteristics including age, body weight or the presence of any medical conditions affecting drug metabolism.

In one embodiment, a dose may be administered as a single dose. In another embodiment, a dose may be administered as multiple doses over a period of time, for example, at specified intervals, such as, daily, bi-weekly, weekly, monthly, and the like. In another embodiment, the dose will be 700 mg/kg/d (Table 4, 2010-04-15). In another embodiment, the dose will be increased over time until early signs of renal or cytotoxicity are observed, in which case the dose level will be decreased to the previous, well-tolerated level.

In one embodiment, the dose of active agent is at least 10 mg, at least 20 mg, at least 30 mg, at least 40 mg, at least 50 mg, at least 75 mg, at least about 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 550 mg, at least 600 mg, at least 650 mg, at least 700 mg, at least 750 mg, at least 800 mg, at least 850 mg, at least 900 mg, at least 950 mg, at least 1000 mg, at least 1200 mg, at least 1500 mg, at least 2000 mg, at least 2500 mg, at least 3000 mg, at least 4000 mg, at least 5000 mg, at least 7500 mg, at least 10,000 mg, at least 15,000 mg, at least 20,000 mg, or at least 25,000 mg. In some such embodiments the above dosages are mg/day or mg/kg/day. In another embodiment, the dose of active agent is in the range of 1 to 10000 mg, 1 to 7500 mg, 1 to 5000 mg, 1 to 2500 mg, 1 to 1000 mg, 1 to 500 mg, 1 to 250 mg, 250 to 10000 mg, 250 to 5000 mg, 250 to 1000 mg, 250 to 500 mg, 500 to 10000 mg, 500 to 5000 mg, 500 to 1000 mg. In some embodiments the above dosages are mg/day or mg/kg/day.

In one embodiment, a single dose may be administered. In another embodiment, multiple doses may be administered over a period of time, for example, at specified intervals, such as, four times per day, twice per day, once a day, weekly, monthly, 4 times over 14 days, 2 times over 21 days, twice per month, 4 times over 21 days, 4 times per month, or 5, 6, 7, 8, 9, 10, 11, 12 or more times per month, per 21 days, per 14 days, or per week, and the like.

In one embodiment, the doses administered intravenously or intrathecally may be 5600 mg/kg/wk or 400 mg/wk (Table 4).

TABLE 4
Hempel twins dose adjustment

| Date | Description |
|---|---|
| 2004 January | Birth |
| 2008 Aug. 14 | confirmed diagnosis of NPC |
| 2009 Feb. 22 | HP-β-CD treatment plan |
| | Initial infusion: 4 d continuous IV of 80 mg/kg/d at |
| | a rate of 20 ml/hr |
| | Next: Weekly 8 h infusions starting at 160 mg/kg/d + |
| | add'l weekly infusions |
| | Next: 320 mg/kg/d |
| | Next: 400 mg/kg/d |
| 2009 Apr. 13 | Approval of i.v. infusion (INDs 104,114 and 104,116) |
| 2009 Jul. 7 | Protocol extension |
| | 400 mg/kg/d administered as a weekly eight hour |
| | infusion |
| 2009 Jul. 16 | Increased dosing frequency (twice/week) and rate of |
| | dose titration (100 mg/kg/infusion) |
| | Week 1: 500 mg/kg/d; 8 hrs × 1 + 3-4 days |
| | 600 mg/kg/d; 8 hrs × 1 |
| | Week 2: 700 mg/kg/d; 8 hrs × 1 + 3-4 days |
| | 800 mg/kg/d; 8 hrs × 1 |
| | Week 3: 900 mg/kg/d; 8 hrs × 1 + 3-4 days |
| | 1000 mg/kg/d; 8 hrs × 1 |
| | Initial infusion: 500 mg/kg/d over 8 hrs at a rate |
| | of 20 ml/hr |
| 2009 Oct. 8 | Pulmonary clinic visit |
| | 2800 mg/kg twice weekly over 8 hr (800 mg/kg/d) |
| 2010 Mar. 7 | “2-Hydroxypropyl-β-Cyclodextrin Raises Hearing |
| | Threshold in . . . Cats.” |
| | (Ward et al. 2010) |
| 2010 Apr. 15 | Hearing unaffected despite receiving steady state |
| | IV doses of 2.5 g/kg bi-weekly for >1 yr |
| | (5 g/kg/wk ≈ 700 mg/kg/d) |
| 2010 May 17 | Orphan-drug designation granted |
| 2010 Aug. 13 | Request for intrathecal delivery filed |
| | 200 mg HP-β-CD intrathecal biweekly (≈60 mg/kg/d) |
| | (Maarup et al. 2015) |

From Hastings C (2009 Feb. 22) Addi and Cassi Hydroxy-Propyl-Beta-Cyclodextrin Plan. Compassionate Use Clinical Study. Treatment Plan Version #2, http://addiandcassi.com/wordpress/wp-content/uploads/2009/09/FDA-Submission-for-Addi-and-Cassi-Cyclodextrin-Treatment-Plan.pdf
NPC: http://www.nnpdf.org/cyclodextrin.html

### D) Subjects

The methods and compositions described herein may be used to treat or prevent a malignant, neurodegenerative, cardiovascular, metabolic, inflammatory, autoimmune, or viral disease or disorder, in any subject in need of such treatment. In one embodiment, the subject is a human. It should be noted that, while in some embodiments the subjects to be treated are post-menopausal women, in other embodiments the methods of treatment described herein are not intended to be limited to such subjects. Rather, in some embodiments the subjects can be of any age, ranging from newborns to older adults. In some embodiments it may be desirable to treat young subjects, for example young infants, particularly where family history or genetic testing indicates that the subject is at risk for developing a malignant, neurodegenerative, cardiovascular, metabolic, inflammatory, autoimmune, or viral disease or disorder. Similarly, in some embodiments it may be desirable to treat much older subjects, particularly where such subjects begin to exhibit indicators or symptoms of a malignant, neurodegenerative, cardiovascular, metabolic, inflammatory, autoimmune, or viral disease or disorder.

The methods and compositions described herein may be employed as prophylactic treatments or therapeutic treatments. For prophylactic treatments, the methods and compositions provided herein can be used preventatively in subjects that do not yet exhibit any clear or detectable clinical indicators or symptoms of a malignant, neurodegenerative, cardiovascular, metabolic, inflammatory, autoimmune, or viral disease or disorder but that are believed to be at risk of developing a malignant, neurodegenerative, cardiovascular, metabolic, inflammatory, autoimmune, or viral disease or disorder, such as MS, prostate canceer or breast cancer, or diseases that are characterized by dysfunctional lysosomes. A subject receiving prophylactic treatment for α-CD, for example, may not exhibit any clinical indicators or symptoms of MS, prostate canceer or breast cancer, or diseases that are characterized by dysfunctional lysosomes. In the case of therapeutic treatments, the methods and compositions provided herein can be used in subjects that already exhibit one or more clinical indicators or symptoms of the disease or disorder, such as MS, prostate canceer or breast cancer, or diseases that are characterized by dysfunctional lysosomes. A subject receiving therapeutic treatment for MS, prostate canceer or breast cancer, or diseases that are characterized by dysfunctional lysosomes, for example, may have been clinically diagnosed with MS, prostate canceer or breast cancer, or diseases that are characterized by dysfunctional lysosomes or may otherwise exhibit one or more clinical indicators or symptoms of MS, prostate canceer or breast cancer, or diseases that are characterized by dysfunctional lysosomes.In some embodiments, the disclosure relates to methods of treating cancers comprising cells that are deficient or substantially deficient in any of the genes identified in the Figures or specification, such that the limited expression or lack of expression of those geens results in lysosomal dysfunction.

In one embodiment , a subject may have been identified as being at risk of developing a malignant, neurodegenerative, cardiovascular, metabolic, inflammatory, autoimmune, or viral disease or disorder. In one embodiment, the subject has a family history of a malignant, neurodegenerative, cardiovascular, metabolic, inflammatory, autoimmune, or viral disease or disorder. In one embodiment, the subject has one or more genetic risk factors associated with a malignant, neurodegenerative, cardiovascular, metabolic, inflammatory, autoimmune, or viral disease or disorder, for example, a genetic mutation in a gene associated with PIP cycling.

### E) Clinical Outcomes

In some embodiments the methods of treatment provided herein (which comprise, for example, administering to a subject an effective amount of a composition according to the present disclosure) result in, or are aimed at achieving, a detectable improvement in one or more clinical indicators or symptoms of cancer, including, but not limited to, changes growth, migration, or invasion. In some embodiment of the present disclosure a symptom or indicator of improvement is selected from the group comprising survival, disease-free survival, distant metastasis-free survival, results of a blood test (including, but not limited to circulating tumor DNA and prostate-specific antigen), an x-ray evaluation, the result of a physical examination (including, but not limited to a palpable tumor), or a tissue biopsy for histological evaluation.

To determine the highest tolerated dose in an individual complete blood count and serum chemistry will be collected and analyzed. The serum chemistries may include, but will not be limited to evaluation of electrolytes, bicarbonate, glucose, BUN, creatinine, magnesium, phosphate, hepatic enzymes (AST and ALT), total protein, albumin, bilirubin, and alkaline phosphatase. In addition, a complete lipid panel may be obtained and shape of erythrocytes may be evaluated microscopically. Bone density may be measured to identify early signs of osteoporosis.

The compositions and methods described herein are illustrative only and are not intended to be limiting. Those of skill in the art will appreciate that various combinations or modifications of the specific compositions and methods described above can be made, and all such combinations and modifications of the compositions and methods described herein may be used in carrying out the present disclosure. Furthermore, certain embodiments of the present disclosure are further described in the following non-limiting Examples, and also in the following Claims.

All publications, including patent applications and journal articles are incorporated by reference in their entireties.

### Related Patents

In the context of AD, decreasing phosphatic acid (PA) has been declared desirable before (U.S. Pat. No. 8,288,378)(T.-W. Kim et al. 2012b): “Agents which decrease PA levels include, but are not limited to, an inhibitor of diacylglycerol kinase, an inhibitor of phospholipase D1, and/or an inhibitor of phospholipase D2. [ . . . ] Non-limiting examples of [such agents] include, but are not limited to, siRNA directed toward phospholipase D1 or D2 or diacylgerycerol [sic].” No such agents were given in (U.S. Pat. No. 8,288,378)(T.-W. Kim et al. 2012b), which has not been cited.

### EXAMPLES

### Example 1

### The PI-Cycle is a Drug Target Against Metastases in Breast Cancer

The methods used to obtain the results presented in Example 1 are further described in Transl Psychiatry, 4, e354, which is hereby incorporated by reference in its entirety

Almost a decade after the completion of the Human Genome Project, the scientific and medical advances hoped for from genome-wide association studies (GWAS) have not yet been realized. Enlarging the sample size to tens of thousands of subjects greatly increases the duration and cost of data collection and, in a nonrandomized study, may somewhat paradoxically increase the risk of false positives. This Example describes combining a novel computational biostatistics approach with decision strategies fine-tuned to the exploratory nature of GWAS. With these methodological advances, disease-relevant functional gene clusters can now be suggested from studies of a few hundred narrowly defined cases only.

Although a history of familial breast cancer being a known risk factor of either breast or prostate cancer attests to a high degree of heritability, the genetic risk factors for breast or prostate cancerin the general population are still poorly understood. As described herein, data from three independent populations (available from NIH's dbGaP) were analyzed using u-statistics for genetically structured wide-locus data to explore epistasis. To account for systematic, but disease-unrelated differences in (non-randomized) genome-wide association studies (GWAS) and for conducting multiple tests in overlapping genetic regions, a novel study-specific criterion for ‘genome-wide significance’ was applied. Transl Psychiatry, 4, e354 Enrichment of the results in all three studies with genes associated with different stages of endocytosis confirms the hypothesis that control of endocytoses through PI cycling is involved in metastases as the process turning breast or prostate cancer into a deadly disease.

The approach used here had been validated in childhood absence epilepsy and then generated a novel testable hypothesis about preventing mutism in autism. See Pharmacogenomics, 14 (4), 391-401; Transl Psychiatry, 4, e354. With the additional evidence in these studies on breast cancer and the genetic data from many more studies are already publicly available, the described computational biostatistics approach will advance personalized medicine and comparative effectiveness research. The genetic data collected over the last decade, could finally yield profound insights into the mechanistic bases of many common diseases and subgroup analyses of phase II and phase III trials can now suggest risk factors for adverse events and novel directions for drug development.

Breast or prostate cancerare among the cancers with the highest mortalitly. Still, the genetic risk factors for the more common disease forms are still poorly understood.

### Subjects

The study was approved as appropriate. No human participants were involved in the research.

The results described herein are based on three studies of breast cancer in the US and Europe. These studies included data from
(1) the Cancer Genetic Markers of Susceptibility (CGEM) breast cancer genome-wide association study (http://www.ncbi.nlm.nih.gov/projects/gap/cgi-bin/study.cgi?study_id=phs000147.v3.p1), which included 1145 cases/1142 controls. See Nature Genetics, 39 (7), 870-74.
   and from two substudies of the nested case-control and one case-control study of estrogen receptor negative breast cancer within the Breast and Prostate Cancer Cohort Consortium (http://www.ncbi.nlm.nih.gov/projects/gap/cgi-bin/study.cgi?study_id=phs000812.v1.p1), both included in (Garcia-Closas et al. 2013):
(2) the European Prospective Investigation into Cancer (EPIC) of 511 estrogen-receptor negative cases and 500 controls, and
(3) the Polish Breast Cancer Case-Control Study (PBCS) of 543 estrogen-receptor negative cases (229 triple-negative) and 511 controls.^{b} ^{b} (http://www.ncbi.nlm.nih.gov/projects/gap/cgi-bin/study.cgi?study_id=phs000812.v1.p1)

### Methods

ssGWAS: After eliminating non-informative or low-quality SNPs, a traditional ssGWAS was performed, using u-statistics for univariate data (also known as the Mann-Whitney test which is equivalent to the Wilcoxon rank-sum test). See Annals of Mathematical Statistics, 18 (1), 50-60; Biometrics, 1, 80-83. By construction, the results of this analysis are very close to those obtained with the traditional Cochran-Armitage trend test. See Biometrics, 11 (3), 375-86.

Annotation: The annotation files available have proven to be inadequate to appropriately distinguish between genes (or splice variants of genes) that are too far away or close enough to be likely related to a SNP or region being implicated. On the other hand, diplotypes may span LD blocks outside of genes or their regulatory regions, in which case it is unlikely that the functional implication of the variation can be identified. Also, there may not be sufficient information avail-able to determine the function of a gene, in which case the funding would not be useful for identifying collections of functionally related genes. Hence, the results returned from the grid/cloud infrastructure, still need to be manually reviewed to resolve ambiguities regarding the annotation.

Wide-locus approach. To overcome several of the shortcomings seen in previous applications of single-SNP GWAS (ssGWAS) applied to common diseases, several strategies were combined at different stages of the analysis process. Wide-loci of up-to six neighboring SNPs were aimed at as a primary outcome and the same non-parametric GWAS approach was applied based on u-statistics for structured multivariate data with genotypic structures (μGWAS) as in the previous childhood absence epilepsy and autism studies. See Annals of Mathematical Statistics, 19, 293-325; Pharmacogenomics, 14 (4), 391-401; Transl Psychiatry, 4, e354. To avoid spurious findings, loci outside of linkage-disequilibrium (LD) blocks containing genes with known function or adjacent to their 5′- end were excluded. Loci highly influenced by a single SNP only were also excluded, unless this SNP was implicated in more than one of the studiers or had been implicated in previous studies.

Information Content: In contrast to traditional regression methods, muGWAS provides an intrinsic measure of “information content”, which can be used to highlight regions with high significance, but low information content as likely artifacts. In the Manhattan plot, below, highly significant results with low information content are highlighted in red and excluded (crossed out in white), unless there is other supportive evidence, such as a nearby SNP that had previously been reported as associated with breast cancer or another cancer. Some regions with low information content are dominated by a single SNP or involve diplotypes spanning LD blocks without being within a gene or its regulatory region. Diplotypes may also be excluded if moving the window by one SNPs results in a large (more than 100 fold) change in significance. Of note, these manual intervention cannot cause false positive results and current research aims at formalizing more of these rules to facilitate interpretation and avoid false negative results.

MAF-significance correlation: With any finite sample size, the significance of a u- or rank test is limited. Hence, more significant results can only be obtained for SNPs with sufficiently high MAF. ssGWAS simulations were performed with 2,500,000 permuted phenotypes, comparing two groups of equal size for various MAFs. The 1-10⁻⁵ quantile of the permutation distribution drops from the expected s=−log₁₀ p=5.26 cut-off, which is routinely met for MAF>0.33, to 4.9 (n=1000 subjects), 4.7 (n=500), and 4.5 (n=300) for a MAF of 0.05. For the 7.5 level, the bias is projected to be even larger. Due to this MAF-significance correlation, the expected diagonal in a ssGWAS QQ plot under the null hypothesis that “no SNP is associated with the trait,” JAMA, 299 (11), 1335-44, turns into an expected curve dropping below the diagonal towards the end. Transl Psychiatry, 4, e354.

Estimating the expected s-value (−log₁₀(p)) distribution from >10⁸ permutations to obtain stable estimates of the 1-10^{-7.5} quantile is neither practical, nor sufficient to avoid a biased selection of SNPs for limited tests. Due to the MAF-significance correlation, any SNP ‘significant’ when comparing observed phenotypes, is also more likely to be ‘significant’ with random phenotype permutations. Transl Psychiatry, 4, e354.

Non-randomization bias: The reason for this curvature often not being recognized is that GWAS subjects are deterministically categorized based on their outcome (e.g., non-verbal vs. verbal), rather than randomly assigned to interventions (as in clinical trials). Any deterministically categorized populations, however, are expected to differ systematically in aspects related to neither the condition of interest nor common ancestry factors (which could potentially be accounted for through stratification). When the downward trend from using a limited test and the upward bias from deterministic selection are similar, the s-values may still appear to follow the diagonal, except for loci suggesting “true association.” JAMA, 299 (11), 1335-44.

Multiplicity adjustments for diplotype length: For multivariate tests of overlapping diplotypes, the estimated quantile-rank (QR) curve needs to be elevated above the diagonal throughout to account for multiple tests conducted around the same SNP. Because most of these tests are highly dependent, the elevation of the estimated QR curve compared to the estimated QQ curve (FIG. 6-FIG. 11) is limited, but the distance is likely to vary across diseases and populations. Transl Psychiatry, 4, e354.

Projected QR curves: The diagonal of the traditional QQ-plot does not depend on any data, including the most ‘significant’ data. The s-values are expected to fit the diagonal for the most part (except for the most significant results), as the vast majority of SNPs are expected not to be associated with the disease. In direct analogy, the QR curve for a multivariate test should be ‘smooth’, with upward deviations indicating ‘true association’, which could be disease-related or not. Based on the above rationale and the simulation results mentioned above, the highest point of the projected QR curve (apex) for each chromosome can be estimated from a smooth projection of the s-values after truncating as many of the highest values as needed for the projection to have a monotone increase and, conservatively for a limited test, a non-positive second derivative. See JAMA, 299 (11), 1335-44. Fitting against the data also reduces the effect of population stratification. See JAMA, 299 (11), 1335-44. (For computational convenience, locally weighted polynomial regression was selected, as implemented in S+ (TIBCO Software Inc.) as ‘loess. smooth ( . . . degree=2, family=“gaussian”)). See Journal of the American Statistical Association, 83 (403), 596-610.

Estimated whole genome QR apex: While chromosomes may differ with respect to their content of related and unrelated risk factors (see, e.g., the HLA region in autoimmune diseases), random errors are expected to have the same distribution across all chromosomes. Hence, the expected WG apex can be estimated as the (winsorized) median projected apex among chromosomes with the smallest deviation of s-values from the projection. (Here, ten chromosomes were selected based on the maximum norm, and the median for robustness, but the strategy to determine the optimal number, including the criteria for ‘optimality’, remains to be determined.) Transl Psychiatry, 4, e354.

Estimated QR curves: The estimated curve for each chromosome is then calculated as the loess projection of this chromosome's s-values with as many of the highest values replaced with the estimated WG apex until the curve's apex is at or below that level. Applied to the WG projection (QR plots, bottom right), this procedure yields the estimated WG curve. See Journal of the American Statistical Association, 83 (403), 596-610. Simulation results demonstrate the low variance of the estimates from phenotype permutations and the similarity of their median apex with the winsorized median apex estimated from the observed s-values. Transl Psychiatry, 4, e354.

Study-specific genome-wide significance: For studies aiming to confirm individual SNPs as associated with a phenotype, the ‘confirmatory’ paradigm requires adjustment for multiplicity. See American Statistician, 34 (1), 23-25. When applied to GWAS, these adjustments are typically based on a ‘customary’ fixed 0.05 level, irrespective of study size or relative risk of type I over type II errors (see (Fisher R A (1956), p. 358) and (Gigerenzer G (2004) Psychol Sci 15:286-7) for a discussion), and the assumption of 1,000,000 independent SNPs, irrespective of chip density (Pearson T A, Manolio T A (2008) JAMA 299:1335-44). Moving from individual SNPs to overlapping diplotypes increases the dependency of any formal multiplicity adjustment on assumptions with questionable biological validity. Transl Psychiatry, 4, e354.

As in most GWAS, however, the studies described in this Example do not aim to confirm hypotheses regarding specific SNPs. Instead, the studies described here aim at picking likely candidates from >40,000 (pseudo-) genes, whose relative importance and epistatic interactions are unknown. Since graphical procedures are particularly useful for such ‘exploratory’ studies, QR plots were chosen to guide with interpretation.See Tukey, John W. (1977), Exploratory data analysis (Reading, Mass.: Addison-Wesley). Exact cut-offs for deviation of s-values from the estimated curve are unknown. When “the knowledge [is] at best approximate[,] an approximate answer to the right question, which is often vague, [is far better] than an exact answer to the wrong question, which can always be made precise” (Tukey J W (1962) Ann Math Stat 33:1-&, p. 13-14). Hence, a heuristic approach is presented that relies on fewer unrealistic assumptions than typical attempts to quantify a particular error rate. Transl Psychiatry, 4, e354.

The expected WG curve needs to be estimated, the s-values have a complex dependency structure, and the appropriate level of significance (α) for the given sample size is unknown. Hence, a heuristic decision rule is proposed based on weak assumptions only. In the long run one would expect most s-values above the apex to be significant at any α>0 (consistency) and regions with the strongest association to have the highest odds at being included (unbiasedness). For a particular a, one could lower the cut-off, but to account for variance in estimating the apex, one would need to raise it. As a compromise, the estimated WG apex is proposed as a cut-off for study-specific GWS. Transl Psychiatry, 4, e354.

Quantile-rank (QR) plots: As is customary with selection procedures, p-values were used mainly for the purpose of ranking loci. As no particular hypotheses regarding specific loci were to be confirmed, the traditional approach of exploring characteristics of the ‘QQ plot’ as decision criteria was modified and formalized. For multivariate tests of overlapping diplotypes, the straight line expected in the traditional ‘QQ plot’ under the univariate WG permutation hypothesis turns into a curve because many tests are performed per SNP. Even though the number of tests performed increases substantially, the increase in s-values shown in the QR curve compared to the QQ line (FIG. 7 v. FIG. 6, FIG. 9 v. FIG. 8, FIG. 11 v. FIG. 10) is limited, because most tests are highly dependent. Transl Psychiatry, 4, e354.

Whole-genome permutation bias: To estimate the expected distribution of s-values, one could average the results of repeated runs with random phenotype permutations. As each μGWAS analysis may require >100,000 hours on a grid/cloud with GPU enabled nodes, however, simulations requiring >108 replications to estimates the 1-10^{-7.5} quantile may not be feasible. The estimate from WG permutations (including computationally efficient approximations) is affected by biases due to subjects being categorized based on their outcome (e.g., non-verbal vs. verbal), rather than randomly assigned to interventions (as in clinical trials), so that the groups are expected to differ systematically in aspects related to neither the condition of interest nor common ancestry factors. With binary outcomes, significant results can also not be caused by a few ‘outliers’ only, so that significance is correlated with high MAF (ssGWAS) or low skewness of the scores (μGWAS). Hence, regions with significant allelotype differences between observed phenotypes have also a larger chance to be significant among random phenotype permutations. Transl Psychiatry, 4, e354.

Selective chromosome permutation: The proposed use of a selected chromosome permutation approach reduces this bias. While chromosomes may differ with respect to their content of disease related and unrelated risk factors, random errors are expected to have the same distribution across all chromosomes. Hence, the above biases are reduced by excluding chromosomes containing regions of high significance when determining the permutation distribution. In particular, the endpoint of the expected distribution for each chromosome can be estimated from the loess projection to the p-values after truncation to ensure a monotone increase and a non-positive second derivative. Similarly, the endpoint of the expected distribution is estimated from the median of the limited set of, e.g., ten, chromosomes with the lowest maximum deviation of the distribution of s-values from the loess projection. Transl Psychiatry, 4, e354.

Formal QR cut-off for deviation: The estimate of the expected distribution for each chromosome is then calculated as the loess fit of the individual chromosomes' data with a sufficient number of results at the high end replaced with the expected endpoint until the curve is curtailed to that level, unless the initial loess fit already remains below this target level. The same procedure, when applied to the WG data, yields the estimation of the WG distribution. Simulation results demonstrate the low variance of the estimates based on random permutations of the phenotypes and that their median is closely resembled by the estimate of the distribution obtained from the observed data. Transl Psychiatry, 4, e354,

### Results

Previously known results from CGEM: Traditionally, GWAS have often identified only a small number of SNPs per study. A previous ssGWAS analysis of the CGEMS data had implicated two loci in trend analysis:

| | | | | |
|---|---|---|---|---|
| chr10: | 124,992,475 | rs10510126: | 6.15 | BUB3, (long EST |
| | | | | not5 identified) |
| chr10: | 123336180 | rs1219648: | 5.49 | FGFR2 |
| | 123341314 | rs2420946 | 5.46 | FGFR2 |

ssGWAS results: Single-SNP GWAS confirmed these findings at essentially equivalent levels of 6.20 and 5.57, respectively. See Nature Genetics, 39 (7), 870-74. Many other results had been dismissed in previous published analyses because p-values did not reach the traditional level of “fixed genome-wide significance” (typically, 7.5). From the ssGWAS QR plots (CGEM: FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG., EPIC: FIG., and PBCS: FIG.) many of the genes above the cut-off for study-specific genome-wide significance fit the paradigm of being involved in signaling at the membrane (GPCRs, Fc receptors, growth factor receptors, ion channels) or processes in the nucleus (cell cycle control, transcription, splicing) (see Table 5, columns Mbrn and Ncls).

muGWAS results: In muGWAS (CGEM: FIG. 7, EPIC: FIG. 10, and PBCS: FIG. 8), the proportion of genes related to membrane signaling and nuclear processes is even higher than in ssGWAS. In addition, a group of genes known to play a role in either in the phosphatidyl-inositol (PI) cycle (FIG. 12) or in endocytosis (FIG. 13A) stands out.

Validation (same intragenic region): PRKCQ (chr 10), was significant by muGWAS in CGEM (mu: 6.70, ss: 3.47, “indicating study-specific genome-wide significance) and by ssGWAS in EPIC (mu: 5.26, ss: 3.87*). The same region (chr10:6,540,724-6,573,883) was implicated in both populations. In PBCS, in contrast, there was no association in this region (<2.00), consistent with the notion of different risk factors for breast cancer in different populations. Validation (same gene): MEGF11 was implicated in both CGEM and PBCS. MEGF11 was even elevated (3.31) in EPIC. A single SNP was highly influential in either population, but it was not the same SNP (CGEM: rs189155, PBCS: rs12903880, EPIC: rs333554). All three SNPs are located in the coding region, but they are not in LD. One other SNP in MEGF11 (rs1477798) has been implicated in colorectal cancer. See Plos One, 7 (5), e38175. Validation (similar function): In a complex disease, populations may differ with respect to the risk factors that are present in each population. In particular, the proportion of risk conferred by different genes with similar function may differ and, even if the same gene is involved, risk may be associated with different SNPs.

One pair of functionally related genes stand out in ssGWAS (Table 5):

BMPR1B (CGEM)—BMP7 (EPIC)

Among muGWAS results (Table 5), there are three more pairs of functionally related genes:

ATP8B1 (CGEM)—ATP8A1 (EPIC)

MEGF11 (CGEM, PBCS)

AGPAT4 (CGEM)—AGPAT3 (EPIC)

Mutations in PI3K, PTEN, and SYNJ2 are known to be associated with breast cancer.

The mechanism commonly believed to be involved is the dysregulation of the AKT/TSC/mTOR growth pathway downstream of PI(3,4,5)P₃ and PI(3,4)P₂. The results of this analysis point to three additional points where the PI cycle is involved (FIG. 12):
PI(4,5)P2 (SCARB2, UNC13C, STXBP1, SDCBP2, MEGF11, SYT17, N4BP3, VAV3) and
PI(3)P (NLRP4, EEA1, RAB32), as well as
overall activation of PI (ATP8A1, ATP8B1, SLC5A3, AGPAT3, AGPAT4, ANXA4)

With the exception of CHMP7, RAPGEF4, and EEA1, all these genes have previously been shown to be associated with breast cancer (http://www.genecards.org).

The novel finding is that breast cancer risk is conferred not only by a variety of variations in
genes involved in nuclear processes causing susceptibility for cancer and
genes involved in membrane processes providing growth signals,
   as well as a few specific variations connecting the two by increasing
PI(3,4,5)P₃ (loss-of-function in PTEN, gain-of-function in PI3K),
PI(3,4)P₂ (gain-of-function in SYNJ1/2 or INPPL1), or
PI(3)P (gain-of-function in INPP4B),
   but by a global dysregulation of the PI cycle, including
entry of phosphatidylinositol (PI) (involving AGPAT3, AGPAT4, and SLC5A3), and
entry of phosphatidylserine/phosphatidylcholine (PS/PC) (involving ATP8A1, ATP8B1, and ANXA4),
   and endocytosis as a critical component of migration and invasion. Endocytosis is known to be controlled by PI signaling, which is consistent with the genes identified in the results presented:
at the plasma membrane stage (PM, eight genes),
at the early endosome stage (EE, four genes), and
at the late endosome stage (LE, two genes).

In some embodiments, the subject has a disease or disorder that is characterized by a dysfunctional lysosomal pathway by deficient expression of any one or combination of genes disclosed above.

### Discussion

The approach used here differs from traditional GWAS in both the statistical method being used and the decision strategy. To address the statistical method challenges specific to GWAS, the novel approach
(a) avoids making assumptions about a particular degree of dominance.
(b) draws for the fact that both SNPs neighboring a disease locus should be in LD, unless they are separated by a recombination hotspot.
(c) can distinguish between SNPs belonging to the same tag sets, but differ in their order along the chromosome.
(d) accounts for different disease loci within the same region having similar effects and for compound heterozygosity within the statistical method (rather than through visual inspection looking for several SNPs within a region having high significance), and
(e) provides additional information (“information content”) that can be used to prioritize results.

The use of a decision rule that accounts for
(a) GWAS being non-randomized,
(b) the aim being selecting sets of genes, knowing that some must have an effect, rather than testing the hypothesis that no gene has an effect, at all,
(c) accounting for differences in MAF in estimating the expected distribution of p-values, and
(d) adjusts for tests in overlapping diplotypes being related.

The validation of μGWAS in CAE demonstrated the ability of μGWAS to identify genes modulating a known disease pathway, where traditional ssGWAS had identified a single SNP (in a pseudo-gene) only. The subsequent application to mutism in autism confirmed the ability of μGWAS to identify clusters of genes related to the same biological function in two independent populations. See Pharmacogenomics, 14 (4), 391-401; Transl Psychiatry, 4, e354.

By using this novel decision rule alone with traditional single-SNP GWAS, the number of “significant” genes rises from none to >20 in each of the three studies considered here. The set of genes seen in ssGWAS using the novel decision rules includes half of the genes associated with the novel PI cycling pathway. The novel non-parametric wide-locus approach then adds the other half of the genes involved in PI cycling.

The PI cycle is critical for many cellular function in eukaryotic cells, including oocyte maturation, fertilization, and embryogenesis, cell growth, cytoskeleton dynamics, membrane trafficking, and nuclear events. See Philos Trans R Soc Lond B Biol Sci, 320 (1199), 415-26; Biochemistry, 49 (2), 312-7. Hence, it is not surprising that the PI cycle is tightly controlled. In particular, PI(4.5)P2, PI(4)P, PI(3,4)P2, and PI(3)P are tightly regulated by both three kinases and three groups of phosphatases. See Biochim Biophys Acta, 1851 (8), 1066-82. Different subsets of phosphatases (FIG., boxes) are counteracting the effects of the kinases, further reducing the impact any variation in a particular phosphatase might have on the system as a whole. Hence, a specific intervention modifying the state of this tightly regulated system might not suffice to achieve a sustained effect.

That “both PtdIns(3,4,5)P₃ and PtdIns(3,4)P₂ are likely required for a cell to achieve and sustain a malignant state”, has been formulated as the “two PIP hypothesis.” See Ann N Y Acad Sci, 1217, 1-17. The results presented here suggest not only that PI(3)P is required, but shift in focus further toward to the PI cycle as a whole. This new focus has direct implications for the development of drugs.
The model of a linear (PI-PIP-PIP2-PIP3) PI system suggested inhibition of PI3K as a strategy to reduce activity along the AKT/TSC/mTOR growth pathway. Only a small proportion of patients, however, benefit from interrupting this linear pathway by blocking PI3K. See Science Translational Medicine, 7 (283), 283ra51-83ra51. The limited success of wortmannin and other drugs blocking PI3K is consistent with the ability of the PI cycle to compensate not only for natural, but also for pharmaceutical disturbance at a particular point.
High levels of PI(3,4,5)P3, PI(3,4)P2, and PI(3)P all are known to correlate with a negative outcome of cancers. “Altered abundance of phosphatidyl inositides (PIs) is a feature of cancer. Various PIs mark the identity of diverse membranes in normal and malignant cells.” Sengelaub, Caitlin A., et al. (2015), ‘PTPRN2 and PLCβ1 promote metastatic breast cancer cell migration through PI(4,5)P2-dependent actin remodeling’, The EMBO Journal. The model of a PI cycle tightly regulated around the PI(4,5)P2-PI(4)P-PI(3,4)P₂-PI(3)P pathway suggests overall downregulation of PI activity as a more successful strategy to correct for excessive activation involving the PI cycle than blocking individual or pairs of kinases or phosphatases.

Neither breast nor prostate cancer per se are lethal; it is metastases spreading to other organs that cause cancer-related death. As seen in treatments involving cytotoxic drugs, reducing cell growth, in general, often causes side-effects (nausea, loss-of-hair, . . . ), without necessarily reducing the risk of metastases, because growth and metastasis may be regulated by different pathways.

To metastasize, “tumor cells must develop motile and invasive phenotypes.” Endocytosis is known to be required for cell migration. See Cold Spring Harbor Perspectives in Biology, 5 (12). As “defective vesicular trafficking of growth factor receptors, as well as unbalanced recycling of integrin- and cadherin-based adhesion complexes, has emerged in the past 5 years as a multifaceted hallmark”, “derail[ing] endocytosis” has been suggested as a strategy to prevent metastases in cancer. Nat Rev Cancer, 8 (11), 835-50. “Activation of signal transduction pathways associated with endocytic trafficking (FIGS. 13a and b) is critical for tumor cell migration. As a consequence, selective targeting endocytic trafficking and signaling could potentially allow for the development of novel cancer therapeutics to prevent metastasis.” Oncotarget, 7 (1), 5-6; see FIGS. 13A and 13B: Known relationship of genes implicated in muGWAS with stages in the process of endocytosis (FIG. 13A) and exocytosis/lysosomal function (FIG. 13B). Boxes: genes identified in the present disclosure by stage of endo-/exocytyosis: Formation of clathrin-coated vesicles (CCVs) and E3 ubiquitination, separation of inactive integrin (fast recycling) from active integrins (slow recycling), sorting between secretory, lysosomal, and (slow) recycling pathway, and lysosomal degradation. Underlined genes are known breast cancer promoters and suppressors, respectively. Clathrin-mediated endocytosis (CME) begins with co-assembly of the heterotetrameric clathrin adaptor complex AP-2 with clathrin at PI(4,5)P2-rich plasma membrane (PM) sites. AP-2 in its open conformation recruits clathrin and additional endocytic proteins, many of which also bind to PI(4,5)P2. Clathrin-coated pit (CCP) maturation may be accompanied by SHIP-2-mediated dephosphorylation of PI(4,5)P2 to PI(4)P. Synthesis of PI(3,4)P2 is required for assembly of the PX-BAR domain protein SNX9 at constricting CCPs and may occur in parallel with PI(4,5)P2 hydrolysis to PI(4)P via synaptojanin, thereby facilitating auxilin-dependent vesicle uncoating by the clathrin-dependent recruitment and activation of PI3KC2α, a class II PI3-kinase. PI(3,4)P2 may finally be converted to PI(3)P en route to endosomes by the 4-phosphatases INPP4A/B, effectors of the endosomal GTPase Rab5. Adapted from Posor, Y., Eichhorn-Grunig, M., and Haucke, V. (2015), ‘Phosphoinositides in endocytosis’, Biochim Biophys Acta, 1851 (6), 794-804. In the early endosome (EE), β1 integrins are sorted. Inactive integrins undergo fast “short loop” recycling; active integrins go to the late endosome (EE)/multivesicular body (MVB) for slow “long group” recycling (RAB11), lysosomal degeneration (unless rescued by RAB25/CLIC3), or secretion via the trans-Golgi-network (TGN) mediated by RAB9. Fast recycling of epidermal growth factor receptor drives proliferation, so one would expect gain-of-function mutations in FIG. 8a. See Tomas, Alejandra, Futter, Clare E., and Eden, Emily R. (2014), ‘EGF receptor trafficking: consequences for signaling and cancer’, Trends in Cell Biology, 24 (1), 26-34. Lysosomal and synaptic vesicle exocytosis share many similarities. Endolysosome-localized PIPs may regulate lysosomal trafficking (derived, in part from Kegg pathways hsa04144 and hsa04721). Adapted from Samie, M. A. and Xu, H. (2014), ‘Lysosomal exocytosis and lipid storage disorders’, J Lipid Res, 55 (6), 995-1009. See Bohdanowicz, M. and Grinstein, S. (2013), ‘Role of phospholipids in endocytosis, phagocytosis, and macropinocytosis’, Physiol Rev, 93 (1), 69-106; Hesketh, G. G., et al. (2014), ‘VARP is recruited on to endosomes by direct interaction with retromer, where together they function in export to the cell surface’, Dev Cell, 29 (5), 591-606; Mosesson, Yaron, Mills, Gordon B., and Yarden, Yosef (2008), ‘Derailed endocytosis: an emerging feature of cancer’, Nat Rev Cancer, 8 (11), 835-50; Schmid, Sandra L. and Mettlen, Marcel (2013), ‘Cell biology: Lipid switches and traffic control’, Nature, 499 (7457), 161-62.

FIG .

### Example 2

### The Genetic Risk Factors in the PI-Cycle and Along the Endocytosis Pathway are Known as Shared Risk Factors for “Derailed Endocytosis” in Breast Cancer and “Deranged Endocytosis” Parkinson's/Alzheimer's Disease

AD and PD are known to share risk factors: Aβ and α-synuclein have been hypothesized to interact, Neurotox Res, 16 (3), 306-17; Plos One, 3 (9), e3135, and “moderate association” of AD with PD was found in a meta analysis of 14 studies conducted 1986-2010, Neuroepidemiology, 42 (2), 69-80, but a meta-analysis of single-SNP summary statistics from two sets of AD and PD GWAS, each imputed to 7,815K SNPs, “resulted in no significant evidence [for SNP] loci that increase the risk of both PD and AD.”.JAMA Neurology, 70 (10), 1268-76. Recently, having disease with Lewis bodies (DLB) diagnosed as either PD or AD was identified as a potential confounder in these studies and the above results suggest that lack of “significant evidence” above may have been because of ssGWAS having lower power than muGWAS for cis-epistatic effects. See Human Molecular Genetics, 23 (23), 6139-46; Neurobiol Aging, 38, 214 e7-10.

Endocytosis is a common component of the etiology of aging and neurodegenerative diseases: The term “derailed endocytosis” has been used to characterize an important component of the etiology of BC (Nat Rev Cancer, 8 (11), 835-50), AD (Biomed Res Int, 2014, 167024), and other “pathological conditions” (Cold Spring Harb Perspect Biol. 2014 August; 6(8): a016865). “New reports implicate altered [vacuolar H⁺]-ATPase activity and lysosomal pH dysregulation in cellular aging, longevity, and adult-onset neurodegenerative diseases, including forms of [PD] and [AD].” Colacurcio, D. J. and Nixon, R. A. (2016), Disorders of lysosomal acidification-The emerging role of v-ATPase in aging and neurodegenerative disease', Ageing Res Rev; see also FIG. 13, FIG. 14, and FIG. 15. In PD, “an age-related pathological depletion of functional endosomes may increase the susceptibility to stochastic molecular defects in this same pathway, which in some individuals may trigger [a] vicious circle. [ . . . ] Disease causing mutations cluster within [the endosomal] pathway and alter receptor recycling and/or α-synuclein degradation. In turn, α-synuclein accumulation [ . . . ] exacerbates defective endosomal processing by impairing the machinery involved in the sorting or fusion of endosomes”. Mol Cell Neurosci, 66 (Pt A), 21-28. In AD, “accelerated endocytosis causes endocytic cargos to accumulate within enlarged [LEs] and impairs lysosomal functions. [ . . . ] Pathogenic endocytosis [ . . . ] could be modulated therapeutically at multiple possible targets.” Mol Psychiatry, 21 (5), 707-16. “The underlying molecular mechanisms [in AD and PD] remain poorly understood, yet dysfunction in endocytic membrane trafficking is a recurrent theme, which may explain the neurodegenerative process.” Schreij, A. M., Fon, E. A., and McPherson, P. S. (2015), ‘Endocytic membrane trafficking and neurodegenerative disease’, Cell Mol Life Sci.

Overlapping epidemiology and etiology of BC and AD/PD: BC has high co-occurrence with PD. See Dermatol Surg, 42 (2), 141-6. Earlier reports that cancers reduce AD risk were linked to statistical models not accounting for competing risks and/or treatment effects of cancer drugs. See J Gerontol B Psychol Sci Soc Sci; Nat Rev Neurol, 12 (3), 126-26. Another reason for limited association between BC and AD may be that mutations may have opposite effects, such as gain-of-function in BC (and PD) and loss-of-function in AD. Overlapping genetic risk factor have already been reported. Mutations in the PD gene PSEN2 were also found in BC and AD. See Bioinformatics, 31 (11), 1701-7. Mutations in MAPT, which encodes the AD microtubule-associated protein Tau, were found in BC (Proceedings of the National Academy of Sciences of the United States of America, 102 (23), 8315-20) and PD (Int J Mol Sci, 17 (2), 206); DJ-1 was seen as elevated (Cancer Sci, 106 (7), 938-43), and the G₂₀₁₉S mutation in endosomal LRRK2 (Biochem Soc Trans, 43 (3)) increases risk. See JAMA Neurol, 72 (1), 58-65. Still, “the etiology of this link continues to be elusive”. Dermatol Surg, 42 (2), 141-6.

EEC is a common risk factor in BC, PD, and AD: In PD (Biochem Soc Trans, 43 (3)) and AD (Mol Psychiatry, 21 (5), 707-16; Schreij, A. M., Fon, E. A., and McPherson, P. S. (2015), ‘Endocytic membrane trafficking and neurodegenerative disease’, Cell Mol Life Sci.; Molecular Neurodegeneration, 9 (1), 1-9), endocytosis of α-synuclein (SNCA, FIGS. 13A and 13B) and amyloid beta precursor protein (APP, Error! Reference source not found.15 and FIG. 13) and respectively, are known to be critical early steps in the etiology leading to formation of plaques. “[G]enes that influence endocytosis are overrepresented as AD risk factors [and] endocytosis-related genes are the earliest known disease-specific neuronal response in AD. They develop early in Down syndrome, a cause of early-onset AD linked to an extra copy of APP.” Mol Psychiatry, 21 (5), 707-16.

Overlap of genetic risk factors for BC (from above and published results) and AD/PD (from published results): The vast majority of genes related to the PI-cycle and EEC genes identified in muGWAS of breast cancer (Table 5, column PI/EC) had already been identified in previous functional studies and gene expression studies of both BC and AD/PD (Table 6 and Table 7).

TABLE 6
PI-Cycle overlap between BC, PD, and AD. Gene: Genes identified in BC
GWAS. EEC function: Known function in PI-cycle and/or EEC. KEGG: KEGG
pathway (http://www.genome.jp/kegg/pathway.html), EC: epithelial
cancer (carcinoma). ND: Neurodegenerative disease.

| Gene | PI-Cycle | KEGG | EC | ND | References |
|---|---|---|---|---|---|
| ATP8A1 | Increasing | | | | (Farge et al. 1999; |
| | extracellular | | | | Levano et al. 2009; |
| | PC and PS enhances | | | | Levano et al. 2012) |
| ATP8B1 | endocytosis | | | | |
| | | | BC | | (da Costa et al. 2012; |
| | | | | | Sjöblom et al. 2006) |
| | | | PC | | (Lee et al. 2013; |
| | | | | | Sekine et al. 2010; |
| | | | | | Trasino et al. 2009) |
| | | | | PD | (Levano et al. 2012) |
| | | | | | ATP8A2: |
| | | | | | (X. Zhu et al. 2012) |
| | | | | AD | (Soderberg et al. 1992) |
| | | | | | ATP8B4: |
| | | | | | (H. Li et al. 2008) |
| ANO4 | Ca+ dependent PL | | | | (Picollo et al. 2015) |
| | scramblase | | SC | | (Weber 2015) |
| | | | | AD | (Sherva et al. 2014) |
| ABCA1 | Regulates cellular | hsa02010 | | | (Hamon et al. 2006) |
| | lipid efflux; | | BC | | (Schimanski et al. 2010; |
| | | | | | Zhao et al. 2016) |
| | interacts with MEGF10 | | PC | | (Lee et al. 2013; |
| | | | | | Sekine et al. 2010) |
| | | | | PD | (Y. Dong et al. 2015; |
| | | | | | X. Dong et al. 2016; |
| | | | | | Loane et al. 2011; |
| | | | | | Pinho et al. 2016) |
| | | | | AD | (Boehm-Cagan et al. 2016; |
| | | | | | Koldamova et al. 2014; |
| | | | | | Nordestgaard et al. 2015; |
| | | | | | Pahnke et al. 2014) |
| | | | | HD | (Valenza et al. 2015) |
| AGPAT3 | converts | hsa00564 | | | (Bradley et al. 2015) |
| | lysophos- | | | | |
| | phatidylinositol | | | | |
| | (LPI) into | | | | |
| AGPAT4 | phosphatidylinositol | | | | |
| | (PI) | | BC | | (Hopkins et al. 2016; |
| | | | | | Sahay et al. 2015) |
| | | | PC | | AGPAT6: |
| | | | | | (Gatto et al. 2015) |
| | | | | PD | (Cheng et al. 2011) |
| | | | | AD | (Sherva et al. 2011) |
| DGKQ | Regenerates PA from | hsa00564, | | | (Sakane and Kanoh 1997) |
| | diacylglycerol (DAG) | hsa04070 | BC | | (Filigheddu et al. 2007) |
| | | | PC | | AGK: (Bektas et al. 2005) |
| | | | | PD | (Lill et al. 2012; |
| | | | | | Nalls et al. 2014) |
| | | | | AD | (X. C. Zhu et al. 2016) |
| LPPR1 | complexes with | | | | LPPR4: (Yu et al. 2015) |
| | LPPR3/4/5, | | | PD | (Moran et al. 2006) |
| | regulates PIS (CDIPT) | | | | |
| SYNJ2 | is recruited to the | hsa04070 | | | (Schmid and Mettlen 2013) |
| | nascent | | BC | | (Ben-Chetrit et al. 2015) |
| | clathrin coated pit | | PC | | (Rossi et al. 2005) |
| | | | | PD | SYNJ1 = PARK20 |
| | | | | AD | (Koran et al. 2014) |
| PTENP1 | PI3K/PTEN and | hsa04070 | | | PTEN: (Emeux et al. 2016) |
| | PI(3,4,5)P3 | | BC | | (H.-Y. Zhang et al. 2013) |
| | are involved in | | PC | | (Pourmand et al. 2007) |
| | endocytosis/cancer | | | PD | PINK1: |
| | | | | | (Choubey et al. 2014) |
| | | | | AD | (Frere and Slutsky 2016) |
| | | | | HD | PINK1: |
| | | | | | (Khalil et al. 2015) |

Sequences for Table 6a Genes.

| | |
|---|---|
| Name | Entrez Accession |

ATP8A1 10396 AF067820

| | |
|---|---|
| 1 | mptmrrtvse irsraegyek tddvsektsl adqeevrtif inqpqltkfc |
| | nnhvstakyn |
| 61 | iitflprfly sqfrraansf flfiallqqi pdvsptgryt tlvpllfila |
| | vaaikeiied |
| 121 | ikrhkadnav nkkqtqvlrn gaweivhwek vavgeivkvt ngehlpadli |
| | slsssepqam |
| 181 | cyietsnldg etnlkirqgl patsdikdvd slmrisgrie cespnrhlyd |
| | fvgnirldgh |
| 241 | gtvplgadqi llrgaqlrnt qwvhgivvyt ghdtklmqns tspplklsnv |
| | eritnvqili |
| 301 | lfciliamsl vcsvgsaiwn rrhsgkdwyl nlnyggasnf glnfltfiil |
| | fnnlipisll |
| 361 | vtlevvkftq ayfinwdldm hyeptdtaam artsnlneel gqvkyifsdk |
| | tgtltcnvmq |
| 421 | fkkctiagva yghvpepedy gcspdewqns qfgdektfsd ssllenlqnn |
| | hptapiicef |
| 481 | ltmmavchta vperegdkii yqaaspdega lvraakqlnf vftgrtpdsv |
| | iidslgqeer |
| 541 | yellnvleft sarkrmsviv rtpsgklrly ckgadtviyd rlaetskyke |
| | itlkhleqfa |
| 601 | teglrticfa vaeisesdfq ewravyqras tsvqnrllkl eesyeliekn |
| | lqllgataie |
| 661 | dklqdqvpet ietlmkadik iwiltgdkqe tainighsck llkknmgmiv |
| | inegsldgtr |
| 721 | etlsrhcttl gdalrkendf aliidgktlk yaltfgvrqy fldlalscka |
| | viccrvsplq |
| 781 | ksevvemvkk qvkvvtlaig dgandvsmiq tahvgvgisg neglqaanss |
| | dysiaqfkyl |
| 841 | knllmihgaw nynrvskcil ycfyknivly iieiwfafvn gfsgqilfer |
| | wciglynvmf |
| 901 | tamppltlgi ferscrkenm lkypelykts qnaldfntkv fwvhclnglf |
| | hsvilfwfpl |
| 961 | kalqygtafg ngktsdylll gnfvytfvvi tvclkaglet sywtwfshia |
| | iwgsialwvv |
| 1021 | ffgiysslwp aipmapdmsg eaamlfssgv fwmgllfipv asllldvvyk |
| | vikrtafktl |
| 1081 | vdevqeleak sqdpgavvlg kslteraqll knvfkknhvn lyrseslqqn |
| | llhgyafsqd |
| 1141 | engivsqsev iraydttkqr pdew |

ATP8B1 5205 AF038007

| | |
|---|---|
| 1 | msterdsett fdedsqpnde vvpysddete delddqgsav epeqnrvnre |
| | aeenrepfrk |
| 61 | ectwqvkand rkyheqphfm ntkflcikes kyannaikty kynaftfipm |
| | nlfeqfkraa |
| 121 | nlyflallil qavpqistla wyttivpllv vlgvtaikdl vddvarhkmd |
| | keinnrtcev |
| 181 | ikdgrfkvak wkeiqvgdvi rlkkndfvpa dilllsssep nslcyvetae |
| | ldgetnlkfk |
| 241 | msleitdqyl qredtlatfd gfieceepnn rldkftgtlf wrntsfplda |
| | dkillrgcvi |
| 301 | rntdfchglv ifagadtkim knsgktrfkr tkidylmnym vytifvvlil |
| | lsaglaigha |
| 361 | yweaqvgnss wylydgeddt psyrgflifw gyiivintmv pislyvsvev |
| | irlgqshfin |
| 421 | wdlqmyyaek dtpakarttt lneqlgqihy ifsdktgtlt qnimtfkkcc |
| | ingqiygdhr |
| 481 | dasqhnhnki eqvdfswnty adgklafydh ylieqiqsgk epevrqfffl |
| | lavchtvmvd |
| 541 | rtdgqlnyqa aspdegalvn aarnfgfafl artqntitis elgtertynv |
| | laildfnsdr |
| 601 | krmsiivrtp egniklyckg adtviyerlh rmnptkqetq daldifanet |
| | lrtlclcyke |
| 661 | ieekeftewn kkfmaasvas tnrdealdkv yeeiekdlil lgataiedkl |
| | qdgvpetisk |
| 721 | lakadikiwv ltgdkketae nigfacellt edtticyged insllharme |
| | nqtrnrggvya |
| 781 | kfappvqesf fppggnrali itgswlneil lekktkrnki lklkfprtee |
| | errmrtqskr |
| 841 | rleakkeqrq knfvdlacec saviccrvtp kqkamvvdlv krykkaitla |
| | igdgandvnm |
| 901 | iktahigvgi sgqegmqavm ssdysfaqfr ylqrlllvhg rwsyirmckf |
| | lryffyknfa |
| 961 | ftlvhfwysf fngysaqtay edwfitlynv lytslpvllm glldqdvsdk |
| | lslrfpglyi |
| 1021 | vgqrdllfny krffvsllhg vltsmilffi plgaylqtvg qdgeapsdyq |
| | sfavtiasal |
| 1081 | vitvnfqigl dtsywtfvna fsifgsialy fgimfdfhsa gihvlfpsaf |
| | qftgtasnal |
| 1141 | rqpyiwltii lavavcl1pv vairflsmti wpsesdkiqk hrkrlkaeeq |
| | wqrrqqvfrr |
| 1201 | gvstrrsaya fshqrgyadl issgrsirkk rspldaivad gtaeyrrtgd s |

ANO4 121601 AK091540

| | |
|---|---|
| 1 | aaaaactcca ttcgaaccca tggagcagaa aaccaccgac atctactcta |
| | tgagtgctgg |
| 61 | gcctcctggg gcgtgtggta taaataccaa cctttggatc ttgtaaggcg |
| | gtactttgga |
| 121 | gagaagattg ggttatattt tgcctggttg ggctggtaca ccggcatgct |
| | cttcccagct |
| 181 | gccttcattg gattgtttgt ctttttgtat ggcgtcacca ctctggatca |
| | cagccaagtc |
| 241 | agtaaagaag tctgccaagc tacagatatc atcatgtgtc ctgtgtgtga |
| | taaatactgt |
| 301 | ccattcatga ggctgtcaga cagctgtgta tatgccaagg taacccacct |
| | ttttgacaat |
| 361 | ggagccactg tcttctttgc tgttttcatg gcagtctggg caacagtttt |
| | cctggagttt |
| 421 | tggaaaagac ggcgagcagt aattgcttat gactgggatt tgatagactg |
| | ggaagaagag |
| 481 | gaggaagaaa tacgacccca gtttgaagcc aagtattcca agaaagagcg |
| | gatgaatcca |
| 541 | atttctggaa agccagaacc ttatcaagca tttacagata aatgcagcag |
| | acttatcgtt |
| 601 | tctgcatctg gaatattttt tatgatctgc gtggtgattg ctgccgtgtt |
| | cgggatcgtc |
| 661 | atttaccggg tggtgactgt cagcactttc gctgccttta agtgggcgtt |
| | aatcaggaat |
| 721 | aactctcagg ttgcaaccac agggactgct gtgtgcatca acttctgtat |
| | cattatgttg |
| 781 | ctgaatgtgc tctatgaaaa agttgccctg cttctgacga atttagaaca |
| | gcctcgcaca |
| 841 | gagtctgagt gggagaacag cttcaccctg aaaatgtttc tttttcagtt |
| | tgtcaatctg |
| 901 | aacagctcca cattttacat cgcattcttc ctcggaagat ttacaggaca |
| | cccaggtgcc |
| 961 | tacttgaggc tgataaacag gtggagacta gaagagtgcc accctagtgg |
| | atgccttatt |
| 1021 | gatctgtgta tgcaaatggg tattataatg gtgctaaagc agacctggaa |
| | taatttcatg |
| 1081 | gaacttggct acccgttaat tcagaattgg tggactagaa gaaaagtacg |
| | acaagaacat |
| 1141 | ggacctgaaa ggaaaataag tttcccacaa tgggaaaagg actataacct |
| | tcagccgatg |
| 1201 | aatgcctatg gactcttcga tgaatactta gaaatgattc ttcagtttgg |
| | attcacaact |
| 1261 | atctttgtgg cagcttttcc cctagcacca cttctggcct tactgaataa |
| | cataattgaa |
| 1321 | attcgacttg atgcttacaa atttgtcaca cagtggagga gacctttagc |
| | ttcaagggcc |
| 1381 | aaagacatag gaatttggta tggaattctt gaaggcattg gaattctctc |
| | tgttatcaca |
| 1441 | aatgcatttg tcatagcgat aacatctgac tttatccctc gcttggtgta |
| | tgcttataag |
| 1501 | tatggacctt gtgcaggcca aggagaagct gggcaaaagt gcatggttgg |
| | ctatgtgaat |
| 1561 | gccagcttgt ctgtatttcg aatttctgac tttgagaacc gatctgagcc |
| | tgaatctgat |
| 1621 | ggcagtgagt tctcggggac tcctcttaag tactgcagat accgggacta |
| | ccgtgacccg |
| 1681 | cctcattcac tggtgcccta tggctacaca ctgcagtttt ggcatgtcct |
| | agctgctcga |
| 1741 | ttagctttta tcattgtctt tgagcacctc gtgttttgta taaagcacct |
| | catttcgtat |
| 1801 | ctgatcccag acctcccaaa agacctaagg gatcgaatga gaagagagaa |
| | gtacttgatt |
| 1861 | caggagatga tgtatgaagc agaactggaa cgtctccaga aggaacgaaa |
| | ggagaggaag |
| 1921 | aagaatggaa aagcacacca caacgagtgg ccgtgaccat aaaatagtcc |
| | ctttccaggc |
| 1981 | caaggacctg aattctgttt acttcttctg gctgtgcaaa agcacactca |
| | agtgaatgac |
| 2041 | taaaaatgca accacagtgc atgttgcaga taccggcggc cgcaggaggg |
| | gcagcatcca |
| 2101 | gtagaggact ggcgttggag tcacactgct gtgaaatcac gttgcagtcc |
| | agcgcacaat |
| 2161 | tgctatctat ccatagacca ttcttgacca agcaagcatg cacattatgg |
| | gcagttacat |
| 2221 | tctcaagttt ttaaaatcaa ggggaacttg tatactgggc ctgtttttca |
| | gcctgtttgc |
| 2281 | tacctttttt gcattctatc ccatgtgaat tttacagaca ctgggctaaa |
| | aagggtattc |
| 2341 | agacacatgg acacacattc ctagaatgtc atcatatggt cctaattcca |
| | tgtcaccaag |
| 2401 | aacacagaca agaccctgtt tacaactttt tctttccttt tttttaattt |
| | tagacctttc |
| 2461 | tgagaagatt attatatatg acatatctat agctatgtgt atggccatag |
| | atgtatttct |
| 2521 | gtgtgtacat atgtatagtc atgtattcct gcatatgtac atacaaatac |
| | agagatatat |
| 2581 | aaagtacata gaaattcctt acttgtaaat agccaaaaag tactgacatg |
| | agtgaatttt |
| 2641 | cacatttaaa tagtcatcaa tatgaagcca tgattaatgc ttgtataatg |
| | tgatgcaata |
| 2701 | aaatttaaaa taaatttctg cacatggaat attttc |

ABCA1 19 AAF86276

| | |
|---|---|
| 1 | macwpqlrll lwknitfrrr qtcqllleva wplfiflili svrlsyppye |
| | qhechfpnka |
| 61 | mpsagtlpwv qgiicnannp cfryptpgea pgvvgnfnks ivarlfsdar |
| | rlllysqkdt |
| 121 | smkdmrkvlr tlqqikksss nlklqdflvd netfsgflyh nlslpkstvd |
| | kmlradvilh |
| 181 | kvflqgyqlh ltslcngsks eemiqlgdqe vselcglpre klaaaervlr |
| | snmdilkpil |
| 241 | rtlnstspfp skelaeatkt llhslgtlaq elfsmrswsd mrqevmfltn |
| | vnssssstqi |
| 301 | yqaysrivcg hpeggglkik slnwyednny kalfggngte edaetfydns |
| | ttpycndlmk |
| 361 | nlessplsri iwkalkpllv gkilytpdtp atrqvmaevn ktfqelavfh |
| | dlegmweels |
| 421 | pkiwtfmens qemdlvrmll dsrdndhfwe qqldgldwta qdivaflakh |
| | pedvqssngs |
| 481 | vytwreafne tnqairtisr fmecvnlnkl epiatevwli nksmellder |
| | kfwagivftg |
| 541 | itpgsielph hvkykirmdi dnvertnkik dgywdpgpra dpfedmryvw |
| | ggfaylqdvv |
| 601 | eqaiirvltg tekktgvymq qmpypcyvdd iflrvmsrsm plfmtlawiy |
| | svaviikgiv |
| 661 | yekearlket mrimgldnsi lwfswfissl ipllvsagll vvilklgnll |
| | pysdpsvvfv |
| 721 | flsvfavvti lqcflistlf sranlaaacg giiyftlylp yvlcvawqdy |
| | vgftlkifax |
| 781 | llspvafgfg ceyfalfeeq gigvqwdnlf espveedgfn lttsysmmlf |
| | dtflygvmtw |
| 841 | yieavfpgqy giprpwyfpc tksywfgees dekshpgsnq kriseicmee |
| | epthlklgvs |
| 901 | iqnlvkvyrd gmkvavdgla lnfyegqits flghngagkt ttmsiltglf |
| | pptsgtayil |
| 961 | gkdirsemst irqnlgvcpq hnvlfdmltv eehiwfyarl kglsekhvka |
| | emeqmaldvg |
| 1021 | lpssklkskt sqlsggmqrk lsvalafvgg skvvildept agvdpysrrg |
| | iwelllkyrq |
| 1081 | grtiilsthh mdeadvlgdr iaiishgklc cvgsslflkn qlgtgyyltl |
| | vkkdvessls |
| 1141 | scrnssstvs ylkkedsysq sssdaglgsd hesdtltidv saisnlirkh |
| | vsearlvedi |
| 1201 | gheltyvlpy eaakegafve lfheiddrls dlgissygis ettleeiflk |
| | vaeesgvdae |
| 1261 | tsdgtlparr nrrafgdkqs clrpftedda adpndsdidp esretdllsg |
| | mdgkgsyqvk |
| 1321 | gwkltqqqfv allwkrllia rrsrkgffaq ivlpavfvci alvfslivpp |
| | fgkypslelq |
| 1381 | pwmyneqytf vsndapedtg tlellnaltk dpgfgtrcme gnpipdtpcq |
| | ageeewttap |
| 1441 | vpqtimdlfq ngnwtmqnps pacqcssdki kkmlpvcppg agglpppqrk |
| | qntadilqdl |
| 1501 | tgrnisdylv ktyvqiiaks lknkiwvnef ryggfslgvs ntqalppsqe |
| | vndaxkqmkk |
| 1561 | hlklakdssa drflnslgrf mtgldtrnnv kvwfnnkgwh aissflnvin |
| | nailranlqk |
| 1621 | genpshygit afnhplnitk qqlsevaxmt tsvdvlvsic vifamsfvpa |
| | sfvvfliqer |
| 1681 | vskakhlqfi sgvkpviywl snfvwdmcny vvpatlviii ficfqqksyv |
| | sstnlpvlal |
| 1741 | llllygwsit plmypasfvf kipstayvvl tsvnlfigin gsvatfvlel |
| | ftdnklnnin |
| 1801 | dilksvflif phfclgrgli dmvknqamad alerfgenrf vsplswdlvg |
| | rnlfamaveg |
| 1861 | vvfflitvli qyrffirprp vnaklsplnd ededvrrerq rildgggqnd |
| | ileikeltki |
| 1921 | yrrkrkpavd ricvgippge cfgllgvnga gksstfkmlt gdttvtrgda |
| | flnxnsilsn |
| 1981 | ihevhqnmgy cpqfdaitel ltgrehveff allrgvpeke vgkvgewair |
| | klglvkygek |
| 2041 | yagnysggnk rklstamali ggppvvflde pttgmdpkar rflwncalsv |
| | vkegrsvvlt |
| 2101 | shsmeeceal ctrmaimvng rfrclgsvqh lknrfgdgyt ivvriagsnp |
| | dlkpvqdffg |
| 2161 | lafpgsvxke khrnmlqyql psslsslari fsilsqskkr lhiedysysq |
| | ttldqvfvnf |
| 2221 | akdqsdddhl kdlslhknqt vvdvavltsf lqdekvkesy v |

AGPAT3 56894 AF156774

| | |
|---|---|
| 1 | tctatgaaac caacatacat ggcgtttgca tcacagttgg agtcagatgt |
| | gagcccggag |
| 61 | ggcaggtgtc tggcttgtcc acccggaagc cctgagggca gctgttccca |
| | ctggctctgc |
| 121 | tgaccttgtg ccttggacgg ctgtcctcag cgaggggccg tgcacccgct |
| | cctgagcagc |
| 181 | gccatgggcc tgctggcctt cctgaagacc cagttcgtgc tgcacctgct |
| | ggtcggcttt |
| 241 | gtcttcgtgg tgagtggtct ggtcatcaac ttcgtccagc tgtgcacgct |
| | ggcgctctgg |
| 301 | ccggtcagca agcagctcta ccgccgcctc aactgccgcc tcgcatactc |
| | actctggagc |
| 361 | caactggtca tgctgctgga gtggtggtcc tgcacggagt gtacactgtt |
| | cacggaccag |
| 421 | gccacggtag agcgctttgg gaaggagcac gcagtcatca tcctcaacca |
| | caacttcgag |
| 481 | atcgacttcc tctgtgggtg gaccatgtgt gagcgcttcg gagtgctggg |
| | gagctccaag |
| 541 | gtcctcgcta agaaggagct gctctacgtg cccctcatcg gctggacgtg |
| | gtactttctg |
| 601 | gagattgtgt tctgcaagcg gaagtgggag gaggaccggg acaccgtggt |
| | cgaagggctg |
| 661 | aggcgcctgt cggactaccc cgagtacatg tggtttctcc tgtactgcga |
| | ggggacgcgc |
| 721 | ttcacggaga ccaagcaccg cgttagcatg gaggtggcgg ctgctaaggg |
| | gcttcctgtc |
| 781 | ctcaagtacc acctgctgcc gcggaccaag ggcttcacca ccgcagtcaa |
| | gtgcctccgg |
| 841 | gggacagtcg cagctgtcta tgatgtaacc ctgaacttca gaggaaacaa |
| | gaacccgtcc |
| 901 | ctgctgggga tcctctacgg gaagaagtac gaggcggaca tgtgcgtgag |
| | gagatttcct |
| 961 | ctggaagaca tcccgctgga tgaaaaggaa gcagctcagt ggcttcataa |
| | actgtaccag |
| 1021 | gagaaggacg cgctccagga gatatataat cagaagggca tgtttccagg |
| | ggagcagttt |
| 1081 | aagcctgccc ggaggccgtg gaccctcctg aacttcctgt cctgggccac |
| | cattctcctg |
| 1141 | tctcccctct tcagttttgt cttgggcgtc tttgccagcg gatcacctct |
| | cctgatcctg |
| 1201 | actttcttgg ggtttgtggg agcagcttcc tttggagttc gcagactgat |
| | aggagtaact |
| 1261 | gagatagaaa aaggctccag ctacggaaac caagagttta agaaaaagga |
| | ataattaatg |
| 1321 | gctgtgactg aacacacgcg gccctgacgg tggtatccag ttaactcaaa |
| | accaacacac |
| 1381 | agagtgcagg aaaagacaat tagaaactat ttttcttatt aactggtgac |
| | taatattaac |
| 1441 | aaaacttgag ccaagagtaa agaattcaga aggcctgtca ggtgaagtct |
| | tcagcctccc |
| 1501 | acagcgcagg gtcccagcat ctccacgcgc gcccgtggga ggtgggtccg |
| | gccggagagg |
| 1561 | cctcccgcgg acgccgtctc tccagaactc cgcttccaag agggaccttt |
| | ggctgctttc |
| 1621 | tctccttaaa cttagatcaa attttaaaaa aaaaaaaaaa |

AGPAT4 56895 AF156776

| | |
|---|---|
| 1 | tgaacccagc cggctccatc tcagcttctg gtttctaagt ccatgtgcca |
| | aaggctgcca |
| 61 | ggaaggagac gccttcctga gtcctggatc tttcttcctt ctggaaatct |
| | ttgactgtgg |
| 121 | gtagttattt atttctgaat aagagcgtcc acgcatcatg gacctcgcgg |
| | gactgctgaa |
| 181 | gtctcagttc ctgtgccacc tggtcttctg ctacgtcttt attgcctcag |
| | ggctaatcat |
| 241 | caacaccatt cagctcttca ctctcctcct ctggcccatt aacaagcagc |
| | tcttccggaa |
| 301 | gatcaactgc agactgtcct attgcatctc aagccagctg gtgatgctgc |
| | tggagtggtg |
| 361 | gtcgggcacg gaatgcacca tcttcacgga cccgcgcgcc tacctcaagt |
| | atgggaagga |
| 421 | aaatgccatc gtggttctca accacaagtt tgaaattgac tttctgtgtg |
| | gctggagcct |
| 481 | gtccgaacgc tttgggctgt tagggggctc caaggtcctg gccaagaaag |
| | agctggccta |
| 541 | tgtcccaatt atcggctgga tgtggtactt caccgagatg gtcttctgtt |
| | cgcgcaagtg |
| 601 | ggagcaggat cgcaagacgg ttgccaccag tttgcagcac ctccgggact |
| | accccgagaa |
| 661 | gtattttttc ctgattcact gtgagggcac acggttcacg gagaagaagc |
| | atgagatcag |
| 721 | catgcaggtg gcccgggcca aggggctgcc tcgcctcaag catcacctgt |
| | tgccacgaac |
| 781 | caagggcttc gccatcaccg tgaggagctt gagaaatgta gtttcagctg |
| | tatatgactg |
| 841 | tacactcaat ttcagaaata atgaaaatcc aacactgctg ggagtcctaa |
| | acggaaagaa |
| 901 | ataccatgca gatttgtatg ttaggaggat cccactggaa gacatccctg |
| | aagacgatga |
| 961 | cgagtgctcg gcctggctgc acaagctcta ccaggagaag gatgcctttc |
| | aggaggagta |
| 1021 | ctacaggacg ggcaccttcc cagagacgcc catggtgccc ccccggcggc |
| | cctggaccct |
| 1081 | cgtgaactgg ctgttttggg cctcgctggt gctctaccct ttcttccagt |
| | tcctggtcag |
| 1141 | catgatcagg agcgggtctt ccctgacgct ggccagcttc atcctcgtct |
| | tctttgtggc |
| 1201 | ctccgtggga gttcgatgga tgattggtgt gacggaaatt gacaagggct |
| | ctgcctacgg |
| 1261 | caactctgac agcaagcaga aactgaatga ctgactcagg gaggtgtcac |
| | catccgaagg |
| 1321 | gaaccttggg gaactggtgg cctctgcata tcctccttag tgggacacgg |
| | tgacaaaggc |
| 1381 | tgggtgagcc cctgctgggc acggcggaag tcacgacctc tccagccagg |
| | gagtctggtc |
| 1441 | tcaaggccgg atggggagga agatgttttg taatcttttt ttccccatgt |
| | gctttagtgg |
| 1501 | gctttggttt tctttttgtg cgagtgtgtg tgagaatggc tgtgtggtga |
| | gtgtgaactt |
| 1561 | tgttctgtga tcatagaaag ggtattttag gctgcagggg agggcagggc |
| | tggggaccga |
| 1621 | aggggacaag ttcccctttc atcctttggt gctgagtttt ctgtaaccct |
| | tggttgccag |
| 1681 | agataaagtg aaaagtgctt taggtgagat gactaaatta tgcctccaag |
| | aaaaaaaaat |
| 1741 | taaagtgctt ttctgggtca aaaaaaaaaa aaaa |

DGKQ 1609 L38707

| | |
|---|---|
| 1 | gggcggacct aaaggggctc gggccgctcg ggccgggaat ggcggcggcg |
| | gccgagcccg |
| 61 | gggcccgcgc ctggctgggc ggcggctccc cgcgccccgg cagcccggcc |
| | tgcagccccg |
| 121 | tgctgggctc aggaggccgc gcgcgcccgg ggccggggcc ggggccggga |
| | cgngaccgag |
| 181 | cgggcggcgt cagagcccgg gcccgtgccg cgccgggaca cagcttccgg |
| | aaggtgacgc |
| 241 | tcaccaagcc caccttctgc cacctctgct ccgacttcat ctgggggctg |
| | gccggcttcc |
| 301 | tgtgcgacgt ctgcaatttc atgtctcatg agaagtgcct gaagcacgtg |
| | aggatcccgt |
| 361 | gcacgagtgt ggcacccagc ctggtccggg ttcctgtagc ccactgcttc |
| | ggcccccggg |
| 421 | ggctccacaa gcgcaagttc tgtgctgtct gccgcaaggt cctggaggca |
| | ccggcgctcc |
| 481 | actgcgaagt gtgtgagctg cacctccacc cagactgtgt gcccttcgcc |
| | tgcagtgact |
| 541 | gccgccagtg ccaccaggat gggcaccagg atcacgacac ccatcaccac |
| | cactggcggg |
| 601 | aggggaacct gccctcggga gcgcgctgcg aggtctgcag gaagacgtgc |
| | ggctcctctg |
| 661 | acgtgctggc cggcgtgcgc tgcgagtggt gcggggtcca ggcgcactcc |
| | ctctgctccg |
| 721 | cggcactggc tcccgagtgt ggcttcgggc gtctgcgctc cctggtcctg |
| | cctcccgcgt |
| 781 | gcgtgcgcct tctgcccggc ggcttcagca agacgcagag cttccgcatc |
| | gtggaggccg |
| 841 | cggagccggg cgaggggggc gacggcgccg acgggagcgc tgccgtgggt |
| | ccaggcagag |
| 901 | agacacaggc aactccggag tccgggaagc aaacgctgaa gatctttgat |
| | ggcgacgacg |
| 961 | cggtgagaag aagccagttc cgcctcgtca cggtgtcccg cctggccggt |
| | gccgaggagg |
| 1021 | tgctggaggc cgcactgcgg gcccaccaca tccccgagga ccctggccac |
| | ctggagctgt |
| 1081 | gccggctgcc cccttcctct caggcctgtg acgcctgggc tgggggcaag |
| | gctgggagtg |
| 1141 | ctgtgatctc ggaggagggc agaagccccg ggtccggcga ggccacgcca |
| | gaggcctggg |
| 1201 | tcatccgggc tctgccgcgg gcccaggagg tcctgaagat ctaccctggc |
| | tggctcaagg |
| 1261 | tgggcgtggc ctacgtgtcc gtgcgagtga cccctaagag cacggctcgc |
| | tctgtggtgc |
| 1321 | tggaggtcct gccgctgctc ggccgccagg ccgagagtcc cgagagcttc |
| | cagctggtgg |
| 1381 | aggtggcgat gggctgcagg cacgtccagc ggacgatgct gatggacgaa |
| | cagcccctgc |
| 1441 | tggaccggct acaggacatc cggcagatgt ctgtgcggca ggtgagccag |
| | acgcggttct |
| 1501 | acgtggcaga gagcagggat gtagccccgc acgtctccct gtttgttggc |
| | ggcctgcctc |
| 1561 | ccggcctgtc tcccgaggag tacagcagcc tgctgcatga ggccggggct |
| | accaaagcca |
| 1621 | ccgtggtgtc cgtgagtcac atctactcct cccaaggcgc ggtagtgttg |
| | gacgttgcct |
| 1681 | gctttgcgga ggccgagcgg ctgtacatgc tgctgaagga catggctgtg |
| | cggggccggc |
| 1741 | tgctcactgc cctggtgctc cccgacctgc tgcacgcgaa gctgccccca |
| | gacagctgtc |
| 1801 | ccctccttgt gttcgtgaac cccaagagtg gaggcctcaa gggccgagac |
| | ctgctctgca |
| 1861 | gcttccggaa gctactgaac cctcatcagg tcttcgacct gaccaacgga |
| | ggtcctcttc |
| 1921 | ccgggctcca cctgttctcc caggtgccct gcttccgggt gctggtgtgt |
| | ggtggcgatg |
| 1981 | gcactgtggg ctgggtgctt ggcgccctgg aggagacacg gtaccgactg |
| | gcctgcccgg |
| 2041 | agccttctgt ggccatcctg cccctgggca cagggaatga ccttggtcga |
| | gtcctccgct |
| 2101 | ggggggcggg ctacagcggc gaggacccgt tctccgtact gctgtctgtg |
| | gacgaggccg |
| 2161 | acgccgtgct catggaccgc tggaccatcc tgctggatgc ccacgaagct |
| | ggcagtgcag |
| 2221 | agaacgacac ggcagacgca gagcccccca agatcgtgca gatgagtaac |
| | tactgtggca |
| 2281 | ttggcatcga cgcggagctg agcctggact tccaccaggc acgggaagag |
| | gagcctggca |
| 2341 | agttcacaag caggctgcac aacaagggtg tgtacgtgcg ggtggggctg |
| | cagaagatca |
| 2401 | gtcactctcg gagcctgcac aagcagatcc ggctgcaggt ggagcggcag |
| | gaggtggagc |
| 2461 | tgcccagtat tgaaggcctc atcttcatca acatccccag ctggggctcg |
| | ggggccgacc |
| 2521 | tgtggggctc cgacagcgac accaggtttg agaagccacg catggacgac |
| | gggctgctgg |
| 2581 | aggttgtggg cgtgacgggc gtcgtgcaca tgggccaggt ccagggtggg |
| | ctgcgctccg |
| 2641 | gaatccggat tgcccagggt tcctacttcc gagtcacgct cctcaaggcc |
| | accccggtgc |
| 2701 | aggtggacgg ggagccctgg gtccaggccc cggggcacat gatcatctca |
| | gctgctggcc |
| 2761 | ctaaggtgca catgctgagg aaggccaagc agaagccgag gagggccggg |
| | accaccaggg |
| 2821 | atgcccgggc ggatcgtgcg cctgcccctg agagcgatcc taggtagggg |
| | tggctggggc |
| 2881 | agcccaaggg ctcgagccat ctctgctccc gccagccttg ttttcaggtg |
| | gtctggaggc |
| 2941 | agctccacgt cacacagtgg ctgtcatata ttgaagttac cttcccactg |
| | gaaaaaaaat |

LPPR1 54886 AY304515

| | |
|---|---|
| 1 | gtggctcgga ccgccgcctg aatgtacctc gctcccggga gccggacggc |
| | ccagtagggc |
| 61 | gcactggagg acgctccgct gcgggagcct ggacagtttt tgacggtgca |
| | gtcttgctat |
| 121 | atggtgtgag aaatggctgt aggaaacaac actcaacgaa gttattccat |
| | catcccgtgt |
| 181 | tttatatttg ttgagcttgt catcatggct gggacagtgc tgcttgccta |
| | ctacttcgaa |
| 241 | tgcactgaca cttttcaggt gcatatccaa ggattcttct gtcaggacgg |
| | agacttaatg |
| 301 | aagccttacc cagggacaga ggaagaaagc ttcatcaccc ctctggtgct |
| | ctattgtgtg |
| 361 | ctggctgcca ccccaactgc tattattttt attggtgaga tatccatgta |
| | tttcataaaa |
| 421 | tcaacaagag aatccctgat tgctcaggag aaaacaattc tgaccggaga |
| | atgctgttac |
| 481 | ctgaacccct tacttcgaag gatcataaga ttcacagggg tgtttgcatt |
| | tggacttttt |
| 541 | gctactgaca tttttgtaaa cgccggacaa gtggtcactg ggcacttaac |
| | gccatacttc |
| 601 | ctgactgtgt gcaagccaaa ctacaccagt gcagactgcc aagcgcacca |
| | ccagtttata |
| 661 | aacaatggga acatttgtac tggggacctg gaagtgatag aaaaggctcg |
| | gagatccttt |
| 721 | ccctccaaac acgctgctct gagcatttac tccgccttat atgccacgat |
| | gtatattaca |
| 781 | agcacaatca agacgaagag cagtcgactg gccaagccgg tgctgtgcct |
| | cggaactctc |
| 841 | tgcacagcct tcctgacagg cctcaaccgg gtctctgagt atcggaacca |
| | ctgctcggac |
| 901 | gtgattgctg gtttcatcct gggcactgca gtggccctgt ttctgggaat |
| | gtgtgtggtt |
| 961 | cataacttta aaggaacgca aggatctcct tccaaaccca agcctgagga |
| | tccccgtgga |
| 1021 | gtacccctaa tggctttccc aaggatagaa agccctctgg aaaccttaag |
| | tgcacagaat |
| 1081 | cactctgcgt ccatgaccga agttacctga gacgactgat gtgtcacaag |
| | ctgtttttta |
| 1141 | aaatcatctt ccaattctat acttcaaaac acacagttgc tcaatgtcaa |
| | actgtgatga |
| 1201 | caaatattac gtttatctag ttagaagcta atgttttgta cattttttgt |
| | atgaggaagt |
| 1261 | gatgtagctt gccctgattt tttttttttt ttttggtcag ctttaatata |
| | tttatgccag |
| 1321 | aattttaaaa ccaacaaaat tttcttgttc aagcgtgcat tgaagaacca |
| | catttattca |
| 1381 | atggttgacg ttgttttgtg atatttgtac acaaattttc ttttctcagt |
| | tttataaaca |
| 1441 | cagaagtaaa tataacaatt cactttaaac ttttattacc acagttgctg |
| | cctcctccag |
| 1501 | aatttttgaa ttttaataaa aggcaaactt ttgagctgca ggaaggacaa |
| | tgttggttaa |
| 1561 | taataaatct caaagtcaat tgtagaaaaa aaattgtctt caaaaagaat |
| | gttgcactct |
| 1621 | gatctcttaa caaattgtta cgttcaaagt ttaaagtgat atattaacaa |
| | agtcacctag |
| 1681 | ttatacaaac aattgtcaga gaattctgga tttggagggt attggggtta |
| | tatgattctt |
| 1741 | tcttagataa tggcctctac taaataactc aagatctttc tggaatgtct |
| | tctggcaggc |
| 1801 | aggtgccact gtcagctttt ctccaaaaag cagccaacat cagcctcccc |
| | tgtcaactca |
| 1861 | acagttttgt atctcatatt atatggactt tatatgaaaa tgaatatttt |
| | acagtttgca |
| 1921 | cagtattatt ttacagaaaa ggaatcagag aatctacaac atagggcccc |
| | agaacaacag |
| 1981 | tttcactttg tggcttttaa ttattctaga attttaactg catctcattt |
| | ttctagcatg |
| 2041 | gtgagaacta atatgtaact cctttgattg aaggagctct tttgtccgta |
| | cctatcagaa |
| 2101 | tgttttcttg acacttccat gttggctctt ctcagctttt tttgtacata |
| | tttttttttt |
| 2161 | ctaaagagaa gaaaaagtta tcacaaaatg taaaaaaaga aaaaaaaaaa aaaaa |

TABLE 7
EEC overlap between BC and PD/AD. (see Table 6 for legend)

| Gene | EEC Function | KEGG | ËC | ND | References |
|---|---|---|---|---|---|
| ASTN2 | regulates trafficking of ASTN1, during early | hsa04144 | | | (Solecki 2012; |
| | clathrin-dependent endocytosis; | | | | Wilson et al. 2010) |
| | binds AP-2z | | BC | | (Kawauchi 2012) |
| | | | | AD | (K. S. Wang et al. 2015b) |
| | | | | ID | ASTN1: (Anazi et al. 2016) |
| TNS1 | controls cell polarization, migration, and | | | | (Burghel et al. 2013; |
| | invasion binds a5b1 integrin during | | | | McCleverty et al. 2007; |
| | endocytosis | | | | Rainero et al. 2015) |
| | | | BC | | (Hall et al. 2009) |
| MEGF11 | In C. elegans, DYN-1 (DNM1) depends | hsa04144 | | | CED-1: (Shen et al. 2013) |
| | on the function of CED-1 (MEGF10/11) | hsa04721 | | AD | MEGF10: (Sherva et al. 2014; |
| | | | | | Singh et al. 2010) |
| SDCBP2 | Syndecans bind PI(4, 5)P2 and are involved in | | | | (Baietti et al. 2012; |
| | both endo- and exocytosis. | | | | Hurley and Odorizzi 2012) |
| | | | BC | | (Y. Yang et al. 2013) |
| | | | | PD | (Tomlinson et al. 2015) |
| | | | | AD | (Leonova and Galzitskaya 2015) |
| N4BP3 | NEDD4 controls growth factor receptor | hsa04144 | | | (Jung et al. 2013; |
| | endocytosis (NEDD9 expression is assoc. | | | | Persaud et al. 2011) |
| | with BC metastasis) | | BC | | (Jung et al. 2013; |
| | | | | | Liao et al. 2015; |
| | | | | | Minn et al. 2005) |
| | | | | PD | Mol Cell Neurosci, |
| | | | | | 66 (Pt A), 21-28 |
| | | | | | (Perrett et al. 2015). |
| | | | | AD | (Rodrigues et al. 2016; |
| | | | | | Salminen et al. 2013) |
| SYNJ2 | is recruited to the nascent clathrin coated pit | hsa04070 | BC | PD | see (Table 6) |
| NLRP4 | and NLRP3 associate with BECN1, a component | | | | (Jounai et al. 2011; |
| | of the PI3K complex that mediates vesicle | | | | Rohatgi and Shaw 2016; |
| | trafficking | | | | Y. Zhang et al. 2014) |
| | | | BC | | (Zhiyu et al. 2016) |
| | | | | PD | (Choubey et al. 2014; |
| | | | | | J. D. Wang et al. 2015a) |
| | | | | AD | (Antonell et al. 2015; |
| | | | | | Swaminathan et al. 2016) |
| PTENP1 | PI3K/PTEN and PI(3, 4, 5)P3 are involved | hsa04070 | | | see (Table 6) |
| | in endocytosis and cancer | | | | |
| VAV3 | VAV . . . promote BCR endocytosis | hsa04666 | | | (Inabe et al. 2002; |
| | | | | | Malhotra et al. 2009) |
| | | | BC | | (X. I. N. Chen et al. 2015) |
| | | | | PD | (Moran et al. 2006) |
| | | | | AD | (Wilkinson et al. 2012) |
| PDE4D* | Binds ARRB2 (fast recycling) | hsa04144 | | | (Haddad et al. 2016) |
| | | | BC | | (Lin et al. 2013) |
| | | | | PD | (L. Yang et al. 2008) |
| | | | | AD | (Gurney et al. 2015) |
| EEA1 | binds to early endosomes in a Rab5 and PI(3)P | hsa04144 | | | (Pfeffer 1999) |
| | dependent manner. | | | PD | (Walter et al. 2001) |
| | | | | AD | (Armstrong et al. 2014) |
| RAB32 | RAB32/RAB38 interact AP-3 and with | hsa05012 | | | (Bultema et al. 2012; |
| | LRRK2 (PARK8) | | | | Hesketh et al. 2014; |
| | | | | | Waschbusch et al. 2014) |
| | | | BC | | (Agalliu et al. 2015) |
| | | | | PD | (M. Fukuda 2016) |
| | | | | AD | (M. Fukuda 2016) |
| SNX32* | Sorting Nexin (late endosome), SNX-BAR | hsa04144 | | | (van Weering et al. 2012; |
| | retromer with other Vps17 orthologs | | | | X. Wang et al. 2014; |
| | SNX5/SNX6 interacts with VPS35 | | | | Q. Y. Zhang et al. 2015) |
| | | | BC | | (Rivera et al. 2010) |
| | | | | PD | (Small and Petsko 2015) |
| | | | | AD | (Reitz 2012) |
| SCARB2 | required for maintenance of endo- and lysosomes, | hsa04142 | | | (Gonzalez et al. 2014) |
| | located in limiting membranes | | BC | | (Nishimura et al. 2003; |
| | | | | | Nishimura et al. 2006) |
| | | | | PD | (Alcalay et al. 2016) |
| | | | | AD | (J. Bras et al. 2014a; |
| | | | | | Shimizu et al. 2008) |
| GLB1 | Galactosidase Beta, related to Galectin 3 | | | | (H. Ahmed and AlSadek 2015) |
| | (LGALS3) | | BC | | (O'Reilly et al. 2015) |
| | | | | PD | (van Dijk et al. 2013) |
| | | | | AD | (Tiribuzi et al. 2011) |
| RAPGEF4 | GEF for RAB1A/1B/2A; involved in excocytosis | | | | (Almahariq et al. 2013; |
| | through RIMS2 | | | | Parnell et al. 2015) |
| | | | BC | | (Jiang et al. 2015) |
| | | | | PD | (Winslow et al. 2010) |
| | | | | AD | (Bereczki et al. 2016; |
| | | | | | Puthiyedth et al. 2016) |
| UNC13C | Interacting with each other and with PI(4, 5)P2. | | | | (Betz et al. 1997; |
| STXBP1 | Involved in docking/priming in exocytosis | hsa04721 | | | Martin 2015) |
| (MUNC18) | | | BC | | (Fernandez-Nogueira |
| | | | | | et al. 2016) |
| | | | | PD | (Campbell et al. 2012; |
| | | | | | Keogh et al. 2015) |
| | | | | AD | (Law et al. 2016; |
| | | | | | Leonova and Galzitskaya 2015; |
| | | | | | Miller et al. 2013; |
| | | | | | M. Takahashi et al. 2000) |
| STXBP4* | Prevents interaction between STX4 | hsa04130, | | | (Q. Y. Zhang et al. 2015) |
| | and VAMP2, | hsa04721 | | | |
| | | | BC | | (Antoniou et al. 2010; |
| | | | | | Day et al. 2011) |
| | | | | PD | (Diac et al. 2013) |
| | | | | AD | (Russell et al. 2012) |
| ANXA4 | Forms exocytotic complexes with SYT1 and | hsa04721 | | | (Lizarbe et al. 2013; |
| | the RAB3A effector RPH3A. | | | | Willshaw et al. 2004) |
| | | | BC | | (Wei et al. 2015; |
| | | | | | H. Yao et al. 2016) |
| | | | | PD | (Matigian et al. 2010) |
| | | | | AD | (Kuzuya et al. 2016; |
| | | | | | Tan et al. 2014) |
| | | | | HD | SYT1: |
| | | | | | (Valencia et al. 2013) |
| SYT17 | “B/K protein may play a role in exocytosis” | | | | (Chin et al. 2006; |
| | | | | | Mitsunori Fukuda 2013) |
| | | | BC | | (Weng et al. 2013) |
| | | | | AD | (V. Gautam et al. 2015) |
| PARK2 | “Loss of parkin promotes . . . endocytosis by | hsa04141 | | | (M. R. Ahmed et al. 2011; |
| | accumulating CAV1”; PARK2 binds AP-2 via | | | | Cha et al. 2015) |
| | arrestin | | BC | | (H.Wang et al. 2009) |
| | | | | PD | (Feng et al. 2015; |
| | | | | | Kitada et al. 1998) |
| | | | | AD | (Martin-Maestro et al. 2016) |
| DNAJC1* | ER membrane protein. DNAJC (Hsp40) controls | hsa04141 | | | |
| | release of proteins via HSPA5 | | BC | | (C.-L. Chen et al. 2009; |
| | (BiP, GRP78); DNAJC13 interacts with SNX-BAR | | | | Michailidou et al. 2013) |
| | | | | PD | DNAJC6/13: |
| | | | | | (Seaman and Freeman 2014; |
| | | | | | Vilarino-Guell et al. 2014) |
| | | | | AD | (Hsu et al. 2008) |

*from previous GWAS.
Underlined: functionally related genes identified in the literature.

### Example 3

### Elevated Endocytosis Combined with Lysosomal Dysfunction is a Common Epistatic Risk Factor in BC, AD/PD, and CAD

Endocytosis is a known component of the etiology of many age-related diseases, In BC, PD (Biochem Soc Trans, 43 (3)) , and AD (Mol Psychiatry, 21 (5), 707-16; Schreij, A. M., Fon, E. A., and McPherson, P. S. (2015), ‘Endocytic membrane trafficking and neurodegenerative disease’, Cell Mol Life Sci.; Molecular Neurodegeneration, 9 (1), 1-9),endocytosis of β1-integrin , α-synuclein (SNCA, and amyloid beta precursor protein (APP) respectively, are known to be critical early steps in the etiology leading to formation of plaques. The terms “derailed endocytosis” and “deranged endocytosis” hav been used to characterize an important component of the etiology of BC (Nat Rev Cancer, 8 (11), 835-50), AD (Biomed Res Int, 2014, 167024), and other “pathological conditions”. Cold Spring Harb Perspect Biol. 2014 August; 6(8): a016865 “[G]enes that influence endocytosis are overrepresented as AD risk factors [and] endocytosis-related genes are the earliest known disease-specific neuronal response in AD. They develop early in Down syndrome, a cause of early-onset AD linked to an extra copy of APP.” Mol Psychiatry, 21 (5), 707-16.

“New reports implicate altered [vacuolar H⁺]-ATPase activity and lysosomal pH dysregulation in cellular aging, longevity, and adult-onset neurodegenerative diseases, including forms of [PD] and [AD].” Colacurcio, D. J. and Nixon, R. A. (2016), ‘Disorders of lysosomal acidification—The emerging role of v-ATPase in aging and neurodegenerative disease’, Ageing Res Rev; see also FIG. 16. In PD, “an age-related pathological depletion of functional endosomes may increase the susceptibility to stochastic molecular defects in this same pathway, which in some individuals may trigger [a] vicious circle. [ . . . ] Disease causing mutations cluster within [the endosomal] pathway and alter receptor recycling and/or α-synuclein degradation. In turn, α-synuclein accumulation [ . . . ] exacerbates defective endosomal processing by impairing the machinery involved in the sorting or fusion of endosomes”. Mol Cell Neurosci, 66 (Pt A), 21-28 In AD, “accelerated endocytosis causes endocytic cargos to accumulate within enlarged [LEs] and impairs lysosomal functions. [ . . . ] Pathogenic endocytosis [ . . . ] could be modulated therapeutically at multiple possible targets.” Mol Psychiatry, 21 (5), 707-16 “The underlying molecular mechanisms [in AD and PD] remain poorly understood, yet dysfunction in endocytic membrane trafficking is a recurrent theme, which may explain the neurodegenerative process.” Schreij, A. M., Fon, E. A., and McPherson, P. S. (2015), ‘Endocytic membrane trafficking and neurodegenerative disease’, Cell Mol Life Sci.

Given the failure of previous GWAS to identify functionally related collections of genes and novel insights into the etiology of common diseases, the surprising results based on the novel GWAS approach (Example 1) were consistent with previous results not only in BC, but also in AD and PD (FIG. 15 and FIG. 16), in general, and with genes involving lysosomal function, in particular (FIGS. 18A-18C) for overlap in lysosomal genetic risk factors.

### Example 4

### βCDs Restore “Derailed Endocytosis” in Cancer and “Deranged Endocytosis” in PD/AD

### In Vitro

A plethora of studies have investigated the effect of βCDs in vitro.

Ca: MβCD suppressed invasion activity in three H7 Lewis lung cancer cell lines and highly metastatic cell lines had more β1 integrin (J Biol Chem, 281 (26), 18145-55) and BC and prostate cancer cell lines were more sensitive to MβCD-induced cell death than their normal counterparts. See Am J Pathol, 168 (4), 1107-18; quiz 404-5. In particular, MβCD treatment induced a substantial decrease (40%) in activity of BC resistance protein (BCRP/ABCG2) (J Pharmacol Exp Ther, 323 (1), 257-64), which transports PS and PC analogues. See J Biol Chem, 282 (2), 821-5. In subsequent functional studies, MβCD inhibited spheroid migration and invasion of MDA-MB-241 and ZR751 BC cells (BMC Cancer, 10, 647) and also endocytosis (Journal of cancer science & therapy, 4 (7), 214-22) and migration (Translational Medicine Communications, 1 (1), 3) of MCF7 BC cells. MβCD was more toxic for invasive than for non-invasive urothelial cancer cells (Plos One, 10 (9), e0137878), interfered with RTK-[PIP2]-PI3K-[PIP3]-AKT signaling in HeLa cells (FEBS Lett, 589 (24 Pt B), 4097-105), and inhibited the growth of leukemia cell lines (Plos One, 10 (11), e0141946).

AD: MβCD inhibits secretion of Aβ from hippocampal neurons of rats infected with recombinant Semliki Forest virus (SFV) carrying APP,(Simons et al. 1998) promotes the non-amylogenic α-secretase pathway,(Kojro et al. 2001) and increased activity of α-secretase while decreasing activity of β-secretase, reducing the level of cell-associated APPsβ (Error! Reference source not found.) “[I]mpaired internalization of APP [ . . . ] is responsible for increased α-secretase cleavage after acute cholesterol depletion by [sic] MβCD”.(Cole et al. 2005) Cell membrane cholesterol accumulation was detected in N2a cells over-expressing Swedish mutant APP (SwN2a), and the level of membrane cholesterol was reduced by HPβCD treatment, which dramatically lowered the levels of Aβ42 in SwN2a cells, and the effects were persistent for 24 h after withdrawal. See The Journal of Experimental Medicine, 209 (13), 2501-13.PD: In HeLa cells transfected with α-synuclein, α-synuclein was shown to colocalize with the lipid raft. Incubation with 20 mM MβCD for 1 hr dramatically reduced this co-location of α-synuclein with the cell membrane. (Fortin et al. 2004)

### In Vivo

The relevance of the above in vitro findings was confirmed by several in vivo studies.

Ca: MβCD had higher concentration in tumor than in other cells (except kidney and liver) and was effective in a mouse model of BC (Br J Cancer, 78 (9), 1165-9), reduced the number of lung metastases in mice implanted with H7-O Lewis lung cancer cells (J Biol Chem, 281 (26), 18145-55), and inhibited growth of primary effusion lymphoma (PEL) in mice (Biochem Biophys Res Commun, 455 (3-4), 285-9). (M-)β-CDs have found to increase the effectiveness of anti-tumor drugs, such as curcumin in a lung cancer mouse model (Br J Cancer, 107 (7), 1083-92) and of raloxifen in a chemically induced tumor mouse model (Agardan, N. B., et al. (2015), ‘The Effectiveness of Raloxifene-Loaded Liposomes and Cochleates in Breast Cancer Therapy’, AAPS PharmSciTech.). HPβCD was necessary in triple combination treatment for tumor regression in mice implanted with renal cancer cells (FEBS Lett, 589 (24 Pt B), 4097-105), and prolonged survival in leukemia mouse models (Plos One, 10 (11), e0141946).

AD: Scavenging of cholesterol and/or binding directly to Aβ or a-synuclein was also believed to be the mode of action for βCD in AD and PD: “HPβCD, which diminishes the pool of both cholesterol and PLs, had “neuroprotective effects [ . . . ] in a transgenic mouse model of AD [by] enhancing clearance mechanisms”. Four months of subcutaneous HPβCD administration significantly improved spatial learning and memory deficits in Tg19959 mice, diminished Aβ plaque deposition, and reduced tau immunoreactive dystrophic neurites (DN). Tg19959 mice are transgenic mice with 2 mutations in the APP gene which have been associated with human AD and beneficial effects were attributed to a reduction in cholesterol. See The Journal of Experimental Medicine, 209 (13), 2501-13. “Toxicity of Aβ1-40/42 was reduced in rats via stereotactical injection [of βCD] into the hippocampus.” CS Chem Neurosci, 3 (11), 807-19.

PD: “Treatment of mice with MβCD resulted in [ . . . ] reduced accumulation of α-synuclein in neuronal cell body and synapses.” J Neurochem, 98 (4), 1032-45. This “possibly transcriptional effect of MβCD,” J Neurochem, 98 (4), 1032-45, was seen as related to βCD preventing aggregation of α-synuclein ex vivo via direct interaction. Biochemistry, 53 (25), 4081-3. The results herein, instead, suggest that CDs act by regulating endocytosis as a common component in the etiology, the same age-related mechanism controlled by CDs in cancer.

### Clinical/Epidemiological

Although Atherosclerosis and AD are comorbid,(Y. Song et al. 2004) An analysis of the Framingham cohort also did not find an association between cholesterol levels and AD.(Z. S. Tan et al. 2003) While HPβCD was effective against tumors in animal models and well tolerated in most peripheral and central organ systems, it was shown to carry the risk of causing permanent hearing loss in mice, cats, and one human. See J Assoc Res Otolaryngol, 16 (5), 599-611; Assoc Res Otolaryngol, 16 (5), 599-611; Sci Transl Med, 7 (276), 276ra26; Pediatr Res, 68 (1), 52-6; Mol Genet Metab, 116 (1-2), 75-9. This ototoxicity is believed to be due to depriving prestin (SLC26A5) in outer hair cells of cholesterol.See Biophysical Journal, 103 (8), 1627-36; Sci Rep, 6, 21973; PLoS Genet, 11 (9), e1005500.

Mechanism: From the mechanism of βCD in NPC and elevated cholesterol levels seen in several cancers, including BC (Plos One, 10 (11), e0141946), βCDs were thought to reduce cancer growth in breast cancer (BC) by lowering cholesterol levels. Early evidence that this might not be the case emerged from the study of exosomes, which play a key role in development of BC. See Clin Chem, 61 (12), 1457-65; Semin Cancer Biol, 21 (2), 139-46. Treatment of MDA-MB-231 BC cells with MβCD inhibited the internalization of exosomes containing integrins, but did so independently of cholesterol.See Nature, 527 (7578), 329-35; Plos One, 6 (9), e24234.

### Example 5

### HPβCD Activates Formation of Autophagolysosomes Through a Mechanism Involving Reduction of Serum Phospholipids

“Genetic variation in lysosomal genes modifies the disease course of sporadic AD”. (Whyte et al. 2017) Autophagy has been linked to human oral diseases, including but not limited to “periapical lesions, peridontal diseases, and oral cancidiasis”.(Y. Q. Tan et al. 2017) “Crosstalk between autophagy and other cellular stresses [have been] implicated in ALS pathogenesis [with] therapeutic implications of regulating autophagy in ALS.” ATG16L1, a regulator of autophagy is consistently associated with inflammatory bowel diseases (including but not limited to Crohn's disease and ulcerative colitis). (Pugazhendhi et al. 2017). Autophagy “is emerging as a core regulator of Central Nervous System (CNS) aging and neurodegeneration, [affecting diseases] including ischemia/stroke, AD, PD, and HD, and MS, [ . . . ] involving microglial phagocytosis of apoptotic cells, Aβ, synaptic material, and myelin debris, and regulate the progression of age-associated ND.”(Plaza-Zabala et al. 2017)

Activation of autophagy has been deemed desirable in PD, (Moors et al. 2017), AD, HD, and ALS,(Moloudizargari et al. 2017), yet no affective drugs are available. “mTOR has been shown to be [a] key regulator but the detailed mechanisms are not satisfactorily solved.”(Y. Chen and Yu 2017) (HPβCD activates autophagy. Administration of Error! Reference source not found. results in activation of transcription factor EB TFEB. Upon translocation from the cytoplasm to the nucleus, TFEB regulates the expression of genes involved in biogenesis and fusion of lyso- and autophago-somes. As a result, Error! Reference source not found. administration results in enhanced clearance of the autophagy substrate ceroid lipo-pigment. The mechanism by which Error! Reference source not found. improves TFEB activation, however, was not understood (adapted from (Song W, Wang F, et al. (2014) J Biol Chem 289:10211-22)))

Treatment of HeLa/TFEB cells with HPβCD up-regulated LC3, which is essential for the formation of autophagy vesicles, SQSTM1/p62 which is essential for cargo recognition, and BECN1, which is required for the formation of autophagy-somes. In LINCL fibroblasts, which lack TPP1, HPβCD treatment resulted in clearance of ceroid lipopigment in a dose- and time-dependent fashion. HPβCD treatment resulted in transcriptional up-regulation of all autosomal (LC3, SQSTM1, BECN1) lysosomal genes tested (GBA, HEXA, LAMP1) through activation of transcription factor EB (TFEB) “HPβCD treatment results in coordinated up-regulation of lysosome biogenesis and autophagy and enhanced clearance of autophagy] material.” “HPβCD treatment results in TFEB-induced activation of the autophagy system, but blockage of downstream steps of the autophagy flux (e.g. blockage of ATG7 expression) prevents clearance of ceroid lipopigment.” “HPβCD activates the pro-survival autophagy pathway, but not apoptosis.” “It was recently appreciated that impairment or deregulation of autophagy is linked to the development and progression of a number of human diseases ranging from neurodegenerative diseases to cancer. Our findings will extend the capability of designing therapeutic solutions based on the use of HPβCD for the treatment of diseases characterized by inefficient autophagy clearance and accumulation of storage material.” FIG. 25; See J Biol Chem, 289 (14), 10211-22. In fact, activation of TFEB in neuroglioma cells with HPβCD promoted clearance of α-syn aggregates via autophagy.(Kilpatrick et al. 2015)

The mechanism by which HPβCD activates TFEB, however, “still needs to be clarified”.(Sardiello 2016) The surprising genetic results of Example 1 provide this clarification. It is already known that PI3K inhibitors promote TFEB nuclear translocation.(J. Cheng et al. 2006) From the novel results, HPβCD scavenging phospholipids reduces PI3K activity and, thus, promotes TFEB nuclear translation in similar fashion as PI3K inhibitors.

This finding is supported by other evidence. It is also known that treatment with paeoniflorin mitigates the disease phenotype of mice with spinobulbar muscular atrophy (SBMA), where autophagy in the spinal cord is diminished. Paeoniflorin is known to reduce serum lipids,(H. O. Yang et al. 2004) and has been successfully tested in animal models of LPC induced inflammation.(J. Z. Li et al. 2013) NAFLD,(Z. Ma et al. 2017b) insulin resistance,(Z. Ma et al. 2017a) and atherosclerosis.(H. Li et al. 2017)

The second part of the solution stemming from this unexpected finding is that the lysosome uses Ca as a positive feedback to it's master regulator, TFEB. Upon “starvation” (i.e., underutilization of the lysosome), (Sardiello 2016)

### Example 6

### Elevated Phagocytosis Combined with Lysosomal Dysfunction is a Known Component in the Etiology of Multiple Sclerosis (MS)

Early studies of MS in the 1970s reported “myelin-like material in lysosomes” and “synthesis of abnormal myelin by diseased glial cells” inspite of “increased lysosomal reaction”, including increased β-glycoronidase activity and “suggest[ed] early changes of glial cells as a basic mechanism of the disease.” J Neurol Sci, 19 (1), 29-36. It was hypothesized “that some acquired exogenous factor like virus infection is the basic cause which will trigger off the disease and the immunopathology.” Z Neurol, 203 (2), 91-104.

In 1977, three lysosomal enzymes, N-acetyl-β-d-glucosaminidase (MGEA4, also known as HEXC), β-galactosidase (GLB1), and cathepsin D (CTSD) were found upregulated, the highest activity in plaque areas.” Biochem Soc Trans, 5 (5), 1416-8; Neuropathol Appl Neurobiol, 5 (5), 405-15. Enzyme levels in serum and CSF, in general, were not elevated in MS. See Acta Neurol Scand, 57 (3), 201-15; Acta Neurol Scand, 59 (1), 23-30.

In 1980, it was suggested that “MΦs were stimulated to increase lysosomal enzyme activity, initiated by stimulation of T-lymphocytes caused by MS-specific antigen,” yet no such antigens were identified, except that “a microglial component” is likely involved. Clin Exp Immunol, 42 (1), 50-6; Neuropathol Appl Neurobiol, 7 (3), 169-82.

In 1983, a “2-step demyelination” hypothesis was proposed: (1) a toxin penetrates into myelin, and its degradation creates antigen. (2) Antigen-activated MΦs settle in and digest myelin, yet the toxin remained elusive. See Med Hypotheses, 12 (2), 129-42; Journal of Neurochemistry, 127 (1), 7-21.

In 1996, still, “little is known about the source of [activated] MΦs in the early stages of plaque evolution . . . . A key issue in understanding the pathogenesis of MS is the reliable identification of phagocytes capable of degrading myelin and presenting autoantigen to T cells at the onset of demyelination, [although] results indicate that microglia are the main population of phagocytes in the early staged of demyelination.” Neuropathol Appl Neurobiol, 22 (3), 207-15.

Aside from confirming HLA-DRB1*15:01 as a risk factor, a recent “metaanalysis” of 7125 cases (George et al. 2016) did not provide any actionable insights.

Microglia have “similar transcriptome pattern” to MΦs; the function of microglia is to “scan the entire volume of the brain over the course of a few hours”. Hence, it has been proposed that dysregulation of microglial function contributes to CNS disorders, and that “targeting the mechanisms that are dysregulated may arrest or reverse neurodevelopmental and neurodegenerative disorders in which microglia play a role.”(Salter and Stevens 2017) During the process of scanning the brain, microglia either targets the substrates for degradation by the lysosome or, in the case of pathogenes, processes it for antigen presentation. “Endocytosis, sorting, transport, compartment acidification, and degradation . . . may be altered during aging”. (Sole-Domenech et al. 2016)

The present disclosure links two recent findings in atherosclerosis and Fabry disease to the etiology of MS. Foamy MΦs, containing myelin degradation products, are abundantly found in active multiple sclerosis (MS) lesions.(Bogie et al. 2013) Fabry disease, a deficiency of lysosomal alpha-galactosidase A (GLA) is often mis-diagnosed as MS (Bottcher et al. 2013; Germain 2010) or both diseases show familial comorbidity.(Cammarata et al. 2015) From the better understanding of the mechanism causing foamy MΦs in atherosclerosis (intake of more LDL then lysosomes can handle) and Fabry disease (lack of lysosomal galactosidase activity), the nature of the “toxin” becomes evident. The process starts, in fact, with the microglia. Z Neurol, 203 (2), 91-104. When the microglial lysosomes cannot degrade all the myelin that is phagocytosed during scanning of the entire brain, the undegraded myelin is accidentally presented to the T-cells waiting for antigens from the microglia as “MS-specific antigen” and the T-cells then move to the circulation to activate the MΦs against myelin. The MΦs return to the CNS where they begin to—mistakenly—endocytose myelin (FIG. 27). See Clin Exp Immunol, 42 (1), 50-6.

Since the process is the same as in AD, PD, and CAD, the solution is the same: Slowing down phagocytosis of myelin by aging microglia prevents the lysosomes from “overflowing” and, thus, myelin to be presented to MΦs as “MS-specific antigen”. In addition, slowing down endocytosis of myelin by MΦs reduces demyelination in case that not all “MS-specific antigens” can be prevented.

In support of shared risk factors, MS and vascular diseases are comorbid. “[H]aving type-2-diabetes, hypertension, dyslipidaemia or peripheral vascular disease at any point in the disease course may be associated with a greater progression in disability . . . . The results of published clinical trials of statins as an intervention in MS were however conflicting.”(Tettey et al. 2014)

MβCD enhancing CD30 shedding has been suggested in Th1-mediated diseases, such as multiple sclerosis and rheumatoid arthritis.(von Tresckow et al. 2004)

### Example 7

### Phospholipids as a Drug Target

The present disclosure provides the phospholipids entering the PI-cycle as the drug target (Example 1) and (a safer version of) a drug that has been demonstrated to be effective in models of AD, PD, and CAD and in humans with other lysosomal function diseases (including, but not limited to NPC).

The results presented here show that the number of combinations of genes involved in different patients is too large for the goal of “targeting endocytosis” to be likely achieved by selectively targeting individual or even pairs of phosphatases or kinases (Table 6) or by targeting individual genes regulated by the PI cycle (Table 7). As discussed above, the PI cycle is designed to compensate for dysregulation of individual kinases or phosphatases. “Decreasing levels of PA” by siRNA directed toward DGKQ or PLD and use of several inhibitors (including Wortmannin) and activators aiming at “increasing intracellular levels of PIP and/or PIP2” were proposed based on the linear PI-PIP-PIP2-PIP3 model and the effect of Aβ on PA and PIPs.See U.S. Pat. 8,288,378 to Kim et al.

Downregulating the entire PI cycle, however, could achieve the goal of reducing endocytosis. The breadth of genes involved in entry of PI and PS/PC into the PI cycle suggests a different strategy as more effective. Extraction of phospholipids reduces the intracellular concentration of phospholipids, which are known to regulate endocytosis during ligand binding (PI(4,5)P2), pit-formation (PI(4)P,), vesicle formation (PI(3,4)P2), fusion to an early endosome (PI(3)P), and sorting into cell organelles, including the lysosomes, which are involved in NPC1 (PI(3,5)P2). “Activation of signal transduction pathways associated with endocytic trafficking is critical for tumor cell migration [and disease progression in PD/AD]. As a consequence, [ . . . ] targeting endocytic trafficking and signaling could potentially allow for the development of novel cancer therapeutics to prevent metastasis [and anti-aging therapeutics to prevent PD and AD].” See Oncotarget, 7 (1), 5-6.

For instance, FAK (integrin-mediated focal adhesion kinase) is overexpressed and activated in tumors, but rarely mutated.(Alanko and Ivaska 2016) FAK inhibitors have been shown to decrease tumor growth, metastasis, and angiogenesis in mice, and are in early clinical trials for non-hematologic cancers, including, but not limited to, pancreatic cancer, lung cancer, mesothelioma, and ovarian cancer, with mixed results (clinicaltrials.gov). Regulating endocytosis of integrins provides an alternative to reduce the activity of integrin-mediated focal adhesion kinase, either alone or in combination with immunotherapy.(Symeonides et al. 2017)

### Example 8

### Lysosomal Dysfunction is a Known Component in the Etiology of Multiple Sclerosis (MS)

Early studies of MS in the 1970s reported “myelin-like material in lysosomes” and “synthesis of abnormal myelin by diseased glial cells” in spite of “increased lysosomal reaction”, including increased β-glycoronidase activity and “suggest[ed] early changes of glial cells as a basic mechanism of the disease.” J Neurol Sci, 19 (1), 29-36. It was hypothesized “that some acquired exogenous factor like virus infection is the basic cause which will trigger off the disease and the immunopathology.” Z Neurol, 203 (2), 91-104.

In 1977, three lysosomal enzymes N-acetyl-β-d-glucosaminidase (MGEA4, also known as HEXC), β-galactosidase (GLB1), and cathepsin D (CTSD) were found upregulated, the highest activity in plaque areas.” Biochem Soc Trans, 5 (5), 1416-8; Neuropathol Appl Neurobiol, 5 (5), 405-15. Enzyme levels in serum and CSF, in general, were not elevated in MS. See Acta Neurol Scand, 57 (3), 201-15; Acta Neurol Scand, 59 (1), 23-30.

In 1980, it was suggested that “macrophages were stimulated to increase lysosomal enzyme activity, initiated by stimulation of T-lymphocytes caused by MS-specific antigen”, yet no such antigens were identified, except that “a microglial component” is likely involved. See Clin Exp Immunol, 42 (1), 50-6; Neuropathol Appl Neurobiol, 7 (3), 169-82.

In 1983, a “2-step demyelination” hypothesis was proposed: (1) a toxin penetrates into myelin, and its degradation creates antigen. (2) Antigen-activated macrophages settle in and digest myelin, yet the toxin remained elusive. See Med Hypotheses, 12 (2), 129-42; Journal of Neurochemistry, 127 (1), 7-21.

In 1996, still, “little is known about the source of [activated] macrophages in the early stages of plaque evolution . . . . A key issue in understanding the pathogenesis of MS is the reliable identification of phagocytes capable of degrading myelin and presenting autoantigen to T cells at the onset of demyelination, [although] results indicate that microglia are the main population of phagocytes in the early staged of demyelination.” Neuropathol Appl Neurobiol, 22 (3), 207-15.

Aside from confirming HLA-DRB1*15:01 as a risk factor, a recent “metaanalysis” of 7125 cases (George et al. 2016) did not provide any actionable insights.

Microglia have “similar transcriptome pattern” to macrophages; the function of microglia is to “scan the entire volume of the brain over the course of a few hours”. Hence, it has been proposed that dysregulation of microglial function contributes to CNS disorders, and that “targeting the mechanisms that are dysregulated may arrest or reverse neurodevelopmental and neurodegenerative disorders in which microglia play a role.”(Salter and Stevens 2017) During the process of scanning the brain, microglia either targets the substrates for degradation by the lysosome or, in the case of pathogenes, processes it for antigen presentation. “Endocytosis, sorting, transport, compartment acidification, and degradation . . . may be altered during aging”. (Sole-Domenech et al. 2016)

The present disclosure links two recent findings in atherosclerosis and Fabry disease to the etiology of MS. Foamy macrophages, containing myelin degradation products, are abundantly found in active multiple sclerosis (MS) lesions.(Bogie et al. 2013) Fabry disease, a deficiency of lysosomal alpha-galactosidase A (GLA) is often mis-diagnosed as with MS (Bottcher et al. 2013; Germain 2010) or shows familial comorbidity.(Cammarata et al. 2015) From the better understanding of the mechanism causing foamy macrophages in atherosclerosis (more LDL then lysosomes can handle) and Fabry disease (lack of lysosomal . . . ), the nature of the “toxin” becomes evident. The process starts, in fact, with the microglia. See Z Neurol, 203 (2), 91-104. When the microglial lysosomes cannot degrade all the myelin that is phagocytosed during scanning of the entire brain, the remaining myelin . . . passes in the circulation, gets presented as “MS-specific antigen” to the T-cells, which activate the macrophages against myelin. See Clin Exp Immunol, 42 (1), 50-6. The macrophages return to the CNS where they begin to—mistakenly—endocytose myelin (FIG. 27)

Since the process is the same as in AD, PD, and CAD, the solution is the same: Slowing down phagocytosis of myelin by aging microglia prevents the lysosomes from “overflowing” and, thus, myelin to be presented to macrophages as “MS-specific antigen”. In addition, slowing down endocytoses of myelin by macrophages reduces demyelination in case that not all “MS-specific antigens” can be prevented.

The ipresent disclosure provides the PI-cycle as the drug target (Example 1) and (a safer version of) a drug that has been demonstrated to be effective in models of AD, PD, and CAD and in humans with a different lysosomal function disease (including, but not limited to Neyman-Pick type C).

### Example 9

### α-Cyclodextrin Restores “Derailed Endocytosis” in BC and “Deranged Endocytosis” in PD/AD

The present disclosure provides α-CD and analogues and derivatives thereof as non-limiting examples of compounds that may be useful for treating age-related conditions including but not limited to conditions involving “derailed endocytosis”, a “hallmark of cancer” (Mitra et al. 2012) also seen in neurodevelopmental diseases. See Biochem Soc Trans, 43 (3); Biomed Res Int, 2014, 167024. In some embodiment of the present disclosure α-CDs are used for treating age-related conditions, such as cancers (including, but not limited, to α-CD) and neurodegenerative diseases (including, but not limited to, PD or AD). CDs lower the amount of PIPs available without directly interfering with their distribution.

A plethora of studies have investigated the effect of methyl-β-cyclodextrin (MβCD) in vitro. MβCD suppressed invasion activity in three H7 Lewis lung cancer cell lines and highly metastatic cell lines had more β1 integrin (J Biol Chem, 281 (26), 18145-55) and BC and prostate cancer cell lines were more sensitive to MβCD-induced cell death than their normal counterparts. See Am J Pathol, 168 (4), 1107-18; quiz 404-5.

In particular, MβCD treatment induced a substantial decrease (40%) in activity of BC resistance protein (BCRP/ABCG2) (J Pharmacol Exp Ther, 323 (1), 257-64), which transports PS and PC analogues.See J Biol Chem, 282 (2), 821-5. In subsequent functional studies, MβCD inhibited spheroid migration and invasion of MDA-MB-241 and ZR751 BC cells (BMC Cancer, 10, 647) and also endocytosis (Journal of cancer science & therapy, 4 (7), 214-22) and migration (Translational Medicine Communications, 1 (1), 3) of MCF7 BC cells. MβCD was more toxic for invasive than for non-invasive urothelial cancer cells (Plos One, 10 (9), e0137878), interfered with RTK-[PIP2]-PI3K-[PIP3]-AKT signaling in HeLa cells (FEBS Lett, 589 (24 Pt B), 4097-105), and inhibited the growth of leukemia cell lines (Plos One, 10 (11), e0141946).

The relevance of the above in vitro findings was confirmed by several in vivo studies. MβCD had higher concentration in tumor than in other cells (except kidney and liver) and was effective in a mouse model of BC (Br J Cancer, 78 (9), 1165-9), reduced the number of lung metastases in mice implanted with H7-O Lewis lung cancer cells (J Biol Chem, 281 (26), 18145-55), and inhibited growth of primary effusion lymphoma (PEL) in mice (Biochem Biophys Res Commun, 455 (3-4), 285-9). HPβCD was necessary in triple combination treatment for tumor regression in mice implanted with renal cancer cells (FEBS Lett, 589 (24 Pt B), 4097-105) and prolonged survival in leukemia mouse models (Plos One, 10 (11), e0141946).

In mouse models of cancer, (M-)β-cyclodextrins have found to increase the effectiveness of anti-tumor drugs, such as curcumin in a lung cancer mouse model ((Br J Cancer, 107 (7), 1083-92) and of raloxifen in a chemically induced tumor mouse model (Agardan, N. B., et al. (2015), ‘The Effectiveness of Raloxifene-Loaded Liposomes and Cochleates in Breast Cancer Therapy’, AAPS PharmSciTech.). As cholesterol has been found elevated in several cancers, the higher efficacy of anti-tumor drugs when delivered with β-cyclodextrin as an expedient has been attributed to the ability of larger cyclodextrins to scavenge cholesterol after the drug is released. More recently, β-cyclodextrin by itself has been proposed as a drug to reduce cancer growth by scavenging cholesterol in mouse models of melanoma (Mohammad et al. 2014), and leukemia (Plos One, 10 (11), e0141946). The genetics results presented here, are consistent with cyclodextrins being effective, but replace the previous hypothesis of scavenging cholesterol (control of cancer growth) with evidence for scavenging phospholipids (control of endocytosis) as a more specific mechanism for reducing the risk of metastases.

Scavenging of cholesterol and/or binding directly to Ab or a-synuclein was also believed to be the mode of action for β-cyclodextrin in AD and PD: “HPβCD, which diminishes the pool of both cholesterol and PLs, had “neuroprotective effects [ . . . ] in a transgenic mouse model of AD [by] enhancing clearance mechanisms”. See The Journal of Experimental Medicine, 209 (13), 2501-13. “Toxicity of Ab1-40/42 was reduced in rats via stereotactical injection [of βCD] into the hippocampus.” CS Chem Neurosci, 3 (11), 807-19. This effect that was attributed to the ability of bCD to interact with Ab. FEBS Lett, 341 (2-3), 256-8. Similarly, in PD, that “treatment of mice with MβCD resulted in [ . . . ] reduced accumulation of α-synuclein in neuronal cell body and synapses” was seen as related to β-cyclodextrin to prevent aggregation of α-synuclein ex vivo via direct interaction. Journal of Neurochemistry, 98 (4), 1032-45; see Biochemistry, 53 (25), 4081-3. The results of the present disclosure, instead, suggest that cyclodextrins act by regulating endocytosis as a common component in the etiology, the same age-related mechanism controlled by cyclodextrins in cancer.

From the mechanism of βCD in NPC and elevated cholesterol levels seen in several cancers, including BC (Plos One, 10 (11), e0141946), βCDs were thought to reduce cancer growth in BC by lowering cholesterol levels. Early evidence that this might not be the case emerged from the study of exosomes, which play a key role in development of BC. See Clin Chem, 61 (12), 1457-65; Semin Cancer Biol, 21 (2), 139-46. Treatment of MDA-MB-231 BC cells with MβCD inhibited the internalization of exosomes containing integrins, but did so independently of cholesterol. See Nature, 527 (7578), 329-35; Plos One, 6 (9), e24234.

While HPβCD was effective against tumors in animal models and well tolerated in most peripheral and central organ systems, it was shown to carry the risk of causing permanent hearing loss in mice, cats, and one human. See Assoc Res Otolaryngol, 16 (5), 599-611; Plos One, 7 (12), e53280; Assoc Res Otolaryngol, 16 (5), 599-611; Sci Transl Med, 7 (276), 276ra26; Pediatr Res, 68 (1), 52-6; Mol Genet Metab, 116 (1-2), 75-9. This ototoxicity is believed to be due to depriving prestin (SLC26A5) in outer hair cells of cholesterol. See Biophysical Journal, 103 (8), 1627-36; Sci Rep, 6, 21973; PLoS Genet, 11 (9), e1005500.

The role of the PIP cycle emerging from our results, however, suggests a different mechanism than scavenging of cholesterol. A different mechanism is consistent with previously reported in vivo results: CAV1 expression in BC stroma increases tumor migration and invasion and CAV1 is required for invadopodia formation specifically by BC cells, where CAV1 knockdown cannot be rescued by cholesterol. See Cell, 146 (1), 148-63; Cancer Res, 69 (22), 8594-602. Growing MDA-MB-231 BC cells in lipoprotein depleted medium resulted in an 85% decrease in cell migration.See Clin Exp Metastasis, 28 (8), 733-41. LPA activates the Arf6-based mesenchymal pathway for migration and invasion of renal cancer cells, which originate, like BC cells, from cells located within epithelial ductal structures. Nat Commun, 7, 10656.

### Example 10

### α-CD is More Efficient than β-CD in Solubilizing Phospholipids

The present disclosure provides α-CD or HPα-CD and analogues and derivatives thereof as non-limiting examples of compounds that may be useful for treating age-related conditions including but not limited to conditions involving “derailed endocytosis,” a “hallmark of cancer” (Mitra et al. 2012) also seen in neurodevelopmental diseases. See Biochem Soc Trans, 43 (3); Biomed Res Int, 2014, 167024. In some embodiment of the present disclosure α-CDs are used for treating age-related conditions, such as cancers (including, but not limited, to prostate and breast cancer) and neurodegenerative diseases (including, but not limited to, PD or AD). CDs lower the amount of PIPs available without directly interfering with their distribution.

Scavenging phospholipids, which regulate endocytosis more specifically (by α-cyclodextrin, six starch molecules), rather than also scavenging larger cholesterol molecules (by the larger β-cyclodextrin, seven sugar molecules) avoids cholesterol-mediated side effects, including ototoxicity. See Assoc Res Otolaryngol, 16 (5), 599-611. “The acryl chain of phospholipids fits tightly into the hydrophobic cavity of the smallest α-CD and more loosely into the larger inner space of β- and γ-CDs, whereas the side chain of cholesterol is preferably included in the β-CD cavity.” J Pharm Sci, 86 (2), 147-62. “Cyclodextrins partially removed phospholipids . . . with a potency of α>β>>γ. Cholesterol . . . was extracted . . . most effectively by β-cyclodextrin . . . , while [the effect] of α-cyclodextrin was negligible even at hemolytic concentrations.” European Journal of Biochemistry, 186 (1-2), 17-22. “β-CD also remove proteins from erythrocyte membranes.” J Pharm Sci, 86 (2), 147-62.

At 10 mM, α- and β-cyclodextrin reduce transferrin endocytosis by 20% and 80%, respectively, which has been interpreted as α-cyclodextrin not having “any significant effect.” Molecular Biology of the Cell, 10 (4), 961-74. The results presented here, however, show that these results are consistent with a more physiologic 30% of phospholipids, rather than an extreme 95% of cholesterol released at this concentration (Error! Reference source not found.). See European Journal of Biochemistry, 186 (1-2), 17-22. Moreover, even increasing the concentration of α-cyclodextrin above 10 mM does not increase release of phospholipids, which might disrupt vital functions, while increasing the concentration of β-cyclodextrin results in a massive increase of protein release. Hence, at the same level where β-cyclodextrin interferes with vital cell function by limiting available cholesterol, α-cyclodextrin merely reduces endocytosis to normal ranges by reducing regulatory phospholipids. See European Journal of Biochemistry, 186 (1-2), 17-22. “PIPs are [also] involved in . . . common neurodegenerative conditions such as Alzheimer's that are becoming more widespread as life expectancy increases”. Biochim Biophys Acta, 1851 (8), 1066-82. Membrane anchored inhibitors of β-secretase have been postulated as a strategy to prevent endocytosis of APP. See Clin Exp Immunol, 42 (1), 50-6. The results presented herein provide reduction of overall endocytosis by attenuation of PI levels via α-CD as an alternative to inhibiting β-secretase which has higher affinity for Neuregulin than for APP. See Cell Rep, 14 (9), 2127-41.
Since cyclodextrins have been successfully applied both intravenously and intrathecally, different routes of administrations can be used to prevent bone and lung metastases v. glioblastoma and neurodegenerative diseases.

### Example 11

### α-CD is More Efficient than β-CD in Solubilizing Phospholipids

Materials: The degree of substitution for α, β, and γ HP-CDs was 5.3, 5.1, and 5.4, respectively. . In preparations of samples, 5% (w/w) NaOH was used as a solvent and catalyst in the condensation reaction of epoxide with cyclodextrin. Radioactive lipids were purchased from NEN Research Products, Boston, Mass., and nonradioactive lipids were purchased from Sigma Chemical Company, St. Louis, Mo. See J Pharm Sci, 81 (6), 524-8.

Methods: Measurement of lipids was accomplished with clinical diagnostic kits (Sigma and Wako Chemicals, Dallas, Tex.). Solubilities of lipids were measured after brief sonication of the suspension of an excess of radioactive sample (in phosphate-buffered isotonic saline, pH 7.4; in a closed vessel under argon atmosphere) and equilibration by shaking (for cholesterol, the period of shaking was 1 week; for cholesterol oleate, L-α-dipalmitoylphosphatidylcholine, and triolein, the period of shaking was 3 days). Thereafter, the suspension was filtered through a membrane filter (Millex; SLGS, 0.25; OS, 0.22 μm), and the radioactivity in the filtrate was measured by liquid scintillation counting. The same process was used for sphingomyelin, except that 1 day of shaking and a clinical kit for measurement of phospholipids were used. These methods are prone to yield somewhat higher values for the solubilities of surface-active compounds in buffer, because the filtrate is enriched by the mono-layers of the surface-active compound that form at the air-buffer interface. However, these methods are suited for measurement of solubilities of lipids in HP-CD solutions. This fact was established by comparing results obtained with the filtration methods described above and those from a method that used titration of a lipid sample with a solution of HP-CD, with the end point of dissolution being determined visually. The same titration method was used to establish that cholesterol and cholesteryl methyl ether dissolve in the solutions of HP-β-CD to about the same extent. See J Pharm Sci, 81 (6), 524-8.

Results: The differential effects of α, β, and γ HP-cyclodextrins on five representative pure lipids are shown in FIG. 32 HP-β-CD preferentially solubilized cholesterol, whereas phospholipids were best solubilized with HP-α-CD. See J Pharm Sci, 81 (6), 524-8.

### Example 12

### α-CD is Safer than β-CD and More Effective in Preventing Migration of Human Tumor Cells

### Introduction

Published results have shown that β-CD inhibits human MDA-MB 231 cell migration. See Translational Medicine Communications, 1 (1), 3. This inhibition was attributed to the ability of β-CD to “deplete cholesterol”. β-CD, however, depletes also phospholipids. To determine whether inhibition of migration is caused by β-CD depleting cholesterol, as commonly assumed, or by β-CD depleting phospholipids, as implicated by the results of the present disclosure, the wound healing experiment was replicated, comparing both HPbCD (Sigma, 389145-5G) and HPαCD (Sigma, 390690-5G) v. control in both MDA-MB 231 (CRM-HTB-26, ER⁻) and MCF-7 (ATCC HTB-2, ER⁺) human breast epithelial cell lines.

### Method:

Cells were cultured in 24-well culture plates (Cytoselect CBA-120, 0.9 mm wound healing/gap closure migration assay) for 24 h with wound healing insert in place. Cells were then treated for 2 h with HPbCD, HPaCD, or control.

Protocol: Warm up the 24-well plate (CBA-120, Cell BioLabs Inc.) with 0.9 mm CytoSelect Wound Healing Inserts at room temperature for 10 minutes. Using sterile forceps, orient the desired number of inserts in the plate wells with their “wound field” aligned in the same direction. Create a cell suspension containing 0.5-1.0×10⁶ cells/ml in media containing 10% fetal bovine serum (FBS). Add 500 μL of cell suspension to each well by carefully inserting the pipette tip through the open end at the top of the insert. For optimal cell dispersion, add 250 μL of cell suspension to either side of the open ends at the top of the insert. Incubate cells in a cell culture incubator for 12-24 hours. Carefully remove the insert from the well to begin the wound healing assay. Use sterile forceps to grab and lift the insert slowly from the plate well. Slowly aspirate and discard the media from the wells. Wash wells with media to remove dead cells and debris. Finally, add media to wells to keep cells hydrated. Repeat wash if wells still have debris or unattached cells. When washing is complete, add media with FBS and/or compounds to continue cell culture and wound healing process. Agents that inhibit or stimulate cell migration can be added directly to the wells. Incubate cells in a cell culture incubator (2 hours) and then wash cells with PBS and then add fresh media without compounds. Incubate 12-24 hours before wash and fixing. Remove the fixation solution and add 400 μL of Cell Stain Solution to each well. Allow the stain to incubate with the cells for 15 minutes at room temperature. Aspirate and discard the solution. Carefully wash each stained well 3× with deionized water. Discard washes and allow cells to dry at room temperature. Cells that migrated into the wounded area or protruded from the border of the wound were visualized and photographed under an inverted microscope. Determine the surface area of the defined wound area: Total Surface Area=0.9 mm×length. Determine the surface area of the migrated cells into the wound area: Migrated Cell Surface Area=length of cell migration (mm)×2×length. Percent Closure (%)=Migrated Cell Surface Area/Total Surface Area×100.

To minimize biases, wells were arranged as shown in Table 8.

TABLE 8
24-well layout. ri: i-th replication

| | r1 | | r2 | | r3 | |
|---|---|---|---|---|---|---|
| | HPaCD | HPbCD | HPaCD | HPbCD | HPaCD | HPbCD |
| mM | 0 | 0 | 4 | 4 | 0 | 0 |
| | 1 | 1 | 2 | 2 | 1 | 1 |
| | 2 | 2 | 1 | 1 | 2 | 2 |
| | 4 | 4 | 0 | 0 | 4 | 4 |

Cell-based scratch assay. Cells were cultured in 24-well culture plates for 24 h up to 90%-100% confluence. Scratched wound lines on the upside of cultured cells were created by 200 μl yellow micropipette tip. The scratched cells were washed with PBS after removal of culture media. The cells were cultured for 2 h with 2 mM MbCD and after the removal of culture media cells were cultured for the next 2, 8, 12 or 24 h. All cell-based scratch assays were performed in the presence of the anti-mitotic reagent cytosine arabinoside (ara-c; Sigma) at a final concentration of 10′5 M in order to inhibit cell proliferation and analyze only cell migration. The wound area was measured from the image taken with an Axiovert 100 microscope (Carl Zeiss, Germany) by Image J program (NIH, USA) at 3 different sites from each wound area of gaps. Three independent experiments were performed.

Other methods used are described in Translational Medicine Communications, 1 (1), 3, “Membrane cholesterol depletion reduces breast tumor cell migration by a mechanism that involves non-canonical Wnt signaling and IL-10 secretion.” Translational Medicine Communications 1:3, and Okada et al. (1995) “Inhibition of Human Vascular Smooth Muscle Cell Migration and Proliferation by β-Cyclodextrin Tetradecasulfate.” The Journal of Pharmacology and Experimental Therapeutics 273(2):948-954, each of which are incorporated herein by reference in their entireties.

### Results

Results are shown in Table 9. In the absence of cyclodextrins, >80% of the wound are is closed. With CDs, wound closure is inhibited in a phospholipid-dependent manner. Extraction of cholesterol does not seem to inhibit wound closure, but has been shown to cause side-effects (ototoxicity).

TABLE 9
Wound healing results. Avg: average wound closure;
st_dev: standard deviation. Results for equivalent
doses (1 mM HPaCD, 2 mM HPbCD) are being underlined.

| | | | | | |
|---|---|---|---|---|---|
| mM | r1 | r2 | r3 | avg | st_dev |

MCF-7 cells

| | | | | | | | |
|---|---|---|---|---|---|---|---|
| HPaCD | 0 | 84.00 | 88.00 | 91.00 | 87.67 | 3.51 | |
| | 1 | 35.00 | 41.00 | 48.00 | 41.33 | 6.51 | * |
| | 2 | 40.00 | 30.00 | 38.00 | 36.00 | 5.29 | * |
| | 4 | 28.00 | 22.00 | 36.00 | 28.67 | 7.02 | * |
| HPbCD | 0 | 93.00 | 85.00 | 89.00 | 89.00 | 4.00 | |
| | 1 | 68.00 | 74.00 | 78.00 | 73.33 | 5.03 | * |
| | 2 | 41.00 | 48.00 | 56.00 | 48.33 | 7.51 | * |
| | 4 | 32.00 | 28.00 | 41.00 | 33.67 | 6.66 | * |

MDA-MB-231 cells

| | | | | | | | |
|---|---|---|---|---|---|---|---|
| HPaCD | 0 | 95.00 | 91.00 | 99.00 | 95.00 | 4.00 | |
| | 1 | 47.00 | 53.00 | 59.00 | 53.00 | 6.00 | * |
| | 2 | 40.00 | 33.00 | 47.00 | 40.00 | 7.00 | * |
| | 4 | 38.00 | 32.00 | 25.00 | 31.67 | 6.51 | * |
| HPbCD | 0 | 92.00 | 98.00 | 100.00 | 96.67 | 4.16 | |
| | 1 | 62.00 | 68.00 | 73.00 | 67.67 | 5.51 | * |
| | 2 | 48.00 | 59.00 | 61.00 | 56.00 | 7.00 | * |
| | 4 | 48.00 | 44.00 | 25.00 | 39.00 | 12.29 | * |

(* <0.05)

The results are highly consistent:
In all cases, migration decreased with higher concentration of either HPaCD or HPbCD.
In all cases, HPaCD had a stronger effect on migration than HPbCD; Inhibition at 1 mM HPαCD was superior to inhibition at 2 mM HPbCD.

### Discussion

The results in human two human tumor cancer cell lines confirm the hypothesis that the primary effect of bCDs on cell migration is not by scavenging cholesterol, but by scavenging phospholipids. The present disclosure has shown that “derailed endocytosis” is caused by genetic risk factors causing excessive influx of glycophospholipids, including PC, into the PI FIG. 35. Hence, the previous studies with bCDs, which did not proceed to clinical trials because of risk of cholesterol-related ototoxicity, can now be resumed after the elimination of cholesterol is avoided, while the phospholipid-related efficacy seen in bCD is increased.

HPaCD has the same toxicity as HPbCD, but is about twice as effective in scavenging phospholipids, in general, and even more effective in scavenging PC. See Langmuir, 29 (47), 14631-8; Molecules, 20 (11), 20269-85; Journal of Pharmacology and Experimental Therapeutics, 310 (2), 745-51. In children with NPC (Table 4, Mol Genet Metab, 108 (1), 76-81) doses of up to 1000 mg/kg/d HP-β-CD were well tolerated. With 700 mg/kg/d HPbCD having been proven as safe in parenteral treatment of NPC, 700 mg/kg/d HPaCD can now be used to continue with the human experiments that were stopped when bCDs were shown to have cholesterol-related ototoxicity. From the results in FIG. 35, 700 mg/kg/d of aCD have the same efficacy in preventing migration of cancer cells as 2000 mg/kg/d bCD.

### Example 13

### α-CD is at Least as Effective as HP-β-CD in Preventing Lysosomal Dysfunction in Cell-Lines of Various Lysosomal Storage Diseases

The results of Example 13 are consistent with findings in US 2015/0216895 A1, FIG. 5, WO2014022841A1 to McKew, John, et al., which found that “alpha-CD, beta-CD, and gamma-CD can reduce cholesterol accumulation, and MBCD [M-β-CD] was most potent.” In particular, the authors noted that “alpha-CD, beta-CD, and gamma-CD can reduce cholesterol accumulation in NPC cells [and] that these CDs increased intracellular Ca2⁺ and enhanced exocytosis.”

What has not been appreciated is that efficacy of α-CD against cholesterol storage disease implies that the effect cannot be due to scavenging of cholesterol, because α-CD cannot fit cholesterol. Our novel genetics results show that the effect is instead mediated by phospholipids and, thus, explain for the first time why about 6 mM α-CD actually was more effective in reducing lysosomal storage than about 8 mM HP-β-CD (FIGS. 38-40).

The results also provide an explanation why intravenous HP-β-CD is effective in NPC, even though neither cyclodextrins nor cholesterol can cross the blood brain barrier. The surprising findings also may end the current “battle” over intravenous v. intrathecal use of HP-β-CD. Science, 354 (6308), 18-19. As phospholipids cross the blood brain barrier more easily, an oral (absorbed from the intestine) version of HP-α-CD may prevent the need for either implanting an intrathecal pump or administering HP-β-CD as an intravenous infusion over several hours to prevent cytoplamic crystals from forming in the tubules of rat kidney. See Am J Pathol, 83 (2), 367-82.

### Example 14

### A Clathrate of HP-α-CD and Sodium Caprate (C-10 Fatty Acid) as an Oral Drug Extracts Phospholipids into Urine

PCs and LPCs form complexes with 4-5 moles and 3 moles of aCD, respectively. See Chem Pharm Bull (Tokyo), 33 (6), 2587-90.

Conjugated linoleic acid (CLA) forms complexes with 4-5 moles of aCD. See J Agric Food Chem, 50 (10), 2977-83. CLA/aCD at 1:4 mole ratio completely protect CLA from oxidation. See Nutrition & Metabolism, 5 (1), 16.

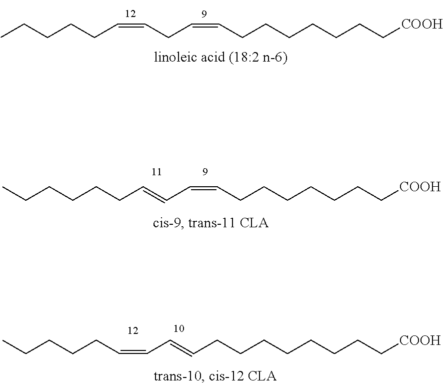 [SMILES: CCCCC/C=C\C/C=C\CCCCCCCC(=O)O ; CCCCCC/C=C/C=C\CCCCCCCC(=O)O ; CCCCC/C=C\C=C\CCCCCCCCC(=O)O]

“Capric acid (C10) regulates the paracellular permeability of the tight junctions in Caco-2 monolayers and in rat and human intestinal segments . . . . In contrast to C10, lauric acid (C12) did not induce detectable changes in tight junction morphology.” C10 acts faster, but 13 mM C10 and 0.75 mM C12 are equally effective after 40 min. J Pharmacol Exp Ther, 284 (1), 362-9.

An randomized n-of-1 dose escalation study was conducted to assess the activity of a clathrate of HP-α-CD and sodium caprate (C-10 fatty acid, capric acid, decanoic acid) in extracting phospholipids from serum into urine. Only at the highest (44 g) dose of HP-α-CD was softening of stool observed, a known side effect that is expected with larger doses of α-CD s. The results are shown in Table 10.

TABLE 10
Complexation with fatty acid improves activity of HP-α-CD. Excretion of phospholipids
into urine within 8 h after oral administration of either HP-α-CD or HP-α-CD/C-10 clathrate.

| Dose | HP-α-CD | | | avg | adj | | Clathrate | | | avg | adj | | |
|---|---|---|---|---|---|---|---|---|---|---|---|---|---|
| [g] | [mM] | [mM] | [mM] | [mM] | [mM] | [mM/g] | [mM] | [mM] | [mM] | [mM] | [mM] | [mM/g] | |
| 18 | 3.8 | 3.5 | 4.3 | 3.87 | 1.05 | 0.06 | 4.2 | 4.7 | 4.1 | 4.33 | 1.51 | 0.08 | 1.45 |
| 22 | 5.2 | 5.2 | 4.4 | 4.93 | 2.11 | 0.10 | 8.6 | 7.9 | 8.4 | 8.30 | 5.48 | 0.25 | 2.59 |
| 26 | 7.2 | 6.3 | 7.1 | 6.87 | 4.05 | 0.16 | 10.4 | 10.7 | 10.5 | 10.53 | 7.71 | 0.30 | 1.91 |
| 30 | 7.5 | 6.8 | 8.1 | 7.47 | 4.65 | 0.15 | 12.4 | 13.8 | 12.9 | 13.03 | 10.21 | 0.34 | 2.20 |
| 36 | 8.2 | 8.8 | 8.7 | 8.57 | 5.75 | 0.16 | 22.5 | 24.2 | 21.5 | 22.73 | 19.91 | 0.55 | 3.47 |
| 44 | 8.0 | 7.6 | 7.5 | 7.70 | 4.88 | 0.11 | 19.7 | 20.3 | 22.1 | 20.70 | 17.88 | 0.41 | 3.66 |

Notes:
Adj.: Expected phospholipid level in healthy subjects of 2.82 μM/L. (Saunders et al. 1997) subtracted.

Oral β-CD is rarely absorbed in mammals (˜1%); of the smaller α-CD, 1.6, 2.0 and 16% were found in urine within 6-8 h post oral administration. J Pharm Sci, 86 (2), 147-62; Biol Pharm Bull, 19 (2), 280-6; Regul Toxicol Pharmacol, 39 Suppl 1, 57-66. The strong dose-response relationship seen for HP-α-CD in Table 10 (except for the highest dose, which may be affected by faster passage due to stool softening) suggests that the higher estimate is more realistic for humans. In rats, instead of 1% for β-CD alone administered orally, up to 5% of β-CD were absorbed when administered rectally in suppositories containing triglycerides (Witepsol H5. See J Pharm Sci, 81 (11), 1119-25. Absorption increased from 5% to 26% when β-CD was replaced by HP-β-CD, which is more water-soluble. See Drug Development and Industrial Pharmacy, 16 (17), 2487-99.

Published results in LDL-receptor knockout mice who were fed a “Western diet (21% milk fat)” showed that adding “2.1% of “nonabsorbable” α-CD (10% of dietary fat content)” decreased plasma PLs by 17.5%. Metabolism, 57 (8), 1046-51. No mechanism of action was identified. From the above genetic and in vitro results, the reduction in plasma PLs seen was not (or not only) due to “saturated fat absorption” in the intestine and “excretion into the feces”, as speculated by the authors, but (also) through scavenging of PLs by α-CD absorbed from the intestine in the presence of milk fatty acids and excreted into urine.

While CDs are widely used as absorption enhancer, the present disclosure utilized a CD as an active ingredient and sodium caprate as an absorption enhancer for the CD. The present disclosure utilizes the smaller α-CD, applies the HP derivatization previously shown to enhance absorption of β-CD, uses a sodium salt of a fatty acid component of tryglycerides as a more effective absorption enhancer for delivery in the intestine. See J Appl Pharmacol Sci, 2 (7), 34-39; J Pharm Pharmacol, 39 (11), 887-91.

The results shown in Table 10 confirm that the novel and non-obvious combination of strategies for delivery of a novel drug, a HP-α-CD/sodium-caprate clathrate, to reduce endocytosis in metastatic, neurodegenerative, metabolic, and cardiovascular diseases yields an intervention that is capable of reducing serum phospholipids by a clinically relevant amount.

A clathrate is not formed by covalent bounds and both components are GRAS. α-CD is GRAS and available as a dietary supplement. Fatty acids are “permitted for direct addition to food for human consumption” [21 CFR 172.860], as “multipurpose additive” [21 CFR 172.860] and as “defoaming agents” [21 CFR 173.340]. Capric and caprylic acid is also GRAS under 21 CFR 184.1025 (GRAS Notice 449).

A randomized blinded n-of-1 PK/PD study was done to demonstrate that the clathrate is absorbed and extracts phospholipids from urine. The substances compared were
HP-α-CD,
the HP-α-CD/sodium caprate clathrate
a mixture of the HP-α-CD clathrate with the same amount of sodium caprate clathrate, also known as C-10 fatty acid, capric acid, decanoic acid.
a mixture of the HP-α-CD clathrate with the oily suspension proposed by (Tuvia S, Pelled D, et al. (2014) Pharmaceutical Research 31:2010-21).

Six samples per substance were taken.

The samples were taken in the evening before bed-time and urine samples were taken

The results are shown in FIG. 44.

### Example 15

### Selectivity of HP-α-CD/Sodium-Caprate Clathrate for Selected Phospholipids (PC/PS) Over PA and LPLs Exerts Effects Similar to Anti-Inflammatory Drugs

sPLA2 inflammatory activity is ongoing in the majority of MS patients, active or stable, regardless of treatment. In rats, EAE symptoms were attenuated by inhibition of sPLA2. (Cunningham et al. 2006) Increased sPLA2 activity is observed in the cerebrospinal fluid of humans with Alzheimer's disease (Chalbot et al. 2011), and may serve as a marker of increases in permeability of the blood-cerebrospinal fluid barrier. (Chalbot et al. 2011) Patients with AD (and MCI) had “aberrant activity of phospholipase A2 (PLA2)”.(Klavins et al. 2015)

Comparing morning urine of HPaCD with HPaCD* (HPaCD/sodium-caprate clathrate) confirmed that HPaCD is not resorbed, but the clathrate is at the expected molecular weight of 973+194=1167. (FIG. 43) Excretion of the phospholipids PC, PS, and PE (mW 730-790) is substantially increased by the clathrate, as expected. Unexpectedly, however, PA (675) and SM (650) are not reduced and neither are lyso-phospholipids (460-550).

The unexpected high specificity of the clathrate for PC/PS/PE implies that the clathrate does not only reduce activity of the PI-cycle, but also reduces inflammation. PC is split by soluble PLA2s into LPC (which is transformed by ATX into LPA) and proinflammatory arachidonic acid (AA). Scavenging lysophospholipids (LPC, LPA), rather than PC would activate sPLA2 and, thus, increase inflammation. Scavenging PC only, instead, deprives sPLA2 of its substrate and, in addition, reduces sPLA2 activity by increasing the LPC/PC ratio.

sPLA2-IIa activates HER2 and HER3, is overexpressed in lung, breast, pancreatic, skin, liver, and prostate cancer.(Z. Dong et al. 2014) activates EGFR in MCF7 human BC cancer cells (Hernandez et al. 2010), and is a marker of metastases in

“Researchers have been considering PLA2s could be a better therapeutic target than the downstream enzymes cyclooxygenase and lipoxygenase [yet]” (Yarla et al. 2016) Human PLA2s are involved in eicosanoic synthesis, phagocytosis by MΦs, airway hypersensitivity, inflammation, tumorigenesis (V) and allergen-induces asthma, myocardial ischemia/reperfusion injury, AA metabolism, inflammation tumorigenesis, atherosclerosis (X). (Yarla et al. 2016)

The unexpected reduction of inflammatory arachidonic acid exerts effects similar to corticosteroids (sPLA2 inhibition), NSAIDs (COX inhibition), and asthma drugs (LOX and leukotrine inhibition), see FIG. 1: Cyclodextrins, Including Hydroxypropyl Cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl. Typical CDs contain 6 (α-CD), 7 (β-CD) or 8 (γ-CD) D-glucose monomers in a ring, creating a cone shape that can accommodate guest molecules into their hydrophobic cavity.

FIG. 2: Clustering analysis of cholesterol interaction with α-CD and n. Influence of cyclodextrins on the proliferation of HaCaT keratinocytes. Mean values after 48 h incubation, normalized to the control, of at least six independent measurements. Top: ATP-Assay, bottom: PicoGreen-Assay). W6: α-CD, W6: β-CD, W7s HP: HP-β-CD. Modified from Hipler, U. C., et al. (2007), ‘Influence of cyclodextrins on the proliferation of HaCaT keratinocytes in vitro’, J Biomed Mater Res A, 83 (1), 70-9.

FIG. 3A through 3C: Specificity of Lipid Release I. Release of phospholipids (3A), cholesterol (3B) and proteins (3C) from intact (3B) or ghost (3A and 3C) erythrocytes treated with cyclodextrins. (◯) α-Cyclodextrin; (●) β-cyclodextrin; (Δ) γ-cyclodextrin. Modified from: (Ohtani Y, Irie T, et al. (1989) European Journal of Biochemistry 186:17-22).

FIG. 4A through 4B: Specificity of Lipid Release II. 4A) Cholesterol released from brain capillary endothelial cell (BCECs) after 2 h of incubation in the presence of various concentrations of α- and β-CDs. Results are expressed as a percentage of cholesterol released from BCECs compared with the control. Each percentage is the mean of three different filters and representative of two series of independent experiments. 4B) Phosphatidylcholine (PC) (light columns) and sphingomyelin (dark columns) released from BCECs after 2 h of incubation in the presence of α- and β-CDs at 0.5, 5, and 50 mM, respectively. Results are expressed as a percentage of phospholipids released from BCECs compared with the control. Each percentage is the mean of three different filters and representative of two series of independent experiments. Modified from: (Monnaert V, Tilloy S, et al. (2004) Journal of Pharmacology and Experimental Therapeutics 310:745-51).

FIG. 5: MβCD, but not αCD protect A2E against oxidation. The bisretinoid A2E was the first compound identified in extracts of ocular lipofuscin. Protection of A2E against oxidation was monitored by changes in the UV-visible absorption spectra of 5 μM A2E solutions. (5A) A2E oxidative status before (●) and after (○) blue-light irradsiation in the presence of indicated cyclodextrins (12 mM). (5B) A2E oxidative status at time 0 (●) and 1 d (○) after incubation at room temperature in the dark in the presence of cyclodecxtrins. Modified from (Nociari M M, Lehmann G L, et al. (2014) Proceedings of the National Academy of Sciences 111:E1402-E8).

FIG. Hence, aCD emerges as a replacement for “baby-aspirin's”

In summary, aCD slows down several processes that are essential during early life, but can cause problems after the age of 50:
migration of cells during pre-natal (tissue) and early-post-natal (neuronal) development,
cellular uptake of nutrients during growth, and
innate immunity (inflammation) as a defense against pathogens not seen, yet, by the immune system.

In the aging adult, these functions become less relevant.

### Example 16

### αCDs are More Efficient in Crossing the Blood-Brain Barrier (BBB) than bCDs

As phospholipids can easily cross the BBB, aCDs would not need to cross the BBB to be effective, but it would help. Published data show that aCDs are consistently twice as efficient as bCDs in crossing the BBB in vitro. After 2 h of incubation, 16.5-43.0% (vs 6.6-26.7%) were transported through the BBB (compared to 96% for caffeine or nicotine). See Journal of Pharmacology and Experimental Therapeutics, 310 (2), 745-51. These results were confirmed in an independent study of βCDs.(Binkowski-Machut et al. 2006)

While αCD was more toxic, HPaCD, bCD, MbCD, and HPbCD were similar. See Journal of Pharmacology and Experimental Therapeutics, 310 (2), 745-51.

### Example 17

### Study Design for Studying Neuroprotection by Alpha and Beta Cyclodextrin in Cell and Mouse Models of Alzheimer's Disease

Abstract. There is extensive evidence that cholesterol and membrane lipids play a key role in Alzheimer disease (AD) pathogenesis. Cyclodextrins (CD) are cyclic oligosaccharide compounds and hydroxypropyl-β-CD (HP-β-CD) is widely used to bind cholesterol. Since CD exerts significant beneficial effects in Niemann-Pick type C disease, which shares neuropathological features with AD, the effects of HP-β-CD were examined in cell and mouse models of AD. Cell membrane cholesterol accumulation was detected in N2a cells over-expressing Swedish mutant APP (SwN2a), and the level of membrane cholesterol was reduced by HP-β-CD treatment. HP-β-CD dramatically lowered the levels of Aβ42 in SwN2a cells, and the effects were persistent for 24 hours after withdrawal. Four months of subcutaneous HP-β-CD administration significantly improved spatial learning and memory deficits in Tg19959 mice, diminished Aβ plaque deposition, and reduced tau immunoreactive dystrophic neurites (DN). These are transgenic mice with 2 mutations in the APP gene which have been associated with human AD and beneficial effects were attributed to a reduction in cholesterol. Recently, a new GWAS analyses was used, which showed an overlap of phospholipid pathway genes in metastatic breast cancer with those found in AD cohorts. HPαCD also binds phospholipids, as does HP-β-CD, but does not bind cholesterol. HPαCD was twice as effective as HP-β-CD in slowing migration of breast cancer cells. It is therefore possible that the efficacy of HP-β-CD is not due to its effects on cholesterol but rather on phospholipids. One way to distinguish these possibilities is to test the efficacy of HPαCD both in vitro and in transgenic mice with APP mutations (Tg19959). In the present proposal we intend to determine whether HPαCD is equally efficacious, or much more efficacious, without having effects on cholesterol levels or membrane cholesterol as determined using filipin staining and mass spectroscopy. We will also determine whether the effects are dose-responsive both on beta-amyloid production in the N2a cells, as well as in vivo in the Tg19959 transgenic mice. These studies will therefore provide the ground work and scientific rationale to pursue this strategy further in clinical trials in patients with AD.

Summary of key supporting data. Previous, the effects of hydroxypropyl-βCD (HPβCD) were examined in cell and mouse models of AD. HPβCD dramatically lowered the levels of Aβ42 in SwN2a cells, and the effects were persistent for 24 h after withdrawal. Four months of subcutaneous HPβCD administration significantly improved spatial learning and memory deficits in Tg19959 mice, diminished Aβ plaque deposition, and reduced tau immunoreactive dystrophic neurites (DN). See The Journal of Experimental Medicine, 209 (13), 2501-13. HPβCD lowered levels of Aβ42 in part by reducing β-cleavage of the APP protein, and it also up-regulated the expression of genes involved in cholesterol transport and Aβ clearance. This study for the first time showed neuroprotective effects of HPβCD in a transgenic mouse model of AD, both by reducing Aβ production and enhancing clearance mechanisms, which suggested that it could be a novel therapeutic strategy for disease modification in AD. Among the upregulated genes was ABCA1, a membrane transporter that exports cholesterol and phospholipids and is considered as a target of agonist peptides for the treatment of AD.(Bielicki 2016)

Recent results, Wittkowski, Knut M., et al. (submitted), ‘Complex Polymorphisms in Endocytosis Genes Suggest alpha-Cyclodextrin against Metastases in Breast Cancer’, Appendix, 10 (2), identified several genes involved in “derailed endocytosis” of breast cancer, most of which (including ABCA1) had already been implicated in functional and gene-expression studies of AD, including

ATP8A1/ATP8B1 (Arch Neurol, 65 (1), 45-53; International Journal of Molecular Sciences, 14 (4), 7897-922; PLOS Genetics, 8 (8), e1002853.).

ANO4 (Alzheimers Dement, 10 (1), 45-52.).

ABCA1 (PLoS One, 11 (11), e0166195; Neurobiol Dis, 72 Pt A, 13-21; Alzheimers & Dementia, 11 (12), 1430-38; Neurobiol Dis, 72 Pt A, 54-60.).

AGPAT3/AGPAT4 (J Alzheimers Dis, 23 (2), 349-59.).(Sherva et al. 2011)

DGKQ (Zhu, X. C., et al. (2016), ‘Association of Parkinson's Disease GWAS-Linked Loci with Alzheimer's Disease in Han Chinese’, Molecular Neurobiology.).

All of these genes are involved in transport and metabolism of phospholipids, while only few are involved in transport of cholesterol, suggesting that the effects seen in the above animal models of AD could be a consequence of reductions in phospholipids rather than cholesterol.

This hypothesis was supported by in vitro experiments in two human breast cancer cell lines, MDA-MB-231 (triple negative) and MCF-7 (estrogen receptor positive), showing 1 mM HPαCD to be more effective than 2 mM HPβCD against migration of both ER− and ER+ tumor cell migration (p=0.0252). See Wittkowski, Knut M., et al. (submitted), ‘Complex Polymorphisms in Endocytosis Genes Suggest alpha-Cyclodextrin against Metastases in Breast Cancer’, Appendix, 10 (2).
Aim 1 (primary): To test efficacy of equimolar HPαCD versus HPβCD (2,000 and 4,000 mg/kg) and control on spatial learning and memory deficits, in Tg19959 mice overexpressing human mutant APP

Innovation: HPβCD is widely believed to exert its activity, including its activity in mouse models of AD, by scavenging cholesterol. See The Journal of Experimental Medicine, 209 (13), 2501-13. Our GWAS results, confirmed in in vitro studies, Wittkowski, Knut M., et al. (submitted), ‘Complex Polymorphisms in Endocytosis Genes Suggest alpha-Cyclodextrin against Metastases in Breast Cancer’, Appendix, 10 (2), showed association with genes already seen in functional and expression studies as implicating derailed endocytosis” in BC (Nat Rev Cancer, 8 (11), 835-50), and “driving pathogenesis in sporadic and familial [AD]”. Biomed Res Int, 2014, 167024. Hence the effect of HPβCD seen in previous in vitro studies (Translational Medicine Communications, 1 (1), 3; BMC Cancer, 10, 647) was likely true to scavenging PLs, rather than cholesterol. “Derailed/deranged signaling and associated endocytosis” in AD. Biomed Res Int, 2014, 167024 If similar results were seen in in vitro models of AD, novel treatments could be sought that target PLs, rather than cholesterol.

Significance: The initial positive in vivo results on AD in mice were not followed up with clinical trials, mainly because HPβCD was shown to carry the risk of causing permanent hearing loss from depriving outer hair cells of cholesterol.See The Journal of Experimental Medicine, 209 (13), 2501-13; (S. Takahashi et al. 2016) The recent GWAS results shift the focus from cholesterol to PLs. This, in turn, could now open novel treatment options for AD, including HPαCD, which may be more effective than HPβCD, while avoiding the risk of permanent hearing loss from scavenging cholesterol. HPαCD is also less toxic than HPβCD in vitro. See International Journal of Pharmaceutics, 101 (1-2), 97-103.

Approach: We will examine effects on cleavage of APP, expression of cholesterol and lipid transport genes, and Aβ clearance as well as levels of cholesterol and PLs. We will determine whether effects are dose-responsive by examining 2 concentrations of both HPαCD and HPβCD, and effects on lysosomes by cathepsin D immunostaining. We will perform these tests in five groups (Control, HPβCD_2g, HPβCD_4g, HPαCD_2g, HPαCD_4g) of 20 mice each.

Pitfalls and alternatives: Not all effects previously seen with HPβCD will be replicated with HPαCD and the differential effect will provide novel insights into the etiology. If, however, too few of the effects of HPβCD can be replicated with HPαCD, the hypothesis that the latter could become a safer treatment against AD will not be further pursued. Still, the results from Aim 2 may still provide novel insights leading to potential alternatives.
Aim 2: To explore the relative activity of scavenging PLs only (HPαCD) versus scavenging both PLs and cholesterol (HPβCD) on AD pathology . . .
Aim 2a: . . . in N2a cells expressing the Swedish APP mutation (membrane cholesterol, Aβ production)
Aim 2b: . . . in Tg19959 mice overexpressing human mutant APP (Aβ deposition, microgliosis, amylogenesis, tau and lysosomal abnormalities, gene expression)

Innovation: Even if HPαCD spatial learning and memory by improving Aβ and tau pathology, the role of PLs v. cholesterol in the etiology of AD remains to be elucidated.

Significance: A single drug is unlikely to become a panacea in AD. The mechanistic studies will provide insights to design studies that could lead to more specific drugs targeting cholesterol.

Approach: We will aim to replicate the successful in vitro studies of HPβCD reducing Aβ production in N2a cells expressing the Swedish APP mutation, this time using HPαCD instead of HPβCD. To further elucidate the mechanism of action, we will use filipin staining to assess the effect of scavenging PLs on membrane cholesterol.

Pitfalls and alternatives: The role of cholesterol in AD is assumed, but not known. Hence, both positive and negative results on the effect of HPαCD on membrane cholesterol and Aβ production will be helpful in refining the understanding of the role of CDs in AD and provide valuable insights for the development of additional drugs.

Experimental Design and Methods

Based on the previous results showing 4,000 mg/kg HPβCD to be effective in vitro and the in vitro results showing HPαCD to be at least twice as effective as HPαCD in regulating EECwe will compare HPαCD_2g v. HPβCD_4g, but also HPαCD_2g v. HPβCD_2g, HPαCD_4g v. HPβCD_4g, HPαCD_4g v. HPαCD_2g, and HPαCD_4g v. HPαCD_2g. See The Journal of Experimental Medicine, 209 (13), 2501-13. As positive controls, we will replicate the previous results comparing Control v. HPβCD_4g.

To guard against regression to the mean (winner's curse) and account for multiplicity (five dependent comparisons) in Aim 1 and to ensure that negative results in the exploratory Aim 2 can be meaningfully interpreted, we will increase the sample size from 10 per group in the previous study of two groups (α=0.05, power=0.80, δ=1.3) to 20 per group in this study of 5 groups (α=0.01, power=0.90). See The Journal of Experimental Medicine, 209 (13), 2501-13.

Experimental animals. Tg19959 mice were obtained from Dr. George Carlson (McLaughlin Research Institute, Great Falls, Mont.). Tg19959 mice are constructed by injecting FVB×129S6 Fl embryos with a cosmid insert containing human APP695 with two familial AD mutations (KM670/671NL and V717F), under control of the hamster PrP promoter. All experiments will be approved by the Institutional Animal Care and Use Committee at Weill-Cornell Medical College.

Cell lines: Mouse N2a neuroblastoma cells stably transfected with human APP695 carrying the 670/671 Swedish mutation (SwN2a) will be grown as described previously (J. Yao et al. 2010).

HP-α-CD/HP-β-CD (HP-x-CD) treatment in cells. 10 mM HPxCD stock solution will be made in 1×PBS. SwN2a and N2a cells will be treated with HPxCD (5 mM) in serum free medium at different incubation times. To measure Aβ levels, SwN2a cells will be recovered in serum free medium for 5 or 24 h after HPxCD treatment.

Filipin staining and analysis. Filipin is prepared in DMSO (50 mg/ml) and stored at −20° C. Cells will be fixed in 4% PFA for 30 min. For filipin staining, separate permeabilization of cells is not needed since filipin itself permeabilizes the cells. The stock solution will be diluted in PBS (1:100-1:500). The cells will be incubated for 15 min at room temperature and then washed in PBS 3× for 5 min. Anti-fading reagent (Fluoromount-G, SouthernBiotech) will be used in the mounting medium. Filipin is detected using λex=360 nm and λem=460 nm. A fluorescence microscope connected to a CCD camera will be used, and images are taken using Metamorph (Molecular Devices). To analyze the images, we will use Image J (NIH) to apply a 70% threshold onto the images and quantify the alteration of filipin intensity of SwN2a cells with and without CD treatment. See Nat Neurosci, 9 (10), 1265-73.

Cholesterol extraction from SwN2a cells. Six-well plates of SwN2a cells will be treated with and without CD for 15 min. Cells will be washed 3× with Hank's buffer and then 800 ul of hexane/isopropanol (3/2 v/v) containing β-sitosterol (internal standard, 5 ug of β-sitosterol per well) will be added. Lipids are extracted for 30 min under gentle shaking at room temperature. Lipid extracts will be transferred to 12×75 mm borosilicate glass culture tubes; and dried under Argon. The extraction will be repeated again and 50 ul hexane will be added per tube, vortexed and then transferred into glass vials for separation and analysis on GC-Mass Spec. The levels of cholesterol/mg protein will be calculated and plotted.

Cyclodextrin administration to mice. Mice will be treated with HPxCD (2000 or 4000 mg/kg) by subcutaneous injection twice weekly. HPxCD will be provided as a 20% (w/v) solution in isotonic saline with isotonic saline alone as the control. The injections of HP-x-CD in the mice will be started at P7, and the duration of the treatment will be four months. Twenty mice will be studied in each group.

Morris water maze. Spatial learning and memory will be analyzed using the Morris water maze. See Learning and Motivation, 12 (2), 239-60. The mice are handled daily, starting 1 wk prior to behavioral testing, in order to habituate them. During the acquisition period, visual cues will be arranged in the room. The hidden platform is located in the middle of the northwest (NW) quadrant. Each day, mice were placed next to and facing the wall of the basin in 4 starting positions: north, east, south, and west, corresponding to four successive trials per day. The duration of a trial will be 60 s with an inter-trial interval of 60 min. Whenever the mouse fails to reach the platform within 60 s, it is placed on the platform by the experimenter for 10 s. Latencies before reaching the platform will be recorded for 5 d and analyzed.

A probe trial will be assessed 24 h after the last trial of the acquisition period, removing the platform from the pool. Mice will be released on the north side for a single trial of 60 s, during which the time spent in the area of the platform will be measured. The velocity will also be measured.

The visible platform testing will be performed over 2 d with 4 trials per day. In this cued test, a pole will be added on the platform, and its location was changed between each trial. The duration of a trial will be 60 s with an inter-trial interval of 60 min. Latencies before reaching the platform will be recorded and averaged.

Sample preparation from brains and cells. Mice will be deeply anesthetized with intraperitoneal sodium pentobarbital and transcardially perfused with ice-cold saline. The brains will be removed and dissected on ice. One hemisphere will be used for histological analysis and the other hemisphere will be used for subsequent protein extraction or Trizol RNA extraction (Invitrogen). Brain tissues will be homogenized in lysis buffer containing 1% SDS+0.5% NP-40 and protease inhibitors (Roche) for Western blot analysis. Cells will be homogenized and prepared in Trizol for RNA extraction and in RIPA buffer or 1% Triton in PBS for protein extraction. Protein concentrations will be determined by BCA protein assay (Thermo Scientific).

Western blot analysis. Samples with equal amount of protein will be separated by Tricine-SDS gel electrophoresis and transferred to PVDF membranes using the iBlot dry blotting system (Invitrogen). Membranes will be blocked with 5% milk/0.1% Tween20 in TBS for 1 h at room temperature, followed by incubation with primary antibodies overnight at 4° C. Signals will be detected using HRP-conjugated secondary antibodies and enhanced chemiluminescence (Thermo Scientific). Blots will be scanned at 600 dpi and densitometry will be performed using ImageJ (NIH). We will use the following antibodies: mouse monoclonal anti-tubulin (Sigma, 1:10000), mouse anti-Aβ1-16 (6E10) monoclonal antibody (Covance, 1:1000), rabbit anti-APP C-terminal antibody (Calbiochem), HRP-conjugated goat anti-mouse IgG (1:2000) and goat anti-rabbit IgG (1:3000) (KPL).

Enzyme linked immunosorbent assay (ELISA). Aβ42 levels will be quantified using a commercial ELISA kit (Invitrogen, KHB3441). The manufacturer's protocol will be followed to measure Aβ levels in cell extracts, medium from SwN2a cells and brain extracts from mice. Medium from SwN2a cells will be diluted 1:1 in diluents, and brain extracts will be diluted 1:10 in diluents, and then loaded onto the plate for analysis. Each sample will be run in duplicate and the experiments will be repeated at least twice.

Immunohistology. The mice which will have been assessed behaviorally will be deeply anesthetized with intraperitoneal sodium pentobarbital and transcardially perfused with ice-cold saline. Brains will be post-fixed in 4% paraformaldehyde in PBS for at least 24 h. The brain tissues will be cut in 35 um sections, and immunostained using the avidin-biotin complex peroxidase method and visualized after DAB (diaminobenzidine) incubation for 5 min (Vector, Burlingame, Calif., USA). For each animal, 5 sections will be analyzed. For amyloid deposits, brain sections will be labeled with the anti-Aβ42 rabbit polyclonal antibody AB5078P (1:1000, Chemicon). For microglial activation, adjacent sections are also labeled with anti-CD-11b rat monoclonal antibody (1:100, Serotec). For phosphorylated tau, sections are labeled with AT8 antibody (1:500, Thermo Scientific). For cathepsin D, sections are labeled with RU4 antibody (generous gift from Dr. Ralph Nixon, New York University School of Medicine/Nathan Kline Institute). Using NIH Image 1.63 software (National Institutes of Health, Bethesda, Md., USA), the percentage areas occupied by AB5078P immunoreactive amyloid plaques and by CD-11b immunoreactive reactive microglia per 0.75 mm² will be calculated.

Thioflavin-S staining. Floating sections from Tg19959 will be washed and incubated in 1% Triton-PBS for 15 min, washed with PBS, and stained for 5 min with a solution of 0.05% thioflavin S (ThS) in 50% ethanol. Finally, sections are washed in 50% ethanol and then in water. The fluorescence of ThS is detected using λex=488 nm with fluorescence microscopy. The area of ThS fluorescence is determined using Image J and expressed as a fraction of total area.

Quantitation of genes by RT-PCR. RNA will be extracted from 4-month-old mouse brains using the Trizol protocol (Invitrogen). Total RNA (1 μg) will then be reverse transcribed into cDNA using the High Capacity cDNA Reverse Transcription kit (Applied Biosystems, USA), with the addition of nuclease-free deionized water. Reverse transcription is performed according to the manufacturer's protocol. A total of 30 ng of cDNA is loaded into each well of the PCR plate. The cDNA is analyzed in duplicate by real-time quantitative PCR using the ABI Prism 7000 Sequence Detection System (Applied Biosystems, USA) and detected with Power SYBR Green Master Mix (Applied Biosystems, USA. Primer sequences were obtained from previous publications. See Biochim Biophys Acta, 1801 (8), 831-8; Proc Natl Acad Sci USA, 106 (7), 2377-82.

ABCA1
(Fwd: 5'-CGTTTCCGGGAAGTGTCCTA-3';
Rev: 5'-GCTAGAGATGACAAGGAGGATGGA-3'),
ABCA2
(Fwd: 5'-AGTGCTCAGCCTTCGTACAG-3',
Rev: 5'-AGGCGCGTACAGGATTTTGG-3'),
ABCG1
(Fwd: 5'-TTTGAGGGATTTGGGTCTGAAC-3',
Rev: 5'-CCCCTTTAATCGTTTTGTCTGCT-3'),
NPC1
(Fwd: 5'-GGGATGCCCGTGCCTGCAAT-3';
Rev: 5'-CTGGCAGCTACATGGCCCCG-3'),

For each sample, the cycle number Ct to reach threshold fluorescence will be determined in duplicate for each mRNA and actin. To determine relative amounts of mRNA in Tg19959 mice vs. wildtype mice, data are presented using the 2-ΔΔCt method.

Statistical methods. Unpaired comparisons between groups will be performed using generalised Friedman/Kruskal-wallis type rank-tests with Scheffe-type multiple comparisons. Journal of the American Statistical Association, 83, 1163-70, 87:258. Calculations will be performed using R (http://www.cran.r-project.org/) and the muStat package (https://cran.r-project.org/package=muStat).

The primary outcome for Aim 1 is a composite u-score comprising
Total time spent reaching the hidden platform on day 5 and
Total time spent in the platform area.

To guard against artifacts (outliers, skewed distributions) in the interpretation (incl. Aim 2), we will plot results as box-and-whiskers plots. See [0726][0734] Tukey, John W. (1977), Exploratory data analysis (Reading, Mass.: Addison-Wesley) (individual data outside of the 10^{th}/90^{th} percentile, i.e., the two extremes at the top and bottom each).

### Example 18

### Study Design for Studying Neuroprotection by Alpha and Beta Cyclodextrin in Cell and Mouse Models of Parkinson's Disease

The aggregation of a-synuclein (a-syn) is believed to play a critical role in the pathogenesis of disorders such as dementia with Lewy bodies and Parkinson's disease. The main objective of a previous study (P. Bar-On et al. 2006a) was to determine whether methyl-b-cyclodextrin (MbCD), interfered with a-syn accumulation in models of synucleinopathy. See J Neurochem, 98 (4), 1032-45. The authors studied the effects of MbCD on the accumulation of a-syn in a transfected neuronal cell line and in transgenic mice. Immunoblot analysis showed that MbCD reduced the level of a-syn in the membrane fraction and detergent-insoluble fraction of transfected cells. In agreement with the in vitro studies, treatment of mice with MbCD resulted in decreased levels of a-syn in membrane fractions and reduced accumulation of a-syn in the neuronal cell body and synapses. Taken together, these results were interpreted as suggesting that changes in cholesterol and lipid composition using MbCD may become a treatment of synucleinopathies. The results of the above examples, however, suggest that the effects seen were primarily due to MbCD, which was seen as a “cholesterol lowering agent” were, in fact, primarily due to MbCD scavenging phospholipids. In the current Example, the study will be replicated, this time using HPaCD, rather than MbCD.

Effects of MbCD on a-syn levels in non-tg and hα-syn tg mice brains. Non-tg and ha-syn tg mice will be treated with MbCD (10 mM) for 1 week and analyzed by western blot and immunocytochemistry.

### Example 19

### Study Design for Testing the Efficacy of a Cyclodextrin in Breast Cancer

Because women after resection of breast cancer have a definitive diagnosis, clinical trials of HPaCD against “derailed endocytosis” should start in this population. As a non-limiting example, a seamless phase 2b/3 clinical trial for HP-α-CD for the prevention of metastases in breast cancer could be conducted in a population of women with
triple negative breast cancer (tamoxifen and herceptin do not work well in this population) and
axillary lymph node metastasis (patients have established the tendency to develop metastases)

The phase 2b part of the seamless design would have futility as an outcome after the first 80 patients have been seen for at least 2 years and continue to be treated without unblinding, so that they can contribute to the primary endpoint, which would have time to distant metastasis as the outcome. As the recurrence rate is high for the first three years only data will be collected for up to 5 yrs. See J Breast Cancer, 18 (4), 371-7. Some of the later recruited patients will be administratively censored when the last patient will have been seen for two years. Gehan's test will be used to compare treated v. placebo patients. See Biometrika, 52, 203-23.

At an effective median observation time of three years, one would expect 30% of women to have a distant recurrence and could detect a reduction in incidence by 50% (to 15%) with the standard 80% power at the 5% level with 125 subjects per group. The placebo-controlled treatment would be given on top of the standard of care (chemotherapy, radiation, . . . ).
As long-term parenteral administration of HP-β-CD (200 mg/kg) was reported to decrease bone mineral density (BMD) in rats (Toxicol Pathol, 40 (5), 742-50), the study should carefully monitor
   Bone density.

Human erythrocytes tolerate α-CD better then β-CD. See WHO/JECFA Food Additive Series, 48, 1030. From animal studies, which have less tolerance for cyclodextrin than humans, the dose-limiting factors are likely
nephrotoxicity and
hemolysis

In animal studies, α-CD did not show ototoxicity (Ann Clin Transl Neurol, 3 (5), 366-80) still, the dose-finding studies should carefully screen for
Ototoxicity.

### Example 20

### Study Design for Testing the Efficacy of a Cyclodextrin in FSGS

In one aspect, the present disclosure provides a method of treating and/or preventing focal segmental glomerulosclerosis (FSGS) and/or nephrotic swelling in a subject in need thereof the method comprising administering to the subject an effective amount of a cyclodextrin, or an analogue or derivative thereof, either alone or in combination with one or more additional active agents. In some embodiment the composition is a clathrate of HP-aCD and sodium caprate or caprylate.

A study will be performed in which HP-alpha-CD with and without clathrate will be administered orally to rats using a model set forth in Fogo, Semin Nephrol. 2003 March; 23(2):161-71, which is incorporated by reference in its entirety. Other oral formulations comrprising and not comprising HP-alpha-CD will be tested along side HP-alpha-CD to measure efficacy.
Other oral formulations may include those described in WO2016105465, ORAL COMPOSITIONS FOR INSOLUBLE COMPOUNDS, also incorporated by reference in its entirety.

We expect that HP-alpha-CD or analogus or derivatives thereof will effectively treat and/or prevent development of FSGS in mammals as well or better than other experimental compounds without known side effects of beta-cyclodextrin.

### REFERENCES

Agalliu, I., et al. (2015), ‘Higher frequency of certain cancers in LRRK2 G2019S mutation carriers with Parkinson disease: a pooled analysis’, JAMA Neurol, 72 (1), 58-65.
Agardan, N. B., et al. (2015), ‘The Effectiveness of Raloxifene-Loaded Liposomes and Cochleates in Breast Cancer Therapy’, AAPS PharmSciTech.
Ahmed, H. and AlSadek, D. M. (2015), ‘Galectin-3 as a Potential Target to Prevent Cancer Metastasis’, Clin Med Insights Oncol, 9, 113-21.
Ahmed, M. R., et al. (2011), ‘Ubiquitin ligase parkin promotes Mdm2-arrestin interaction but inhibits arrestin ubiquitination’, Biochemistry, 50 (18), 3749-63.
Alanko, J. and Ivaska, J. (2016), ‘Endosomes: Emerging Platforms for Integrin-Mediated FAK Signalling’, Trends Cell Biol, 26 (6), 391-8.
Alcalay, R. N., et al. (2016), ‘SCARB2 variants and glucocerebrosidase activity in Parkinson's disease’, NPJ Parkinson Dis, 2.
Allen, I. V. (1981), ‘The pathology of multiple sclerosis—fact, fiction and hypothesis’, Neuropathol Appl Neurobiol, 7 (3), 169-82.
Almahariq, M., et al. (2013), ‘A novel EPAC-specific inhibitor suppresses pancreatic cancer cell migration and invasion’, Mol Pharmacol, 83 (1), 122-8.
American Cancer Society (2015), Cancer Facts & Figures 2015 (Atlanta: American Cancer Society).
Anazi, S., et al. (2016), ‘Clinical genomics expands the morbid genome of intellectual disability and offers a high diagnostic yield’, Mol Psychiatry.
Antalis, C. J., et al. (2011), ‘Migration of MDA-MB-231 breast cancer cells depends on the availability of exogenous lipids and cholesterol esterification’, Clin Exp Metastasis, 28 (8), 733-41.
Antonell, A., et al. (2015), ‘Altered Blood Gene Expression of Tumor-Related Genes (PRKCB, BECN1, and CDKN2A) in Alzheimer's Disease’, Molecular Neurobiology.
Antoniou, A. C., et al. (2010), ‘Common breast cancer susceptibility alleles and the risk of breast cancer for BRCA1 and BRCA2 mutation carriers: implications for risk prediction’, Cancer Res, 70 (23), 9742-54.
Arima, H., et al. (1992), ‘Enhanced rectal absorption and reduced local irritation of the anti-inflammatory drug ethyl 4-biphenylylacetate in rats by complexation with water-soluble beta-cyclodextrin derivatives and formulation as oleaginous suppository’, J Pharm Sci, 81 (11), 1119-25.
Armitage, Peter (1955), ‘Tests for linear trends in proportions and frequencies’, Biometrics, 11 (3), 375-86.
Armstrong, A., et al. (2014), ‘Lysosomal network proteins as potential novel CSF biomarkers for Alzheimer's disease’, Neuromolecular Med, 16 (1), 150-60.
Baietti, M. F., et al. (2012), ‘Syndecan-syntenin-ALIX regulates the biogenesis of exosomes’, Nat Cell Biol, 14 (7), 677-85.
Balla, Tamas (2013), ‘Phosphoinositides: Tiny Lipids With Giant Impact on Cell Regulation’, Physiological Reviews, 93 (3), 1019-137.
Bar-On, P., et al. (2006a), ‘Effects of the cholesterol-lowering compound methyl-beta-cyclodextrin in models of alpha-synucleinopathy’, J Neurochem, 98 (4), 1032-45.
Bar-On, Pazit, et al. (2006b), ‘Effects of the cholesterol-lowering compound methyl-β-cyclodextrin in models of α-synucleinopathy’, Journal of Neurochemistry, 98 (4), 1032-45.
Beebe-Dimmer, J. L., et al. (2015), ‘Familial clustering of breast and prostate cancer and risk of postmenopausal breast cancer in the Women's Health Initiative Study’, Cancer 121 (8), 1265-72.
Bektas, M., et al. (2005), ‘A novel acylglycerol kinase that produces lysophosphatidic acid modulates cross talk with EGFR in prostate cancer cells’, J Cell Biol, 169 (5), 801-11.
Ben-Chetrit, N., et al. (2015), ‘Synaptojanin 2 is a druggable mediator of metastasis and the gene is overexpressed and amplified in breast cancer’, Sci Signal, 8 (360), ra7.
Ben Halima, S., et al. (2016), ‘Specific Inhibition of beta-Secretase Processing of the Alzheimer Disease Amyloid Precursor Protein’, Cell Rep, 14 (9), 2127-41.
Bereczki, E., et al. (2016), ‘Synaptic proteins predict cognitive decline in Alzheimer's disease and Lewy body dementia’, Alzheimers Dement.
Betz, A., et al. (1997), ‘Direct interaction of the rat unc-13 homologue Munc13-1 with the N terminus of syntaxin’, J Biol Chem, 272 (4), 2520-6.
Bielicki, J. K. (2016), ‘ABCA1 agonist peptides for the treatment of disease’, Curr Opin Lipidol, 27 (1), 40-6.
Boehm-Cagan, A., et al. (2016), ‘Differential Effects of apoE4 and Activation of ABCA1 on Brain and Plasma Lipoproteins’, PLoS One, 11 (11), e0166195.
Bogie, J. F., et al. (2013), ‘Myelin alters the inflammatory phenotype of macrophages by activating PPARs’, Acta Neuropathol Commun, 1, 43.
Bohdanowicz, M. and Grinstein, S. (2013), ‘Role of phospholipids in endocytosis, phagocytosis, and macropinocytosis’, Physiol Rev, 93 (1), 69-106.
Bosch, Ana, et al. (2015), ‘PI3K inhibition results in enhanced estrogen receptor function and dependence in hormone receptor-positive breast cancer’, Science Translational Medicine, 7 (283), 283ra51-83ra51.
Bottcher, T., et al. (2013), ‘Fabry disease—underestimated in the differential diagnosis of multiple sclerosis?’, PLoS One, 8 (8), e71894.
Bradley, Ryan M., et al. (2015), ‘Acylglycerophosphate acyltransferase 4 (AGPAT4) is a mitochondrial lysophosphatidic acid acyltransferase that regulates brain phosphatidylcholine, phosphatidylethanolamine, and phosphatidylinositol levels’, Biochimica et Biophysica Acta (BBA)—Molecular and Cell Biology of Lipids, 1851 (12), 1566-76.
Bras, J., et al. (2014a), ‘Genetic analysis implicates APOE, SNCA and suggests lysosomal dysfunction in the etiology of dementia with Lewy bodies’, Hum Mol Genet, 23 (23), 6139-46.
Bras, Jose, et al. (2014b), ‘Genetic analysis implicates APOE, SNCA and suggests lysosomal dysfunction in the etiology of dementia with Lewy bodies’, Human Molecular Genetics, 23 (23), 6139-46.
Brewster, M. E. and Loftsson, T. (2007), ‘Cyclodextrins as pharmaccutical solubilizers’, Advanced Drug Delivery Reviews, 59 (7), 645-66.
Bultema, J. J., et al. (2012), ‘BLOC-2, AP-3, and AP-1 proteins function in concert with Rab38 and Rab32 proteins to mediate protein trafficking to lysosome-related organelles’, J Biol Chem, 287 (23), 19550-63.
Burghel, George J., et al. (2013), ‘Identification of Candidate Driver Genes in Common Focal Chromosomal Aberrations of Microsatellite Stable Colorectal Cancer’, Plos One, 8 (12), e83859.
Busa, W. B. (1988), ‘Roles for the phosphatidylinositol cycle in early development’, Philos Trans R Soc Lond B Biol Sci, 320 (1199), 415-26.
Cai, R., et al. (2015), ‘Deterministic identification of specific individuals from GWAS results’, Bioinformatics, 31 (11), 1701-7.
Cai, W., et al. (2013), ‘Adenylyl cyclase 6 activation negatively regulates TLR4 signaling through lipid raft-mediated endocytosis’, J Immunol, 191 (12), 6093-100.
Camilleri, P., Haskins, N. J., and Howlett, D. R. (1994), ‘beta-Cyclodextrin interacts with the Alzheimer amyloid beta-A4 peptide’, FEBS Lett, 341 (2-3), 256-8.
Cammarata, G, et al. (2015), ‘High variability of Fabry disease manifestations in an extended Italian family’, Biomed Res Int, 2015, 504784.
Campbell, I. M., et al. (2012), ‘Novel 9q34.11 gene deletions encompassing combinations of four Mendelian disease genes: STXBP1, SPTAN1, ENG, and TOR1A’, Genet Med, 14 (10), 868-76.
Castro, E. and Eeles, R. (2012), ‘The role of BRCA1 and BRCA2 in prostate cancer’, Asian J Androl, 14 (3), 409-14.
Cataldo, A. M., et al. (2000), ‘Endocytic pathway abnormalities precede amyloid beta deposition in sporadic Alzheimer's disease and Down syndrome: differential effects of APOE genotype and presenilin mutations’, Am J Pathol, 157 (1), 277-86.
Cataldo, Didier, et al. (2015), ‘Use Of Cyclodextrin For Treatment And Prevention Of Bronchial Inflammatory Diseases’, U.S. Pat. No. 9,034,846 B2 (US: UNIV LIEGE).
Cha, S. H., et al. (2015), ‘Loss of parkin promotes lipid rafts-dependent endocytosis through accumulating caveolin-1: implications for Parkinson's disease’, Mol Neurodegener, 10, 63.
Chen, Chun-Lin, et al. (2009), ‘Inhibitors of clathrin-dependent endocytosis enhance TGFβ signaling and responses’, Journal of Cell Science, 122 (11), 1863-71.
Chen, X., et al. (2014), ‘Altered Cholesterol Intracellular Trafficking and the Development of Pathological Hallmarks of Sporadic AD’, J Parkinsons Dis Alzheimers Dis, 1 (1).
Chen, X. I. N., et al. (2015), ‘Vav3 oncogene is upregulated and a poor prognostic factor in breast cancer patients’, Oncology Letters, 9 (5), 2143-48.
Cheng, Danni, et al. (2011), ‘Lipid Pathway Alterations in Parkinson's Disease Primary Visual Cortex’, Plos One, 6 (2), e17299.
Chew, C. L., Chen, M., and Pandolfi, P. P. (2016), ‘Endosome and INPP4B’, Oncotarget, 7 (1), 5-6.
Chin, H., et al. (2006), ‘Protein kinase A-dependent phosphorylation of B/K protein’, Exp Mol Med, 38 (2), 144-52.
Choubey, V., et al. (2014), ‘BECN1 is involved in the initiation of mitophagy: it facilitates PARK2 translocation to mitochondria’, Autophagy, 10 (6), 1105-19.
Cicek, M. S., et al. (2012), ‘Colorectal cancer linkage on chromosomes 4q21, 8q13, 12q24, and 15q22’, Plos One, 7 (5), e38175.
Cirrito, J. R., et al. (2008), ‘Endocytosis is required for synaptic activity-dependent release of amyloid-beta in vivo’, Neuron, 58 (1), 42-51.
Clear, Kasey J. and Smith, Bradley D. (2015), ‘Synthetic Receptors for polar Lipids: Design Principles and Applications’, in Bradley D. Smith (ed.), Monographs in Supramolecular Chemistry (14: Royal Society of Chemistry), 404-36.
Cleveland, William S. and Devlin, Susan J. (1988), ‘Locally Weighted Regression: An Approach to Regression Analysis by Local Fitting’, Journal of the American Statistical Association, 83 (403), 596-610.
Coisne, C., et al. (2016), ‘Cyclodextrins as Emerging Therapeutic Tools in the Treatment of Cholesterol-Associated Vascular and Neurodegenerative Diseases’, Molecules, 21 (12).
Colacurcio, D. J. and Nixon, R. A. (2016), ‘Disorders of lysosomal acidification—The emerging role of v-ATPase in aging and neurodegenerative disease’, Ageing Res Rev.
Coleman, J. A., Quazi, F., and Molday, R. S. (2013), ‘Mammalian P4-ATPases and ABC transporters and their role in phospholipid transport’, Biochim Biophys Acta, 1831 (3), 555-74.
Committee for Human Medicinal Products (CHMP) (2014), ‘Background review for cyclodextrins used as excipients’, Eurpean Medicines Agency, EMA/CHMP/333892/2013.
Crews, L., et al. (2009), ‘Role of synucleins in Alzheimer's disease’, Neurotox Res, 16 (3), 306-17.
Cronin, S., et al. (2015), ‘Hearing Loss and Otopathology Following Systemic and Intracerebroventricular Delivery of 2-Hydroxypropyl-Beta-Cyclodextrin’, J Assoc Res Otolaryngol, 16 (5), 599-611.
Crumling, M. A., et al. (2012), ‘Hearing loss and hair cell death in mice given the cholesterol-chelating agent hydroxypropyl-beta-cyclodextrin’, Plos One, 7 (12), e53280.
Cuzner, M. L. and Davison, A. N. (1973), ‘Changes in cerebral lysosomal enzyme activity and lipids in multiple sclerosis’, J Neurol Sci, 19 (1), 29-36.
da Costa, A., et al. (2012), ‘Identification of six potential markers for the detection of circulating canine mammary tumour cells in the peripheral blood identified by microarray analysis’, J Comp Pathol, 146 (2-3), 143-51.
Daleke, D. L. (2007), ‘Phospholipid flippases’, J Biol Chem, 282 (2), 821-5.
Danthi, P. and Chow, M. (2004), ‘Cholesterol removal by methyl-beta-cyclodextrin inhibits poliovirus entry’, J Virol, 78 (1), 33-41.
Davidson, C. D., et al. (2016), ‘Efficacy and ototoxicity of different cyclodextrins in Niemann-Pick C disease’, Ann Clin Transl Neurol, 3 (5), 366-80.
Davis, Mark E. and Brewster, Marcus E. (2004), ‘Cyclodextrin-based pharmaceutics: past, present and future’, Nat Rev Drug Discov, 3 (12), 1023-35.
Day, P., et al. (2011), ‘Syntaxins 3 and 4 mediate vesicular trafficking of alpha5beta1 and alpha3beta1 integrins and cancer cell migration’, Int J Oncol, 39 (4), 863-71.
Deli, Maria A. (2009), ‘Potential use of tight junction modulators to reversibly open membranous barriers and improve drug delivery’, Biochimica et Biophysica Acta (BBA)—Biomembranes, 1788 (4), 892-910.
Di Fiore, Pier Paolo and von Zastrow, Mark (2014), ‘Endocytosis, Signaling, and Beyond’, Cold Spring Harbor Perspectives in Biology, 6 (8).
Diao, J., et al. (2013), ‘Native alpha-synuclein induces clustering of synaptic-vesicle mimics via binding to phospholipids and synaptobrevin-2/VAMP2’, Elife, 2, e00592.
Disse, M., et al. (2016), ‘A Review of the Association Between Parkinson Disease and Malignant Melanoma’, Dermatol Surg, 42 (2), 141-6.
Dong, Xiaoliu, et al. (2016), ‘The relevance of ABCA1 R219K polymorphisms and serum ABCA1 protein concentration to Parkinson's disease pathogenesis and classification: a case-control study’, Genes & Genomics, 38 (3), 243-50.
Dong, Y., et al. (2015), ‘Synaptojanin cooperates in vivo with endophilin through an unexpected mechanism’, Elife, 4.
Dos Santos, A. G, et al. (2017), ‘Changes in membrane biophysical properties induced by the Budesonide/Hydroxypropyl-beta-cyclodextrin complex’, Biochim Biophys Acta, 1859 (10), 1930-40.
Duchene, D. and Wouessidjewe, D. (1990), ‘Pharmaceutical uses of cyclodextrins and derivatives’, Drug Development and Industrial Pharmacy, 16 (17), 2487-99.
Erneux, C., et al. (2016), ‘New Functions of the Inositol Polyphosphate 5-Phosphatases in Cancer’, Curr Pharm Des, 22 (16), 2309-14.
Falasca, Marco (ed.), (2012), Phosphoinositides and Disease, ed. Peter K. Vogt (Current topics in microbiology and Immunology, Dordrecht: Springer).
Farge, E., et al. (1999), ‘Enhancement of endocytosis due to aminophospholipid transport across the plasma membrane of living cells’, Am J Physiol, 276 (3 Pt 1), C725-33.
Fauvelle, F., et al. (1997), ‘Mechanism of alpha-cyclodextrin-induced hemolysis. 1. The two-step extraction of phosphatidylinositol from the membrane’, J Pharm Sci, 86 (8), 935-43.
Feldman, A. L., et al. (2014), ‘Familial coaggregation of Alzheimer's disease and Parkinson's disease: systematic review and meta-analysis’, Neuroepidemiology, 42 (2), 69-80.
Feng, D. D., Cai, W., and Chen, X. (2015), ‘The associations between Parkinson's disease and cancer: the plot thickens’, Transl Neurodegener, 4, 20.
Fernandez-Nogueira, P., et al. (2016), ‘Differential expression of neurogenes among breast cancer subtypes identifies high risk patients’, Oncotarget, 7 (5), 5313-26.
Filigheddu, N., et al. (2007), ‘Diacylglycerol kinase is required for HGF-induced invasiveness and anchorage-independent growth of MDA-MB-231 breast cancer cells’, Anticancer Res, 27 (3B), 1489-92.
Finn, R. S., Aleshin, A., and Slamon, D. J. (2016), ‘Targeting the cyclin-dependent kinases (CDK) 4/6 in estrogen receptor-positive breast cancers’, Breast Cancer Res, 18 (1), 17.
Frank, D. W., Gray, J. E., and Weaver, R. N. (1976), ‘Cyclodextrin nephrosis in the rat’, Am J Pathol, 83 (2), 367-82.
Frere, S. and Slutsky, I. (2016), ‘Targeting PTEN interactions for Alzheimer's disease’, Nat Neurosci, 19 (3), 416-8.
Fukuda, M. (2016), ‘Multiple Roles of VARP in Endosomal Trafficking: Rabs, Retromer Components and R-SNARE VAMP7 Meet on VARP’, Traffic, 17 (7), 709-19.
Fukuda, Mitsunori (2013), ‘The role of synaptotagmin and synaptotagmin-like protein (Slp) in regulated exocytosis’, Madame Curie Regulated Database [Internet].
Fulop, T., et al. (2012), ‘Aging, immunosenescence and membrane rafts: the lipid connection’, Longev Healthspan, 1, 6.
Garcia-Closas, M., et al. (2013), ‘Genome-wide association studies identify four ER negative-specific breast cancer risk loci’, Nat Genet, 45 (4), 392-8, 98e1-2.
Gatto, F., et al. (2015), ‘Flux balance analysis predicts essential genes in clear cell renal cell carcinoma metabolism’, Sci Rep, 5, 10738.
Gautam, S., et al. (2014), ‘beta-cyclodextrin and curcumin, a potent cocktail for disaggregating and/or inhibiting amyloids: a case study with alpha-synuclein’, Biochemistry, 53 (25), 4081-3.
Gautam, V., et al. (2015), ‘Synaptotagmins interact with APP and promote Abeta generation’, Mol Neurodegener, 10, 31.
Gehan, E. A. (1965), ‘A generalised Wilcoxon test for comparing arbitrarily singly censored samples’, Biometrika, 52, 203-23.
George, M. F., et al. (2016), ‘Multiple sclerosis risk loci and disease severity in 7,125 individuals from 10 studies’, Neurol Genet, 2 (4), e87.
Germain, D. P. (2010), ‘Fabry disease’, Orphanet J Rare Dis, 5, 30.
Gimpl, G and Gehrig-Burger, K. (2011), ‘Probes for studying cholesterol binding and cell biology’, Steroids, 76 (3), 216-31.
Goetz, J. G, et al. (2011), ‘Biomechanical remodeling of the microenvironment by stromal caveolin-1 favors tumor invasion and metastasis’, Cell, 146 (1), 148-63.
Gonzalez, A., et al. (2014), ‘Lysosomal integral membrane protein-2: a new player in lysosome-related pathology’, Mol Genet Metab, 111 (2), 84-91.
Gotoh, K., et al. (2014), ‘The antitumor effects of methyl-beta-cyclodextrin against primary effusion lymphoma via the depletion of cholesterol from lipid rafts’, Biochem Biophys Res Commun, 455 (3-4), 285-9.
Gould, S. and Scott, R. C. (2005), ‘2-Hydroxypropyl-beta-cyclodextrin (HP-beta-CD): a toxicology review’, Food Chem Toxicol, 43 (10), 1451-9.
Grosse, P. Y., Bressolle, F., and Pinguet, F. (1998), ‘Antiproliferative effect of methyl-beta-cyclodextrin in vitro and in human tumour xenografted athymic nude mice’, Br J Cancer 78 (9), 1165-9.
Guerra, Fabiana Sélos, et al. (2016), ‘Membrane cholesterol depletion reduces breast tumor cell migration by a mechanism that involves non-canonical Wnt signaling and IL-10 secretion’, Translational Medicine Communications, 1 (1), 3.
Guerreiro, R., et al. (2016), ‘Genome-wide analysis of genetic correlation in dementia with Lewy bodies, Parkinson's and Alzheimer's diseases’, Neurobiol Aging, 38, 214 e7-10.
Gunasekara, L., et al. (2017), ‘Pulmonary surfactant dysfunction in pediatric cystic fibrosis: Mechanisms and reversal with a lipid-sequestering drug’, J Cyst Fibros.
Gupta, A. and Iadecola, C. (2015), ‘Impaired Abeta clearance: a potential link between atherosclerosis and Alzheimer's disease’, Front Aging Neurosci, 7, 115.
Gurney, M. E., D'Amato, E. C., and Burgin, A. B. (2015), ‘Phosphodiesterase-4 (PDE4) molecular pharmacology and Alzheimer's disease’, Neurotherapeutics, 12 (1), 49-56.
Haddad, S. A., et al. (2016), ‘An exome-wide analysis of low frequency and rare variants in relation to risk of breast cancer in African American Women: the AMBER Consortium’, Carcinogenesis.
Hall, E. H., et al. (2009), ‘Tensinl requires protein phosphatase-1alpha in addition to RhoGAP DLC-1 to control cell polarization, migration, and invasion’, J Biol Chem, 284 (50), 34713-22.
Hamon, Yannick, et al. (2006), ‘Cooperation between Engulfment Receptors: The Case of ABCA1 and MEGF10’, Plos One, 1 (1), e120.
Hanson, H. A., et al. (2016), ‘Is Cancer Protective for Subsequent Alzheimer's Disease Risk? Evidence From the Utah Population Database’, J Gerontol B Psychol Sci Soc Sci.
Hashimoto, S., et al. (2016), ‘Lysophosphatidic acid activates Arf6 to promote the mesenchymal malignancy of renal cancer’, Nat Commun, 7, 10656.
Hesketh, G G, et al. (2014), ‘VARP is recruited on to endosomes by direct interaction with retromer, where together they function in export to the cell surface’, Dev Cell, 29 (5), 591-606.
Hipler, U. C., et al. (2007), ‘Influence of cyclodextrins on the proliferation of HaCaT keratinocytes in vitro’, J Biomed Mater Res A, 83 (1), 70-9.
Hoeffding, Wassily (1948), ‘A class of statistics with asymptotically normal distribution’, Annals of Mathematical Statistics, 19, 293-325.
Hopkins, M. M., et al. (2016), ‘Eicosopentaneoic Acid and Other Free Fatty Acid Receptor Agonists Inhibit Lysophosphatidic Acid- and Epidermal Growth Factor-Induced Proliferation of Human Breast Cancer Cells’, J Clin Med, 5 (2).
Hoshino, Ayuko, et al. (2015), ‘Tumour exosome integrins determine organotropic metastasis’, Nature, 527 (7578), 329-35.
Hsu, W. C., et al. (2008), ‘Promoter polymorphisms modulating HSPAS expression may increase susceptibility to Taiwanese Alzheimer's disease’, J Neural Transm (Vienna), 115 (11), 1537-43.
Huang, Z. and London, E. (2013), ‘Effect of cyclodextrin and membrane lipid structure upon cyclodextrin-lipid interaction’, Langmuir 29 (47), 14631-8.
Hultberg, B. and Olsson, J. E. (1978), ‘Diagnostic value of determinations of lysosomal hydrolases in CSF of patients with neurological diseases’, Acta Neurol Scand, 57 (3), 201-15.
—(1979), ‘Lysosomal hydrolases in CSF of patients with multiple sclerosis’, Acta Neurol Scand, 59 (1), 23-30.
Hunter, David J., et al. (2007), ‘A genome-wide association study identifies alleles in FGFR2 associated with risk of sporadic postmenopausal breast cancer’, Nature Genetics, 39 (7), 870-74.
Hurley, J. H. and Odorizzi, G (2012), ‘Get on the exosome bus with ALIX’, Nat Cell Biol, 14 (7), 654-5.
Iacopetta, Domenico, Rechoum, Yassine, and Fuqua, Suzanne A. W. (2012), ‘The Role of Androgen Receptor in Breast Cancer’, Drug discovery today. Disease mechanisms, 9 (1-2), e19-e27.
Inabe, K., et al. (2002), ‘Vav3 modulates B cell receptor responses by regulating phosphoinositide 3-kinase activation’, J Exp Med, 195 (2), 189-200.
Irie, T. and Uekama, K. (1997), ‘Pharmaceutical applications of cyclodextrins. III. Toxicological issues and safety evaluation’, J Pharm Sci, 86 (2), 147-62.
Irie, T., et al. (1992), ‘Hydroxypropylcyclodextrins in parenteral use. II: Effects on transport and disposition of lipids in rabbit and humans’, J Pharm Sci, 81 (6), 524-8.
Irie, T., et al. (1982), ‘Cyclodextrin-induced hemolysis and shape changes of human erythrocytes in vitro’, J Pharmacobiodyn, 5 (9), 741-4.
Jameson, L. P., Smith, N. W., and Dzyuba, S. V. (2012), ‘Dye-binding assays for evaluation of the effects of small molecule inhibitors on amyloid (abeta) self-assembly’, ACS Chem Neurosci, 3 (11), 807-19.
Jiang, H. L., et al. (2015), ‘Loss of RAB1B promotes triple-negative breast cancer metastasis by activating TGF-beta/SMAD signaling’, Oncotarget, 6 (18), 16352-65.
Jounai, N., et al. (2011), ‘NLRP4 negatively regulates autophagic processes through an association with beclin1’, J Immunol, 186 (3), 1646-55.
Jung, S., et al. (2013), ‘Oncogenic function of p34SEI-1 via NEDD41mediated PTEN ubiquitination/degradation and activation of the PI3K/AKT pathway’, Int J Oncol, 43 (5), 1587-95.
Kagedal, K., et al. (2010), ‘Increased expression of the lysosomal cholesterol transporter NPC1 in Alzheimer's disease’, Biochim Biophys Acta, 1801 (8), 831-8.
Kamar, R. I., Organ-Darling, L. E., and Raphael, R. M. (2012), ‘Membrane Cholesterol Strongly Influences Confined Diffusion of Prestin’, Biophysical Journal, 103 (8), 1627-36.
Kameyama, K., et al. (2017), ‘Induction of mitophagy-mediated antitumor activity with folate-appended methyl-beta-cyclodextrin’, Int J Nanomedicine, 12, 3433-46.
Kanai, A., et al. (1989), ‘The effect on the cornea of alpha cyclodextrin vehicle for cyclosporin eye drops’, Transplant Proc, 21 (1 Pt 3), 3150-2.
Kantner, I. and Erben, R. G. (2012), ‘Long-term parenteral administration of 2-hydroxypropyl-beta-cyclodextrin causes bone loss’, Toxicol Pathol, 40 (5), 742-50.
Kawate, T., et al. (2015), ‘High levels of DJ-1 protein and isoelectric point 6.3 isoform in sera of breast cancer patients’, Cancer Sci, 106 (7), 938-43.
Kawauchi, T. (2012), ‘Cell adhesion and its endocytic regulation in cell migration during neural development and cancer metastasis’, Int J Mol Sci, 13 (4), 4564-90.
Keogh, M. J., et al. (2015), ‘A novel de novo STXBP1 mutation is associated with mitochondrial complex I deficiency and late-onset juvenile-onset parkinsonism’, Neurogenetics, 16 (1), 65-7.
Kerr, W. G. (2011), ‘Inhibitor and activator: dual functions for SHIP in immunity and cancer’, Ann N Y Acad Sci, 1217, 1-17.
Khalil, B., et al. (2015), ‘PINK1-induced mitophagy promotes neuroprotection in Huntington's disease’, Cell Death Dis, 6, e1617.
Kim, H. J., et al. (2015), ‘Metastasis-Free Interval Is Closely Related to Tumor Characteristics and Has Prognostic Value in Breast Cancer Patients with Distant Relapse’, J Breast Cancer 18 (4), 371-7.
Kim, S., et al. (2016), ‘Evidence that the rab5 effector APPL1 mediates APP-betaCTF-induced dysfunction of endosomes in Down syndrome and Alzheimer's disease’, Mol Psychiatry, 21 (5), 707-16.
Kim, T. W., et al. (2012a), ‘Phosphoinositide modulation for the treatment of neurodegenerative diseases’, (Columbia University), U.S. Pat. No. 8,288,378.
Kim, Tae-Wan, et al. (2012b), ‘Phosphoinositide Modulation For The Treatment Of Neurodegenerative Diseases’, U.S. Pat. No. 8,288,378 (US: KIM, TAE-WAN
DI PAOLO, GILBERT
KANG MIN, SUK
BERMAN, DIEGO
MCINTIRE LAURA BETH, JOHNSON
UNIV COLUMBIA,).
Kitada, T., et al. (1998), ‘Mutations in the parkin gene cause autosomal recessive juvenile parkinsonism’, Nature, 392 (6676), 605-8.
Koldamova, R., Fitz, N. F., and Lefterov, I. (2014), ‘ATP-binding cassette transporter A1: from metabolism to neurodegeneration’, Neurobiol Dis, 72 Pt A, 13-21.
Kondo, T., Irie, T., and Uekama, K. (1996), ‘Combination effects of alpha-cyclodextrin and xanthan gum on rectal absorption and metabolism of morphine from hollow-type suppositories in rabbits’, Biol Pharm Bull, 19 (2), 280-6.
Koran, M. E., et al. (2014), ‘Genetic interactions within inositol-related pathways are associated with longitudinal changes in ventricle size’, J Alzheimers Dis, 38 (1), 145-54.
Koumangoye, Rainelli B., et al. (2011), ‘Detachment of Breast Tumor Cells Induces Rapid Secretion of Exosomes Which Subsequently Mediate Cellular Adhesion and Spreading’, Plos One, 6 (9), e24234.
Krug, S. M., et al. (2013), ‘Sodium caprate as an enhancer of macromolecule permeation across tricellular tight junctions of intestinal cells’, Biomaterials, 34 (1), 275-82.
Kuzuya, A., et al. (2016), ‘Identification of the novel activity-driven interaction between synaptotagmin 1 and presenilin 1 links calcium, synapse, and amyloid beta’, BMC Biol, 14, 25.
Law, C., et al. (2016), ‘Normal Molecular Specification and Neurodegenerative Disease-Like Death of Spinal Neurons Lacking the SNARE-Associated Synaptic Protein Munc18-1’, J Neurosci, 36 (2), 561-76.
Lee, B. H., et al. (2013), ‘Dysregulation of cholesterol homeostasis in human prostate cancer through loss of ABCA1’, Cancer Res, 73 (3), 1211-8.
Leonova, E. I. and Galzitskaya, O. V. (2015), ‘Role of Syndecans in Lipid Metabolism and Human Diseases’, Adv Exp Med Biol, 855, 241-58.
Leroy-Lechat, F., et al. (1994), ‘Evaluation of the cytotoxicity of cyclodextrins and hydroxypropylated derivatives’, International Journal of Pharmaceutics, 101 (1-2), 97-103.
Levano, K., et al. (2009), ‘A genetic strategy involving a glycosyltransferase promoter and a lipid translocating enzyme to eliminate cancer cells’, Glycoconj J, 26 (6), 739-48.
Levano, K., et al. (2012), ‘Atp8a1 deficiency is associated with phosphatidylserine externalization in hippocampus and delayed hippocampus-dependent learning’, J Neurochem, 120 (2), 302-13.
Li, H., Cuzner, M. L., and Newcombe, J. (1996), ‘Microglia-derived macrophages in early multiple sclerosis plaques’, Neuropathol Appl Neurobiol, 22 (3), 207-15.
Li, H., et al. (2008), ‘Candidate single-nucleotide polymorphisms from a genomewide association study of Alzheimer disease’, Arch Neurol, 65 (1), 45-53.
Li, Y. C., et al. (2006), ‘Elevated levels of cholesterol-rich lipid rafts in cancer cells are correlated with apoptosis sensitivity induced by cholesterol-depleting agents’, Am J Pathol, 168 (4), 1107-18; quiz 404-5.
Liao, C. J., et al. (2015), ‘A novel small-form NEDD4 regulates cell invasiveness and apoptosis to promote tumor metastasis’, Oncotarget, 6 (11), 9341-54.
Lill, C. M., et al. (2012), ‘Comprehensive research synopsis and systematic meta-analyses in Parkinson's disease genetics: The PDGene database’, PLoS Genet, 8 (3), e1002548.
Lim, S., et al. (2014), ‘Amyloid-beta precursor protein promotes cell proliferation and motility of advanced breast cancer’, BMC Cancer 14, 928.
Lin, De-Chen, et al. (2013), ‘Genomic and functional characterizations of phosphodiesterase subtype 4D in human cancers’, Proceedings of the National Academy of Sciences of the United States of America, 110 (15), 6109-14.
Lindmark, T., Kimura, Y., and Artursson, P. (1998), ‘Absorption enhancement through intracellular regulation of tight junction permeability by medium chain fatty acids in Caco-2 cells’, J Pharmacol Exp Ther, 284 (1), 362-9.
Liu, B., et al. (2009), ‘Reversal of defective lysosomal transport in NPC disease ameliorates liver dysfunction and neurodegeneration in the npc1−/− mouse’, Proc Natl Acad Sci USA, 106 (7), 2377-82.
Liu, Xin-Ming, et al. (2008), ‘Osteotropic β-cyclodextrin for local bone regeneration’, Biomaterials, 29 (11), 1686-92.
Lizarbe, M. A., et al. (2013), ‘Annexin-phospholipid interactions. Functional implications’, Int J Mol Sci, 14 (2), 2652-83.
Llanos, P., et al. (2015), ‘The cholesterol-lowering agent methyl-beta-cyclodextrin promotes glucose uptake via GLUT4 in adult muscle fibers and reduces insulin resistance in obese mice’, Am J Physiol Endocrinol Metab, 308 (4), E294-305.
Loane, D. J., et al. (2011), ‘Modulation of ABCA1 by an LXR agonist reduces beta-amyloid levels and improves outcome after traumatic brain injury’, J Neurotrauma, 28 (2), 225-36.
Loftsson, T. and Brewster, M. E. (1996), ‘Pharmaceutical applications of cyclodextrins. 1. Drug solubilization and stabilization’, J Pharm Sci, 85 (10), 1017-25.
—(2010), ‘Pharmaceutical applications of cyclodextrins: basic science and product development’, J Pharm Pharmacol, 62 (11), 1607-21.
Loftsson, T., et al. (2016), ‘Pharmacokinetics of cyclodextrins and drugs after oral and parenteral administration of drug/cyclodextrin complexes’, J Pharm Pharmacol, 68 (5), 544-55.
Lopez Gonzalez, I., et al. (2016), ‘Genetic and Transcriptomic Profiles of Inflammation in Neurodegenerative Diseases: Alzheimer, Parkinson, Creutzfeldt-Jakob and Tauopathies’, Int J Mol Sci, 17 (2), 206.
Lowry, M. C., Gallagher, W. M., and O'Driscoll, L. (2015), ‘The Role of Exosomes in Breast Cancer’, Clin Chem, 61 (12), 1457-65.
Luo, C., et al. (2017), ‘The role of microglia in multiple sclerosis’, Neuropsychiatr Dis Treat, 13, 1661-67.
Ma, K., et al. (2008), ‘PI(3,4,5)P3 and PI(3,4)P2 levels correlate with PKB/akt phosphorylation at Thr308 and Ser473, respectively; PI(3,4)P2 levels determine PKB activity’, Cell Signal, 20 (4), 684-94.
Maarup, T. J., et al. (2015), ‘Intrathecal 2-hydroxypropyl-beta-cyclodextrin in a single patient with Niemann-Pick C1’, Mol Genet Metab, 116 (1-2), 75-9.
Majerus, Philip W. and York, John D. (2009), ‘Phosphoinositide phosphatases and disease’, Journal of Lipid Research, 50 (Suppl), S249-S54.
Malhotra, Shikha, et al. (2009), ‘Vav and Rac Activation in B Cell Antigen Receptor Endocytosis Involves Vav Recruitment to the Adapter Protein LAB’, The Journal of Biological Chemistry, 284 (52), 36202-12.
Malkki, Hemi (2016), ‘Alzheimer disease: Cancer immunotherapy drug reduces symptoms of Alzheimer disease in mice’, Nat Rev Neurol, 12 (3), 126-26.
Mann, H. B. and Whitney, D. R. (1947), ‘On a test of whether one of two random variables is stochastically larger than the other’, Annals of Mathematical Statistics, 18 (1), 50-60.
Martin-Maestro, P., et al. (2016), ‘PARK2 enhancement is able to compensate mitophagy alterations found in sporadic Alzheimer's disease’, Hum Mol Genet, 25 (4), 792-806.
Martin, T. F. (2015), ‘PI(4,5)P(2)-binding effector proteins for vesicle exocytosis’, Biochim Biophys Acta, 1851 (6), 785-93.
Matigian, Nicholas, et al. (2010), ‘Disease-specific, neurosphere-derived cells as models for brain disorders’, Disease Models & Mechanisms, 3 (11-12), 785-98.
Matsuo, M., et al. (2013), ‘Effects of cyclodextrin in two patients with Niemann-Pick Type C disease’, Mol Genet Metab, 108 (1), 76-81.
Maxwell, M. J., et al. (2012), ‘Attenuation of phosphoinositide 3-kinase delta signaling restrains autoimmune disease’, J Autoimmun, 38 (4), 381-91.
Mayer, I. A. and Arteaga, C. L. (2016), ‘The PI3K/AKT Pathway as a Target for Cancer Treatment’, Annu Rev Med, 67, 11-28.
McCleverty, Clare J., Lin, Diane C., and Liddington, Robert C. (2007), ‘Structure of the PTB domain of tensin1 and a model for its recruitment to fibrillar adhesions’, Protein Science: A Publication of the Protein Society, 16 (6), 1223-29.
McKeown, S. R. and Allen, I. V. (1977), ‘Lysosomal involvement in the pathogenesis of multiple sclerosis [proceedings]’, Biochem Soc Trans, 5 (5), 1416-8.
—(1979), ‘The fragility of cerebral lysosomes in multiple sclerosis’, Neuropathol Appl Neurobiol, 5 (5), 405-15.
McKew, John, et al. (2014), ‘Cyclodextrin For The Treatment Of Lysosomal Storage Diseases’, (WO: US HEALTH).
Mellman, Ira and Yarden, Yosef (2013), ‘Endocytosis and Cancer’, Cold Spring Harbor Perspectives in Biology, 5 (12).
Michailidou, K., et al. (2013), ‘Large-scale genotyping identifies 41 new loci associated with breast cancer risk’, Nat Genet, 45 (4), 353-61, 61e1-2.
Miller, J. A., et al. (2013), ‘Genes and pathways underlying regional and cell type changes in Alzheimer's disease’, Genome Med, 5 (5), 48.
Minn, A. J., et al. (2005), ‘Genes that mediate breast cancer metastasis to lung’, Nature, 436 (7050), 518-24.
Mitra, S., Cheng, K. W., and Mills, G. B. (2012), ‘Rab25 in cancer: a brief update’, Biochem Soc Trans, 40 (6), 1404-8.
Miyajima, Koichiro, Tomita, Keiko, and Nakagaki, Masayuki (1985), ‘Complex formation between di-and monophosphatidylcholines and cyclodextrins in water’, Chem Pharm Bull (Tokyo), 33 (6), 2587-90.
Mohammad, N., et al. (2014), ‘Cholesterol depletion by methyl-beta-cyclodextrin augments tamoxifen induced cell death by enhancing its uptake in melanoma’, Mol Cancer 13, 204.
Monnaert, V., et al. (2004), ‘Behavior of α-, β-, and γ-Cyclodextrins and Their Derivatives on an in Vitro Model of Blood-Brain Barrier’, Journal of Pharmacology and Experimental Therapeutics, 310 (2), 745-51.
Moon, Hyun-Seuk, et al. (2008), ‘Physico-chemical modifications of conjugated linoleic acid for ruminal protection and oxidative stability’, Nutrition & Metabolism, 5 (1), 16.
Moran, L. B., et al. (2006), ‘Whole genome expression profiling of the medial and lateral substantia nigra in Parkinson's disease’, Neurogenetics, 7 (1), 1-11.
Morris, Richard G. M. (1981), ‘Spatial localization does not require the presence of local cues’, Learning and Motivation, 12 (2), 239-60.
Mosesson, Yaron, Mills, Gordon B., and Yarden, Yosef (2008), ‘Derailed endocytosis: an emerging feature of cancer’, Nat Rev Cancer 8 (11), 835-50.
Moskvina, V., et al. (2013), ‘Analysis of genome-wide association studies of alzheimer disease and of parkinson disease to determine if these 2 diseases share a common genetic risk’, JAMA Neurology, 70 (10), 1268-76.
Motoyama, K., et al. (2015), ‘Evaluation of antitumor effects of folate-conjugated methyl-beta-cyclodextrin in melanoma’, Biol Pharm Bull, 38 (3), 374-9.
Nalls, M. A., et al. (2014), ‘Large-scale meta-analysis of genome-wide association data identifies six new risk loci for Parkinson's disease’, Nat Genet, 46 (9), 989-93.
NIH (2015), ‘Study of 2-hydroxypropyl-β-cyclodextrin (VTS-270) to Treat Niemann-Pick Type C1 (NPC1) Disease’, ClinicalTrials.gov, (Mar. 6, 2016), NCT02534844.
Nishimura, Y., et al. (2003), ‘Overexpression of ROCK in human breast cancer cells: evidence that ROCK activity mediates intracellular membrane traffic of lysosomes’, Pathol Oncol Res, 9 (2), 83-95.
Nishimura, Y., et al. (2006), ‘A role of LIM kinase 1/cofilin pathway in regulating endocytic trafficking of EGF receptor in human breast cancer cells’, Histochem Cell Biol, 126 (5), 627-38.
Nordestgaard, L. T., et al. (2015), ‘Loss-of-function mutation in ABCA1 and risk of Alzheimer's disease and cerebrovascular disease’, Alzheimers & Dementia, 11 (12), 1430-38.
O'Neill, C. (2013), ‘PI3-kinase/Akt/mTOR signaling: impaired on/off switches in aging, cognitive decline and Alzheimer's disease’, Exp Gerontol, 48 (7), 647-53.
O'Reilly, E. A., et al. (2015), ‘The fate of chemoresistance in triple negative breast cancer (TNBC)’, BBA Clin, 3, 257-75.
Ohtani, Yoshiro, et al. (1989), ‘Differential effects of α-, β- and γ-cyclodextrins on human erythrocytes’, European Journal of Biochemistry, 186 (1-2), 17-22.
Ono, N., et al. (2001), ‘A moderate interaction of maltosyl-alpha-cyclodextrin with Caco-2 cells in comparison with the parent cyclodextrin’, Biol Pharm Bull, 24 (4), 395-402.
Ooms, Lisa M, et al. (2015), ‘The Inositol Polyphosphate 5-Phosphatase PIPP Regulates AKT1-Dependent Breast Cancer Growth and Metastasis’, Cancer cell, 28 (2), 155-69.
Pahnke, J., Langer, O., and Krohn, M. (2014), ‘Alzheimer's and ABC transporters—new opportunities for diagnostics and treatment’, Neurobiol Dis, 72 Pt A, 54-60.
Palaniyandi, Kanagaraj, et al. (2012), ‘Human Breast Cancer Stem Cells Have Significantly Higher Rate of Clathrin-Independent and Caveolin-Independent Endocytosis than the Differentiated Breast Cancer Cells’, Journal of cancer science & therapy, 4 (7), 214-22.
Pandey, P., et al. (2016), ‘Amyloid precursor protein and amyloid precursor-like protein 2 in cancer’, Oncotarget, 10430-44.
Park, C. W., et al. (2002), ‘Inclusion complex of conjugated linoleic acid (CLA) with cyclodextrins’, J Agric Food Chem, 50 (10), 2977-83.
Parnell, E., Palmer, T. M., and Yarwood, S. J. (2015), ‘The future of EPAC-targeted therapies: agonism versus antagonism’, Trends in Pharmacological Sciences, 36 (4), 203-14.
Pearson, Thomas A. and Manolio, Teri A. (2008), ‘How to interpret a genome-wide association study’, JAMA, 299 (11), 1335-44.
Peinado, H., Lavotshkin, S., and Lyden, D. (2011), ‘The secreted factors responsible for pre-metastatic niche formation: old sayings and new thoughts’, Semin Cancer Biol, 21 (2), 139-46.
Perrett, R. M., Alexopoulou, Z., and Tofaris, G. K. (2015), ‘The endosomal pathway in Parkinson's disease’, Mol Cell Neurosci, 66 (Pt A), 21-8.
Persaud, A., et al. (2011), ‘Nedd4-1 binds and ubiquitylates activated FGFR1 to control its endocytosis and function’, EMBO J, 30 (16), 3259-73.
Pfeffer, Suzanne R. (1999), ‘Motivating endosome motility’, Nat Cell Biol, 1 (6), E145-E47.
Picollo, A., Malvezzi, M., and Accardi, A. (2015), ‘TMEM16 proteins: unknown structure and confusing functions’, J Mol Biol, 427 (1), 94-105.
Pinho, Raquel, et al. (2016), ‘Gene Expression Differences in Peripheral Blood of Parkinson's Disease Patients with Distinct Progression Profiles’, PLOS ONE, 11 (6), e0157852.
Pitha, J and Szente, L (1982), ‘Cyclodextrins and Congeners in Parenteral Applications’, Proceedings of the First International Symposium on Cyclodextrins (Springer Netherlands), 457-66.
Posor, Y., Eichhorn-Grunig, M., and Haucke, V. (2015), ‘Phosphoinositides in endocytosis’, Biochim Biophys Acta, 1851 (6), 794-804.
Pourmand, G, et al. (2007), ‘Role of PTEN gene in progression of prostate cancer’, Urol J, 4 (2), 95-100.
Prakash, A. S. and Abbott, P. J. (2001), ‘Safety evaluation of certain food additives and contaminants: alpha-cyclodextrin’, WHO/JECFA Food Additive Series, 48, 1030.
Prinz, M. and Priller, J. (2014), ‘Microglia and brain macrophages in the molecular age: from origin to neuropsychiatric disease’, Nat Rev Neurosci, 15 (5), 300-12.
Puthiyedth, N., et al. (2016), ‘Identification of Differentially Expressed Genes through Integrated Study of Alzheimer's Disease Affected Brain Regions’, Plos One, 11 (4), e0152342.
Raghu, H., et al. (2010), ‘Localization of uPAR and MMP-9 in lipid rafts is critical for migration, invasion and angiogenesis in human breast cancer cells’, BMC Cancer 10, 647.
Rainero, E., et al. (2015), ‘Ligand-Occupied Integrin Internalization Links Nutrient Signaling to Invasive Migration’, Cell Rep, 10, 398-413.
Rajendran, Lawrence, Knolker, Hans-Joachim, and Simons, Kai (2010), ‘Subcellular targeting strategies for drug design and delivery’, Nat Rev Drug Discov, 9 (1), 29-42.
Rastogi, S. C. and Clausen, J. (1980), ‘Loss of lysosomal neutral proteinase from leucocytes induced by the action of multiple sclerosis-specific brain antigens’, Clin Exp Immunol, 42 (1), 50-6.
Reitz, C. (2012), ‘The role of intracellular trafficking and the VPS10d receptors in Alzheimer's disease’, Future Neurol, 7 (4), 423-31.
Renukuntla, Jwala, et al. (2013), ‘Approaches for Enhancing Oral Bioavailability of Peptides and Proteins’, International journal of pharmaceutics, 447 (0), 75-93.
Resnik, N., et al. (2015), ‘Highly Selective Anti-Cancer Activity of Cholesterol-Interacting Agents Methyl-beta-Cyclodextrin and Ostreolysin A/Pleurotolysin B Protein Complex on Urothelial Cancer Cells’, Plos One, 10 (9), e0137878.
Riekkinen, P. J., Rinne, U. K., and Arstila, A. U. (1972), ‘Neurochemical and morphological studies on demyelination in multiple sclerosis with special reference to etiological aspects’, Z Neurol, 203 (2), 91-104.
Risbridger, Gail P., et al. (2010), ‘Breast and prostate cancer: more similar than different’, Nat Rev Cancer 10 (3), 205-12.
Rivera, J., Megias, D., and Bravo, J. (2010), ‘Sorting nexin 6 interacts with breast cancer metastasis suppressor-1 and promotes transcriptional repression’, J Cell Biochem, 111 (6), 1464-72.
Rivero-Rios, P., et al. (2015), ‘Alterations in late endocytic trafficking related to the pathobiology of LRRK2-linked Parkinson's disease’, Biochem Soc Trans, 43 (3), 390-5.
Rivers, J. R., Maggo, S. D., and Ashton, J. C. (2012), ‘Neuroprotective effect of hydroxypropyl-beta-cyclodextrin in hypoxia-ischemia’, Neuroreport, 23 (3), 134-8.
Rocks, N., et al. (2012), ‘Curcumin-cyclodextrin complexes potentiate gemcitabine effects in an orthotopic mouse model of lung cancer’, Br J Cancer 107 (7), 1083-92.
Rodal, Siv Kjersti, et al. (1999), ‘Extraction of Cholesterol with Methyl-β-Cyclodextrin Perturbs Formation of Clathrin-coated Endocytic Vesicles’, Molecular Biology of the Cell, 10 (4), 961-74.
Rodrigues, E. M., et al. (2016), ‘Abeta-Induced Synaptic Alterations Require the E3 Ubiquitin Ligase Nedd4-1’, J Neurosci, 36 (5), 1590-5.
Rohatgi, R. A. and Shaw, L. M. (2016), ‘An autophagy-independent function for Beclin 1 in cancer’, Mol Cell Oncol, 3 (1).
Roka, E., et al. (2015), ‘Evaluation of the Cytotoxicity of alpha-Cyclodextrin Derivatives on the Caco-2 Cell Line and Human Erythrocytes’, Molecules, 20 (11), 20269-85.
Rossi, M. R., et al. (2005), ‘Identification of inactivating mutations in the JAK1, SYNJ2, and CLPTM1 genes in prostate cancer cells using inhibition of nonsense-mediated decay and microarray analysis’, Cancer Genet Cytogenet, 161 (2), 97-103.
Rouzier, Roman, et al. (2005), ‘Microtubule-associated protein tau: A marker of paclitaxel sensitivity in breast cancer’, Proceedings of the National Academy of Sciences of the United States of America, 102 (23), 8315-20.
Runowicz, C. D., et al. (2016), ‘American Cancer Society/American Society of Clinical Oncology Breast Cancer Survivorship Care Guideline’, J Clin Oncol, 34 (6), 611-35.
Russell, C. L., et al. (2012), ‘Amyloid-beta acts as a regulator of neurotransmitter release disrupting the interaction between synaptophysin and VAMP2’, Plos One, 7 (8), e43201.
Sahay, D., et al. (2015), ‘The LPA1/ZEB1/miR-21-activation pathway regulates metastasis in basal breast cancer’, Oncotarget, 6 (24), 20604-20.
Sakane, F. and Kanoh, H. (1997), ‘Molecules in focus: diacylglycerol kinase’, Int J Biochem Cell Biol, 29 (10), 1139-43.
Salminen, A., et al. (2013), ‘Impaired autophagy and APP processing in Alzheimer's disease: The potential role of Beclin 1 interactome’, Prog Neurobiol, 106-107, 33-54.
Salter, M. W. and Stevens, B. (2017), ‘Microglia emerge as central players in brain disease’, Nat Med, 23 (9), 1018-27.
Samie, M. A. and Xu, H. (2014), ‘Lysosomal exocytosis and lipid storage disorders’, J Lipid Res, 55 (6), 995-1009.
Saunders, D. A., et al. (1997), ‘Measurement of total phospholipids in urine of patients treated with gentamicin’, Br J Clin Pharmacol, 43 (4), 435-40.
Schimanski, S., et al. (2010), ‘Expression of the lipid transporters ABCA3 and ABCA1 is diminished in human breast cancer tissue’, Horm Metab Res, 42 (2), 102-9.
Schmid, Sandra L. and Mettlen, Marcel (2013), ‘Cell biology: Lipid switches and traffic control’, Nature, 499 (7457), 161-62.
Schreij, A. M., Fon, E. A., and McPherson, P. S. (2015), ‘Endocytic membrane trafficking and neurodegenerative disease’, Cell Mol Life Sci.
Schwyzer, R. U. and Henzi, H. (1983), ‘Multiple sclerosis: plaques caused by 2-step demyelination?’, Med Hypotheses, 12 (2), 129-42.
Seaman, M Nj and Freeman, C. L. (2014), ‘Analysis of the Retromer complex-WASH complex interaction illuminates new avenues to explore in Parkinson disease’, Commun Integr Biol, 7, e29483.
Sekine, Y., et al. (2010), ‘High-density lipoprotein induces proliferation and migration of human prostate androgen-independent cancer cells by an ABCA1-dependent mechanism’, Mol Cancer Res, 8 (9), 1284-94.
Sengelaub, Caitlin A., et al. (2015), ‘PTPRN2 and PLCβ1 promote metastatic breast cancer cell migration through PI(4,5)P2□dependent actin remodeling’, The EMBO Journal.
Shaikh, I., et al. (2012), ‘Permeability Enhancement Techniques for Poorly Permeable Drugs: A Review’, J Appl Pharmacol Sci, 2 (7), 34-39.
Shen, Q., et al. (2013), ‘Phagocytic receptor signaling regulates clathrin and epsin-mediated cytoskeletal remodeling during apoptotic cell engulfment in C. elegans’, Development, 140 (15), 3230-43.
Sherva, R., et al. (2011), ‘Identification of novel candidate genes for Alzheimer's disease by autozygosity mapping using genome wide SNP data’, J Alzheimers Dis, 23 (2), 349-59.
Sherva, R., et al. (2014), ‘Genome-wide association study of the rate of cognitive decline in Alzheimer's disease’, Alzheimers Dement, 10 (1), 45-52.
Shimizu, E., et al. (2008), ‘IL-4-induced selective clearance of oligomeric beta-amyloid peptide(1-42) by rat primary type 2 microglia’, J Immunol, 181 (9), 6503-13.
Shityakov, S., et al. (2016), ‘Evaluation of the potential toxicity of unmodified and modified cyclodextrins on murine blood-brain barrier endothelial cells’, J Toxicol Sci, 41 (2), 175-84.
Shulga, Y. V., et al. (2010), ‘Molecular species of phosphatidylinositol-cycle intermediates in the endoplasmic reticulum and plasma membrane’, Biochemistry, 49 (2), 312-7.
Singh, T. D., et al. (2010), ‘MEGF10 functions as a receptor for the uptake of amyloid-beta’, FEBS Lett, 584 (18), 3936-42.
Sjöblom, Tobias, et al. (2006), ‘The Consensus Coding Sequences of Human Breast and Colorectal Cancers’, Science, 314 (5797), 268-74.
Small, Scott A. and Petsko, Gregory A. (2015), ‘Retromer in Alzheimer disease, Parkinson disease and other neurological disorders’, Nat Rev Neurosci, 16 (3), 126-32.
Soderberg, M., et al. (1992), ‘Lipid composition in different regions of the brain in Alzheimer's disease/senile dementia of Alzheimer's type’, J Neurochem, 59 (5), 1646-53.
Sole-Domenech, S., et al. (2016), ‘The endocytic pathway in microglia during health, aging and Alzheimer's disease’, Ageing Res Rev, 32, 89-103.
Solecki, D. J. (2012), ‘Sticky situations: recent advances in control of cell adhesion during neuronal migration’, Curr Opin Neurobiol, 22 (5), 791-8.
Song, W., et al. (2014), ‘2-Hydroxypropyl-beta-cyclodextrin promotes transcription factor EB-mediated activation of autophagy: implications for therapy’, J Biol Chem, 289 (14), 10211-22.
Stella, V. J. and He, Q. (2008), ‘Cyclodextrins’, Toxicol Pathol, 36 (1), 30-42.
Storch, C. H., et al. (2007), ‘Localization of the human breast cancer resistance protein (BCRP/ABCG2) in lipid rafts/caveolae and modulation of its activity by cholesterol in vitro’, J Pharmacol Exp Ther, 323 (1), 257-64.
Swaminathan, G, Zhu, W., and Plowey, E. D. (2016), ‘BECN1/Beclin 1 sorts cell-surface APP/amyloid beta precursor protein for lysosomal degradation’, Autophagy, 12 (12), 2404-19.
Symeonides, S. N., Anderton, S. M., and Serrels, A. (2017), ‘FAK-inhibition opens the door to checkpoint immunotherapy in Pancreatic Cancer’, J Immunother Cancer 5, 17.
Szejtli, J., et al. (1981), ‘Influencing of resorption and side-effects of salicylic acid by complexing with beta-cyclodextrin’, Pharmazie, 36 (4), 283-6.
Takahashi, M., Iseki, E., and Kosaka, K. (2000), ‘Cdk5 and munc-18/p67 co-localization in early stage neurofibrillary tangles-bearing neurons in Alzheimer type dementia brains’, J Neurol Sci, 172 (1), 63-9.
Takahashi, S., et al. (2016), ‘Susceptibility of outer hair cells to cholesterol chelator 2-hydroxypropyl-beta-cyclodextrine is prestin-dependent’, Sci Rep, 6, 21973.
Tan, M. G, et al. (2014), ‘Decreased rabphilin 3A immunoreactivity in Alzheimer's disease is associated with Abeta burden’, Neurochem Int, 64, 29-36.
Telarico, T. and Perl, A. (2012), ‘The role of endocytic recycling in autoimmunity’, Methods Mol Biol, 900, 91-107.
Tiribuzi, R., et al. (2011), ‘Lysosomal beta-galactosidase and beta-hexosaminidase activities correlate with clinical stages of dementia associated with Alzheimer's disease and type 2 diabetes mellitus’, J Alzheimers Dis, 24 (4), 785-97.
Tomas, Alejandra, Futter, Clare E., and Eden, Emily R. (2014), ‘EGF receptor trafficking: consequences for signaling and cancer’, Trends in Cell Biology, 24 (1), 26-34.
Tomlinson, P. R., et al. (2015), ‘Identification of distinct circulating exosomes in Parkinson's disease’, Ann Clin Transl Neurol, 2 (4), 353-61.
Trasino, S. E., Kim, Y. S., and Wang, T. T. (2009), ‘Ligand, receptor, and cell type-dependent regulation of ABCA1 and ABCG1 mRNA in prostate cancer epithelial cells’, Mol Cancer Ther, 8 (7), 1934-45.
Tsigelny, I. F., et al. (2008), ‘Mechanisms of hybrid oligomer formation in the pathogenesis of combined Alzheimer's and Parkinson's diseases’, Plos One, 3 (9), e3135.
Tukey, J. W. (1980), ‘We Need Both Exploratory and Confirmatory’, American Statistician, 34 (1), 23-25.
Tukey, John W. (1977), Exploratory data analysis (Reading, Mass.: Addison-Wesley).
Tulpule, K. and Dringen, R. (2013), ‘Formaldehyde in brain: an overlooked player in neurodegeneration?’, Journal of Neurochemistry, 127 (1), 7-21.
Valencia, A., et al. (2013), ‘Striatal synaptosomes from Hdh140Q/140Q knock-in mice have altered protein levels, novel sites of methionine oxidation, and excess glutamate release after stimulation’, J Huntingtons Dis, 2 (4), 459-75.
Valenza, M., et al. (2015), ‘Disruption of astrocyte-neuron cholesterol cross talk affects neuronal function in Huntington's disease’, Cell Death Differ, 22 (4), 690-702.
van der Mark, Vincent A., Oude Elferink, Ronald P. J., and Paulusma, Coen C. (2013), ‘P4 ATPases: Flippases in Health and Disease’, International Journal of Molecular Sciences, 14 (4), 7897-922.
van Dijk, K. D., et al. (2013), ‘Changes in endolysosomal enzyme activities in cerebrospinal fluid of patients with Parkinson's disease’, Mov Disord, 28 (6), 747-54.
Van Dooren, T., et al. (2014), ‘Derailed intraneuronal signalling drives pathogenesis in sporadic and familial Alzheimer's disease’, Biomed Res Int, 2014, 167024.
Van Ommen, B., De Bie, A. T., and Bar, A. (2004), ‘Disposition of 14C-alpha-cyclodextrin in germ-free and conventional rats’, Regul Toxicol Pharmacol, 39 Suppl 1, 57-66.
van Weering, J. R., Verkade, P., and Cullen, P. J. (2012), ‘SNX-BAR-mediated endosome tubulation is co-ordinated with endosome maturation’, Traffic, 13 (1), 94-107.
Vance, Jean E. (2012), ‘Dysregulation of cholesterol balance in the brain: contribution to neurodegenerative diseases’, Disease Models and Mechanisms, 5 (6), 746-55.
Vance, Jean E. and Karten, Barbara (2014), ‘Niemann-Pick C disease and mobilization of lysosomal cholesterol by cyclodextrin’, Journal of Lipid Research, 55 (8), 1609-21.
Vecsernyes, M., et al. (2014), ‘Cyclodextrins, blood-brain barrier, and treatment of neurological diseases’, Arch Med Res, 45 (8), 711-29.
Viaud, Julien, et al. (2016), ‘Phosphoinositides: Important lipids in the coordination of cell dynamics’, Biochimie, 125, 250-58.
Vilarino-Guell, C., et al. (2014), ‘DNAJC13 mutations in Parkinson disease’, Hum Mol Genet, 23 (7), 1794-801.
Vite, C. H., et al. (2015), ‘Intracisternal cyclodextrin prevents cerebellar dysfunction and Purkinje cell death in feline Niemann-Pick type C1 disease’, Sci Transl Med, 7 (276), 276ra26.
Wadman, M. (2016), ‘Battle over rare disease drug ensnares NIH’, Science, 354 (6308), 18-19.
Wagner, E. M., et al. (2008), ‘Dietary alpha-cyclodextrin lowers low-density lipoprotein cholesterol and alters plasma fatty acid profile in low-density lipoprotein receptor knockout mice on a high-fat diet’, Metabolism, 57 (8), 1046-51.
Walenbergh, S. M., et al. (2015), ‘Weekly Treatment of 2-Hydroxypropyl-beta-cyclodextrin Improves Intracellular Cholesterol Levels in LDL Receptor Knockout Mice’, Int J Mol Sci, 16 (9), 21056-69.
Walter, J., et al. (2001), ‘Phosphorylation regulates intracellular trafficking of beta-secretase’, J Biol Chem, 276 (18), 14634-41.
Wang, H., et al. (2009), ‘Parkin regulates paclitaxel sensitivity in breast cancer via a microtubule-dependent mechanism’, J Pathol, 218 (1), 76-85.
Wang, J. D., et al. (2015a), ‘A pivotal role of FOS-mediated BECN1/Beclin 1 upregulation in dopamine D2 and D3 receptor agonist-induced autophagy activation’, Autophagy, 11 (11), 2057-73.
Wang, K. S., et al. (2015b), ‘Polymorphisms within ASTN2 gene are associated with age at onset of Alzheimer's disease’, J Neural Transm (Vienna), 122 (5), 701-8.
Wang, Xin, et al. (2014), ‘Dysregulation of protein trafficking in neurodegeneration’, Molecular Neurodegeneration, 9 (1), 1-9.
Ward, S., et al. (2010), ‘2-hydroxypropyl-beta-cyclodextrin raises hearing threshold in normal cats and in cats with Niemann-Pick type C disease’, Pediatr Res, 68 (1), 52-6.
Waschbusch, D., et al. (2014), ‘LRRK2 transport is regulated by its novel interacting partner Rab32’, Plos One, 9 (10), e111632.
Waugh, M. G. (2015), ‘PIPs in neurological diseases’, Biochim Biophys Acta, 1851 (8), 1066-82.
Weber, G. F. (2015), ‘Molecular Analysis of a Recurrent Sarcoma Identifies a Mutation in FAF1’, Sarcoma, 2015, 839182.
Wei, B., et al. (2015), ‘Annexin A4 and cancer’, Clin Chim Acta, 447, 72-8.
Weng, L., et al. (2013), ‘Genome-wide discovery of genetic variants affecting tamoxifen sensitivity and their clinical and functional validation’, Annals of Oncology.
Wilcoxon, Frank (1954), ‘Individual comparisons by ranking methods’, Biometrics, 1, 80-83.
Wilkinson, Brandy L., et al. (2012), ‘Ibuprofen attenuates oxidative damage through NOX2 inhibition in Alzheimer's Disease’, Neurobiology of Aging, 33 (1), 197.e21-97.e32.
Willshaw, A., et al. (2004), ‘Identification of a novel protein complex containing annexin A4, rabphilin and synaptotagmin’, FEBS Lett, 559 (1-3), 13-21.
Wilson, Perrin M., et al. (2010), ‘Astn2, A Novel Member of the Astrotactin Gene Family, Regulates the Trafficking of ASTN1 during Glial-Guided Neuronal Migration’, The Journal of Neuroscience, 30 (25), 8529-40.
Winslow, A. R., et al. (2010), ‘alpha-Synuclein impairs macroautophagy: implications for Parkinson's disease’, J Cell Biol, 190 (6), 1023-37.
Wittkowski, Knut M. (1988), ‘Friedman-type statistics and consistent multiple comparisons for unbalanced designs’, Journal of the American Statistical Association, 83, 1163-70, 87:258.
—(2008), ‘Statistical methods for hierarchical multivariate ordinal data which are used for data base driven decision support ’, U.S. Pat. No. 7,664,616 (U.S.A.: The Rockefeller University).
—(2014), ‘Treatment and prevention of autism and autism spectrum disorders’, US 2016/0206581 A1 (U.S.: Wittkowski, Knut M.).
Wittkowski, Knut M., et al. (2013), ‘From single-SNP to wide-locus: genome-wide association studies identifying functionally related genes and intragenic regions in small sample studies’, Pharmacogenomics, 14 (4), 391-401.
Wittkowski, Knut M., et al. (submitted), ‘Complex Polymorphisms in Endocytosis Genes Suggest alpha-Cyclodextrin against Metastases in Breast Cancer’, Appendix, 10 (2).
Wittkowski, Knut M., et al. (2014), ‘A novel computational biostatistics approach implies impaired dephosphorylation of growth factor receptors as associated with severity of autism’, Transl Psychiatry, 4, e354.
Woolley, J. F., Dzneladze, I., and Salmena, L. (2015), ‘Phosphoinositide signaling in cancer: INPP4B Akt(s) out’, Trends Mol Med, 21 (9), 530-2.
Yamaguchi, H., et al. (2009), ‘Lipid rafts and caveolin-1 are required for invadopodia formation and extracellular matrix degradation by human breast cancer cells’, Cancer Res, 69 (22), 8594-602.
Yamaguchi, R., Perkins, G, and Hirota, K. (2015), ‘Targeting cholesterol with beta-cyclodextrin sensitizes cancer cells for apoptosis’, FEBS Lett, 589 (24 Pt B), 4097-105.
Yamashita, T., et al. (2015), ‘Outer Hair Cell Lateral Wall Structure Constrains the Mobility of Plasma Membrane Proteins’, PLoS Genet, 11 (9), e1005500.
Yang, L., et al. (2008), ‘Attenuation of MPTP neurotoxicity by rolipram, a specific inhibitor of phosphodiesterase IV’, Exp Neurol, 211 (1), 311-4.
Yang, Y., et al. (2013), ‘Elevated expression of syntenin in breast cancer is correlated with lymph node metastasis and poor patient survival’, Breast Cancer Res, 15 (3), R50.
Yao, H., et al. (2016), ‘The role of annexin A4 in cancer’, Front Biosci (Landmark Ed), 21, 949-57.
Yao, J., et al. (2006), ‘An essential role for beta-actin mRNA localization and translation in Ca2+-dependent growth cone guidance’, Nat Neurosci, 9 (10), 1265-73.
Yao, J., et al. (2010), ‘MicroRNA-related cofilin abnormality in Alzheimer's disease’, PLoS One, 5 (12), e15546.
Yao, Jiaqi, et al. (2012), ‘Neuroprotection by cyclodextrin in cell and mouse models of Alzheimer disease’, The Journal of Experimental Medicine, 209 (13), 2501-13.
Yokoo, M., et al. (2015), ‘2-Hydroxypropyl-beta-Cyclodextrin Acts as a Novel Anticancer Agent’, Plos One, 10 (11), e0141946.
Yoshitomi, H., et al. (1987), ‘Effect of triglyceride on small intestinal absorption of cefoxitin in rats’, J Pharm Pharmacol, 39 (11), 887-91.
Yu, Panpan, et al. (2015), ‘Cooperative interactions of LPPR family members in membrane localization and alteration of cellular morphology’, Journal of Cell Science, 128 (17), 3210-22.
Zhang, Hong-Yan, et al. (2013), ‘PTEN mutation, methylation and expression in breast cancer patients’, Oncology Letters, 6 (1), 161-68.
Zhang, Q., et al. (2006), ‘Metastatic potential of mouse Lewis lung cancer cells is regulated via ganglioside GM1 by modulating the matrix metalloprotease-9 localization in lipid rafts’, J Biol Chem, 281 (26), 18145-55.
Zhang, Q. Y., et al. (2015), ‘The Role of Retromer in Alzheimer's Disease’, Molecular Neurobiology.
Zhang, Yi, et al. (2014), ‘Endothelial PINK1 Mediates the Protective Effects of NLRP3 Deficiency during Lethal Oxidant Injury’, The Journal of Immunology, 192 (11), 5296-304.
Zhao, W., et al. (2016), ‘Candidate Antimetastasis Drugs Suppress the Metastatic Capacity of Breast Cancer Cells by Reducing Membrane Fluidity’, Cancer Res, 76 (7), 2037-49.
Zhiyu, W., et al. (2016), ‘The inflammasome: an emerging therapeutic oncotarget for cancer prevention’, Oncotarget, 7 (31), 50766.
Zhu, X. C., et al. (2016), ‘Association of Parkinson's Disease GWAS-Linked Loci with Alzheimer's Disease in Han Chinese’, Molecular Neurobiology.
Zhu, Xianjun, et al. (2012), ‘Mutations in a P-Type ATPase Gene Cause Axonal Degeneration’, PLOS Genetics, 8 (8), e1002853.
Zimmer, S., et al. (2016), ‘Cyclodextrin promotes atherosclerosis regression via macrophage reprogramming’, Sci Transl Med, 8 (333), 333ra50.

1-45. (canceled)

46. A pharmaceutical composition comprising an alpha-cyclodextrin, or a salt thereof, a medium chain fatty acid, or a salt thereof.

47. The pharmaceutical composition of claim 46, wherein the alpha-cyclodextrin and the medium chain fatty acid together comprise a clathrate.

48. The pharmaceutical composition of claim 47, comprising hydroxypropyl-alpha-cyclodextrin in an amount of from about 85 wt % to about 95% and sodium caprate in an amount of from about 5 wt % to about 15 wt %.

49. The pharmaceutical composition of claim 47, wherein the alpha-cyclodextrin is present in an amount of from about 10 wt % to about 95 wt %, based on the total weight of the composition.

50. The pharmaceutical composition of claim 47, wherein the alpha-cyclodextrin is present in an amount of from about 85 wt % to about 95 wt %, based on the total weight of the composition.

51. The pharmaceutical composition of claim 47, wherein the alpha-cyclodextrin is 2-hydroxypropyl-alpha-cyclodextrin.

52. The pharmaceutical composition of claim 47, wherein the medium chain fatty acid is present in an amount of from about 5 wt % to about 90 wt %, based on the total weight of the composition.

53. The pharmaceutical composition of claim 47, wherein the medium chain fatty acid is present in an amount of from about 5 wt % to about 15 wt %, based on the total weight of the composition.

54. The pharmaceutical composition of claim 47, wherein the medium chain fatty acid comprises a saturated aliphatic tail.

55. The pharmaceutical composition of claim 47, wherein the medium chain fatty acid comprises an aliphatic tail having from 3 to 70 carbon atoms.

56. The pharmaceutical composition of claim 47, wherein the medium chain fatty acid comprises an aliphatic tail having from 6 to 12 carbon atoms.

57. The pharmaceutical composition of claim 47, wherein the medium chain fatty acid is caproic acid.

58. The pharmaceutical composition of claim 47, wherein the medium chain fatty acid is capric acid.

59. The pharmaceutical composition of claim 13, wherein the medium chain fatty acid is sodium caprate.

60. The pharmaceutical composition of claim 47, wherein the composition is formulated as an oral dosage form.

61. A method of reducing levels of serum phospholipids in a subject in need thereof, the method comprising administering to the subject a therapeutically effective amount of the composition of claim 47.

62. The method of claim 61, wherein the subject has been diagnosed as having a malignant, neurodegenerative, cardiovascular, metabolic, inflammatory, autoimmune, age-related, or viral disease or disorder.

63. The method of claim 61, wherein the composition comprises hydroxypropyl-alpha-cyclodextrin in an amount of from about 85 wt % to about 95% and sodium caprate in an amount of from about 5 wt % to about 15 wt %, and wherein the hydroxypropyl-alpha-cyclodextrin and the sodium caprate together comprise a clathrate.

64. A method of treating a malignant, neurodegenerative, cardiovascular, metabolic, inflammatory, autoimmune, age-related, or viral disease or disorder in a subject in need thereof, the method comprising administering to the subject a therapeutically effective amount of the composition of claim 47.

65. The method of claim 64, wherein the composition comprises hydroxypropyl-alpha-cyclodextrin in an amount of from about 85 wt % to about 95% and sodium caprate in an amount of from about 5 wt % to about 15 wt %, and wherein the hydroxypropyl-alpha-cyclodextrin and the sodium caprate together comprise a clathrate.
